(19) United States
(12) Patent Application Publication
(10) Pub. No.: US 2025/0295788 A1
(43) Pub. Date: Sep. 25, 2025

(54) SILOXANE-BASED LIPIDS, LIPID NANOPARTICLE COMPOSITIONS COMPRISING THE SAME, AND METHODS OF USE THEREOF FOR TARGETED DELIVERY

(71) Applicant: The Trustees of the University of Pennsylvania, Philadelphia, PA (US)

(72) Inventors: Michael J. Mitchell, Philadelphia, PA (US); Lulu Xue, Philadelphia, PA (US)

(21) Appl. No.: 18/862,766

(22) PCT Filed: May 3, 2023
(86) PCT No.: PCT/US2023/066564

§ 371 Date: Nov. 4, 2024

Related U.S. Application Data
(60) Provisional application No. 63/338,272, filed on May 4, 2022.
(60) Provisional application No. 63/378,832, filed on Oct. 7, 2022.

(51) Int. Cl. A61K 47/54; A61K 47/69; C07F 7/21; C08G 77/392

(52) U.S. Cl. CPC A61K 47/541; A61K 47/543; A61K 47/6929; C07F 7/21; C08G 77/392

(57) ABSTRACT

The present disclosure relates, in part, to siloxane-based lipids or lipidoids, lipid nanoparticles (LNPs) comprising the same, and pharmaceutical compositions thereof. In certain embodiments, the LNPs of the present disclosure selectively target cells (e.g., hepatocytes, epithelial cells, endothelial cells, and immune cells, inter alia) and/or organs of interest (e.g., liver, spleen, heart, and lungs, inter alia). In another aspect, the present disclosure relates to methods of treating, preventing, and/or ameliorating one or more diseases and/or disorders in a subject, the method comprising administering to the subject at least one LNP of the present disclosure and/or at least one pharmaceutical composition of the present disclosure.

### CROSS-REFERENCE TO RELATED APPLICATIONS

This application claims priority under 35 U.S.C. § 119(e) to U.S. Provisional Patent Application No. 63/338,272, filed May 4, 2022, and U.S. Provisional Patent Application No. 63/378,832, filed Oct. 7, 2022, both of which are incorporated herein by reference in their entireties.

### STATEMENT REGARDING FEDERALLY SPONSORED RESEARCH OR DEVELOPMENT

This invention was made with government support under TR002776 awarded by National Institutes of Health. The government has certain rights in the invention.

### SEQUENCE LISTING

The Extensible Markup Language (XML) file named “046483-7381WO1 Sequence Listing.xml” created on May 3, 2023, comprising 12.4 Kbytes, is hereby incorporated by reference in its entirety.

### BACKGROUND

Messenger RNA (mRNA)-based therapeutics have the potential to revolutionize treatments for currently undruggable genetic diseases and can be applied to a wide range of applications for vaccination, protein replacement therapy, cancer immunotherapy and CRISPR-Cas-based gene editing. Recently, the US Food and Drug Administration (FDA) authorized COVID-19 mRNA vaccines, enabled by lipid nanoparticles (LNPs) delivery systems comprised of ionizable lipids, phospholipids, cholesterol, and poly(ethylene glycol) (PEG) lipids. In clinical trials, LNPs encapsulating Cas9 mRNA and a single guide RNA (sgRNA) targeting transthyretin (TTR) have demonstrated durable knockout of TTR to treat hereditary transthyretin amyloidosis. Additionally, emerging LNP formulations such as biodegradable LNPs, vitamin LNPs, imidazole LNPs, dendrimer-like LNPs, heterocyclic LNPs, bisphosphonate LNPs, and biomimetic LNPs, have been developed and evaluated in preclinical studies to increase potency and decrease the side effects of LNPs. These promising advancements highlight the importance and necessity of developing ionizable lipids, or lipidoids, for desired applications in vivo. However, when administered systemically, LNPs preferentially accumulate in the liver, making extrahepatic delivery of mRNA cargo for novel therapeutic treatments challenging.

Recently, a selective organ targeting (SORT) approach was reported to engineer LNPs that precisely tune mRNA delivery profiles in the liver, lung, and spleen through the incorporation of a fifth lipid component. In this approach, charge interactions can finely regulate mRNA delivery to target specific organs. For example, positively charged lipid molecules can be added to LNP formulations to specifically deliver RNA therapeutics to the lung, while negatively charged components can enable RNA delivery to the spleen. It has been recently demonstrated that lipid combinations for targeted gene delivery to organs other than the liver through complement receptors. Further, it has been shown that N-series ionizable lipids can potentially assist RNA delivery to the lung. However, even with these significant developments, tissue-specific gene delivery is not fully developed. Specifically, the structure-activity relationship (SAR) of ionizable lipids to achieve tissue-specific tropism for mRNA delivery is unknown.

The literature is silent regarding leveraging the connection between lipidoid chemical structure and tissue-specific mRNA delivery after systemic administration. Thus, there is a need in the art for novel lipid-like materials with easily altered chemical structures to guide tissue-tropic delivery for next-generation cargo (e.g., mRNA) delivery systems and therapeutics. The present disclosure addresses this need.

### BRIEF DESCRIPTION OF THE DRAWINGS

The following detailed description of exemplary embodiments of the invention will be better understood when read in conjunction with the appended drawings. For the purpose of illustrating the invention, shown in the drawings are illustrative embodiments. It should be understood, however, that the invention is not limited to the precise arrangements and instrumentalities of the embodiments shown in the drawings.

FIGS. 1A-1E depict a combinatorial library of siloxane based ionizable lipids which were chemically synthesized and formulated into siloxane LNPs (SiLNPs) with tunable structures for tissue-specific nucleic acid delivery. FIG. TA provides a schematic showing formulation of SiLNPs via a microfluidic mixing device with different ionizable lipids (siloxane lipidoids), helper lipid (e.g., DOPE), cholesterol, and PEG-lipid (e.g., C14PEG2000). The resulting SiLNPs with different siloxane lipidoid structures guide in vivo tissue-specific mRNA delivery profiles to the liver, lung, and spleen. FIG. 1B depicts representative cryogenic transmission electron microscopy (cryo-TEM) images of SiLNP morphology. Scale bar: 100 nm. FIG. 1C depicts hydrodynamic size distribution of representative SiLNPs. FIG. 1D depicts a list of 12 siloxane amines used to synthesize 252 exemplary siloxane lipidoids. FIG. 1E depicts a list of 21 tails used to synthesize 252 exemplary siloxane lipidoids.

FIGS. 2A-2G depict aspects of the structure-activity relationship (SAR) of SiLNPs for firefly luciferase (Fluc) mRNA delivery in vitro. FIG. 2A depicts a heat map of luciferase expression following treatment of HepG2 cells with SiLNPs (10 ng luciferase mRNA, n≥3 replicates). Relative light units (RLU) of >200 were counted for hit rate calculation. FIG. 2B depicts a relative hit rate of SiLNPs with different amounts of Si per siloxane lipidoid. FIG. 2C depicts a relative hit rate of SiLNPs with different tail substitution numbers. FIG. 2D depicts a relative hit rate of SiLNPs with different tail lengths. FIG. 2E depicts a relative hit rate of SiLNPs with different tail types (e.g., epoxide, α,β-unsaturated ester, or α,β-unsaturated amide derived groups). For example, epoxide-based hit rate was acquired among the epoxide-associated formulations. FIG. 2F depicts a relative hit rate of SiLNPs with different siloxane amine core morphologies (e.g., linear or cyclic) among their core morphology-associated formulations. FIG. 2G depicts a relative hit rate of representative SiLNPs with and without sulfur atoms incorporated into the siloxane amine scaffold. Adding sulfur significantly enhanced in vitro efficacy. 2Si—X indicate representative siloxane lipidoids with 2 Si atoms and 1 amine group (i.e., Si₁₋ vs. Si₂₋), and X-2Si-X indicate representative siloxane lipidoids with 2 Si atoms and 2 amine groups (i.e., Si₅₋ vs. Si₆₋).

FIGS. 3A-3J: siloxane moiety incorporation improves cellular internalization and endosomal escape. FIG. 3A: chemical structures of Si₅—N14 and 213-N14 lipidoids. Si₅—N14 lipidoid possesses higher A Log P value than that of 213-N14 lipidoid, indicating greater hydrophobic property. A Log P was predicted from atomic physiochemical properties. FIG. 3B: representative gating strategy for identifying Cy5-tagged mRNA-LNPs endocytosis by immortalized human lung microvascular endothelial cells (iMVECs). Cells were treated with Si₅—N14 and 213-N14 LNPs delivering Cy5-tagged mRNA at different mRNA dose for 3 h, in which Si₅—N14 LNP exhibited faster cellular uptake than 213-N14 LNP. FIG. 3C: Cy5 positive cell populations of Si₅—N14 and 213-N14 LNPs delivering Cy5-tagged mRNA treated iMVECs. FIG. 3D: Cy5 MFI of Si₅—N14 and 213-N14 LNPs mediated cellular endocytosis at different post-treatment time. iMVECs were treated with Si₅—N14 and 213-N14 LNPs delivering Cy5-tagged mRNA at mRNA dose of 200 ng/mL. FIG. 3E: relative fluorescent intensity v.s. post-treatment time demonstrated not only faster, but also greater endocytosis of Si₅—N14 LNPs than 213-N14 LNPs. Curves were calculated from (FIG. 3D). FIG. 3F: schematic illustration of different lipid accumulation to membrane fluidity. Incorporation of siloxane domain increase the radius of amine head, which may loosen lipid accumulation to improve membrane fluidity for gene delivery. Radius of amine head from Si₅—N14 and 213-N14 lipidoids were calculated based on molecular dynamic stimulation. FIG. 3G: membrane fluidity (1/P) of Si₅—N14 and 213-N14 LNPs was performed by fluorescence polarization measurements. FIG. 3H: representative confocal laser scanning microscope (CLSM) images of cellular uptake and quantification of endosomal escape of Si₅—N14 and 213-N14 LNPs. iMVECs cells were treated with Cy5 mRNA-LNPs (mRNA dose: 600 ng/mL) for 3 h before staining with LysoTracker Green and Hoechst 33342. Scale bar, 50 m. FIGS. 3I-3J: hemolysis of Si₅—N14 and 213-N14 LNPs at pH 5.5 and 7.4. RBCs were incubated with LNPs (dose: 2 g/mL, 4 μg/mL, and 8 μg/mL) at 37° C. for 1 h before the supernatant was transferred into a clear bottom 96-well plate (insert pictures) to determine the adsorption at 540 nm. Statistical significance in (FIG. 3C, FIG. 3G, FIG. 3I, and FIG. 3J) was calculated using a Student's t test with unpaired design. ****p<0.0001; ***p<0.001; *p<0.05. Data are presented as mean±s.e.m. (n=3).

FIGS. 4A-4G depict aspects of the structure-activity studies of siloxane lipidoid structure for controlled in vivo mRNA delivery efficacy and organ selectivity to liver, lung, and spleen. FIGS. 4A-4D depict in vivo evaluation of 36 representative SiLNPs encapsulating Fluc mRNA (0.25 mg·kg⁻¹ dose). Bioluminescence images of various organs were recorded 6 h after i.v. injection of SiLNPs into C57BL/6 mice. H, heart; Li, liver; Lu, lung; K, kidney; S, spleen. FIGS. 4E-4G depict mRNA expression in liver (FIG. 4E), lung (FIG. 4F), and spleen (FIG. 4G) by SiLNPs shown in FIGS. 4A-4D. The pie charts in FIGS. 4E-4G represent in vivo transfection intensity of organs of the top-performing liver-, lung-, and spleen-targeted SiLNP formulations. All data are presented as mean±s.e.m. (n=3 mice).

FIGS. 5A-5N depict liver-targeted mRNA delivery and CRISPR-Cas9 gene editing by SiLNPs. FIG. 5A depicts whole body imaging of luciferase expression by liver-targeted Si₆-C14b LNPs and MC3 LNPs 6 h post-injection (Fluc mRNA, 0.15 mg kg⁻¹). FIG. 5B depicts ex vivo imaging of luciferase expression of different organs from FIG. 5A. The specific high liver luminescence indicated liver-targeting efficacy. H: heart; Li: liver; S: spleen; Lu: lung; K: kidney. FIG. 5C depicts quantification of luciferase expression of organs from FIG. 5B. FIG. 5D provides a schematic of Ai14 mouse model which expresses tdTomato by delivering Cre mRNA to delete the stop cassette. FIG. 5E depicts tdTomato+ cell type evaluation of PBS and Si₆-C14b treated Ai14 mice by flow cytometry (Cre mRNA, 0.3 mg kg⁻¹), including liver sinusoidal endothelial cells (LSECs), hepatocytes, and Kupffer cells. FIG. 5F depicts representative immunostaining of liver histology shows tdTomato fluorescence activation. White arrows in the dashed zone represent transfected LSECs. DAPI was used for nuclear staining. Vascular endothelial cadherin (VECad) was used for labeling LSECs. Scale bar: 100 μm. FIG. 5G provides a schematic demonstration of CRISPR-Cas9 gene editing for transthyretin amyloidosis (TTR) gene of C57BL/6 mice. C57BL/6 mice were systemically injected with Si₆-C14b LNPs co-formulated with Cas9 mRNA and TTR sgRNA (wt:wt, 4:1) with a single dose at 1.0, 2.0, and 3.0 mg kg⁻¹ of total RNA. MC3 LNPs co-delivering Cas9 mRNA and TTR sgRNA (wt:wt, 4:1) were used as positive control. Serum was collected 1 day before and 7 days post-injection. FIG. 5H depicts serum TTR concentration of mice following injections of LNPs co-delivering Cas9 mRNA and TTR sgRNA. FIG. 5I depicts TTR on-target indel frequency in the liver after LNP-mediated CRISPR-Cas9 knockout. FIG. 5J depicts visualization of the reduction of TTR transcript by in situ hybridization (ISH) analysis in liver sections from mice treated with PBS or Si₆-C14b LNPs delivering Cas9 mRNA and TTR sgRNA at different doses. With increased dosing, visualization of TTR transcript weakened. FIGS. 5K-5N provide bar graphs showing blood chemistry assays which demonstrated low toxicity of Si₆-C14b LNPs co-formulated with Cas9 mRNA and TTR sgRNA at an RNA dose of 3.0 mg kg⁻¹ total RNA as compared to PBS treated groups with relatively low values of AST (FIG. 5K), ALT (FIG. 5L), BUN (FIG. 5M), and Creatinine (FIG. 5N). Statistical significance in FIG. 5C, FIG. 5E, FIG. 5H, and FIG. 5I was calculated using a Student's t test with unpaired design. ****P<0.0001; ***P<0.001; **P<0.01. Data are presented as mean s.e.m. (n=3-5 mice).

FIGS. 6A-6O depict lung-targeted mRNA delivery and CRISPR-Cas9 gene editing by SiLNPs. FIG. 6A depicts whole body and ex vivo imaging of luciferase expression by lung-targeted Si₅—N14 LNP 6 h post-injection (Fluc mRNA, dose: 0.3 mg kg⁻¹). The specific high lung luminescence indicated lung-targeting efficacy. H: heart; Li: liver; S: spleen; Lu: lung; K: kidney. FIG. 6B depicts quantification of luciferase expression in organs from FIG. 6A. FIG. 6C depicts quantification of the percent of total corona proteins for the top 5 proteins in the corona of Si₅—N14 LNP. Vtn: vitronectin; Alb: serum albumin; Apob: apolipoprotein B-100; C3: complement C3; Hbb-b1: hemoglobin subunit beta-1. FIG. 6D depicts LNP size variation when incubated in water and fetal bovine serum (FBS) respectively. Amide bond-associated (i.e., α,β-unsaturated amide derived) Si₅—N14 LNPs showed obvious particle size increase in FBS, while ester-based (i.e., α,β-unsaturated ester derived) Si₅—O14 LNPs did not change in terms of particle size. FIG. 6E depicts potential mechanism of the uptake of Si₅—N14 LNPs by lung cells. After injection, Si₅—N14 LNPs accumulate in the narrow lung blood vasculature owing to the generation of larger sized particles. FIG. 6F shows Ai14 mice dosing, wherein mice were administered Si₅—N14 LNP formulated with Cre mRNA 3 days prior to analysis (Cre mRNA, 0.3 mg kg⁻¹). Lungs were digested into single cell suspensions and stained to quantify cell populations for tdTomato+ expression. FIG. 6G depicts representative gating strategy for identification of tdTomato+ ECs (CD45-/CD31+/tdTomato+). FIG. 6H provides a bar graph showing categorization of the top 20 most abundant corona proteins on the basis of calculated molecular weight and isoelectric point; n=3 replicates. FIG. 6I depicts the proportion of tdTomato+ cells in the lung by flow cytometry, including immune cells, endothelial cells, epithelial cells, and other cells. FIG. 6J depicts the distribution of total tdTomato+ cells in each cell type. FIG. 6K provides representative immunostaining, which showed significant overlap between tdTomato+ cells and the EC marker platelet endothelial cell adhesion molecule 1 (PECAM1). DAPI was used for nuclear staining. FIG. 6L provides a schematic demonstration of in vivo gene editing of the GFP gene in the lung of GFP-expressing mice treated by Si₅—N14 LNPs co-formulated with Cas9 mRNA and GFP sgRNA. Mice were repeatedly injected with SiLNPs (i.e., 4 injections, RNA dose: 0.5 mg kg⁻¹ per injection), then were necropsied to quantify GFP knockout in the lung. FIG. 6M shows the proportion of GFP-cells in the lung by flow cytometry, including immune cells, endothelial cells, epithelial cells, and other cells. FIG. 6N depicts representative immunostaining, which showed GFP knockout in lung ECs treated by Si₅—N14 LNPs co-formulated with Cas9 mRNA and GFP sgRNA. Editing occurs in the microvasculature, rather than large vasculature and airway. DAPI was used for staining nuclei. PECAM1 was used for labeling ECs. ERG was used for staining EC nuclei. FIG. 6O depicts quantitative real-time PCR (qPCR) analysis of GFP in sorted ECs from GFP mice 7 days after systemic administration of Si₅—N14 LNPs co-formulated with Cas9 mRNA and GFP sgRNA. Statistical significance in FIG. 5G, FIG. 5K, and FIG. 5M was calculated using a Student's t test with unpaired design. ****P<0.0001; ***P<0.001; **P<0.01; *P<0.05. Data are presented as mean s.e.m. (n=3 mice).

FIGS. 7A-7F: depict utility of lung-targeted SiLNPs for efficient vascular damage regeneration. FIG. 7A: schematic illustration of endothelial repair for lung recovery through delivering angiogenic factors by LNPs in virus-induced lung damage model. FIG. 7B: schematic timeline for LNP administration and sampling. Influenza virus A/H1N1/PR/8 was administered intranasally at 50 to 60 TCID50 units to female C57BL/6J mice. After injection, mice were treated with control (PBS or FLuc mRNA Si₅—N14 LNPs) or FGF-2 mRNA Si₅—N14 LNPs (0.5 mg kg⁻¹) on day 15, and lungs were harvested on day 25. Dexamethasone-21-Phosphate (DEX) was injected intraperitoneally (i.p., 2 mg kg⁻¹) into the mice 30 mins prior to LNP injection in all mice. Time course changes in weight loss (FIG. 7C) and capillary oxygen saturation (FIG. 7D) was observed in infectious C57BL/6J mice treated with either control (PBS or FLuc mRNA Si₅—N14 LNPs) or FGF-2 mRNA Si₅—N14 LNPs. FIG. 7E: analysis of body weight and blood oxygen levels on day 25 after treatment with either control (PBS or FLuc mRNA Si₅—N14 LNPs) or FGF-2 mRNA Si₅—N14 LNPs to lung-damaged mice. FIG. 7F: histological changes in the lungs of mice after receiving control (PBS or FLuc mRNA Si₅—N14 LNPs) or FGF-2 mRNA Si₅—N14 LNPs at day 25 after infection. White areas in H&E staining are pulmonary alveoli, airway and large vessels, while dark spots represent the nuclei. Accumulated dark regions indicate large amounts of immune cell infiltration that leads to damaged inflammatory area (*). Asterisk region represents normal alveoli. Scale bars: 100 μm. Statistical significance in FIGS. 7C-7E was calculated using a Student's t test with unpaired design. *p<0.05. Data are presented as mean s.e.m. (n=3-4 mice).

FIG. 8 depicts cell viability after 24 h transfection of SiLNPs on HepG2 cells. 5000 cells were plated per well and treated with 10 ng mRNA. All data are presented as mean s.e.m. (n=3 replicates).

FIGS. 9A-9B depict in vitro transfection of HepG2 cells by representative SiLNPs without and with the incorporation of sulfur atoms. FIG. 9A: Transfection by representative two tails based SiLNPs without and with sulfur incorporation. FIG. 9B: Transfection by representative four tails based SiLNPs without and with sulfur incorporation. Luminescence intensity was measured after 24 h transfection of SiLNPs on HepG2 cells. 5000 cells were plated per well and treated with 10 ng mRNA. All data are presented as mean s.e.m. (n=3 replicates).

FIGS. 10A-10B depict in vitro transfection efficacy of HepG2 cells by top performing Si₇—N12 LNP. FIG. 10A: Si₇—N12 LNP displayed higher in vitro transfection efficiency than the gold standard MC3 LNP. 5000 cells were plated per well and treated with 10 ng mRNA. FIG. 10B: mRNA dose dependent transfection of HepG2 cells by Si₇—N12 LNP. 5000 cells were plated per well and treated with mRNA doses of 10 ng, 20 ng. 40 ng, 80 ng, and 160 ng, respectively. Luminescence intensity was measured after 24 h transfection of Si₇—N12 LNP on HepG2 cells. All data are presented as mean s.e.m. (n=3 replicates).

FIG. 11 depicts confocal imaging of LNP uptake by HeLa cells. 5×10⁴ HeLa cells were planted per well and treated with MC3 LNP and Si₇—N12 LNP. LNPs were labelled by DiD fluorescent dye. Cell uptake was measured after incubating LNPs with cells for 3 h. Scale bar: 20 μm.

FIGS. 12A-12E depicts characterization of lead liver- (Si₆-C14b), lung- (Si₅—N14), and spleen- (Si₁₂—C10) targeted LNPs, including particle size measurements (FIG. 12A), zeta potential (FIG. 12B), and representative pKa results for Si₆-C14b (FIG. 12C), Si₅—N14 (FIG. 12D), and Si₁₂—C10 (FIG. 12E). All data are presented as mean s.e.m. (n=3 replicates).

FIGS. 13A-13B shows that liver-, lung-, and spleen-targeted lead SiLNPs loading luciferase mRNA are well tolerated in vivo. FIG. 13A depicts ALT levels evaluation. FIG. 13B depicts AST level evaluation. C57BL/6 mice were administered Si₆-C14b, Si₅—N14, and Si₁₂—C10 LNPs with RNA dosage of 3.0 mg kg⁻¹, 0.5 mg kg⁻¹, and 1.0 mg kg⁻¹, respectively. Measurements are tested 12 h post-injection. Data are presented as mean s.e.m. (n=3 mice).

FIG. 14 depicts tissue section histology of PBS, liver-, lung-, and spleen-targeted SiLNPs loading luciferase mRNA. SiLNPs were administered intravenously to C57BL/6 mice at a high mRNA dosage (liver: 3.0 mg kg⁻¹; lung: 0.5 mg kg⁻¹; spleen: 1.0 mg kg⁻¹), and PBS treated group was examined as the negative control. Tissue sections of heart, liver, spleen, lung, and kidney were acquired 12 h post-injection and prepared for H&E staining (n=3 mice). Scale bar=100 μm.

FIG. 15 depicts a representative gating strategy of tdTomato expression in liver cells. Draq7 was used to distinguish liver and dead cells. CD45+ antibody was used to distinguish immune cells, then CD45+/F4/80+ was used for Kupffer cells, CD45−/CD31+ was used for liver sinusoidal endothelial cells (LSECs), and the rest CD45− cell was hepatocytes. Gates for tdTomato+ in cell types was drawn based on PBS treated mice. Ai14 mice were injected by PBS or Si₆-C14b LNP (Cre mRNA: 0.3 mg kg⁻¹), which were necropsied at day 3 for flow cytometry. Data are presented as mean±s.e.m. (n=3 mice).

FIG. 16 depicts representative immunostaining of liver histology showed tdTomato fluorescence in hepatocytes and Kupffer cells. Yellow arrows in the dashed zone represent transfected Kupffer cells. DAPI was used for nuclear staining. F4/80 was used for staining Kupffer cells. Scale bar: 100 μm.

FIG. 17 depicts time-dependent TTR editing of C57BL/6 mice after treatment with a single dose of Si₆-C14b LNPs encapsulating Cas9 mRNA/TTR sgRNA (RNA dose: 3.0 mg kg⁻¹). TTR on-targeting indel at time points of 6 h, 24 h, and 7 days post-injection. PBS treated group was used as control. Data are presented as mean±s.e.m. (n=3-4 mice).

FIG. 18 depicts Long-term TTR editing of C57BL/6 mice after treatment with a single dose of Si₆-C14b LNPs encapsulating Cas9 mRNA/TTR sgRNA (RNA dose: 3.0 mg·kg⁻¹). Serum TTR protein concentration was measured at time points of day 7, day 14, day 21, day 42, and day 56 post-injection. PBS treated group was used as control. Data are presented as mean±s.e.m. (n=3-4 mice).

FIGS. 19A-19C depict a representative gating strategy of tdTomato expression in lung cells. Draq7 was used to distinguish liver and dead cells. CD45+ antibody was used to distinguish immune cells, then CD45−/CD31+ was used for endothelial cells, CD45−/CD31−/EPCAM+ was used for epithelial cells, and the rest CD45−/CD31−/EPCAM− was used for others. Gates for tdTomato+ in cell types were drawn based on PBS treated mice. Ai14mice were injected by PBS or Si₅—N14 LNP (Cre mRNA: 0.3 mg kg⁻¹), which were necropsied at day 3 for flow cytometry. Data are presented as mean s.e.m. (n=3 mice).

FIG. 20 depicts Representative immunostaining showed tdTomato expression mainly in microvasculature, rather than large vasculature and the airway. DAPI was used for nuclear staining, PECAM1 was used for staining endothelial cells. Scale bar: 50 μm.

FIGS. 21A-21B depict in vitro CRISPR-Cas9 gene editing of GFP-HepG2 cells. FIG. 21A depicts quantification of GFP intensity from flow cytometry. Si₅—N14 LNP co-formulated with Cas9 mRNA/GFP sgRNA at mass ratio of 4:1, 3:1, 2:1, 1:1 were added into GFP-HepG2 cell media. 2×10⁴ GFP-HepG2 cells were plated per well and treated with RNA doses of 400 ng, 800 ng, and 1200 ng, respectively. GFP intensity was measured after 7 days of editing with Si₅—N14 LNPs on GFP-HepG2 cells. DMEM media treated group was used as negative control, while Lipofectamine CRISPR MAX loaded with Cas9 mRNA/GFP sgRNA was used as positive control. All data are presented as mean±s.e.m. (n=3 replicates). FIG. 21B depicts representative GFP expression of GFP-HepG2 cells, which was assessed by flow cytometry. Cytometry plots showed decreased GFP expression in cells treated with 0.6 μg/mL Si₅—N14 LNP at Cas9 mRNA/GFP sgRNA of 4:1 and Lipofectamine CRISPR MAX.

FIG. 22 depicts confocal imaging of in vitro CRISPR-Cas9 gene editing of GFP-HepG2 cell. 2×10⁴ GFP-HepG2 cells were plated per well and treated with DMEM media, Lipofectamine CRISPR MAX loading Cas9 mRNA/GFP sgRNA, and Si₅—N14 LNP loading Cas9 mRNA/GFP sgRNA (wt:wt, 4:1). GFP imaging was captured after 7 days of editing of GFP-HepG2 cells. DMEM media treated group was used as negative control, while Lipofectamine CRISPR MAX loading with Cas9 mRNA/GFP sgRNA was used as positive control. All data are presented as mean s.e.m. (n=3 replicates). Scale bar: 20 μm.

FIGS. 23A-23C depict a representative gating strategy of GFP knockout in lung cells. Draq7 was used to distinguish liver and dead cells. CD45+ antibody was used to distinguish immune cell, then CD45−/CD31+ was used for endothelial cells, CD45−/CD31−/EPCAM+ was used for epithelial cells, and the rest CD45−/CD31−/EPCAM− was used for other cell types. Gates for GFP− cell types was drawn based on PBS treated mice. GFP mice were injected with PBS or Si₅—N14 LNP (Cas9 mRNA-GFP sgRNA, total 2.0 mg kg⁻¹), which were necropsied at day 11 for flow cytometry. Data are presented as mean±s.e.m. (n=3 mice).

FIG. 24 depicts representative immunostaining which showed GFP knockout mainly in microvasculature, rather than large vasculature and the airway. DAPI was used for nuclear staining, PECAM1 was used for staining endothelial cells, and ERG was used for staining endothelial cell nuclear. Scale bar: 50 m.

FIGS. 25A-25B depict quantification of FGF-2 expression. FIG. 25A depicts a standard curve of FGF-2 concentration. FIG. 25B depicts serum FGF-2 level levels in mice administered Si₅—N14 LNP, wherein a PBS treated group was used as a control. Statistical significance in FIG. 25B was calculated using a Student's t test with unpaired design. *p<0.05. Data are presented as mean±standard deviation (n=3 mice).

FIGS. 26A-26B depict a representative gating strategy of tdTomato expression in spleen cells. FIG. 26A depicts a gating strategy for tdTomato+ in cell types. CD19+ antibody was used to distinguish B cells, then CD19−/CD3+ was used for T cells, CD11c+ was used for dendritic cells (DC), and F4/80+ was used for macrophages. Gates for tdTomato+ was drawn based on PBS treated mice. Ai14 mice were injected with PBS or Si₁₂—C10 LNPs (Cre mRNA: 0.3 mg kg⁻¹), which were necropsied 3 days post-injection for flow cytometry. FIG. 26B depicts the cell population of tdTomato+ for different cell types. Data are presented as mean s.e.m. (n=3 mice).

FIGS. 27A-27B depict stability of mRNA-LNPs. FIG. 27A depicts particle size evaluation of lead SiLNPs and MC3 LNP over time at 4° C. and room temperature. FIG. 27B depicts relative luminescence evaluation to its original levels of lead SiLNPs and MC3 LNP after 12 post-treatment. Data are presented as mean±standard deviation (n=3 replicates).

### BRIEF SUMMARY

The present disclosure provides a compound of Formula (I), or a salt, solvate, stereoisomer, or isotopologue thereof, wherein the substituents in (I) are defined elsewhere herein:

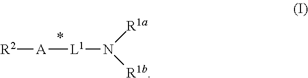

The present disclosure further provides a lipid nanoparticle (LNP). In certain embodiments, the LNP comprises at least one compound of Formula (I), or a salt, solvate, stereoisomer, or isotopologue thereof. In certain embodiments, the LNP comprises at least one neutral phospholipid. In certain embodiments, the LNP comprises at least one cholesterol lipid. In certain embodiments, the LNP comprises at least one selected from the group consisting of polyethylene glycol (PEG) and a PEG-conjugated lipid.

The present disclosure further provides a pharmaceutical composition comprising the LNP of the present disclosure and at least one pharmaceutically acceptable carrier.

The present disclosure further provides a method of delivering a cargo to a subject in need thereof, the method comprising administering to the subject a therapeutically effective amount of at least one LNP of the present disclosure or the pharmaceutical composition of the present disclosure

The present disclosure further provides a method of treating, preventing, and/or ameliorating a disease or disorder in a subject in need thereof, the method comprising administering to the subject a therapeutically effective amount of the LNP of the present disclosure or the pharmaceutical composition of the present disclosure.

The present disclosure further provides a method of inducing an immune response in a subject in need thereof, the method comprising administering to the subject a therapeutically effective amount of at least one LNP of the present disclosure or the pharmaceutical composition of the present disclosure.

The present disclosure further provides a method of treating, preventing, and/or ameliorating cancer in a subject in need thereof, the method comprising administering to the subject a therapeutically effective amount at least one LNP of the present disclosure or the pharmaceutical composition of the present disclosure.

### DETAILED DESCRIPTION

The present disclosure is based, in part, on the unexpected discovery of lipid and/or lipidoid compounds having the structure of Formula (I) that selectively targets at least one liver cell, lung cell, spleen cell, or any combination thereof. In one aspect, the present disclosure provides a lipid nanoparticle (LNP) comprising at least one compound of the present invention. In various embodiments, the LNP comprises one or more compounds of the present disclosure in a concentration range of about 0.1 mol % to about 100 mol %. In some embodiments, the LNP comprises one or more compounds of the present disclosure in a concentration range of about 0.1 mol % to about 99 mol %. In some embodiments, the LNP comprises one or more compounds of the present disclosure in a concentration range of about 1 mol % to about 95 mol %. In some embodiments, the LNP comprises one or more compounds of the present disclosure in a concentration range of about 10 mol % to about 50 mol %.

In various embodiments, the LNP comprises at least one agent for delivery to a cell of interest (e.g., a tissue cell, muscle cell, immune cell, endothelial cell, epithelial cell, hematopoietic stem cell (HSC), heart cell, brain cell, bone marrow cell, hepatocytes, liver cell, spleen cell, lung cell, podocytes, and/or kidney cell, etc.).

In some embodiments, the invention provides a new class of lipid that enables targeted delivery of LNPs to a range of cells without the requirement of a targeting ligand to be immobilized onto the surface to enable targeted delivery. This is because the composition of the invention incorporates targeting capabilities directly into the lipid component itself, through the incorporation of functional groups within the lipids themselves during their synthesis. That is, in some aspects, the chemical structure of the lipid itself and LNP thereof can enable targeted delivery.

In some embodiments, the LNP of the invention is able to target a cell of interest. For example, such cells include, but are not limited to, a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, and the likes.

In another aspect, the present disclosure provides a LNP, comprising at least one compound of the present invention, that selectively targets a cell of interest and is formulated for in vivo stability as well as methods of use thereof for in vivo delivery of an encapsulated agent to the cell of interest. Exemplary agents that can be encapsulated in the compositions of the invention include, but are not limited to, diagnostic agents, detectable agents, and therapeutic agents. In certain embodiments, the present disclosure provides a composition comprising a LNP encapsulating a nucleic acid molecule (e.g., mRNA, siRNA, microRNA, DNA, pDNA, antisense oligonucleotides, etc.).

In one aspect, the composition of the present disclosure comprises one or more LNP formulated for targeted delivery of an agent to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.).

In another aspect, the present disclosure provides a method of inducing an immune response in a subject in need thereof. In some embodiments, the method comprises administering a therapeutically effectively amount of at least one LNP or composition described herein to a subject. In some embodiments, the therapeutically effectively amount of at least one LNP or composition described herein induces an immune response against cancer in the subject.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

The term “about” as used herein can allow for a degree of variability in a value or range, for example, within 10%, within 5%, or within 1% of a stated value or of a stated limit of a range, and includes the exact stated value or range.

The term “adjuvant” as used herein is defined as any molecule to enhance an antigen-specific adaptive immune response.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms or, in some embodiments, from 2 to 8 carbon atoms. Examples include, but are not limited to vinyl, —CH═C═CCH₂, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The term “alkyl” as used herein refers to straight chain and branched alkyl groups and cycloalkyl groups having from 1 to 40 carbon atoms, 1 to about 20 carbon atoms, 1 to 12 carbons or, in some embodiments, from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons or, in some embodiments, from 2 to 8 carbon atoms. Examples include, but are not limited to —C═CH, —C═C(CH₃), —C≡C(CH₂CH₃), —CH₂C≡CH, —CH₂C≡C(CH₃), and —CH₂C≡C(CH₂CH₃) among others.

The term “alkylene” or “alkylenyl” as used herein refers to a bivalent saturated aliphatic radical (e.g., —CH₂—, —CH₂CH₂—, and —CH₂CH₂CH₂—, inter alia). In certain embodiments, the term may be regarded as a moiety derived from an alkene by opening of the double bond or from an alkane by removal of two hydrogen atoms from the same (e.g., —CH₂—) different (e.g., —CH₂CH₂—) carbon atoms. Similarly, the terms “heteroalkylenyl”, “cycloalkylenyl”, “heterocycloalkylenyl”, and the like, as used herein refer to a divalent radical of the moiety corresponding to the base group (e.g., heteroalkyl, cycloalkyl, and/or heterocycloalkyl). A divalent radical possesses two open valencies at any position(s) of the group. Thus, the divalent radical may form a single bond to two distinct atoms or groups, or may form a double bond with one atom.

The term “amine” as used herein refers to primary, secondary, and tertiary amines having, e.g., the formula N(group)₃ wherein each group can independently be H or non-H, such as alkyl, aryl, and the like. Amines include but are not limited to R—NH₂, for example, alkylamines, arylamines, alkylarylamines; R₂NH wherein each R is independently selected, such as dialkylamines, diarylamines, aralkylamines, heterocyclylamines and the like; and R₃N wherein each R is independently selected, such as trialkylamines, dialkylarylamines, alkyldiarylamines, triarylamines, and the like. The term “amine” also includes ammonium ions as used herein.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative can also be a small molecule that differs in structure from the reference molecule, but retains the essential properties of the reference molecule. An analog or derivative may change its interaction with certain other molecules relative to the reference molecule. An analog or derivative molecule may also include a salt, an adduct, tautomer, isomer, prodrug, or other variant of the reference molecule.

The term “amino group” as used herein refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” within the meaning herein can be a primary, secondary, tertiary, or quaternary amino group. An “alkylamino” group includes a monoalkylamino, dialkylamino, and trialkylamino group.

The term “anionic lipid” refers to any lipid that is negatively charged at physiological pH. These lipids include phosphatidylglycerol, cardiolipin, diacylphosphatidylserine, diacylphosphatidic acid, N-dodecanoylphosphatidylethanolamines, N-succinylphosphatidylethanolamines, N-glutarylphosphatidylethanolamines, lysylphosphatidylglycerols, palmitoyloleyolphosphatidylglycerol (POPG), and other anionic modifying groups joined to neutral lipids.

The term “antibody,” as used herein, refers to an immunoglobulin molecule, which specifically binds with an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present disclosure may exist in a variety of forms including, for example, polyclonal antibodies, monoclonal antibodies, Fv, Fab and F(ab)₂, as well as single chain antibodies and humanized antibodies (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426).

The term “antigen” or “Ag” as used herein is defined as a molecule that provokes an adaptive immune response. This immune response may involve either antibody production, or the activation of specific immunogenically-competent cells, or both. The skilled artisan will understand that any macromolecule, including virtually all proteins or peptides, can serve as an antigen. Furthermore, antigens can be derived from recombinant or genomic DNA or RNA. A skilled artisan will understand that any DNA or RNA, which comprises a nucleotide sequences or a partial nucleotide sequence encoding a protein that elicits an adaptive immune response therefore encodes an “antigen” as that term is used herein. Furthermore. one skilled in the art will understand that an antigen need not be encoded solely by a full length nucleotide sequence of a gene. It is readily apparent that the present disclosure includes, but is not limited to, the use of partial nucleotide sequences of more than one gene and that these nucleotide sequences are arranged in various combinations to elicit the desired immune response. Moreover, a skilled artisan will understand that an antigen need not be encoded by a “gene” at all. It is readily apparent that an antigen can be generated synthesized or can be derived from a biological sample. Such a biological sample can include, but is not limited to a tissue sample, a tumor sample, a cell or a biological fluid.

The term “aralkyl” as used herein refers to a radical of the formula —R_{b}—R_{c} where R_{b} is an alkylene group as defined elsewhere herein and Re is one or more aryl radicals as defined elsewhere herein, for example, benzyl, diphenylmethyl and the like. Unless stated otherwise specifically in the specification, an aralkyl group can be optionally substituted.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “cationic lipid” refers to any of a number of lipid species that carry a net positive charge at a selected pH, such as physiological pH (e.g., pH of about 7.0). It has been found that cationic lipids comprising alkyl chains with multiple sites of unsaturation, e.g., at least two or three sites of unsaturation, are particularly useful for forming lipid particles with increased membrane fluidity. A number of cationic lipids and related analogs, which are also useful in the present disclosure, have been described in U.S. Patent Publication Nos. 20060083780 and 20060240554; U.S. Pat. Nos. 5,208,036; 5,264,618; 5,279,833; 5,283,185; 5,753,613; and 5,785,992; and PCT Publication No. WO 96/10390, the disclosures of which are herein incorporated by reference in their entirety for all purposes. Non-limiting examples of cationic lipids are described in detail herein. In some cases, the cat-ionic lipids comprise a protonatable tertiary amine (e.g., pH titratable) head group, Cis alkyl chains, ether linkages between the head group and alkyl chains, and 0 to 3 double bonds. Such lipids include. e.g., DSDMA, DLinDMA, DLenDMA, and DODMA.

The term “cycloalkyl” as used herein refers to cyclic alkyl groups such as, but not limited to, cyclopropyl, cyclobutyl, cyclopentyl. cyclohexyl, cycloheptyl, and cyclooctyl groups. In some embodiments, the cycloalkyl group can have 3 to about 8-12 ring members, whereas in other embodiments the number of ring carbon atoms range from 3 to 4, 5, 6, or 7.

Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like. Cycloalkyl groups also include rings that are substituted with straight or branched chain alkyl groups as defined herein. Representative substituted cycloalkyl groups can be mono-substituted or substituted more than once, such as, but not limited to, 2,2-, 2,3-, 2,4-2,5- or 2,6-disubstituted cyclohexyl groups or mono-, di- or tri-substituted norbornyl or cycloheptyl groups, which can be substituted with, for example, amino, hydroxy, cyano, carboxy, nitro, thio, alkoxy, and halogen groups. The term “cycloalkenyl” alone or in combination denotes a cyclic alkenyl group.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

As used herein, the terms “effective amount,” “pharmaceutically effective amount” and “therapeutically effective amount” refer to a nontoxic but sufficient amount of an agent to provide the desired biological result. That result may be reduction and/or alleviation of the signs, symptoms, or causes of a disease, or any other desired alteration of a biological system. An appropriate therapeutic amount in any individual case may be determined by one of ordinary skill in the art using routine experimentation.

In particular, in the case of a mRNA, and “effective amount” or “therapeutically effective amount” of a therapeutic nucleic acid as relating to a mRNA is an amount sufficient to produce the desired effect, e.g., mRNA-directed expression of an amount of a protein that causes a desirable biological effect in the organism within which the protein is expressed. For example, in some embodiments, the expressed protein is an active form of a protein that is normally expressed in a cell type within the body, and the therapeutically effective amount of the mRNA is an amount that produces an amount of the encoded protein that is at least 50% (e.g., at least 60%, or at least 70%, or at least 80%, or at least 90%) of the amount of the protein that is normally expressed in the cell type of a healthy individual. For example, in some embodiments, the expressed protein is a protein that is normally expressed in a cell type within the body, and the therapeutically effective amount of the mRNA is an amount that produces a similar level of expression as observed in a healthy individual in an individual with aberrant expression of the protein (i.e., protein deficient individual). Suitable assays for measuring the expression of an mRNA or protein include, but are not limited to dot blots, Northern blots, in situ hybridization, ELISA, immunoprecipitation, enzyme function, as well as phenotypic assays known to those of skill in the art.

The term “encode” as used herein refers to the product specified (e.g., protein and RNA) by a given sequence of nucleotides in a nucleic acid (i.e., DNA and/or RNA), upon transcription or translation of the DNA or RNA, respectively. In certain embodiments, the term “encode” refers to the RNA sequence specified by transcription of a DNA sequence. In certain embodiments, the term “encode” refers to the amino acid sequence (e.g., polypeptide or protein) specified by translation of mRNA. In certain embodiments, the term “encode” refers to the amino acid sequence specified by transcription of DNA to mRNA and subsequent translation of the mRNA encoded by the DNA sequence. In certain embodiments, the encoded product may comprise a direct transcription or translation product. In certain embodiments, the encoded product may comprise post-translational modifications understood or reasonably expected by one skilled in the art.

The term “fully encapsulated” indicates that the active agent or therapeutic agent in the lipid particle is not significantly degraded after exposure to serum or a nuclease or protease assay that would significantly degrade free DNA, RNA, or protein. In a fully encapsulated system, preferably less than about 25% of the active agent or therapeutic agent in the particle is degraded in a treatment that would normally degrade 100% of free active agent or therapeutic agent, more preferably less than about 10%, and most preferably less than about 5% of the active agent or therapeutic agent in the particle is degraded. In the context of nucleic acid therapeutic agents, full encapsulation may be determined by an OLIGREEN® assay. OLIGREEN® is an ultra-sensitive fluorescent nucleic acid stain for quantitating oligonucleotides and single-stranded DNA or RNA in solution (available from Invitrogen Corporation; Carlsbad, Calif.). “Fully encapsulated” also indicates that the lipid particles are serum stable, that is, that they do not rapidly decompose into their component parts upon in vivo administration.

The terms “halo,” “halogen,” or “halide” group, as used herein, by themselves or as part of another substituent, mean, unless otherwise stated, a fluorine, chlorine, bromine, or iodine atom.

The term “haloalkyl” group, as used herein, includes mono-halo alkyl groups, poly-halo alkyl groups wherein all halo atoms can be the same or different, and per-halo alkyl groups, wherein all hydrogen atoms are replaced by halogen atoms, such as fluoro. Examples of haloalkyl include trifluoromethyl, 1,1-dichloroethyl, 1,2-dichloroethyl, 1,3-dibromo-3,3-difluoropropyl, perfluorobutyl, and the like.

The term “heteroalkyl” as used herein by itself or in combination with another term, means, unless otherwise stated, a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) (e.g., O, N, P, and S) may be placed at any interior position of the heteroalkyl group or at either terminal position at which the group is attached to the remainder of the molecule, and the heteroatom may be adjacent to a carbonyl (e.g. —C(═O)O— and —C(═O)NH—, inter alia).

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure. A heteroaryl group designated as a C₂-heteroaryl can be a 5-ring with two carbon atoms and three heteroatoms, a 6-ring with two carbon atoms and four heteroatoms and so forth. Likewise a C₄-heteroaryl can be a 5-ring with one heteroatom, a 6-ring with two heteroatoms, and so forth. The number of carbon atoms plus the number of heteroatoms sums up to equal the total number of ring atoms. Heteroaryl groups include, but are not limited to, groups such as pyrrolyl, pyrazolyl, triazolyl, tetrazolyl, oxazolyl, isoxazolyl, thiazolyl, pyridinyl, thiophenyl, benzothiophenyl, benzofuranyl, indolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Heteroaryl groups can be unsubstituted. or can be substituted with groups as is discussed herein. Representative substituted heteroaryl groups can be substituted one or more times with groups such as those listed herein.

Additional examples of aryl and heteroaryl groups include but are not limited to phenyl, biphenyl, indenyl, naphthyl (1-naphthyl, 2-naphthyl), N-hydroxytetrazolyl, N-hydroxytriazolyl, N-hydroxyimidazolyl, anthracenyl (1-anthracenyl, 2-anthracenyl, 3-anthracenyl), thiophenyl (2-thienyl, 3-thienyl), furyl (2-furyl, 3-furyl), indolyl, oxadiazolyl, isoxazolyl, quinazolinyl, fluorenyl, xanthenyl, isoindanyl, benzhydryl, acridinyl, thiazolyl, pyrrolyl (2-pyrrolyl), pyrazolyl (3-pyrazolyl), imidazolyl (1-imidazolyl, 2-imidazolyl, 4-imidazolyl, 5-imidazolyl), triazolyl (1,2,3-triazol-1-yl, 1,2,3-triazol-2-yl 1,2,3-triazol-4-yl, 1,2,4-triazol-3-yl), oxazolyl (2-oxazolyl, 4-oxazolyl, 5-oxazolyl), thiazolyl (2-thiazolyl, 4-thiazolyl, 5-thiazolyl), pyridyl (2-pyridyl, 3-pyridyl, 4-pyridyl), pyrimidinyl (2-pyrimidinyl, 4-pyrimidinyl, 5-pyrimidinyl, 6-pyrimidinyl), pyrazinyl, pyridazinyl (3-pyridazinyl, 4-pyridazinyl, 5-pyridazinyl), quinolyl (2-quinolyl, 3-quinolyl, 4-quinolyl, 5-quinolyl, 6-quinolyl, 7-quinolyl, 8-quinolyl), isoquinolyl (1-isoquinolyl, 3-isoquinolyl, 4-isoquinolyl, 5-isoquinolyl, 6-isoquinolyl, 7-isoquinolyl, 8-isoquinolyl), benzo[b]furanyl (2-benzo[b]furanyl, 3-benzo[b]furanyl, 4-benzo[b]furanyl, 5-benzo[b]furanyl, 6-benzo[b]furanyl, 7-benzo[b]furanyl), 2,3-dihydro-benzo[b]furanyl (2-(2,3-dihydro-benzo[b]furanyl), 3-(2,3-dihydro-benzo[b]furanyl), 4-(2,3-dihydro-benzo[b]furanyl), 5-(2,3-dihydro-benzo[b]furanyl), 6-(2,3-dihydro-benzo[b]furanyl), 7-(2,3-dihydro-benzo[b]furanyl), benzo[b]thiophenyl (2-benzo[b]thiophenyl, 3-benzo[b]thiophenyl, 4-benzo[b]thiophenyl, 5-benzo[b]thiophenyl, 6-benzo[b]thiophenyl, 7-benzo[b]thiophenyl), 2,3-dihydro-benzo[b]thiophenyl, (2-(2,3-dihydro-benzo[b]thiophenyl), 3-(2,3-dihydro-benzo[b]thiophenyl), 4-(2,3-dihydro-benzo[b]thiophenyl), 5-(2,3-dihydro-benzo[b]thiophenyl), 6-(2,3-dihydro-benzo[b]thiophenyl), 7-(2,3-dihydro-benzo[b]thiophenyl), indolyl (1-indolyl, 2-indolyl, 3-indolyl, 4-indolyl, 5-indolyl, 6-indolyl, 7-indolyl), indazole (1-indazolyl, 3-indazolyl, 4-indazolyl, 5-indazolyl, 6-indazolyl, 7-indazolyl), benzimidazolyl (1-benzimidazolyl, 2-benzimidazolyl, 4-benzimidazolyl, 5-benzimidazolyl, 6-benzimidazolyl, 7-benzimidazolyl, 8-benzimidazolyl), benzoxazolyl (1-benzoxazolyl, 2-benzoxazolyl), benzothiazolyl (1-benzothiazolyl, 2-benzothiazolyl, 4-benzothiazolyl, 5-benzothiazolyl, 6-benzothiazolyl, 7-benzothiazolyl), carbazolyl (1-carbazolyl, 2-carbazolyl, 3-carbazolyl, 4-carbazolyl), 5H-dibenz[b,f]azepine (5H-dibenz[b,f]azepin-1-yl, 5H-dibenz[b,f]azepine-2-yl, 5H-dibenz[b,f]azepine-3-yl, 5H-dibenz[b,f]azepine-4-yl, 5H-dibenz[b,f]azepine-5-yl), 10,11-dihydro-5H-dibenz[b,f]azepine (10,11-dihydro-5H-dibenz[b,f]azepine-1-yl, 10,11-dihydro-5H-dibenz[b,f]azepine-2-yl, 10,11-dihydro-5H-dibenz[b,f]azepine-3-yl, 10,11-dihydro-5H-dibenz[b,f]azepine-4-yl, 10,11-dihydro-5H-dibenz[b,f]azepine-5-yl), and the like.

The term “heterocycloalkyl” as used herein refers to an aliphatic, partially unsaturated or fully saturated, 3- to 14-membered ring system, including single rings of 3 to 8 atoms and bi- and tricyclic ring systems where at least one of the carbon atoms of the ring is replaced with a heteroatom such as, but not limited to, nitrogen, oxygen, sulfur, or phosphorus. A heterocycloalkyl can include one to four heteroatoms independently selected from oxygen, nitrogen, and sulfur, wherein a nitrogen and sulfur heteroatom optionally can be oxidized and a nitrogen heteroatom optionally can be substituted. Representative heterocycloalkyl groups include, but are not limited, to the following exemplary groups: pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “heterocyclyl” as used herein refers to aromatic and non-aromatic ring compounds containing three or more ring members, of which one or more is a heteroatom such as, but not limited to, N, O, and S. Thus, a heterocyclyl can be a cycloheteroalkyl, or a heteroaryl, or if polycyclic, any combination thereof. In some embodiments, heterocyclyl groups include 3 to about 20 ring members, whereas other such groups have 3 to about 15 ring members. A heterocyclyl group designated as a C₂-heterocyclyl can be a 5-ring with two carbon atoms and three heteroatoms, a 6-ring with two carbon atoms and four heteroatoms and so forth. Likewise a C₄-heterocyclyl can be a 5-ring with one heteroatom, a 6-ring with two heteroatoms, and so forth. The number of carbon atoms plus the number of heteroatoms equals the total number of ring atoms. A heterocyclyl ring can also include one or more double bonds. A heteroaryl ring is an embodiment of a heterocyclyl group. The phrase “heterocyclyl group” includes fused ring species including those that include fused aromatic and non-aromatic groups. For example, a dioxolanyl ring and a benzdioxolanyl ring system (methylenedioxyphenyl ring system) are both heterocyclyl groups within the meaning herein. The phrase also includes polycyclic ring systems containing a heteroatom such as, but not limited to, quinuclidyl. Heterocyclyl groups can be unsubstituted, or can be substituted as discussed herein. Heterocyclyl groups include, but are not limited to, pyrrolidinyl, piperidinyl, piperazinyl, morpholinyl, pyrrolyl, pyrazolyl, triazolyl, tetrazolyl, oxazolyl, isoxazolyl, thiazolyl, pyridinyl, thiophenyl, benzothiophenyl, benzofuranyl, dihydrobenzofuranyl, indolyl, dihydroindolyl, azaindolyl, indazolyl, benzimidazolyl, azabenzimidazolyl, benzoxazolyl, benzothiazolyl, benzothiadiazolyl, imidazopyridinyl, isoxazolopyridinyl, thianaphthalenyl, purinyl, xanthinyl, adeninyl, guaninyl, quinolinyl, isoquinolinyl, tetrahydroquinolinyl, quinoxalinyl, and quinazolinyl groups. Representative substituted heterocyclyl groups can be mono-substituted or substituted more than once, such as, but not limited to, piperidinyl or quinolinyl groups, which are 2-, 3-, 4-, 5-, or 6-substituted, or disubstituted with groups such as those listed herein.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups.

As used herein, the term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain embodiments there is no hydrocarbyl group.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

The term “ionizable lipid” as used herein refers to a lipid (e.g., a cationic lipid) having at least one protonatable or deprotonatable group, such that the lipid is positively charged at a pH at or below physiological pH (e.g., pH 7.4), and neutral at a second pH, preferably at or above physiological pH. It will be understood by one of ordinary skill in the art that the addition or removal of protons as a function of pH is an equilibrium process, and that the reference to a charged or neutral lipid refers to the nature of the predominant species and does not require that all of the lipid be present in the charged or neutral form. Generally, ionizable lipids have a pKa of the protonatable group in the range of about 4 to about 7.

The term “local delivery,” as used herein, refers to delivery of an active agent or therapeutic agent such as a messenger RNA directly to a target site within an organism. For example, an agent can be locally delivered by direct injection into a disease site such as a tumor or other target site such as a site of inflammation or a target organ such as the liver, heart, pancreas, kidney, and the like.

The term “lipid” refers to a group of organic compounds that include, but are not limited to, esters of fatty acids and are characterized by being insoluble in water, but soluble in many organic solvents. They are usually divided into at least three classes: (1) “simple lipids,” which include fats and oils as well as waxes; (2) “compound lipids,” which include phospholipids and glycolipids; and (3) “derived lipids” such as steroids.

The term “lipid conjugate” refers to a conjugated lipid that inhibits aggregation of lipid particles. Such lipid conjugates include, but are not limited to, polyamide oligomers (e.g., ATTA-lipid conjugates), PEG-lipid conjugates, such as PEG coupled to dialkyloxypropyls, PEG coupled to diacylglycerols, PEG coupled to cholesterol, PEG coupled to phosphatidylethanolamines, PEG conjugated to ceramides (e.g., U.S. Pat. No. 5,885,613, the disclosure of which is herein incorporated by reference in its entirety for all purposes), cationic PEG lipids, and mixtures thereof. PEG can be conjugated directly to the lipid or may be linked to the lipid via a linker moiety. Any linker moiety suitable for coupling the PEG to a lipid can be used including, e.g., non-ester containing linker moieties and ester-containing linker moieties. In preferred embodiments, non-ester containing linker moieties are used.

As used herein, “lipid encapsulated” can refer to a lipid particle that provides an active agent or therapeutic agent, such as a nucleic acid (e.g., a protein cargo), with full encapsulation, partial encapsulation, or both. In a preferred embodiment, the nucleic acid is fully encapsulated in the lipid particle (e.g., to form an SPLP, pSPLP, SNALP, or other nucleic acid-lipid particle).

The term “lipid nanoparticle” refers to a particle having at least one dimension on the order of nanometers (e.g., 1-1,000 nm) which includes one or more lipids and/or additional agents.

The term “lipid particle” is used herein to refer to a lipid formulation that can be used to deliver an active agent or therapeutic agent, such as a nucleic acid (e.g., mRNA), to a target site of interest. In the lipid particle of the disclosure, which is typically formed from a cationic lipid, a non-cationic lipid, and a conjugated lipid that prevents aggregation of the particle, the active agent or therapeutic agent may be encapsulated in the lipid, thereby protecting the agent from enzymatic degradation.

The term “monovalent” as used herein refers to a substituent connecting via a single bond to a substituted molecule. When a substituent is monovalent, such as, for example, F or Cl, it is bonded to the atom it is substituting by a single bond.

The term “natural amino acid” as used herein refers to an amino acid (with the usual three letter codes & one letter codes in parenthesis) selected from the group consisting of Glycine (Gly & G), proline (Pro & P), alanine (Ala & A), valine (Val & V), leucine (Leu & L), isoleucine (Ile & I), methionine (Met & M), cysteine (Cys & C), phenylalanine (Phe & F), tyrosine (Tyr & Y), tryptophan (Trp & W), histidine (His & H), lysine (Lys & K), arginine (Arg & R), glutamine (Gln & Q), asparagine (Asn & N), glutamic acid (Glu & E), aspartic acid (Asp & D), serine (Ser & S) and threonine (Thr & T).

The term “neutral lipid” refers to any of a number of lipid species that exist either in an uncharged or neutral zwitterionic form at a selected pH. At physiological pH, such lipids include, for example, diacylphosphatidylcholine, diacylphosphatidylethanolamine, ceramide, sphingomyelin, cephalin, cholesterol, cerebrosides, and diacylglycerols.

The term “non-cationic lipid” refers to any amphipathic lipid as well as any other neutral lipid or anionic lipid.

The term “nucleic acid” as used herein refers to a polymer containing at least two deoxyribonucleotides or ribonucleotides in either single- or double-stranded form and includes DNA and RNA. DNA may be in the form of, e.g., antisense molecules, plasmid DNA, pre-condensed DNA, a PCR product, vectors (Pl, PAC, BAC, YAC, artificial chromosomes), expression cassettes, chimeric sequences, chromosomal DNA, or derivatives and combinations of these groups. RNA may be in the form of siRNA, asymmetrical interfering RNA (aiRNA), microRNA (miRNA), mRNA, tRNA, rRNA, tRNA, viral RNA (vRNA), and combinations thereof. Nucleic acids include nucleic acids containing known nucleotide analogs or modified backbone residues or linkages, which are synthetic, naturally occurring, and non-naturally occurring, and which have similar binding properties as the reference nucleic acid. Examples of such analogs include, without limitation, phosphorothioates, phosphoramidates, methyl phosphonates, chiral-methyl phosphonates, 2′-O-methyl ribonucleotides, and peptide-nucleic acids (PNAs). Unless specifically limited, the term encompasses nucleic acids containing known analogues of natural nucleotides that have similar binding properties as the reference nucleic acid. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mal. Cell. Probes, 8:91-98 (1994)).

As used herein, the term “nucleic acid” includes any oligonucleotide or polynucleotide, with fragments containing up to 60 nucleotides generally termed oligonucleotides, and longer fragments termed polynucleotides. In particular embodiments, oligonucleotides of the disclosure are from about 15 to about 60 nucleotides in length. Nucleic acid may be administered alone in the lipid particles of the disclosure, or in combination (e.g., co-administered) with lipid particles of the disclosure comprising peptides, polypeptides, or small molecules such as conventional drugs. In other embodiments, the nucleic acid may be administered in a viral vector.

“Nucleotides” contain a sugar deoxyribose (DNA) or ribose (RNA), a base, and a phosphate group. Nucleotides are linked together through the phosphate groups. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkyl halides.

Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res., 19:5081 (1991); Ohtsuka et al., J. Biol. Chem., 260:2605-2608 (1985); Rossolini et al., Mol. Cell. Probes, 8:91-98 (1994)).

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The terms “patient,” “subject,” or “individual” are used interchangeably herein, and refer to any animal, or cells thereof whether in vitro or in situ, amenable to the methods described herein. In a non-limiting embodiment, the patient, subject or individual is a human.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the language “pharmaceutically acceptable salt” refers to a salt of the administered compounds prepared from pharmaceutically acceptable non-toxic acids or bases, including inorganic acids or bases, organic acids or bases, solvates, hydrates, or clathrates thereof.

The term “sgRNA” typically refers to a single-guide RNA (i.e., a single, contiguous polynucleotide sequence) that essentially comprises a crRNA connected at its 3′ end to the 5′ end of a tracrRNA through a “loop” sequence (see, e.g., U.S. Published Patent Application No. 20140068797). sgRNA interacts with a cognate Cas protein essentially as described for tracrRNA/crRNA polynucleotides, as discussed above. Similar to crRNA, sgRNA has a spacer, a region of complementarity to a potential DNA target sequence, adjacent a second region that forms base-pair hydrogen bonds that form a secondary structure, typically a stem structure. The term includes truncated single-guide RNAs (tru-sgRNAs) of approximately 17-18 nt (see e.g., Fu, Y. et. al., “Improving CRISPR-Cas nuclease specificity using truncated guide RNAs,” Nat Biotechnol. (2014) 32:279-284). The term also encompasses functional miniature sgRNAs with expendable features removed, but that retain an essential and conserved module termed the “nexus” located in the portion of sgRNA that corresponds to tracrRNA (not crRNA). See, e.g., U.S. Published Patent Application No. 20140315985, published 23 Oct. 2014, incorporated herein by reference in its entirety; Briner et al., “Guide RNA Functional Modules Direct Cas9 Activity and Orthogonality,” Molecular Cell (2014) 56:333-339. The nexus is located immediately downstream of (i.e., located in the 3′ direction from) the lower stem in Type II CRISPR-Cas9 systems. The nexus confers the binding of a sgRNA or a tracrRNA to its cognate Cas9 protein and confers an apoenzyme to haloenzyme conformational transition.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxybenzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxybutyric, salicylic, galactaric and galacturonic acid.

Suitable pharmaceutically acceptable base addition salts of compounds described herein include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (N-methylglucamine) and procaine. All of these salts may be prepared from the corresponding compound by reacting, for example, the appropriate acid or base with the compound.

As used herein, the term “pharmaceutically acceptable carrier” or “pharmaceutically acceptable excipient” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound described herein within or to the patient such that it may perform its intended function. Typically, such compounds are carried or transported from one organ, or portion of the body, to another organ, or portion of the body. Each carrier must be “acceptable” in the sense of being compatible with the other ingredients of the formulation, including the compound(s) described herein, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound(s) described herein, and are physiologically acceptable to the patient. Supplementary active compounds may also be incorporated into the compositions. The “pharmaceutically acceptable carrier” may further include a pharmaceutically acceptable salt of the compound(s) described herein. Other additional ingredients that may be included in the pharmaceutical compositions used with the methods or compounds described herein are known in the art and described, for example in Remington's Pharmaceutical Sciences (Genaro, Ed., Mack Publishing Co., 1985, Easton, PA), which is incorporated herein by reference.

The terms “peptide,” “polypeptide,” and “protein” are used interchangeably herein, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “polymer conjugated lipid” refers to a molecule comprising both a lipid portion and a polymer portion. An example of a polymer conjugated lipid is a pegylated lipid. The term “pegylated lipid” refers to a molecule comprising both a lipid portion and a polyethylene glycol portion. Pegylated lipids are known in the art and include 1-(monomethoxy-polyethyleneglycol)-2,3-dimyristoylglycerol (PEG-s-DMG), DSPE-PEG-DBCO, DOPE-PEG-Azide, DSPE-PEG-Azide, DPPE-PEG-Azide, DSPE-PEG-Carboxy-NHS, DOPE-PEG-Carboxylic Acid, DSPE-PEG-Carboxylic acid and the like.

The term “room temperature” as used herein refers to a temperature of about 15° C. to 28° C.

The term “solvent” as used herein refers to a liquid that can dissolve a solid, liquid, or gas. Non-limiting examples of solvents are silicones, organic compounds, water, alcohols, ionic liquids, and supercritical fluids.

By the term “specifically binds,” as used herein with respect to an antibody, is meant an antibody which recognizes a specific antigen, but does not substantially recognize or bind other molecules in a sample. For example, an antibody that specifically binds to an antigen from one species may also bind to that antigen from one or more other species. But, such cross-species reactivity does not itself alter the classification of an antibody as specific. In another example, an antibody that specifically binds to an antigen may also bind to different allelic forms of the antigen. However, such cross reactivity does not itself alter the classification of an antibody as specific. In some instances, the terms “specific binding” or “specifically binding,” can be used in reference to the interaction of an antibody, a protein, or a peptide with a second chemical species, to mean that the interaction is dependent upon the presence of a particular structure (e.g., an antigenic determinant or epitope) on the chemical species; for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that the composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less. The term “substantially free of” can mean having a trivial amount of, such that a composition is about 0 wt % to about 5 wt % of the material, or about 0 wt % to about 1 wt %, or about 5 wt % or less, or less than, equal to, or greater than about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀) hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

A “therapeutic” treatment is a treatment administered to a subject who exhibits signs of pathology, for the purpose of diminishing or eliminating those signs.

The term “therapeutic protein” as used herein refers to a protein or peptide which has a positive or advantageous effect on a condition or disease state of a subject when provided to the subject in a therapeutically effective amount. In some embodiments, a therapeutic protein or peptide has curative or palliative properties and may be administered to ameliorate, relieve, alleviate, reverse, delay onset of or lessen the severity of one or more symptoms of a disease or disorder. A therapeutic protein or peptide may have prophylactic properties and may be used to delay the onset of a disease or to lessen the severity of such disease or pathological condition. The term “therapeutic protein” includes entire proteins or peptides, and can also refer to therapeutically active fragments thereof. It can also include therapeutically active variants of a protein. Exemplary therapeutic proteins include, but are not limited to, an analgesic protein, an anti-inflammatory protein, an anti-proliferative protein, an proapoptotic protein, an anti-angiogenic protein, a cytotoxic protein, a cytostatic protein, a cytokine, a chemokine, a growth factor, a wound healing protein, a pharmaceutical protein, or a pro-drug activating protein. Therapeutic proteins may include growth factors (EGF, TGF-α, TGF-β, TNF, HGF, IGF, and IL-1-8, inter alia) cytokines, paratopes, Fabs (fragments, antigen binding), and antibodies.

The terms “treat,” “treating” and “treatment,” as used herein, means reducing the frequency or severity with which symptoms of a disease or condition are experienced by a subject by virtue of administering an agent or compound to the subject.

The term “unnatural amino acid” as used herein refers to any amino acid, modified amino acid, and/or amino acid analogue, that is not one of the 20 common naturally occurring amino acids.

The term “vector” as used herein refers to a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus.

The term should also be construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

### Description

Systemic delivery of messenger RNA (mRNA) for tissue-specific targeting with high selectivity and efficacy using lipid nanoparticles (LNPs) holds enormous therapeutic potential for applications in vaccines, protein replacement therapy, cancer immunotherapy and gene editing. However, ensuring potency and safety remains challenging for traditional four-component LNP formulations to target specific cells and tissues in vivo.

The present disclosure describes the design a class of siloxane-based lipid-like materials into siloxane LNPs (SiLNPs) and, through alteration of the siloxane ionizable lipid structure, control of the mRNA delivery profile to target the liver, lung, and spleen in mice in vivo. By co-delivering Cas9 mRNA and single guide RNA (sgRNA) in a single SiLNP formulation, liver-specific SiLNPs targeting the transthyretin (TTR) gene to treat hereditary transthyretin amyloidosis significantly reduced serum TTR concentration and indels in the liver of C57BL/6 mice. Furthermore, lung-specific SiLNPs co-delivering Cas9 mRNA and GFP sgRNA demonstrated efficient and potent editing of lung endothelial and epithelial cells in a GFP mouse model. The SiLNPs of the present disclosure may aid in the translational application of mRNA therapeutics for next-generation tissue-specific protein replacement therapies, regenerative medicine, and gene editing.

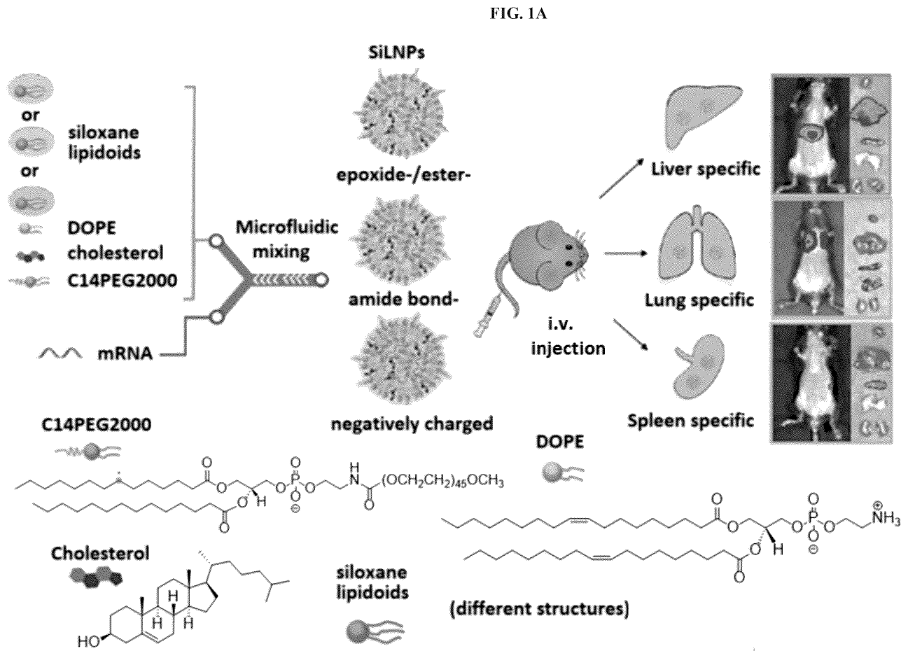

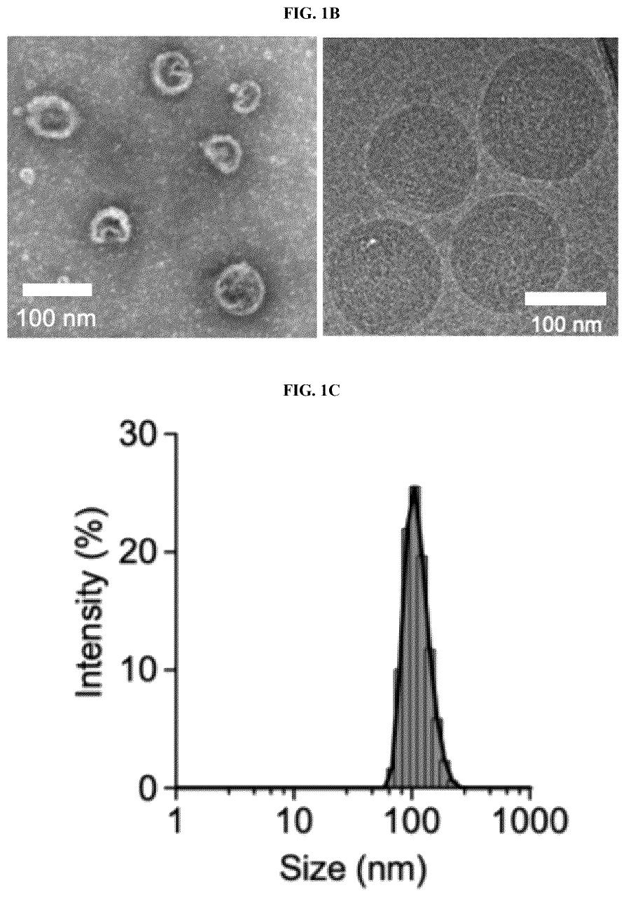

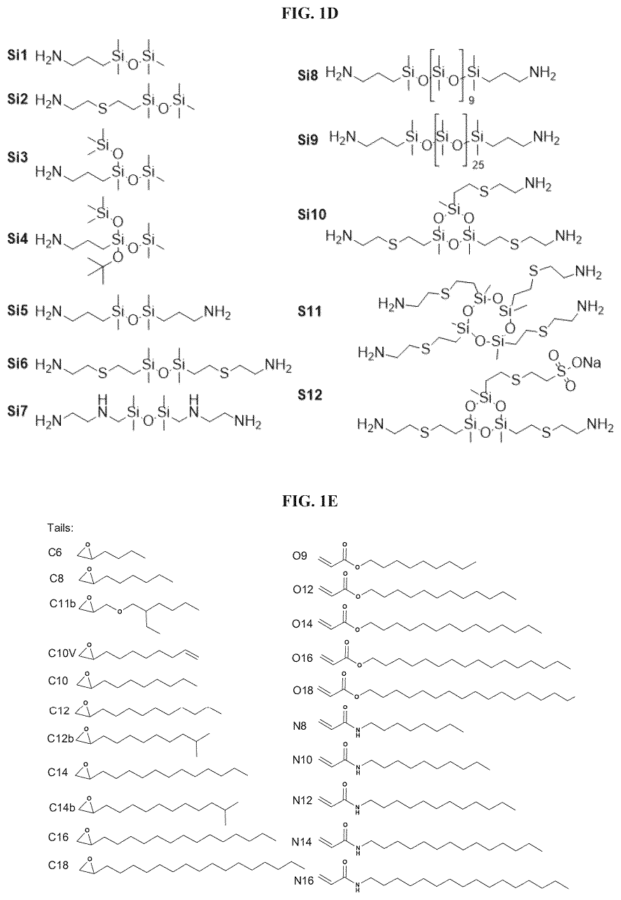

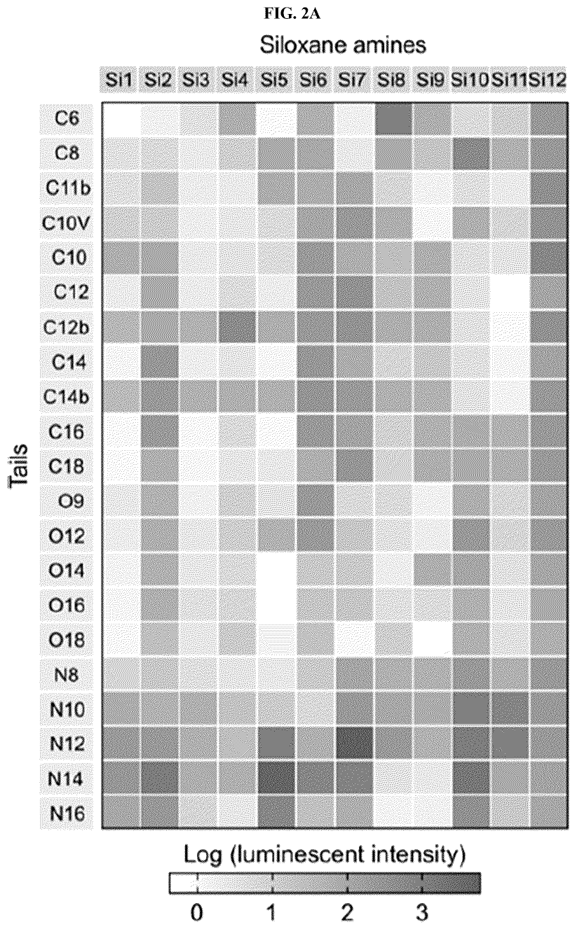

Herein is described the development of siloxane-based lipid-like materials, including the development of a library of 252 siloxane-based ionizable lipids (i.e., siloxane lipidoids) with varied siloxane amine core compositions and alkyl chain structures, and the formulation of these siloxane lipidoids into siloxane LNPs (SiLNPs) to demonstrate structure-guided in vivo systemic mRNA delivery profiles for tissue-specific gene delivery and CRISPR-Cas9 editing. Following an initial screen, SiLNPs featuring siloxane lipidoids comprising four tertiary amines and six amine-linked linear alkyl chains demonstrated the highest mRNA transfection, mediating up to 6-fold greater protein expression compared to a clinically-relevant ionizable lipid (DLin-MC3-DMA, referred to herein as “MC3”). Interestingly, based on the organized SAR evaluation in vivo, an in-depth understanding of the relationship between the chemical structures of siloxane lipidoids and tissue-tropic mRNA delivery was proposed. A structure-guided tissue-targeted mRNA delivery profile was demonstrated. Specifically, epoxide- and ester tail-based siloxane lipidoids delivered firefly luciferase (Flue) mRNA to the liver, siloxane lipidoids with amide tails delivered Fluc mRNA to the lung, and negatively charged cyclic siloxane lipidoids enabled Fluc mRNA delivery to the spleen (FIGS. 1A-1C). Liver-, lung-, and spleen-targeted SiLNPs were used to deliver Cre recombinase mRNA (Cre mRNA) to an Ai14 mice model, which resulted in organ-specific transfection in various cell types including hepatocytes, Kupffer cells, endothelial cells, dendritic cells, and splenic macrophages.

To demonstrate the potential of this technology for CRISPR-Cas9 gene editing, liver-targeted SiLNPs mediated co-delivery of Cas9 mRNA and mouse transthyretin (mTTR) single guide RNA (sgRNA) to significantly reduce serum TTR protein levels in the livers of wild-type C57BL/6 mice. Additionally, lung-specific SiLNPs co-delivering Cas9 mRNA and GFP sgRNA efficiently edited lung endothelial cells and epithelial cells in a transgene GFP mouse model. These results demonstrate the importance of discovering novel lipid-like materials and investigating SAR of these lipid-like materials for next-generation tissue-specific protein replacement and gene editing therapeutics.

### Siloxane-Lipidoid Compounds

In one aspect, the present disclosure provides a compound of Formula (I), or a salt, solvate, stereoisomer, or isotopologue thereof:

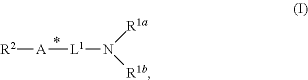

wherein:
A is

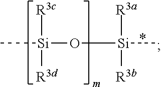

R² is selected from the group consisting of

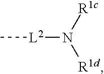

optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{1a}, R^{1b}, R^{1c}, and R^{1d}, if present, are each independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
L¹ and L² are each independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₈ cycloalkylenyl, and optionally substituted C₂-C₈ heterocycloalkylenyl;
each occurrence of R^{3a}, R^{3b}, R^{3c}, and R^{3d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl,
   wherein two occurrences of R³ or two occurrences of R^{3d} can combine with the atoms to which they are bound to form an optionally substituted 6-10 membered cyclosiloxane, or
   wherein R^{3a} and R^{3c}, R^{3d} and R^{3a}, R^{3b} and R^{3c}, or R^{3b} and R^{3d} can combine with the atoms to which they are bound to form an optionally substituted 6-10 membered cyclosiloxane;
each occurrence of R^{A} is independently selected from the group consisting of optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl; and
m is an integer ranging from 0 to 50.

In certain embodiments, A is selected from the group consisting of

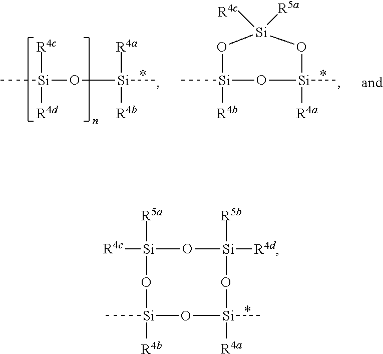

wherein:
R^{4a}, R^{4b}, R^{4c}, and R^{4d}, if present, are each independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{5a} and R^{5b}, if present, are each independently selected from the group consisting of

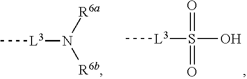

optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of L³ is independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₈ cycloalkylenyl, and optionally substituted C₂-C₈ heterocycloalkylenyl;
each occurrence of R^{6a} and R^{6b} is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl; and
n is an integer ranging from 0 to 30.

In certain embodiments, R^{4a} is Me. In certain embodiments, R^{4a} is OSiMe₃. In certain embodiments, R^{4b} is Me. In certain embodiments, R^{4b} is OSiMe₃. In certain embodiments, R^{4c} is Me. In certain embodiments, R^{4c} is OSiMe₃. In certain embodiments, R^{4d} is Me. In certain embodiments, R^{4d} is OSiMe₃.

In certain embodiments, A is

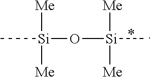

In certain embodiments, A is

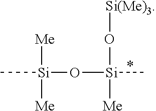

In certain embodiments, A is

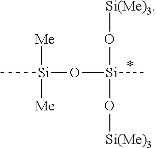

In certain embodiments, A is

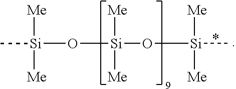

In certain embodiments, A is

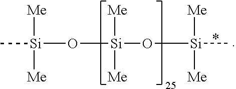

In certain embodiments, A is

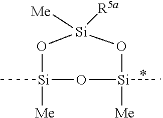

In certain embodiments, A is

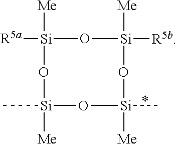

In certain embodiments, L¹ is —(CH₂)₁₋₅S(CH₂)₁₋₅—. In certain embodiments, L¹ is —(CH₂)₁₋₅—. In certain embodiments, L¹ is —(CH₂)₁₋₅N(R^{6a})(CH₂)₁₋₅—.

In certain embodiments, L² is —(CH₂)₁₋₅S(CH₂)₁₋₅—. In certain embodiments, L² is —(CH₂)₁₋₅—. In certain embodiments, L² is —(CH₂)₁₋₅N(R₆ₐ)(CH₂)₁₋₅—.

In certain embodiments, L³ is —(CH₂)₁₋₅S(CH₂)₁₋₅—. In certain embodiments, L³ is —(CH₂)₁₋₅—. In certain embodiments, L³ is —(CH₂)₁₋₅N(R^{6a})(CH₂)₁₋₅—.

In certain embodiments, L¹ is —(CH₂)₃—. In certain embodiments, L¹ is —(CH₂)₂S(CH₂)₂—. In certain embodiments, L¹ is —(CH₂)NR^{6a}(CH₂)₂—.

In certain embodiments, L² is —(CH₂)₃—. In certain embodiments, L² is —(CH₂)₂S(CH₂)₂—. In certain embodiments, L² is —(CH₂)NR^{6a}(CH₂)₂—.

In certain embodiments, L³ is —(CH₂)₃—. In certain embodiments, L³ is —(CH₂)₂S(CH₂)₂—. In certain embodiments, L³ is —(CH₂)NR^{6a}(CH₂)₂—.

In certain embodiments, the compound of Formula (I) is

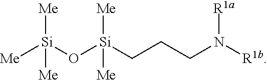

In certain embodiments, the compound of Formula (I) is

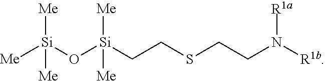

In certain embodiments, the compound of Formula (I) is

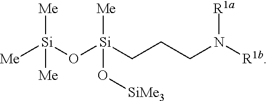

In certain embodiments, the compound of Formula (I) is

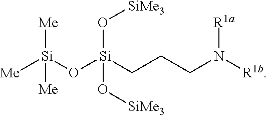

In certain embodiments, the compound of Formula (I) is

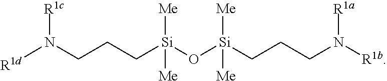

In certain embodiments, the compound of Formula (I) is

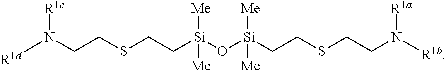

In certain embodiments, the compound of Formula (I) is

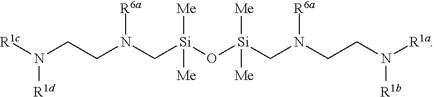

In certain embodiments, the compound of Formula (I) is

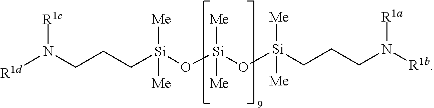

In certain embodiments, the compound of Formula (I) is

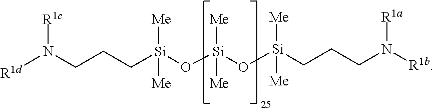

In certain embodiments, the compound of Formula (I) is

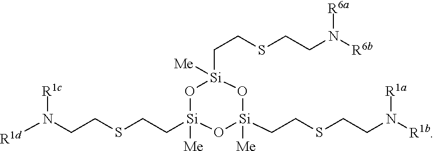

In certain embodiments, the compound of Formula (I) is

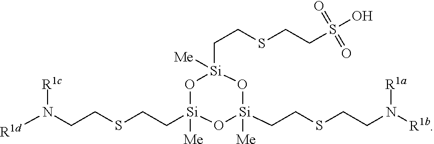

In certain embodiments, the compound of Formula (I) is

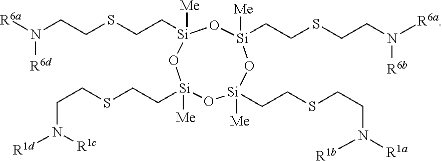

In certain embodiments, R^{1a} is —CH₂CHOH-(optionally substituted C₁-C₂₀ alkyl). In certain embodiments, R^{1a} is —CH₂CHOH-(optionally substituted C₁-C₂₀ heteroalkyl). In certain embodiments, R^{1a} is —CH₂CHOH-(optionally substituted C₂-C₂₀ alkenyl). In certain embodiments, R^{1a} is —CH₂CH₂C(═O)O(optionally substituted C₁-C₂₀ alkyl). In certain embodiments, R^{1a} is —CH₂CH₂C(═O)NH(optionally substituted C₁-C₂₀ alkyl).

In certain embodiments, R^{1b} is —CH₂CHOH-(optionally substituted C₁-C₂₀ alkyl). In certain embodiments, R^{1b} is —CH₂CHOH-(optionally substituted C₁-C₂₀ heteroalkyl). In certain embodiments, R^{1b} is —CH₂CHOH-(optionally substituted C₂-C₂₀ alkenyl). In certain embodiments, R^{1b} is —CH₂CH₂C(═O)O(optionally substituted C₁-C₂₀ alkyl). In certain embodiments, R^{1b} is —CH₂CH₂C(═O)NH(optionally substituted C₁-C₂₀ alkyl).

In certain embodiments, R^{1b} is —CH₂CHOH-(optionally substituted C₁-C₂₀ alkyl). In certain embodiments, R^{1b} is —CH₂CHOH-(optionally substituted C₁-C₂₀ heteroalkyl). In certain embodiments, R^{1b} is —CH₂CHOH-(optionally substituted C₂-C₂₀ alkenyl). In certain embodiments, R^{1b} is —CH₂CH₂C(═O)O(optionally substituted C₁-C₂₀ alkyl). In certain embodiments, R^{1c} is —CH₂CH₂C(═O)NH(optionally substituted C₁-C₂₀ alkyl).

In certain embodiments, R^{1d} is —CH₂CHOH-(optionally substituted C₁-C₂₀ alkyl). In certain embodiments, R^{1d} is —CH₂CHOH-(optionally substituted C₁-C₂₀ heteroalkyl). In certain embodiments, R^{1d} is —CH₂CHOH-(optionally substituted C₂-C₂₀ alkenyl). In certain embodiments, R^{1d} is —CH₂CH₂C(═O)O(optionally substituted C₁-C₂₀ alkyl). In certain embodiments, R^{1d} is —CH₂CH₂C(═O)NH(optionally substituted C₁-C₂₀ alkyl).

In certain embodiments, R^{1a} is

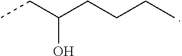

In certain embodiments, R^{1a} is

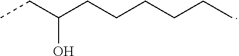

In certain embodiments, R^{1a} is

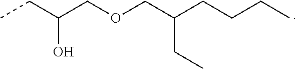

In certain embodiments, R^{1a} is

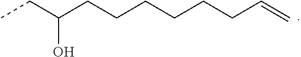

In certain embodiments, R^{1a} is

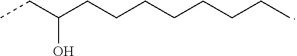

In certain embodiments, R^{1a} is

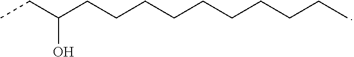

In certain embodiments, R^{1a} is

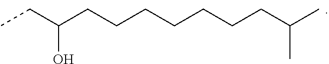

In certain embodiments, R^{1a} is

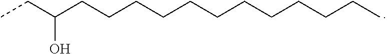

In certain embodiments, R^{1a} is

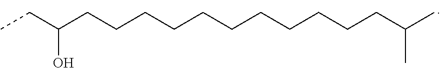

In certain embodiments, R^{1a} is

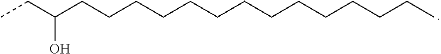

In certain embodiments, R^{1a} is

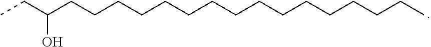

In certain embodiments, R^{1a} is

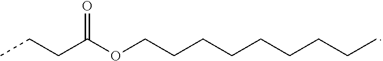

In certain embodiments, R^{1a} is

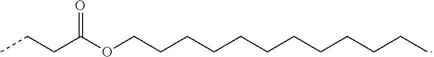

In certain embodiments, R^{1a} is

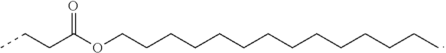

In certain embodiments, R^{1a} is

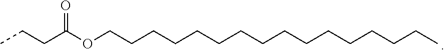

In certain embodiments, R^{1a} is

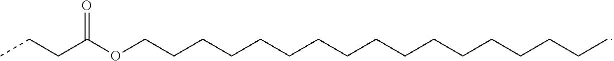

In certain embodiments, R^{1a} is

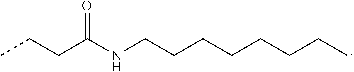

In certain embodiments, R^{1a} is

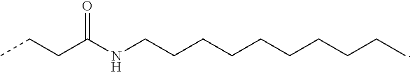

In certain embodiments, R^{1a} is

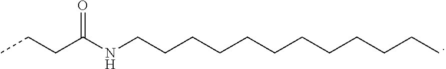

In certain embodiments, R^{1a} is

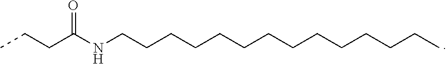

In certain embodiments, R^{1a} is

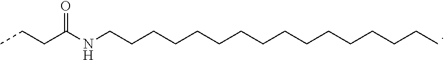

In certain embodiments, R^{1b} is

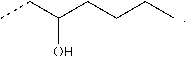

In certain embodiments, R^{1b} is

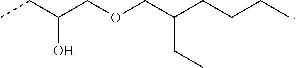

In certain embodiments, R^{1b} is

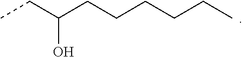

In certain embodiments, R^{1b} is

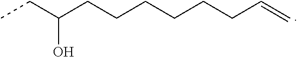

In certain embodiments, R^{1b} is

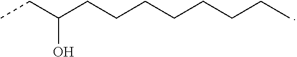

In certain embodiments, R^{1b} is

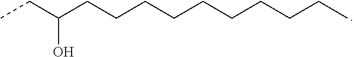

In certain embodiments, R^{1b} is

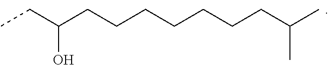

In certain embodiments, R^{1b} is

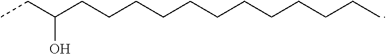

In certain embodiments, R^{1b} is

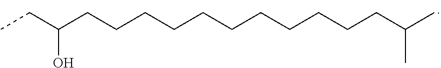

In certain embodiments, R^{1b} is

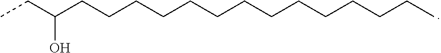

In certain embodiments, R^{1b} is

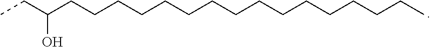

In certain embodiments, R^{1b} is

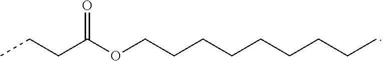

In certain embodiments, R^{1b} is

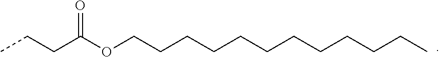

In certain embodiments, R^{1b} is

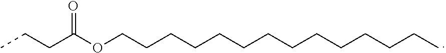

In certain embodiments, R^{1b} is

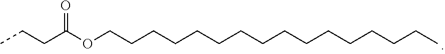

In certain embodiments, R^{1b} is

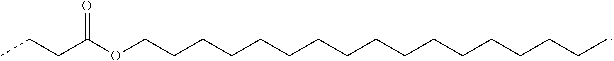

In certain embodiments, R^{1b} is

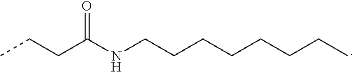

In certain embodiments, R^{1b} is

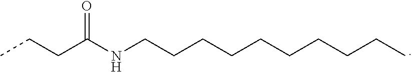

In certain embodiments, R^{1b} is

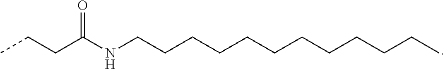

In certain embodiments, R^{1b} is

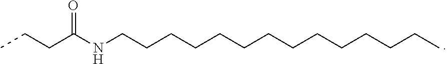

In certain embodiments, R^{1b} is

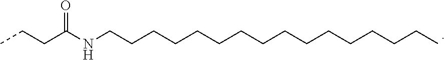

In certain embodiments, R^{1c} is

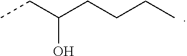

In certain embodiments, R^{1c} is

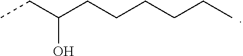

In certain embodiments, R^{1c} is

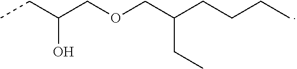

In certain embodiments, R^{1c} is

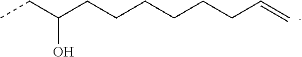

In certain embodiments, R^{1c} is

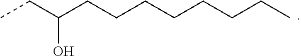

In certain embodiments, R^{1c} is

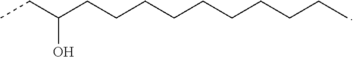

In certain embodiments, R^{1c} is

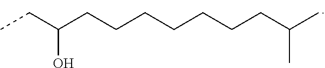

In certain embodiments, R^{1c} is

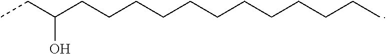

In certain embodiments, R^{1c} is

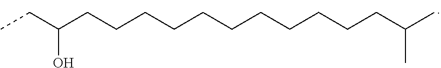

In certain embodiments, R^{1c} is

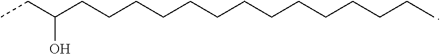

In certain embodiments, R^{1c} is

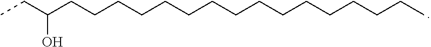

In certain embodiments, R^{1c} is

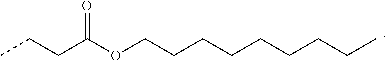

In certain embodiments, R^{1c} is

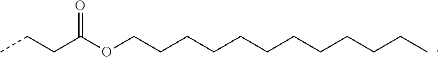

In certain embodiments, R^{1c} is

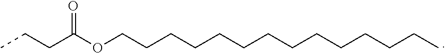

In certain embodiments, R^{1c} is

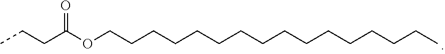

In certain embodiments, R^{1c} is

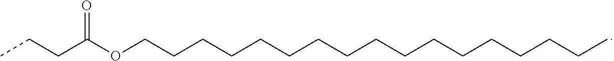

In certain embodiments, R^{1c} is

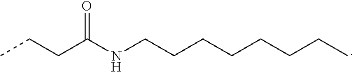

In certain embodiments, R^{1c} is

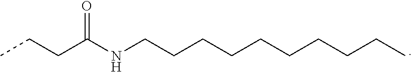

In certain embodiments, R^{1c} is

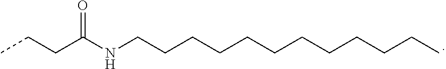

In certain embodiments, R^{1c} is

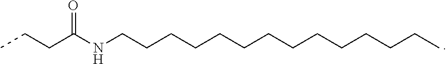

In certain embodiments, R^{1c} is

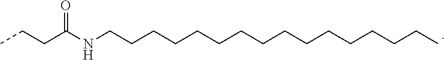

In certain embodiments, R^{1d} is

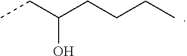

In certain embodiments, R^{1d} is

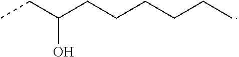

In certain embodiments, R^{1d} is

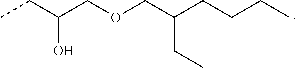

In certain embodiments R^{1d} is

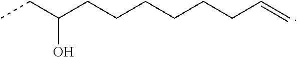

In certain embodiments, R^{1d} is

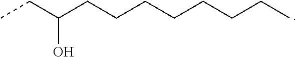

In certain embodiments, R^{1d} is

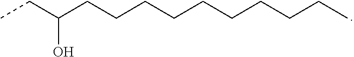

In certain embodiments, R^{1d} is

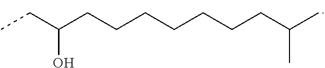

In certain embodiments, R^{1d} is

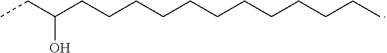

In certain embodiments, R^{1d} is

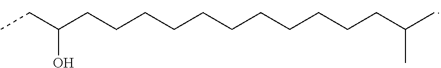

In certain embodiments, R^{1d} is

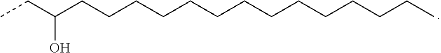

In certain embodiments, R^{1d} is

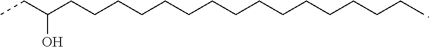

In certain embodiments, R^{1d} is

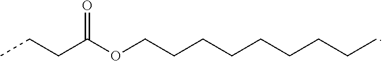

In certain embodiments, R^{1d} is

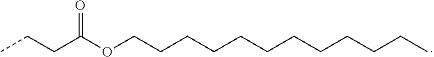

In certain embodiments, R^{1d} is

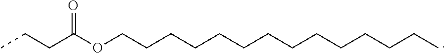

In certain embodiments, R^{1d} is

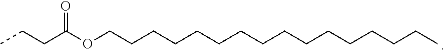

In certain embodiments, R^{1d} is

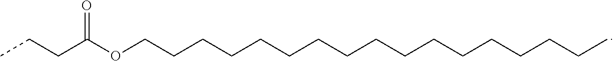

In certain embodiments, R^{1d} is

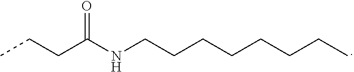

In certain embodiments, R^{1d} is

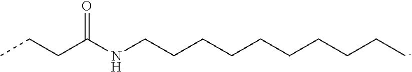

In certain embodiments, R^{1d} is

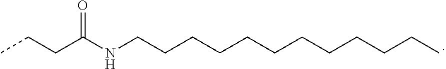

In certain embodiments, R^{1d} is

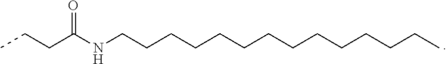

In certain embodiments, R^{1d} is

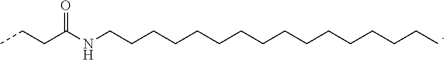

In certain embodiments, R^{6a} is —CH₂CHOH-(optionally substituted C₁-C₂₀ alkyl). In certain embodiments, R^{6a} is —CH₂CHOH-(optionally substituted C₁-C₂₀ heteroalkyl). In certain embodiments, R^{6a} is —CH₂CHOH-(optionally substituted C₂-C₂₀ alkenyl). In certain embodiments, R^{6a} is —CH₂CH₂C(═O)O(optionally substituted C₁-C₂₀ alkyl). In certain embodiments, R^{6a} is —CH₂CH₂C(═O)NH(optionally substituted C₁-C₂₀ alkyl).

In certain embodiments, R^{6b} is —CH₂CHOH-(optionally substituted C₁-C₂₀ alkyl). In certain embodiments, R^{6b} is —CH₂CHOH-(optionally substituted C₁-C₂₀ heteroalkyl). In certain embodiments, R^{6b} is —CH₂CHOH-(optionally substituted C₂-C₂₀ alkenyl). In certain embodiments, R^{6b} is —CH₂CH₂C(═O)O(optionally substituted C₁-C₂₀ alkyl). In certain embodiments, R^{6b} is —CH₂CH₂C(═O)NH(optionally substituted C₁-C₂₀ alkyl).

In certain embodiments, R^{6a} is

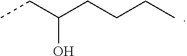

In certain embodiments, R^{6a} is

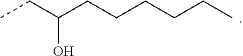

In certain embodiments, R^{6a} is

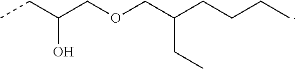

In certain embodiments, R^{6a} is

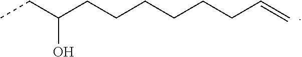

In certain embodiments, R^{6a} is

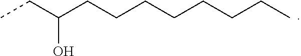

In certain embodiments, R^{6a} is

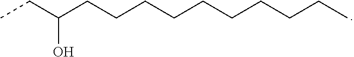

In certain embodiments, R^{6a} is

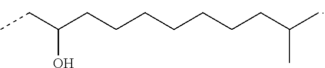

In cerin embodiments R^{6a} is

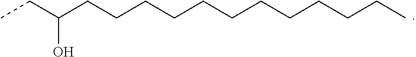

In certain embodiments, R^{6a} is

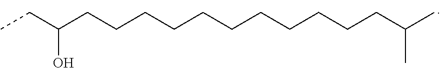

In certain embodiments, R^{6a} is

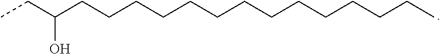

In certain embodiments, R^{6a} is

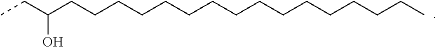

In certain embodiments, R^{6a} is

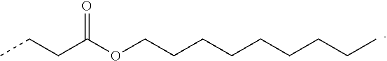

In certain embodiments, R^{6a} is

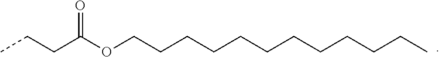

In certain embodiments, R^{6a} is

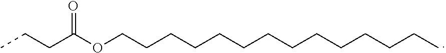

In certain embodiments, R^{6a} is

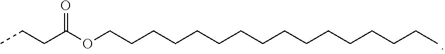

In certain embodiments, R^{6a} is

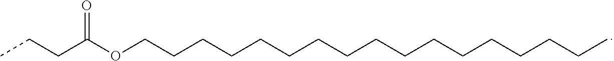

In certain embodiments, R^{6a} is

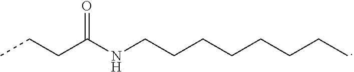

In certain embodiments, R^{6a} is

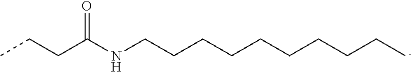

In certain embodiments, R^{6a} is

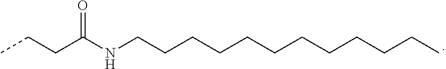

In certain embodiments, R^{6a} is

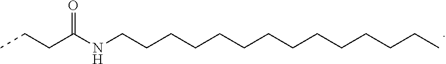

In certain embodiments, R^{6a} is

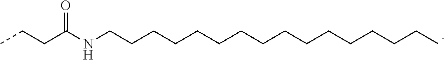

In certain embodiments, R^{6b} is

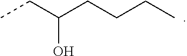

In certain embodiments, R^{6b} is

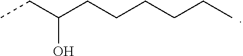

In certain embodiments, R^{6b} is

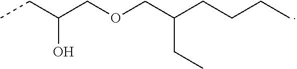

In certain embodiments, R^{6b} is

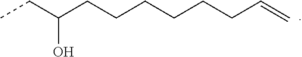

In certain embodiments, R^{6b} is

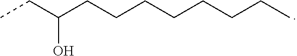

In certain embodiments, R^{6b} is

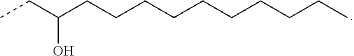

In certain embodiments, R^{6b} is

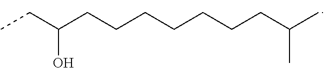

In certain embodiments, R^{6b} is

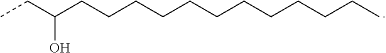

In certain embodiments, R^{6b} is

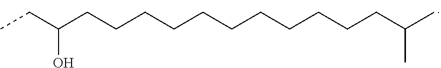

In certain embodiments, R^{6b} is

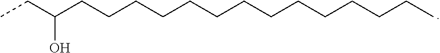

In certain embodiments, R^{6b} is

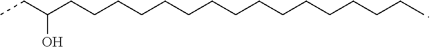

In certain embodiments, R^{6b} is

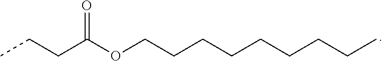

In certain embodiments, R^{6b} is

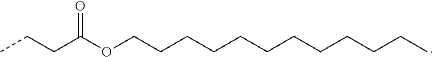

In certain embodiments, R^{6b} is

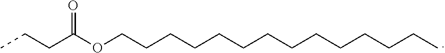

In certain embodiments, R^{6b} is

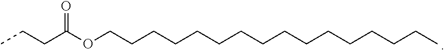

In certain embodiments, R^{6b} is

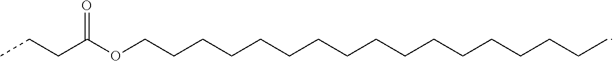

In certain embodiments, R^{6b} is

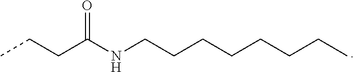

In certain embodiments, R^{6b} is

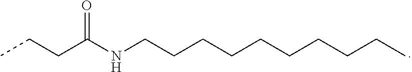

In certain embodiments, R^{6b} is

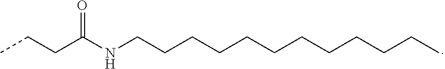

In certain embodiments, R^{6b} is

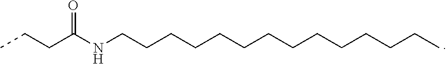

In certain embodiments, R^{6b} is

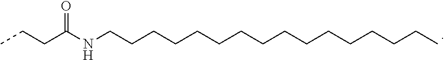

In certain embodiments, each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aralkyl, optionally substituted aryl, optionally substituted heteroaryl, and optionally substituted cyclosiloxane, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂OR′, S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of Formula (I) is:

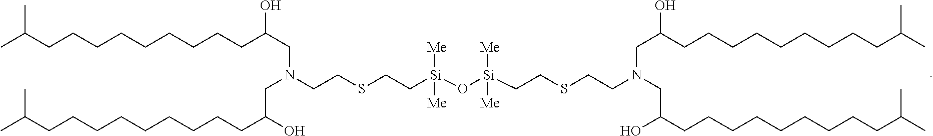 [SMILES: CC(C)CCCCCCCCCC(O)CN(CCSCC[Si](C)(C)O[Si](C)(C)CCSCCN(CC(O)CCCCCCCCCC(C)C)CC(O)CCCCCCCCCC(C)C)CC(O)CCCCCCCCCC(C)C]

In certain embodiments, the compound of Formula (I) is:

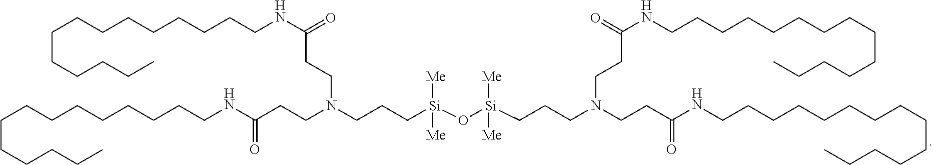 [SMILES: CCCCCCCCCCCCCCNC(=O)CCN(CCC[Si](C)(C)O[Si](C)(C)CCCN(CCC(=O)NCCCCCCCCCCCCCC)CCC(=O)NCCCCCCCCCCCCCC)CCC(=O)NCCCCCCCCCCCCCC]

In certain embodiments, the compound of Formula (I) is:

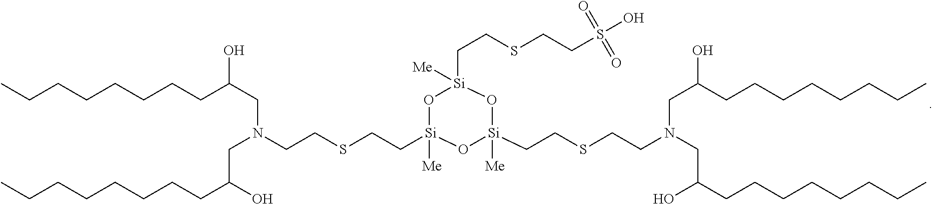 [SMILES: CCCCCCCCC(O)CN(CCSCC[Si]1(C)O[Si](C)(CCSCCN(CC(O)CCCCCCCC)CC(O)CCCCCCCC)O[Si](C)(CCSCCS(=O)(=O)O)O1)CC(O)CCCCCCCC]

In certain embodiments, the compound of Formula (I) is:

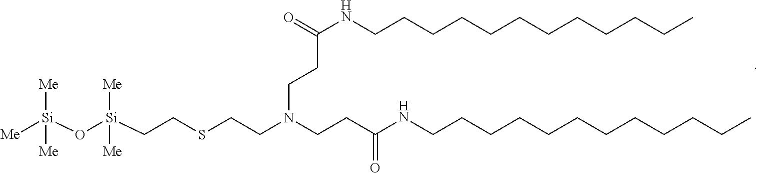 [SMILES: CCCCCCCCCCCCNC(=O)CCN(CCSCC[Si](C)(C)O[Si](C)(C)C)CCC(=O)NCCCCCCCCCCCC]

In certain embodiments, the compound of Formula (I) is:

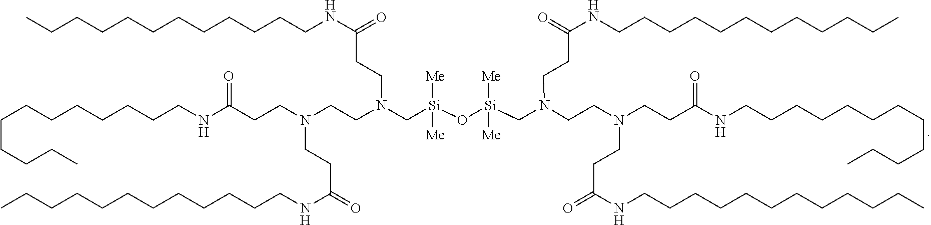 [SMILES: CCCCCCCCCCCCNC(=O)CCN(CCC(=O)NCCCCCCCCCCCC)CCN(CCC(=O)NCCCCCCCCCCCC)C[Si](C)(C)O[Si](C)(C)CN(CCC(=O)NCCCCCCCCCCCC)CCN(CCC(=O)NCCCCCCCCCCCC)CCC(=O)NCCCCCCCCCCCC]

As indicated elsewhere herein, the present disclosure relates to siloxane-based lipids and/or lipidoids comprising conjugated amino siloxanes (e.g., Si1, Si2, Si3, Si4, Si5, Si6, Si7, Si8, Si9, Si10, Si11, and Si12) and one or more tail groups (e.g., C6, C8, C11b, C10V, C10, C12, C12b, C14, C14b, C16, C18, O9, O12, O14, O16, O18, N8, N10, N12, N14, and N16).

The siloxane-based lipids and/or lipidoids disclosed herein my be described by reference to the amino siloxane and a tail groups, wherein the siloxane-based lipid and/or lipidoid comprises the product of nucleophilic addition of the amine of the amino siloxane to the epoxide moiety of the tail group (e.g., for epoxides) or the product of [1,4]-conjugate addition of the amine of the amino siloxane to the α,β-unsaturated moiety (e.g., for acrylates and/or acrylamides). For example, the terms “Si₅—N14” or “Si₅—N14 LNP”, as used herein, refers to a siloxane-based lipid and/or lipidoid comprising the Si5 amino siloxane and N14 acrylamide (see FIG. 1E), the structure of which is provided herein:

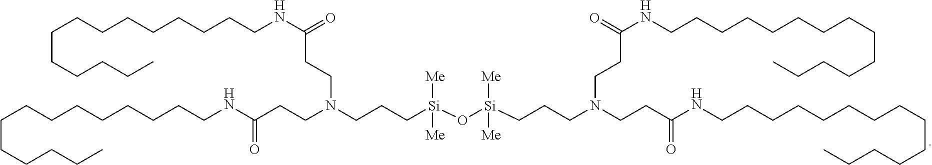 [SMILES: CCCCCCCCCCCCCCNC(=O)CCN(CCC[Si](C)(C)O[Si](C)(C)CCCN(CCC(=O)NCCCCCCCCCCCCCC)CCC(=O)NCCCCCCCCCCCCCC)CCC(=O)NCCCCCCCCCCCCCC]

Further, it is understood that siloxane-based lipids and/or lipids comprising each combination of amino siloxane groups Si1, Si2, Si3, Si4, Si5, Si6, Si7, Si8, Si9, Si10, Si11, and Si12, and tail groups C6, C8, C11b, C10V, C10, C12, C12b, C14, C14b, C16, C18, O9, O12, O14, O16, O18, N8, N10, N12, N14, and N16 is contemplated herein.

### Lipids

### Ionizable Lipids and or Cationic Lipids

The term “cationic lipid” as used herein refers to a lipid that is cationic or becomes cationic (protonated) as the pH is lowered below the pKa of the ionizable group of the lipid, but is progressively more neutral at higher pH values. At pH values below the pKa, the lipid is then able to associate with negatively charged nucleic acids. In certain embodiments, the cationic lipid comprises a zwitterionic lipid that assumes a positive charge on pH decrease.

In some embodiments, the cationic lipid comprises any of a number of lipid species which carry a net positive charge at a selective pH, such as physiological pH. Such lipids include, but are not limited to, N,N-dioleyl-N,N-dimethylammonium chloride (DODAC); N-(2,3-dioleyloxy)propyl)-N,N,N-trimethylammonium chloride (DOTMA); N,N-distearyl-N,N-dimethylammonium bromide (DDAB); N-(2,3-dioleoyloxy)propyl)-N,N,N-trimethylammonium chloride (DOTAP); 3-(N—(N′,N′-dimethylaminoethane)-carbamoyl)cholesterol (DC-Chol), N-(1-(2,3-dioleoyloxy)propyl)-N-2-(sperminecarboxamido)ethyl)-N,N-dimethylammonium trifluoracetate (DOSPA), dioctadecylamidoglycyl carboxyspermine (DOGS), 1,2-dioleoyl-3-dimethylammonium propane (DODAP), N,N-dimethyl-2,3-dioleoyloxy)propylamine (DODMA), and N-(1,2-dimyristyloxyprop-3-yl)-N,N-dimethyl-N-hydroxyethyl ammonium bromide (DMRIE). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (DOPE), from GIBCO/BRL, Grand Island, N.Y.); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (DOSPA) and (DOPE), from GIBCO/BRL); and TRANSFECTAM® (commercially available cationic lipids comprising dioctadecylamidoglycyl carboxyspermine (DOGS) in ethanol from Promega Corp., Madison, Wis.). The following lipids are cationic and have a positive charge at below physiological pH: DODAP, DODMA, DMDMA, 1,2-dilinoleyloxy-N,N-dimethylaminopropane (DLinDMA), 1,2-dilinolenyloxy-N,N-dimethylaminopropane (DLenDMA).

In some embodiments, the cationic lipid is an amino lipid. Suitable amino lipids useful in the invention include those described in WO 2012/016184, incorporated herein by reference in its entirety. Representative amino lipids include, but are not limited to, 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanediol (DOAP), 1,2-dilinoleyloxo-3-(2-N,N-dimethylamino)ethoxypropane (DLin-EG-DMA), and 2,2-dilinoleyl-4-dimethylaminomethyl-[1,3]-dioxolane (DLin-K-DMA).

### Neutral or Non-Cationic Lipid

In the nucleic acid-lipid particles of the present disclosure, the “neutral” or “non-cationic” lipid may comprise, e.g., one or more anionic lipids and/or neutral lipids. In some embodiments, the non-cationic lipid comprises one of the following neutral lipid components: (1) cholesterol or a derivative thereof (2) a phospholipid; or (3) a mixture of a phospholipid and cholesterol or a derivative thereof.

Examples of cholesterol derivatives include, but are not limited to, cholestanol, cholestanone, cholestenone, coprostanol, cholesteryl-2′-hydroxyethyl ether, cholesteryl-4′-hydroxybutyl ether, and mixtures thereof. The synthesis of cholesteryl-2′-hydroxyethyl ether is known to one skilled in the art and described in U.S. Pat. Nos. 8,058,069, 8,492,359, 8,822,668, 9,364,435, 9,504,651, and 11,141,378, all of which are hereby incorporated herein in their entireties for all purposes.

Non-limiting examples of non-cationic lipids include phospholipids such as lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidic acid, cerebrosides, dicetylphosphate, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), ioleoylphosphatidylethanolamine (DOPE), palmitoyloleoylphosphatidylcholine (POPC), palmitoyloleoylphosphatidylethanolamine (POPE), palmitoyloleyolphosphatidylglycerol (POPG), dioleoylphosphatidylethanolamine 4-(N-maleimidomethyl)-cyclohexane-1-carboxylate DOPE-mal), dipalmitoylphosphatidylethanolamine (DPPE), dimyristoylphosphatidylethanolamine (DMPE), distearoylphosphatidylethanolamine (DSPE), monomethylphosphatidylethanolamine, dimethylphosphatidylethanolamine, dielaidoylphosphatidylethanolamine (DEPE), stearoyloleoylphosphatidylethanolamine (SOPE), lysophosphatidylcholine, dilinoleoylphosphatidylcholine, and mixtures thereof.

Other diacylphosphatidylcholine and diacylphosphatidylethanolamine phospholipids can also be used. The acyl groups in these lipids can be, for example, acyl groups derived from fatty acids having C₁₀-C₂₄ carbon chains, e.g., lauroyl, myristoyl, palmitoyl, stearoyl, or oleoyl. Additional examples of non-cationic lipids include sterols such as cholesterol and derivatives thereof such as cholestanol, cholestanone, cholestenone, coprostanol, cholesteryl-2′-hydroxyethyl ether, cholesteryl-4′-hydroxybutyl ether, and mixtures thereof. In certain embodiments, the phospholipid is DPPC, DSPC, or mixtures thereof.

### Conjugated Lipid

In the nucleic acid-lipid particles of the present disclosure, the conjugated lipid that inhibits aggregation of particles may comprise, e.g., one or more of the following: a polyethyleneglycol (PEG) lipid conjugate, a polyamide (ATTA)-lipid conjugate, a cationic-polymer-lipid conjugates (CPLs), or mixtures thereof. In some embodiments, the nucleic acid-lipid particles comprise either a PEG-lipid conjugate or an ATTA-lipid conjugate.

PEG is a linear, water-soluble polymer of ethylene PEG repeating units with two terminal hydroxyl groups. PEGs are classified by their molecular weights; for example, PEG 2000 has an average molecular weight of about 2,000 daltons, and PEG 5000 has an average molecular weight of about 5,000 daltons. PEGs are commercially available from Sigma Chemical Co. and other companies and include, for example, the following: monomethoxypolyethylene glycol (MePEGOH), monomethoxypolyethylene glycolsuccinate (MePEGS), monomethoxypolyethylene glycolsuccinimidyl succinate (MePEG-S-NHS), monomethoxypolyethylene glycolamine (MePEG-NH₂), monomethoxypolyethylene glycoltresylate (MePEG-TRES), and monomethoxypolyethylene glycolimidazolylcarbonyl (MePEG-IM). Other PEGs such as those described in U.S. Pat. Nos. 6,774,180 and 7,053,150 (e.g., mPEG (20 KDa) amine) are also useful for preparing the PEG-lipid conjugates of the present disclosure. The disclosures of these patents are herein incorporated by reference in their entirety for all purposes. In addition, monomethoxypolyethyleneglycolacetic acid (MePEG-CH₂COOH) is particularly useful for preparing PEG-lipid conjugates including, e.g., PEG-DAA conjugates.

In certain embodiments, the PEG-lipid conjugate or ATTA-lipid conjugate is used together with a CPL. The conjugated lipid that inhibits aggregation of particles may comprise a PEG-lipid including, e.g., a PEG-diacylglycerol (DAG), a PEG dialkyloxypropyl (DAA), a PEG-phospholipid, a PEG-ceramide (Cer), or mixtures thereof. The PEGDAA conjugate may be PEG-dilauryloxypropyl (C₁₂), a PEG-dimyristyloxypropyl (C₁₄), a PEG-dipalmityloxypropyl (C₁₆), a PEG-distearyloxypropyl (C₁₈), or mixtures thereof.

Additional PEG-lipid conjugates suitable for use in the disclosure include, but are not limited to, mPEG2000-1,2-diO-alkyl-sn3-carbomoylglyceride (PEG-C-DOMG). The synthesis of PEG-C-DOMG is described in PCT Application No. PCT/US08/88676, filed Dec. 31, 2008, the disclosure of which is herein incorporated by reference in its entirety for all purposes. Yet additional PEG-lipid conjugates suitable for use in the disclosure include, without limitation, 1-[8′-(1,2-dimyristoyl-3-propanoxy)-carboxamido-3′,6′-dioxaoctanyl]carbamoyl-methyl-poly(ethylene glycol) (2 KPEG-DMG). The synthesis of 2 KPEG-DMG is described in U.S. Pat. No. 7,404,969, the disclosure of which is herein incorporated by reference in its entirety for all purposes.

The PEG moiety of the PEG-lipid conjugates described herein may comprise an average molecular weight ranging from about 550 daltons to about 10,000 daltons. In certain instances, the PEG moiety has an average molecular weight of from about 750 daltons to about 5,000 daltons (e.g., from about 1,000 daltons to about 5,000 daltons, from about 1,500 daltons to about 3,000 daltons, from about 750 daltons to about 3,000 daltons, from about 750 daltons to about 2,000 daltons, etc.). In some embodiments, the PEG moiety has an average molecular weight of about 2,000 daltons or about 750 daltons.

In addition to the foregoing, it will be readily apparent to those of skill in the art that other hydrophilic polymers can be used in place of PEG. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses such as hydroxymethylcellulose or hydroxyethylcellulose.

In addition to the foregoing components, the particles (e.g., LNP) of the present disclosure can further comprise cationic poly(ethylene glycol) (PEG) lipids or CPLs (e.g., Chen et al., Bioconj. Chem., 11:433-437 (2000)). Suitable SPLPs and SPLP-CPLs for use in the present disclosure, and methods of making and using SPLPs and SPLP-CPLs, are disclosed, e.g., in U.S. Pat. No. 6,852,334 and PCT Publication No. WO 00/62813, the disclosures of which are herein incorporated by reference in their entirety for all purposes.

In certain instances, the conjugated lipid that inhibits aggregation of particles (e.g., PEG-lipid conjugate) may comprise from about 0.1 mol % to about 2 mol %, from about 0.5 mol % to about 2 mol %, from about 1 mol % to about 2 mol %, from about 0.6 mol % to about 1.9 mol %, from about 0.7 mol % to about 1.8 mol %, from about 0.8 mol % to about 1.7 mol %, from about 1 mol % to about 1.8 mol %, from about 1.2 mol % to about 1.8 mol %, from about 1.2 mol % to about 1.7 mol %, from about 1.3 mol % to about 1.6 mol %, from about 1.4 mol % to about 1.5 mol %, or about 1, 1.1, 1.2, 1.3, 1.4, 1.5, 1.6, 1.7, 1.8, 1.9, or 2 mol % (or any fraction thereof or range therein) of the total lipid present in the particle.

In the nucleic acid-lipid particles of the present disclosure, the active agent or therapeutic agent may be fully encapsulated within the lipid portion of the particle, thereby protecting the active agent or therapeutic agent from enzymatic degradation. In some embodiments, a nucleic acid-lipid particle comprising a nucleic acid such as a messenger RNA (i.e., mRNA) is fully encapsulated within the lipid portion of the particle, thereby protecting the nucleic acid from nuclease degradation. In certain instances, the nucleic acid in the nucleic acid-lipid particle is not substantially degraded after exposure of the particle to a nuclease at 37° C. for at least about 20, 30, 45, or 60 minutes. In certain other instances, the nucleic acid in the nucleic acid-lipid particle is not substantially degraded after incubation of the particle in serum at 37° C. for at least about 30, 45, or 60 minutes or at least about 2, 3, 4, 5, 6, 7, 8, 9, 10, 12, 14, 16, 18, 20, 22, 24, 26, 28, 30, 32, 34, or 36 hours. In other embodiments, the active agent or therapeutic agent (e.g., nucleic acid such as mRNA) is complexed with the lipid portion of the particle. One of the benefits of the formulations of the present disclosure is that the lipid particle compositions are substantially non-toxic to mammals such as humans.

### Lipid Nanoparticle (LNP) Compositions

The present disclosure relates, in another aspect, to LNP compositions which selectively target at least one cell (e.g., tissue cell, muscle cell, immune cell, endothelial cell, and epithelial cell, inter alia) or organ of interest (e.g., liver, heart, lungs, spleen, and intestine, inter alia), wherein the LNP comprises:
(a) at least one compound of Formula (I), or a salt, solvate, stereoisomer, or isotopologue thereof:

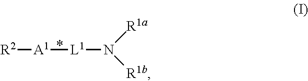

wherein:
A is

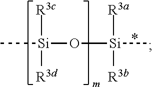

R² is selected from the group consisting of

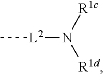

optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of R^{1a}, R^{1b}, R^{1c}, and R^{1d}, if present, is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂ alkynyl;
L¹ and L² are each independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocycloalkylenyl;
each occurrence of R^{3a}, R^{3b}, R^{3c}, and R^{3d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl,
   wherein two occurrences of R^{3c} or two occurrences of R^{3d} can combine with the atoms to which they are bound to form an optionally substituted 6-10 membered cyclosiloxane, or
   wherein R^{3a} and R^{3c}, R^{3a} and R^{3d}, R^{3b} and R^{3c}, or R^{3b} and R^{3d} can combine with the atoms to which they are bound to form an optionally substituted 6-10 membered cyclosiloxane;
each occurrence of R^{A} is independently selected from the group consisting of optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl; and
m is an integer ranging from 0 to 50;
(b) at least one neutral phospholipid;
(c) at least one cholesterol lipid; and
(d) at least one selected from the group consisting of polyethylene glycol (PEG) and a PEG-conjugated lipid.

In certain embodiments, A is selected from the group consisting of:

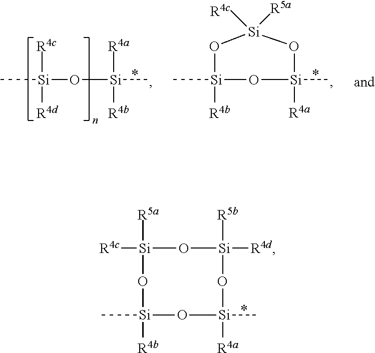

wherein:
R^{4a}, R^{4b}, R^{4c}, and R^{4d}, if present, are each independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{5a} and R^{5b}, if present, are each independently selected from the group consisting of

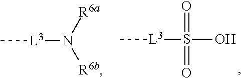

optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of L³ is independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{6a} and R^{6b} is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl; and
n is an integer ranging from 0 to 30.

In certain embodiments, R^{4a} is Me. In certain embodiments, R^{4a} is OSiMe₃. In certain embodiments, R^{4b} is Me. In certain embodiments, R^{4b} is OSiMe₃. In certain embodiments, R^{4c} is Me. In certain embodiments, R^{4c} is OSiMe₃. In certain embodiments, R^{4d} is Me. In certain embodiments, R^{4d} is OSiMe₃.

In certain embodiments, A is

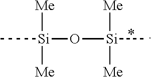

In certain embodiments, A is

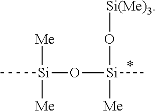

In certain embodiments, A is

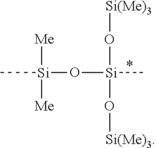

In certain embodiments, A is

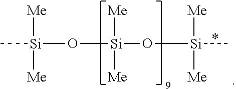

In certain embodiments, A is

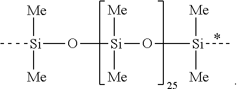

In certain embodiments, A is

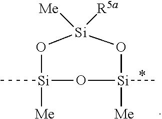

In certain embodiments, A is

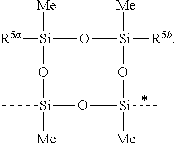

In certain embodiments, L¹ is —(CH₂)₁₋₅S(CH₂)₁₋₅—. In certain embodiments, L¹ is —(CH₂)₁₋₅—. In certain embodiments, L¹ is —(CH₂)₁₋₅N(R^{6a})(CH₂)₁₋₅—.

In certain embodiments, L² is —(CH₂)₁₋₅S(CH₂)₁₋₅—. In certain embodiments, L² is —(CH₂)₁₋₅—. In certain embodiments, L² is —(CH₂)₁₋₅N(R^{6a})(CH₂)₁₋₅—.

In certain embodiments, L³ is —(CH₂)₁₋₅S(CH₂)₁₋₅—. In certain embodiments, L³ is —(CH₂)₁₋₅—. In certain embodiments, L³ is —(CH₂)₁₋₅N(R^{6a})(CH₂)₁₋₅—.

In certain embodiments, L¹ is —(CH₂)₃—. In certain embodiments, L¹ is —(CH₂)₂S(CH₂)₂—. In certain embodiments, L¹ is —(CH₂)NR^{6a}(CH₂)₂—.

In certain embodiments, L² is —(CH₂)₃—. In certain embodiments, L² is —(CH₂)₂S(CH₂)₂—. In certain embodiments, L² is —(CH₂)NR^{6a}(CH₂)₂—.

In certain embodiments, L³ is —(CH₂)₃—. In certain embodiments, L³ is —(CH₂)₂S(CH₂)₂—. In certain embodiments, L³ is —(CH₂)NR^{6a}(CH₂)₂—.

In certain embodiments, the compound of Formula (I) is

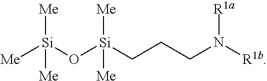

In certain embodiments, the compound of Formula (I) is

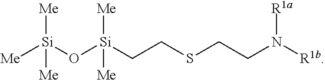

In certain embodiments, the compound of Formula (I) is

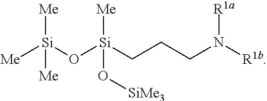

In certain embodiments, the compound of Formula (I) is

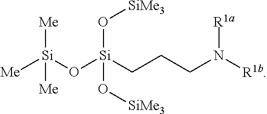

In certain embodiments, the compound of Formula (I) is

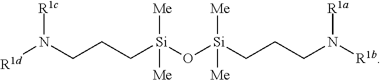

In certain embodiments, the compound of Formula (I) is

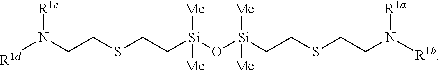

In certain embodiments, the compound of Formula (I) is

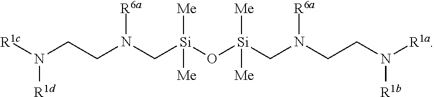

In certain embodiments, the compound of Formula (I) is

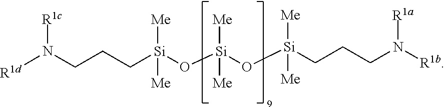

In certain embodiments, the compound of Formula (I) is

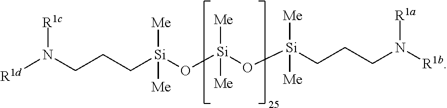

In certain embodiments, the compound of Formula (I) is

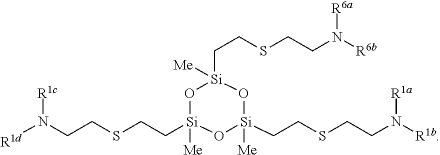

In certain embodiments, the compound of Formula (I) is

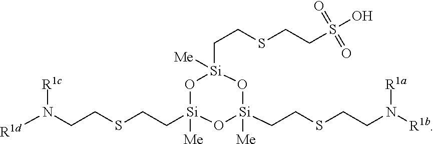

In certain embodiments, the compound of Formula (I) is

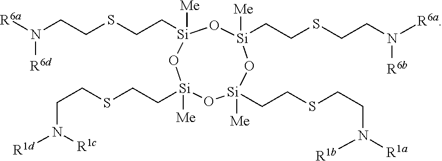

In certain embodiments, R^{1a} is —CH₂CHOH-(optionally substituted C₁-C₂₀ alkyl). In certain embodiments, R^{1a} is —CH₂CHOH-(optionally substituted C₁-C₂₀ heteroalkyl). In certain embodiments, R^{1a} is —CH₂CHOH-(optionally substituted C₂-C₂₀ alkenyl). In certain embodiments, R^{1a} is —CH₂CH₂C(═O)O(optionally substituted C₁-C₂₀ alkyl). In certain embodiments, R^{1a} is —CH₂CH₂C(═O)NH(optionally substituted C₁-C₂₀ alkyl).

In certain embodiments, R^{1b} is —CH₂CHOH-(optionally substituted C₁-C₂₀ alkyl). In certain embodiments, R^{1b} is —CH₂CHOH-(optionally substituted C₁-C₂₀ heteroalkyl). In certain embodiments, R^{1b} is —CH₂CHOH-(optionally substituted C₂-C₂₀ alkenyl). In certain embodiments, R^{1b} is —CH₂CH₂C(═O)O(optionally substituted C₁-C₂₀ alkyl). In certain embodiments, R^{1b} is —CH₂CH₂C(═O)NH(optionally substituted C₁-C₂₀ alkyl).

In certain embodiments, R^{1c} is —CH₂CHOH-(optionally substituted C₁-C₂₀ alkyl). In certain embodiments, R^{1b} is —CH₂CHOH-(optionally substituted C₁-C₂₀ heteroalkyl). In certain embodiments, R^{1b} is —CH₂CHOH-(optionally substituted C₂-C₂₀ alkenyl). In certain embodiments, R^{1c} is —CH₂CH₂C(═O)O(optionally substituted C₁-C₂₀ alkyl). In certain embodiments, R^{1c} is —CH₂CH₂C(═O)NH(optionally substituted C₁-C₂₀ alkyl).

In certain embodiments, R^{1d} is —CH₂CHOH-(optionally substituted C₁-C₂₀ alkyl). In certain embodiments, R^{1d} is —CH₂CHOH-(optionally substituted C₁-C₂₀ heteroalkyl). In certain embodiments, R^{1d} is —CH₂CHOH-(optionally substituted C₂-C₂₀ alkenyl). In certain embodiments, R^{1d} is —CH₂CH₂C(═O)O(optionally substituted C₁-C₂₀ alkyl). In certain embodiments, R^{1d} is —CH₂CH₂C(═O)NH(optionally substituted C₁-C₂₀ alkyl).

In certain embodiments, R^{1a} is

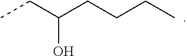

In certain embodiments, R^{1a} is

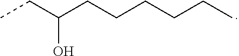

In certain embodiments, R^{1a} is

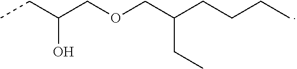

In certain embodiments, R^{1a} is

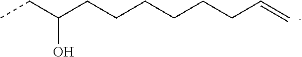

In certain embodiments R^{1a} is

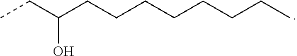

In certain embodiments, R^{1a} is

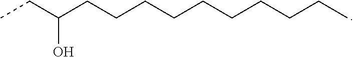

In certain embodiments, R^{1a} is

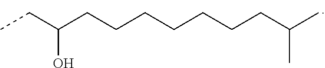

In certain embodiments, R^{1a} is

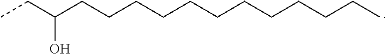

In certain embodiments, R^{1a} is

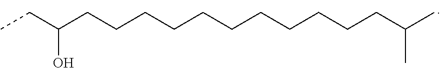

In certain embodiments, R^{1a} is

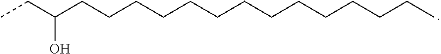

In certain embodiments, R^{1a} is

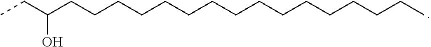

In certain embodiments, R^{1a} is

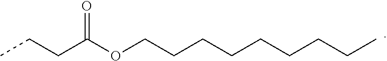

In certain embodiments, R^{1a} is

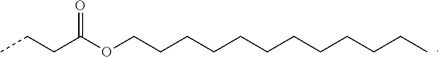

In certain embodiments, R^{1a} is

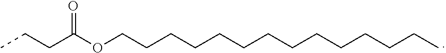

In certain embodiments, R^{1a} is

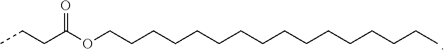

In certain embodiments, R^{1a} is

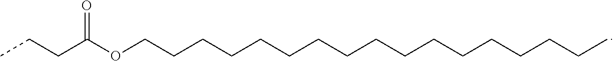

In certain embodiments, R^{1a} is

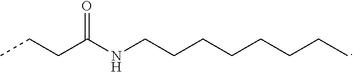

In certain embodiments, R^{1a} is

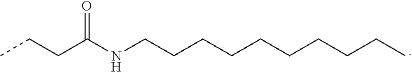

In certain embodiments, R^{1a} is

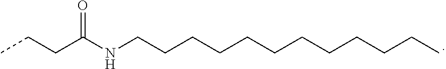

In certain embodiments, R^{1a} is

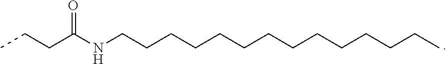

In certain embodiments, R^{1a} is

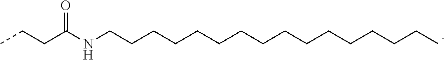

In certain embodiments, R^{1b} is

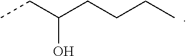

In certain embodiments, R^{1b} is

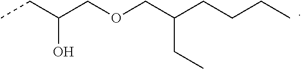

In certain embodiments, R^{1b} is

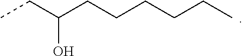

In certain embodiments R^{1b} is

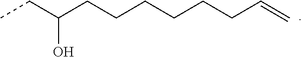

In certain embodiments, R^{1b} is

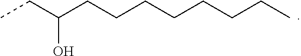

In certain embodiments, R^{1b} is

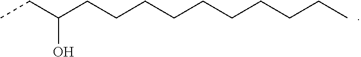

In certain embodiments, R^{1b} is

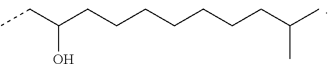

In certain embodiments, R^{1b} is

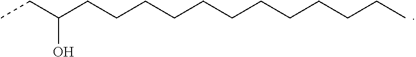

In certain embodiments, R^{1b} is

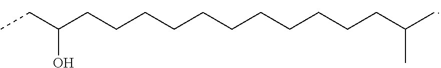

In certain embodiments, R^{1b} is

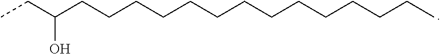

In certain embodiments, R^{1b} is

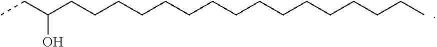

In certain embodiments, R^{1b} is

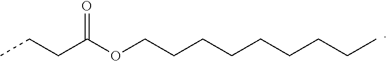

In certain embodiments, R^{1b} is

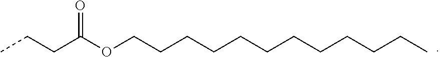

In certain embodiments, R^{1b} is

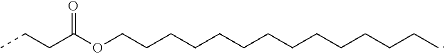

In certain embodiments, R^{1b} is

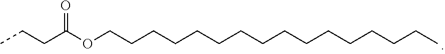

In certain embodiments, R^{1b} is

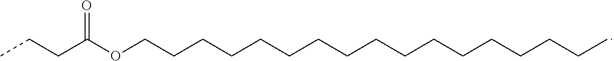

In certain embodiments, R^{1b} is

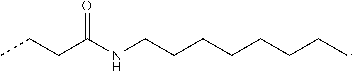

In certain embodiments, R^{1b} is

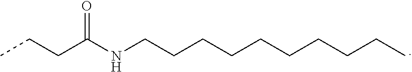

In certain embodiments, R^{1b} is

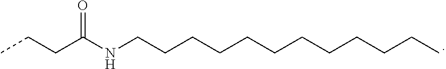

In certain embodiments, R^{1b} is

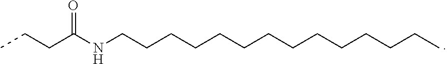

In certain embodiments, R^{1b} is

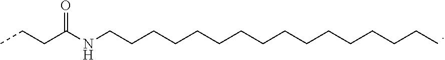

In certain embodiments, R^{1c} is

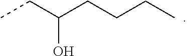

In certain embodiments, R^{1c} is

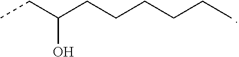

In certain embodiments, R^{1c} is

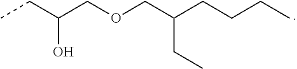

In certain embodiments, R^{1c} is

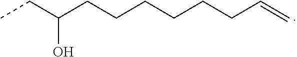

In certain embodiments, R^{1c} is

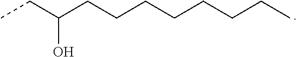

In certain embodiments, R^{1b} is

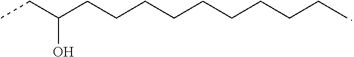

In certain embodiments, R^{1c} is

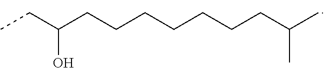

In certain embodiments, R^{1c} is

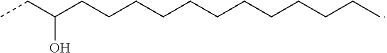

In certain embodiments, R^{1c} is

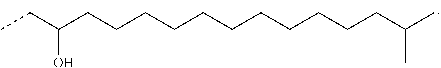

In certain embodiments, R^{1c} is

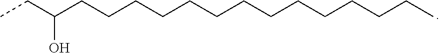

In certain embodiments, R^{1c} is

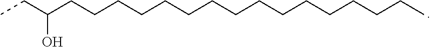

In certain embodiments, R^{1c} is

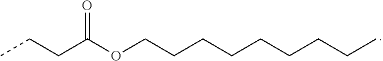

In certain embodiments, R^{1c} is

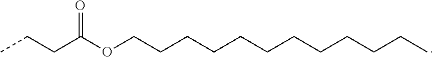

In certain embodiments, R^{1c} is

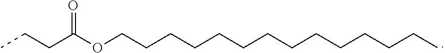

In certain embodiments, R^{1c} is

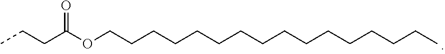

In certain embodiments, R^{1c} is

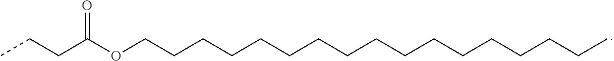

In certain embodiments, R^{1c} is

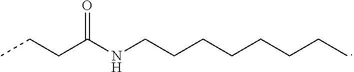

In certain embodiments, R^{1c} is

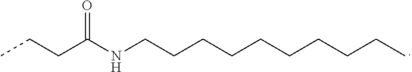

In certain embodiments, R^{1c} is

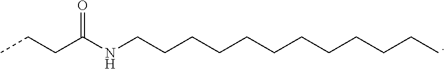

In certain embodiments, R^{1c} is

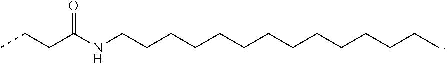

In certain embodiments, R^{1c} is

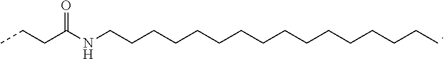

In certain embodiments, R^{1d} is

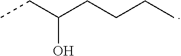

In certain embodiments, R^{1d} is

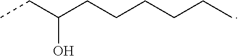

In certain embodiments, R^{1d} is

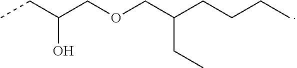

In certain embodiments, R^{1d} is

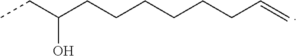

In certain embodiments, R^{1d} is

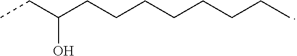

In certain embodiments R^{1d} is

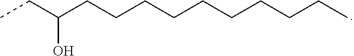

In certain embodiments, R^{1d} is

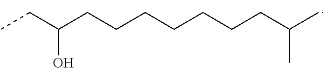

In certain embodiments, R^{1d} is

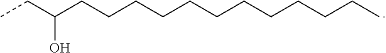

In certain embodiments, R^{1d} is

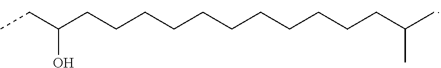

In certain embodiments, R^{1d} is

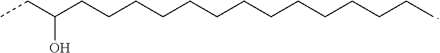

In certain embodiments, R^{1d} is

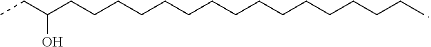

In certain embodiments, R^{1d} is

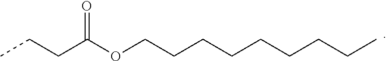

In certain embodiments, R^{1d} is

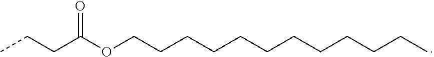

In certain embodiments, R^{1d} is

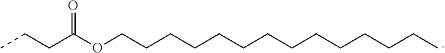

In certain embodiments, R^{1d} is

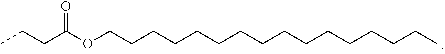

In certain embodiments, R^{1d} is

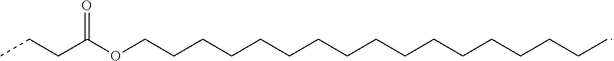

In certain embodiments, R^{1d} is

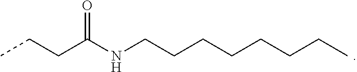

In certain embodiments, R^{1d} is

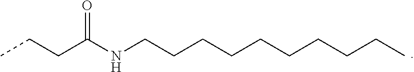

In certain embodiments, R^{1d} is

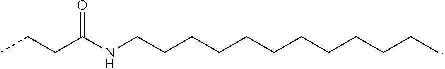

In certain embodiments, R^{1d} is

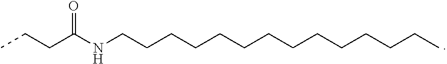

In certain embodiments, R^{1d} is

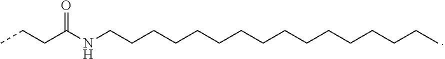

In certain embodiments, R^{6a} is —CH₂CHOH-(optionally substituted C₁-C₂₀ alkyl). In certain embodiments, R^{6a} is —CH₂CHOH-(optionally substituted C₁-C₂₀ heteroalkyl). In certain embodiments, R^{6a} is —CH₂CHOH-(optionally substituted C₂-C₂₀ alkenyl). In certain embodiments, R^{6a} is —CH₂CH₂C(═O)O(optionally substituted C₁-C₂₀ alkyl). In certain embodiments, R^{6a} is —CH₂CH₂C(═O)NH(optionally substituted C₁-C₂₀ alkyl).

In certain embodiments, R^{6b} is —CH₂CHOH-(optionally substituted C₁-C₂₀ alkyl). In certain embodiments, R^{6b} is —CH₂CHOH-(optionally substituted C₁-C₂₀ heteroalkyl). In certain embodiments, R^{6b} is —CH₂CHOH-(optionally substituted C₂-C₂₀ alkenyl). In certain embodiments, R^{6b} is —CH₂CH₂C(═O)O(optionally substituted C₁-C₂₀ alkyl). In certain embodiments, R^{6b} is —CH₂CH₂C(═O)NH(optionally substituted C₁-C₂₀ alkyl).

In certain embodiments, R^{6a} is

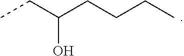

In certain embodiments, R^{6a} is

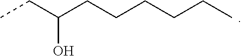

In certain embodiments, R^{6a} is

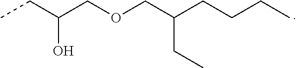

In certain embodiments, R^{6a} is

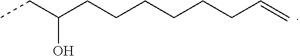

In certain embodiments R^{6a} is

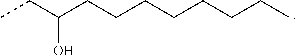

In certain embodiments, R^{6a} is

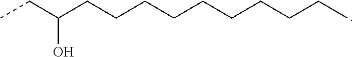

In certain embodiments, R^{6a} is

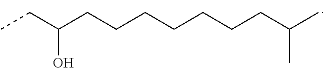

In certain embodiments, R^{6a} is

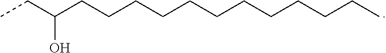

In certain embodiments, R^{6a} is

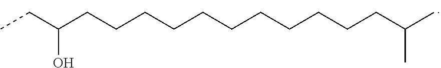

In certain embodiments, R^{6a} is

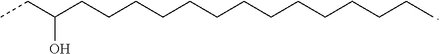

In certain embodiments, R^{6a} is

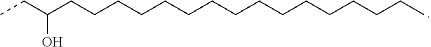

In certain embodiments, R^{6a} is

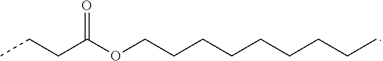

In certain embodiments, R^{6a} is

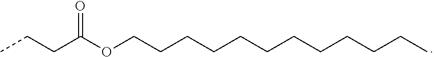

In certain embodiments, R^{6a} is

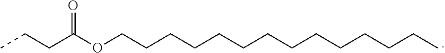

In certain embodiments, R^{6a} is

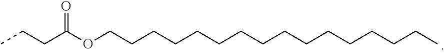

In certain embodiments, R^{6a} is

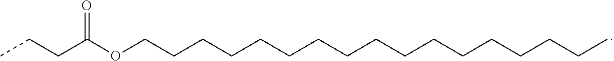

In certain embodiments, R^{6a} is

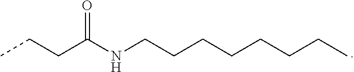

In certain embodiments, R^{6a} is

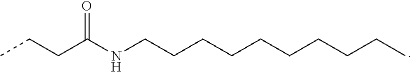

In certain embodiments, R^{6a} is

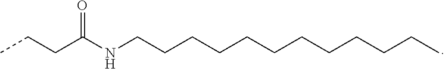

In certain embodiments, R^{6a} is

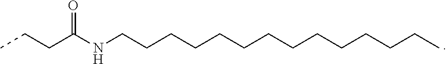

In certain embodiments, R^{6a} is

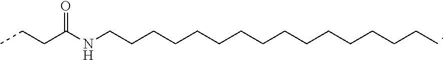

In certain embodiments, R^{6b} is

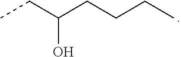

In certain embodiments, R^{6b} is

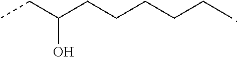

In certain embodiments, R^{6b} is

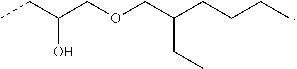

In certain embodiments, R^{6b} is

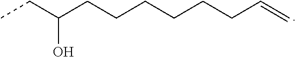

In certain embodiments, R^{6b} is

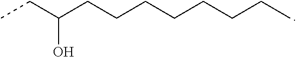

In certain embodiments, R^{6b} is

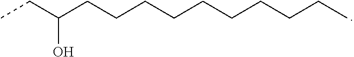

In certain embodiments, R^{6b} is

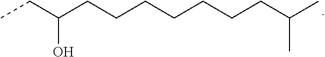

In certain embodiments, R^{6b} is

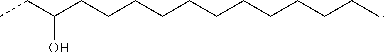

In certain embodiments, R^{6b} is

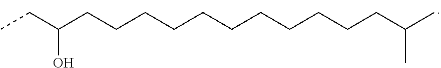

In certain embodiments, R^{6b} is

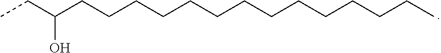

In certain embodiments, R^{6b} is

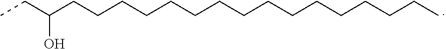

In certain embodiments, R^{6b} is

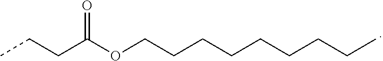

In certain embodiments, R^{6b} is

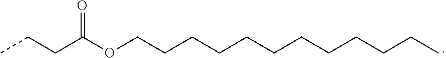

In certain embodiments, R^{6b} is

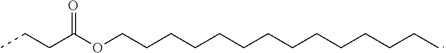

In certain embodiments, R^{6b} is

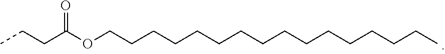

In certain embodiments, R^{6b} is

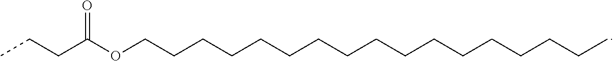

In certain embodiments, R^{6b} is

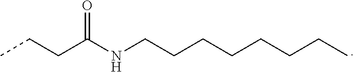

In certain embodiments, R^{6b} is

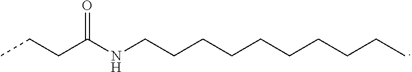

In certain embodiments, R^{6b} is

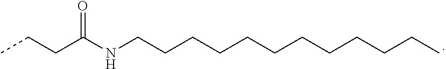

In certain embodiments, R^{6b} is

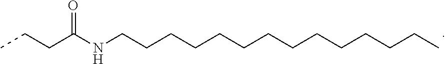

In certain embodiments, R^{6b} is

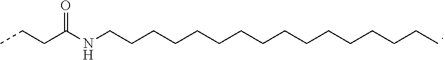

In certain embodiments, each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aralkyl, optionally substituted aryl, optionally substituted heteroaryl, and optionally substituted cyclosiloxane, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂OR′, S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

In certain embodiments, the compound of Formula (I) is:

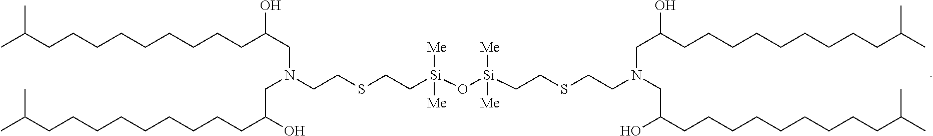 [SMILES: CC(C)CCCCCCCCCC(O)CN(CCSCC[Si](C)(C)O[Si](C)(C)CCSCCN(CC(O)CCCCCCCCCC(C)C)CC(O)CCCCCCCCCC(C)C)CC(O)CCCCCCCCCC(C)C]

In certain embodiments, the compound of Formula (I) is:

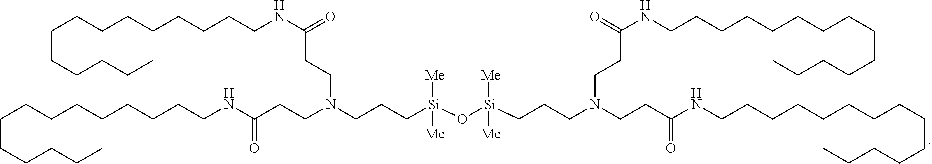 [SMILES: CCCCCCCCCCCCCCNC(=O)CCN(CCC[Si](C)(C)O[Si](C)(C)CCCN(CCC(=O)NCCCCCCCCCCCCCC)CCC(=O)NCCCCCCCCCCCCCC)CCC(=O)NCCCCCCCCCCCCCC]

In certain embodiments the compound of Formula (I) is:

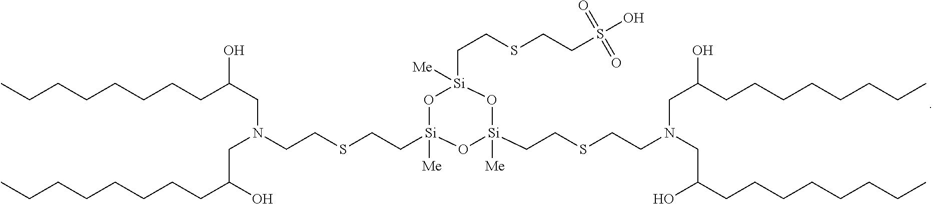 [SMILES: CCCCCCCCC(O)CN(CCSCC[Si]1(C)O[Si](C)(CCSCCN(CC(O)CCCCCCCC)CC(O)CCCCCCCC)O[Si](C)(CCSCCS(=O)(=O)O)O1)CC(O)CCCCCCCC]

In certain embodiments, the compound of Formula (I) is:

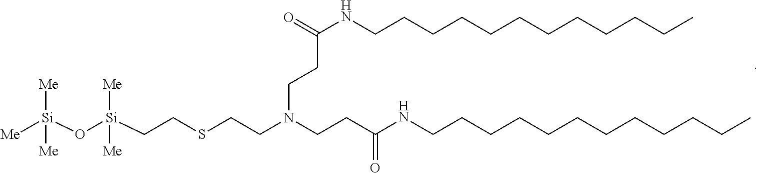 [SMILES: CCCCCCCCCCCCNC(=O)CCN(CCSCC[Si](C)(C)O[Si](C)(C)C)CCC(=O)NCCCCCCCCCCCC]

In certain embodiments, the compound of Formula (I) is:

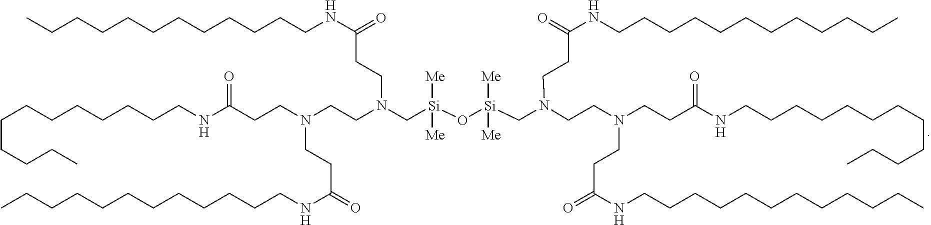 [SMILES: CCCCCCCCCCCCNC(=O)CCN(CCC(=O)NCCCCCCCCCCCC)CCN(CCC(=O)NCCCCCCCCCCCC)C[Si](C)(C)O[Si](C)(C)CN(CCC(=O)NCCCCCCCCCCCC)CCN(CCC(=O)NCCCCCCCCCCCC)CCC(=O)NCCCCCCCCCCCC]

In certain embodiments, the LNP further comprises at least one cargo.

In certain embodiments, the cargo is partially encapsulated by the LNP. In certain embodiments, the cargo is fully encapsulated by the LNP.

In certain embodiments, the cargo is a nucleic acid molecule. In certain embodiments, the cargo is a small molecule. In certain embodiments, the cargo is a protein. In certain embodiments, the cargo is a therapeutic agent. In certain embodiments, the cargo is an antibody.

In certain embodiments, the nucleic acid molecule is a DNA molecule. In certain embodiments, the nucleic acid molecule is a RNA molecule.

In certain embodiments, the nucleic acid molecule is mRNA. In certain embodiments, the nucleic acid molecule is cDNA. In certain embodiments, the nucleic acid molecule is pDNA. In certain embodiments, the nucleic acid molecule is microRNA. In certain embodiments, the nucleic acid molecule is siRNA. In certain embodiments, the nucleic acid molecule is modified RNA. In certain embodiments, the nucleic acid molecule is an antagomir. In certain embodiments, the nucleic acid molecule is an antisense molecule. In certain embodiments, the nucleic acid molecule is a targeted nucleic acid.

In certain embodiments, the compound of Formula (I) comprises about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or about 99 mol % of the LNP.

In certain embodiments, the compound of Formula (I) comprises about 35 mol % of the LNP. In certain embodiments, the compound of Formula (I) comprises less than about 35 mol % of the LNP. In certain embodiments, the compound of Formula (I) comprises more than about 35 mol % of the LNP.

In certain embodiments, the neutral phospholipid comprises dioleoylphosphatidylethanolamine (DOPE). In certain embodiments, the neutral phospholipid comprises distearoylphosphatidylcholine (DSPC). In certain embodiments, the neutral phospholipid comprises dioleoylphosphatidylcholine (DOPC). In certain embodiments, the neutral phospholipid comprises distearoyl-phosphatidylethanolamine (DSPE). In certain embodiments, the neutral phospholipid comprises stearoyloleoylphosphatidylcholine (SOPC). In certain embodiments, the neutral phospholipid comprises 1-stearioyl-2-oleoyl-phosphatidyethanol amine (SOPE). In certain embodiments, the neutral phospholipid comprises N-(2,3-dioleoyloxy)propyl)-N,N,N-trimethylammonium chloride (DOTAP).

In certain embodiments, the neutral phospholipid is dioleoylphosphatidylethanolamine (DOPE).

In certain embodiments, the at least one neutral phospholipid comprises about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the at least one neutral phospholipid comprises less than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP. In certain embodiments, the at least one neutral phospholipid comprises more than about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, or about 45 mol % of the LNP.

In certain embodiments, the at least one neutral phospholipid comprises about 16 mol % of the LNP. In certain embodiments, the at least one neutral phospholipid comprises less than about 16 mol % of the LNP. In certain embodiments, the at least one neutral phospholipid comprises more than about 16 mol % of the LNP.

In certain embodiments, the cholesterol lipid is cholesterol.

In certain embodiments, the cholesterol lipid comprises about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the cholesterol lipid comprises less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP. In certain embodiments, the cholesterol lipid comprises more than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or about 50 mol % of the LNP.

In certain embodiments, the cholesterol lipid comprises about 46.5 mol % of the LNP.

In certain embodiments, the cholesterol lipid comprises less than about 46.5 mol % of the LNP. In certain embodiments, the cholesterol lipid comprises more than about 46.5 mol % of the LNP.

In certain embodiments, the polyethylene glycol (PEG) or PEG-conjugated lipid comprises C14PEG2000.

In certain embodiments, the polyethylene glycol (PEG) or PEG-conjugated lipid comprises about 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0 or about 12.5 mol % of the LNP. In certain embodiments, the polyethylene glycol (PEG) or PEG-conjugated lipid comprises less than about 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0 or about 12.5 mol % of the LNP. In certain embodiments, the polyethylene glycol (PEG) or PEG-conjugated lipid comprises more than about 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0 or about 12.5 mol % of the LNP.

In certain embodiments, the polyethylene glycol (PEG) or PEG-conjugated lipid comprises about 2.5 mol % of the LNP. In certain embodiments, the polyethylene glycol (PEG) or PEG-conjugated lipid comprises less than about 2.5 mol % of the LNP. In certain embodiments, the polyethylene glycol (PEG) or PEG-conjugated lipid comprises more than about 2.5 mol % of the LNP.

In some embodiments, the total cholesterol comprises a substituted cholesterol lipid. In some embodiments, the total cholesterol comprises a mixture of cholesterol and one or more substituted cholesterol lipid. In some embodiments, the LNP molecule comprises total cholesterol at a ratio of 50% cholesterol:50% substituted cholesterol. In some embodiments, the LNP molecule comprises total cholesterol at a ratio of 75% cholesterol:25% substituted cholesterol. In some embodiments, the LNP molecule comprises total cholesterol at a ratio of 87.5% cholesterol:12.5% substituted cholesterol. In some embodiments, the LNP molecule comprises total cholesterol at a ratio of 0% cholesterol: 100% substituted cholesterol.

Exemplary substituted cholesterol lipids that can be incorporated into the LNP of the invention include, but are not limited to, a hydroxy substituted cholesterol, an epoxy substituted cholesterol and a keto substituted cholesterol.

In some embodiments, the substituted cholesterol lipid is 7α-hydroxycholesterol, 7β-hydroxycholesterol, 19-hydroxycholesterol, 20(S)-hydroxycholesterol, 24(S)-hydroxy cholesterol, 25-hydroxy cholesterol, 7-ketocholesterol, 5,6-epoxycholesterol, 3β, 5α, 6β-trihydroxycholesterol, 4β-hydroxycholesterol, 27-hydroxycholesterol or 22(R)-hydroxy cholesterol.

By way of example, In some embodiments, the LNP molecule comprises a mixture of 50% cholesterol:50% 7α-hydroxycholesterol. In some embodiments, the LNP molecule comprises a mixture of 75% cholesterol:25% 7α-hydroxycholesterol.

In some embodiments, the LNP formulated for stability for in vivo cell targeting comprises total PEG in a concentration range of about 0.5 mol % to about 12.5 mol %. In some embodiments, the total PEG is present in a molar ratio of about 2.5, or at a molar percentage of about 2.5%.

In some embodiments, the LNP formulated for stability for in vivo cell targeting comprises the compound of the present invention, DOPE, total cholesterol and PEG, wherein the compound of the present invention:DOPE:total cholesterol:PEG are present in a molar ratio of about 30:16:46.5:2.5 or at a molar percentage of about 30%:16%:46.5%:2.5%.

In some embodiments, the PEG comprises a mixture of PEG maleimide PEG (mPEG).

In various embodiments, the LNP targets at least one cell of interest. For example, in some embodiments, the LNP targets at least one tissue cell. In some embodiments, the LNP targets at least one liver cell, lung cell, spleen cell, or any combination thereof.

In certain embodiments, the LNP has a ratio of (a):(b):(c):(d) of about 35:16:46.5:2.5.

In certain embodiments, the LNP selectively targets at least one cell type of interest.

In certain embodiments, the cell of interest is a tissue cell. In certain embodiments, the cell of interest is muscle cell. In certain embodiments, the cell of interest is an immune cell. In certain embodiments, the cell of interest is endothelial cell. In certain embodiments, the cell of interest is epithelial cell. In certain embodiments, the cell of interest is hematopoietic stem cell (HSC). In certain embodiments, the cell of interest is heart cell. In certain embodiments, the cell of interest is brain cell. In certain embodiments, the cell of interest is bone marrow cell. In certain embodiments, the cell of interest is hepatocyte. In certain embodiments, the cell of interest is liver cell. In certain embodiments, the cell of interest is spleen cell. In certain embodiments, the cell of interest is lung cell. In certain embodiments, the cell of interest is podocyte. In certain embodiments, the cell of interest is kidney cell.

In one aspect, the invention is not limited to any particular cargo or otherwise agent for which the LNP is able to carry or transport. Rather, the invention includes can agent that can be carried by the LNP. For example, agents that can be carried by the LNP of the invention include, but are not limited to, diagnostic agents, detectable agents, and therapeutic agents.

In various embodiments, the composition comprises an in vitro transcribed (IVT) RNA molecule. For example, in certain embodiments, the composition of the invention comprises an IVT RNA molecule, which encodes an agent. In certain embodiments, the IVT RNA molecule of the present composition is a nucleoside-modified mRNA molecule.

In some embodiments, the composition comprises at least one RNA molecule encoding a combination of at least two agents. In some embodiments, the composition comprises a combination of two or more RNA molecules encoding a combination of two or more agents.

In some embodiments, the present disclosure provides a method for gene editing of a cell of interest of a subject. For example, the method can be used to provide one or more component of a gene editing system (e.g., a component of a CRISPR system) to a cell of interest of a subject. In some embodiments, the method comprises administering to the subject a composition comprising one or more ionizable LNP molecule formulated for targeted delivery comprising one or more nucleoside-modified RNA molecule for gene editing.

In some embodiments, the method comprises administration of the composition to a subject. In certain embodiments, the method comprises administering a plurality of doses to the subject. In another embodiment, the method comprises administering a single dose of the composition, where the single dose is effective in delivery of the target therapeutic agent.

In one aspect, the composition of the present disclosure comprises one or more LNP formulated for targeted delivery of an agent to a cell of interest (e.g., liver cell, lung cell, spleen cell, or any combination thereof).

For example, in some embodiments, the composition of the present disclosure comprises at least one therapeutic agent. In some embodiments, the therapeutic agent is a hydrophobic therapeutic agent. In some embodiments, the therapeutic agent is a hydrophilic therapeutic agent. Examples of such therapeutic agents include, but are not limited to, one or more drugs, proteins, amino acids, peptides, antibodies, antibiotics, small molecules, anti-cancer agents, chemotherapeutic agents, immunomodulatory agents, RNA molecules, siRNA molecules, DNA molecules, gene editing agents, gene-silencing agents, CRISPR-associated agents (e.g., guide RNA molecules, endonucleases, and variants thereof), medical imaging agents, therapeutic moieties, one or more non-therapeutic moieties or a combination to target cancer or atherosclerosis, selected from folic acid, peptides, proteins, aptamers, antibodies, siRNA, poorly water soluble drugs, anti-cancer drugs, antibiotics, analgesics, vaccines, anticonvulsants; anti-diabetic agents, antifungal agents, antineoplastic agents, anti-parkinsonian agents, anti-rheumatic agents, appetite suppressants, biological response modifiers, cardiovascular agents, central nervous system stimulants, contraceptive agents, dietary supplements, vitamins, minerals. lipids, saccharides, metals, amino acids (and precursors), nucleic acids and precursors, contrast agents, diagnostic agents, dopamine receptor agonists, erectile dysfunction agents, fertility agents, gastrointestinal agents, hormones, inmunomodulators, antihypercalcemia agents, mast cell stabilizers, muscle relaxants, nutritional agents, ophthalmic agents, osteoporosis agents, psychotherapeutic agents, parasympathomimetic agents, parasympatholytic agents, respiratory agents, sedative hypnotic agents, skin and mucous membrane agents, smoking cessation agents, steroids, sympatholytic agents, urinary tract agents, uterine relaxants, vaginal agents, vasodilator, anti-hypertensive, hyperthyroids, anti-hyperthyroids, anti-asthmatics and vertigo agents, or any combinations thereof.

In some embodiments, the therapeutic agent is one or more non-therapeutic moieties. In some embodiments, the nanoparticle comprises one or more therapeutic moieties, one or more non-therapeutic moieties, or any combination thereof. In some embodiments, the therapeutic moiety targets cancer. In some embodiments, the composition comprises folic acid, peptides, proteins, aptamers, antibodies, small RNA molecules, miRNA, shRNA, siRNA, poorly water-soluble therapeutic agents, anti-cancer agents, or any combinations thereof.

In some embodiments, the therapeutic agent may be an anti-cancer agent. Any suitable anti-cancer agent may be used in the compositions and methods of the present disclosure. The selection of a suitable anti-cancer agent may depend upon, among other things, the type of cancer to be treated and the nanoparticle compositions of the present disclosure. In certain embodiments, the anti-cancer agent may be effective for treating one or more of pancreatic cancer, esophageal cancer, rectal cancer, colon cancer, prostate cancer, kidney cancer, liver cancer, breast cancer, ovarian cancer, and stomach cancer. Examples of anti-cancer agents include, but are not limited to, chemotherapeutic agents, antiproliferative agents, anti-tumor agents, checkpoint inhibitors, and anti-angiogenic agents. For example, In some embodiments, the anti-cancer agent is gemcitabine, doxorubicin, 5-Fu, tyrosine kinase inhibitors, sorafenib, trametinib, rapamycin, fulvestrant, ezalutamide, or paclitaxel.

Chemotherapeutic agents include cytotoxic agents (e.g., 5-fluorouracil, cisplatin, carboplatin, methotrexate, daunorubicin, doxorubicin, vincristine, vinblastine, oxorubicin, carmustine (BCNU), lomustine (CCNU), cytarabine USP, cyclophosphamide, estramucine phosphate sodium, altretamine, hydroxyurea, ifosfamide, procarbazine, mitomycin, busulfan, cyclophosphamide, mitoxantrone, carboplatin, cisplatin, interferon alfa-2a recombinant, paclitaxel, teniposide, and streptozoci), cytotoxic alkylating agents (e.g., busulfan, chlorambucil, cyclophosphamide, melphalan, or ethylesulfonic acid), alkylating agents (e.g., asaley, AZQ, BCNU, busulfan, bisulphan, carboxyphthalatoplatinum, CBDCA, CCNU, CHIP, chlorambucil, chlorozotocin, cis-platinum, clomesone, cyanomorpholinodoxorubicin, cyclodisone, cyclophosphamide, dianhydrogalactitol, fluorodopan, hepsulfam, hycanthone, iphosphamide, melphalan, methyl CCNU, mitomycin C, mitozolamide, nitrogen mustard, PCNU, piperazine, piperazinedione, pipobroman, porfiromycin, spirohydantoin mustard, streptozotocin, teroxirone, tetraplatin, thiotepa, triethylenemelamine, uracil nitrogen mustard, and Yoshi-864), antimitotic agents (e.g., allocolchicine, Halichondrin M, colchicine, colchicine derivatives, dolastatin 10, maytansine, rhizoxin, paclitaxel derivatives, paclitaxel, thiocolchicine, trityl cysteine, vinblastine sulfate, and vincristine sulfate), plant alkaloids (e.g., actinomycin D, bleomycin, L-asparaginase, idarubicin, vinblastine sulfate, vincristine sulfate, mitramycin, mitomycin, daunorubicin, VP-16-213, VM-26, navelbine and taxotere), biologicals (e.g., alpha interferon, BCG, G-CSF, GM-CSF, and interleukin-2), topoisomerase I inhibitors (e.g., camptothecin, camptothecin derivatives, and morpholinodoxorubicin), topoisomerase II inhibitors (e.g., mitoxantron, amonafide, m-AMSA, anthrapyrazole derivatives, pyrazoloacridine, bisantrene HCL, daunorubicin, deoxydoxorubicin, menogaril, N,N-dibenzyl daunomycin, oxanthrazole, rubidazone, VM-26 and VP-16), and synthetics (e.g., hydroxyurea, procarbazine, o,p′-DDD, dacarbazine, CCNU, BCNU, cis-diamminedichloroplatimun, mitoxantrone, CBDCA, levamisole, hexamethylmelamine, all-trans retinoic acid, gliadel and porfimer sodium).

Antiproliferative agents are compounds that decrease the proliferation of cells. Antiproliferative agents include alkylating agents, antimetabolites, enzymes, biological response modifiers, miscellaneous agents, hormones and antagonists, androgen inhibitors (e.g., flutamide and leuprolide acetate), antiestrogens (e.g., tamoxifen citrate and analogs thereof, toremifene, droloxifene and roloxifene), Additional examples of specific antiproliferative agents include, but are not limited to levamisole, gallium nitrate, granisetron, sargramostim strontium-89 chloride, filgrastim, pilocarpine, dexrazoxane, and ondansetron.

The inhibitors of the invention can be administered alone or in combination with other anti-tumor agents, including cytotoxic/antineoplastic agents and anti-angiogenic agents. Cytotoxic/anti-neoplastic agents are defined as agents which attack and kill cancer cells. Some cytotoxic/anti-neoplastic agents are alkylating agents, which alkylate the genetic material in tumor cells, e.g., cis-platin, cyclophosphamide, nitrogen mustard, trimethylene thiophosphoramide, carmustine, busulfan, chlorambucil, belustine, uracil mustard, chlomaphazin, and dacabazine. Other cytotoxic/anti-neoplastic agents are antimetabolites for tumor cells, e.g., cytosine arabinoside, fluorouracil, methotrexate, mercaptopuirine, azathioprime, and procarbazine. Other cytotoxic/anti-neoplastic agents are antibiotics, e.g., doxorubicin, bleomycin, dactinomycin, daunorubicin, mithramycin, mitomycin, mytomycin C, and daunomycin. There are numerous liposomal formulations commercially available for these compounds. Still other cytotoxic/anti-neoplastic agents are mitotic inhibitors (vinca alkaloids). These include vincristine, vinblastine and etoposide. Miscellaneous cytotoxic/anti-neoplastic agents include taxol and its derivatives, L-asparaginase, anti-tumor antibodies, dacarbazine, azacytidine, amsacrine, melphalan, VM-26, ifosfamide, mitoxantrone, and vindesine.

Anti-angiogenic agents are well known to those of skill in the art. Suitable anti-angiogenic agents for use in the methods and compositions of the present disclosure include anti-VEGF antibodies, including humanized and chimeric antibodies, anti-VEGF aptamers and antisense oligonucleotides. Other known inhibitors of angiogenesis include angiostatin, endostatin, interferons, interleukin 1 (including alpha and beta) interleukin 12, retinoic acid, and tissue inhibitors of metalloproteinase-1 and -2. (TIMP-1 and -2). Small molecules, including topoisomerases such as razoxane, a topoisomerase II inhibitor with anti-angiogenic activity, can also be used.

Other anti-cancer agents that can be used in combination with the disclosed compounds include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-1b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride. Other anti-cancer drugs include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; docetaxel; docosanol; dolasetron; doxifluridine; droloxifene; dronabinol; duocarmycin SA; ebselen; ecomustine; edelfosine; edrecolomab; eflornithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer. In some embodiments, the anti-cancer drug is 5-fluorouracil, taxol, or leucovorin.

In some embodiments, the anti-cancer agent may be a prodrug form of an anti-cancer agent. As used herein, the term “prodrug form” and its derivatives is used to refer to a drug that has been chemically modified to add and/or remove one or more substituents in such a manner that, upon introduction of the prodrug form into a subject, such a modification may be reversed by naturally occurring processes, thus reproducing the drug. The use of a prodrug form of an anti-cancer agent in the compositions, among other things, may increase the concentration of the anti-cancer agent in the compositions of the present disclosure. In certain embodiments, an anti-cancer agent may be chemically modified with an alkyl or acyl group or some form of lipid. The selection of such a chemical modification, including the substituent(s) to add and/or remove to create the prodrug, may depend upon a number of factors including, but not limited to, the particular drug and the desired properties of the prodrug. One of ordinary skill in the art, with the benefit of this disclosure, will recognize suitable chemical modifications.

In some embodiments, the nanoparticle further comprises one or more gene components, such as siRNA or therapeutic DNA fragments. In some embodiments, the gene component is encapsulated in the nanoparticle. In some embodiments, the gene component is on the surface of the nanoparticle, for example, attached to or within the coating material.

In some embodiments, the nanoparticle further comprises a biocompatible metal. Examples of biocompatible metals include, but are not limited to, copper, copper sulfide, iron oxide, cobalt and noble metals, such as gold and/or silver. One of ordinary skill in the art will be able to select of a suitable type of nanoparticle taking into consideration at least the type of imaging and/or therapy to be performed.

Cargo and/or Agents

### Small Molecule

In various embodiments, the agent or cargo is a small molecule. In various embodiments, the agent or cargo is a therapeutic agent. In various embodiments, the therapeutic agent is a small molecule. When the therapeutic agent is a small molecule, a small molecule may be obtained using standard methods known to the skilled artisan. Such methods include chemical organic synthesis or biological means. Biological means include purification from a biological source, recombinant synthesis, and in vitro translation systems, using methods well known in the art. In some embodiments, a small molecule therapeutic agents comprises an organic molecule, inorganic molecule, biomolecule, synthetic molecule, and the like.

Combinatorial libraries of molecularly diverse chemical compounds potentially useful in treating a variety of diseases and conditions are well known in the art, as are method of making the libraries. The method may use a variety of techniques well-known to the skilled artisan including solid phase synthesis, solution methods, parallel synthesis of single compounds, synthesis of chemical mixtures, rigid core structures, flexible linear sequences, deconvolution strategies, tagging techniques, and generating unbiased molecular landscapes for lead discovery vs. biased structures for lead development. In some embodiments of the invention, the therapeutic agent is synthesized and/or identified using combinatorial techniques.

In a general method for small library synthesis, an activated core molecule is condensed with a number of building blocks, resulting in a combinatorial library of covalently linked, core-building block ensembles. The shape and rigidity of the core determines the orientation of the building blocks in shape space. The libraries can be biased by changing the core, linkage, or building blocks to target a characterized biological structure (“focused libraries”) or synthesized with less structural bias using flexible cores. In some embodiments of the invention, the therapeutic agent is synthesized via small library synthesis.

The small molecule and small molecule compounds described herein may be present as salts even if salts are not depicted, and it is understood that the invention embraces all salts and solvates of the therapeutic agents depicted here, as well as the non-salt and non-solvate form of the therapeutic agents, as is well understood by the skilled artisan. In some embodiments, the salts of the therapeutic agents of the invention are pharmaceutically acceptable salts.

Where tautomeric forms may be present for any of the therapeutic agents described herein, each and every tautomeric form is intended to be included in the present invention, even though only one or some of the tautomeric forms may be explicitly depicted. For example, when a 2-hydroxypyridyl moiety is depicted, the corresponding 2-pyridone tautomer is also intended.

The invention also includes any or all of the stereochemical forms, including any enantiomeric or diastereomeric forms of the therapeutic agents described. The recitation of the structure or name herein is intended to embrace all possible stereoisomers of therapeutic agents depicted. All forms of the therapeutic agents are also embraced by the invention, such as crystalline or non-crystalline forms of the therapeutic agent. Compositions comprising a therapeutic agents of the invention are also intended, such as a composition of substantially pure therapeutic agent, including a specific stereochemical form thereof, or a composition comprising mixtures of therapeutic agents of the invention in any ratio, including two or more stereochemical forms, such as in a racemic or non-racemic mixture.

The invention also includes any or all active analog or derivative, such as a prodrug, of any therapeutic agent described herein. In some embodiments, the therapeutic agent is a prodrug. In some embodiments, the small molecules described herein are candidates for derivatization. As such, in certain instances, the analogs of the small molecules described herein that have modulated potency, selectivity, and solubility are included herein and provide useful leads for drug discovery and drug development. Thus, in certain instances, during optimization new analogs are designed considering issues of drug delivery, metabolism, novelty, and safety.

In some instances, small molecule therapeutic agents described herein are derivatives or analogs of known therapeutic agents, as is well known in the art of combinatorial and medicinal chemistry. The analogs or derivatives can be prepared by adding and/or substituting functional groups at various locations. As such, the small molecules described herein can be converted into derivatives/analogs using well known chemical synthesis procedures. For example, all of the hydrogen atoms or substituents can be selectively modified to generate new analogs. Also, the linking atoms or groups can be modified into longer or shorter linkers with carbon backbones or hetero atoms. Also, the ring groups can be changed so as to have a different number of atoms in the ring and/or to include hetero atoms. Moreover, aromatics can be converted to cyclic rings, and vice versa. For example, the rings may be from 5-7 atoms, and may be carbocyclic or heterocyclic.

As used herein, the term “analog,” “analogue,” or “derivative” is meant to refer to a chemical compound or molecule made from a parent compound or molecule by one or more chemical reactions. As such, an analog can be a structure having a structure similar to that of the small molecule therapeutic agents described herein or can be based on a scaffold of a small molecule therapeutic agents described herein, but differing from it in respect to certain components or structural makeup, which may have a similar or opposite action metabolically. An analog or derivative of any of a small molecule inhibitor in accordance with the present disclosure can be used to treat a disease or disorder.

In some embodiments, the small molecule therapeutic agents described herein can independently be derivatized, or analogs prepared therefrom, by modifying hydrogen groups independently from each other into other substituents. That is, each atom on each molecule can be independently modified with respect to the other atoms on the same molecule. Any traditional modification for producing a derivative/analog can be used. For example, the atoms and substituents can be independently comprised of hydrogen, an alkyl, aliphatic, straight chain aliphatic, aliphatic having a chain hetero atom, branched aliphatic, substituted aliphatic, cyclic aliphatic, heterocyclic aliphatic having one or more hetero atoms, aromatic, heteroaromatic, polyaromatic, polyamino acids, peptides, polypeptides, combinations thereof, halogens, halo-substituted aliphatics, and the like. Additionally, any ring group on a compound can be derivatized to increase and/or decrease ring size as well as change the backbone atoms to carbon atoms or hetero atoms.

### Nucleic Acid Molecule

In other related aspects, the agent or cargo is a nucleic acid molecule. In various embodiments, the agent or cargo is an isolated nucleic acid. Thus, In some embodiments, an isolated nucleic acid, including for example a DNA oligonucleotide and a RNA oligonucleotide can be incorporated in the composition of the invention. In other related aspects, the therapeutic agent is an isolated nucleic acid. In certain embodiments, the isolated nucleic acid molecule is one of a DNA molecule or an RNA molecule. In certain embodiments, the isolated nucleic acid molecule is a DNA, cDNA, pDNA, mRNA, siRNA, shRNA, miRNA, or antisense oligonucleotide molecule. In some embodiments, the isolated nucleic acid molecule encodes a therapeutic peptide such a thrombomodulin, endothelial protein C receptor (EPCR), anti-thrombotic proteins including plasminogen activators and their mutants, antioxidant proteins including catalase, superoxide dismutase (SOD) and iron-sequestering proteins. In some embodiments, the therapeutic agent is an siRNA, miRNA, shRNA, or an antisense molecule, which inhibits a targeted nucleic acid including those encoding proteins that are involved in aggravation of the pathological processes.

In some embodiments, the nucleic acid comprises a promoter/regulatory sequence such that the nucleic acid is capable of directing expression of the nucleic acid. Thus, the invention encompasses expression vectors and methods for the introduction of exogenous nucleic acid into cells with concomitant expression of the exogenous nucleic acid in the cells such as those described, for example, in Sambrook et al. (2012, Molecular Cloning: A Laboratory Manual, Cold Spring Harbor Laboratory, New York), and in Ausubel et al. (1997, Current Protocols in Molecular Biology, John Wiley & Sons, New York) and as described elsewhere herein.

In some embodiments, siRNA is used to decrease the level of a targeted protein. RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559; Fire et al., 1998, Nature 391(19):306-311; Timmons et al., 1998, Nature 395:854; Montgomery et al., 1998, TIG 14 (7):255-258; David R. Engelke, Ed., RNA Interference (RNAi) Nuts & Bolts of RNAi Technology, DNA Press, Eagleville, PA (2003); and Gregory J. Hannon, Ed., RNAi A Guide to Gene Silencing, Cold Spring Harbor Laboratory Press, Cold Spring Harbor, NY (2003). Soutschek et al. (2004, Nature 432:173-178) describe a chemical modification to siRNAs that aids in intravenous systemic delivery. Optimizing siRNAs involves consideration of overall G/C content, C/T content at the termini, Tm and the nucleotide content of the 3′ overhang. See, for instance, Schwartz et al., 2003, Cell, 115:199-208 and Khvorova et al., 2003, Cell 115:209-216.

In one aspect, the invention includes a vector comprising an siRNA or an antisense polynucleotide. Preferably, the siRNA or antisense polynucleotide is capable of inhibiting the expression of a target polypeptide. The incorporation of a desired polynucleotide into a vector and the choice of vectors are well-known in the art as described in, for example, Sambrook et al. (2012), and in Ausubel et al. (1997), and elsewhere herein.

In certain embodiments, the expression vectors described herein encode a short hairpin RNA (shRNA) therapeutic agents. shRNA molecules are well known in the art and are directed against the mRNA of a target, thereby decreasing the expression of the target. In certain embodiments, the encoded shRNA is expressed by a cell, and is then processed into siRNA. For example, in certain instances, the cell possesses native enzymes (e.g., dicer) that cleave the shRNA to form siRNA.

In order to assess the expression of the siRNA, shRNA, or antisense polynucleotide, the expression vector to be introduced into a cell can also contain either a selectable marker gene or a reporter gene or both to facilitate identification of expressing cells from the population of cells sought to be transfected or infected using the delivery vehicle of the invention. In other embodiments, the selectable marker may be carried on a separate piece of DNA and also be contained within the delivery vehicle. Both selectable markers and reporter genes may be flanked with appropriate regulatory sequences to enable expression in the host cells. Useful selectable markers are known in the art and include, for example, antibiotic-resistance genes, such as neomycin resistance and the like.

Therefore, in one aspect, the delivery vehicle may contain a vector, comprising the nucleotide sequence or the construct to be delivered. The choice of the vector will depend on the host cell in which it is to be subsequently introduced. In a particular embodiment, the vector of the invention is an expression vector. Suitable host cells include a wide variety of prokaryotic and eukaryotic host cells. In specific embodiments, the expression vector is selected from the group consisting of a viral vector, a bacterial vector and a mammalian cell vector. Prokaryote- and/or eukaryote-vector based systems can be employed for use with the present disclosure to produce polynucleotides, or their cognate polypeptides. Many such systems are commercially and widely available.

By way of illustration, the vector in which the nucleic acid sequence is introduced can be a plasmid, which is or is not integrated in the genome of a host cell when it is introduced in the cell. Illustrative, non-limiting examples of vectors in which the nucleotide sequence of the invention or the gene construct of the invention can be inserted include a tet-on inducible vector for expression in eukaryote cells.

The vector may be obtained by conventional methods known by persons skilled in the art (Sambrook et al., 2012). In a particular embodiment, the vector is a vector useful for transforming animal cells.

In some embodiments, the recombinant expression vectors may also contain nucleic acid molecules, which encode a peptide or peptidomimetic.

A promoter may be one naturally associated with a gene or polynucleotide sequence, as may be obtained by isolating the 5′ non-coding sequences located upstream of the coding segment and/or exon. Such a promoter can be referred to as “endogenous.” Similarly, an enhancer may be one naturally associated with a polynucleotide sequence, located either downstream or upstream of that sequence. Alternatively, certain advantages will be gained by positioning the coding polynucleotide segment under the control of a recombinant or heterologous promoter, which refers to a promoter that is not normally associated with a polynucleotide sequence in its natural environment. A recombinant or heterologous enhancer refers also to an enhancer not normally associated with a polynucleotide sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (U.S. Pat. Nos. 4,683,202, 5,928,906). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know how to use promoters, enhancers, and cell type combinations for protein expression, for example, see Sambrook et al. (2012). The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

The recombinant expression vectors may also contain a selectable marker gene, which facilitates the selection of host cells. Suitable selectable marker genes are genes encoding proteins such as G418 and hygromycin, which confer resistance to certain drugs, D-galactosidase, chloramphenicol acetyltransferase, firefly luciferase, or an immunoglobulin or portion thereof such as the Fc portion of an immunoglobulin preferably IgG. The selectable markers may be introduced on a separate vector from the nucleic acid of interest.

Following the generation of the siRNA polynucleotide, a skilled artisan will understand that the siRNA polynucleotide will have certain characteristics that can be modified to improve the siRNA as a therapeutic compound. Therefore, the siRNA polynucleotide may be further designed to resist degradation by modifying it to include phosphorothioate, or other linkages, methylphosphonate, sulfone, sulfate, ketyl, phosphorodithioate, phosphoramidate, phosphate esters, and the like (see, e.g., Agrawal et al., 1987, Tetrahedron Lett. 28:3539-3542; Stec et al., 1985 Tetrahedron Lett. 26:2191-2194; Moody et al., 1989 Nucleic Acids Res. 12:4769-4782; Eckstein, 1989 Trends Biol. Sci. 14:97-100; Stein, In: Oligodeoxynucleotides. Antisense Inhibitors of Gene Expression, Cohen, ed., Macmillan Press, London, pp. 97-117 (1989)).

Any polynucleotide may be further modified to increase its stability in vivo. Possible modifications include, but are not limited to, the addition of flanking sequences at the 5′ and/or 3′ ends; the use of phosphorothioate or 2′ O-methyl rather than phosphodiester linkages in the backbone; and/or the inclusion of nontraditional bases such as inosine, queuosine, and wybutosine and the like, as well as acetyl- methyl-, thio- and other modified forms of adenine, cytidine, guanine, thymine, and uridine.

In some embodiments of the invention, an antisense nucleic acid sequence, which is expressed by a plasmid vector is used as a therapeutic agent to inhibit the expression of a target protein. The antisense expressing vector is used to transfect a mammalian cell or the mammal itself, thereby causing reduced endogenous expression of the target protein.

Antisense molecules and their use for inhibiting gene expression are well known in the art (see, e.g., Cohen, 1989, In: Oligodeoxyribonucleotides, Antisense Inhibitors of Gene Expression, CRC Press). Antisense nucleic acids are DNA or RNA molecules that are complementary, as that term is defined elsewhere herein, to at least a portion of a specific mRNA molecule (Weintraub, 1990, Scientific American 262:40). In the cell, antisense nucleic acids hybridize to the corresponding mRNA, forming a double-stranded molecule thereby inhibiting the translation of genes.

The use of antisense methods to inhibit the translation of genes is known in the art, and is described, for example, in Marcus-Sakura (1988, Anal. Biochem. 172:289). Such antisense molecules may be provided to the cell via genetic expression using DNA encoding the antisense molecule as taught by Inoue, 1993, U.S. Pat. No. 5,190,931.

Alternatively, antisense molecules of the invention may be made synthetically and then provided to the cell. Antisense oligomers of between about 10 to about 30, and more preferably about 15 nucleotides, are preferred, since they are easily synthesized and introduced into a target cell (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.). Synthetic antisense molecules contemplated by the invention include oligonucleotide derivatives known in the art which have improved biological activity compared to unmodified oligonucleotides (see U.S. Pat. No. 5,023,243).

In some embodiments of the invention, a ribozyme is used as a therapeutic agent to inhibit expression of a target protein. Ribozymes useful for inhibiting the expression of a target molecule may be designed by incorporating target sequences into the basic ribozyme structure. which are complementary. for example, to the mRNA sequence encoding the target molecule. Ribozymes targeting the target molecule, may be synthesized using commercially available reagents (Applied Biosystems, Inc., Foster City, CA) or they may be genetically expressed from DNA encoding them.

In some embodiments, the therapeutic agent may comprise one or more components of a CRISPR-Cas system, where a guide RNA (gRNA) targeted to a gene encoding a target molecule, and a CRISPR-associated (Cas) peptide form a complex to induce mutations within the targeted gene. In some embodiments, the therapeutic agent comprises a gRNA or a nucleic acid molecule encoding a gRNA. In some embodiments, the therapeutic agent comprises a Cas peptide or a nucleic acid molecule encoding a Cas peptide.

In some embodiments, the agent or cargo comprises a miRNA or a mimic of a miRNA. In some embodiments, the agent or cargo comprises a nucleic acid molecule that encodes a miRNA or mimic of a miRNA.

miRNAs are small non-coding RNA molecules that are capable of causing post-transcriptional silencing of specific genes in cells by the inhibition of translation or through degradation of the targeted mRNA. A miRNA can be completely complementary or can have a region of non-complementarity with a target nucleic acid, consequently resulting in a “bulge” at the region of non-complementarity. A miRNA can inhibit gene expression by repressing translation, such as when the miRNA is not completely complementary to the target nucleic acid, or by causing target RNA degradation, which is believed to occur only when the miRNA binds its target with perfect complementarity. The disclosure also can include double-stranded precursors of miRNA. A miRNA or pri-miRNA can be 18-100 nucleotides in length, or from 18-80 nucleotides in length. Mature miRNAs can have a length of 19-30 nucleotides, or 21-25 nucleotides, particularly 21, 22, 23, 24, or 25 nucleotides. MiRNA precursors typically have a length of about 70-100 nucleotides and have a hairpin conformation. miRNAs are generated in vivo from pre-miRNAs by the enzymes Dicer and Drosha, which specifically process long pre-miRNA into functional miRNA. The hairpin or mature microRNAs, or pri-microRNA agents featured in the disclosure can be synthesized in vivo by a cell-based system or in vitro by chemical synthesis.

In various embodiments, the agent or cargo comprises an oligonucleotide that comprises the nucleotide sequence of a disease-associated miRNA. In certain embodiments, the oligonucleotide comprises the nucleotide sequence of a disease-associated miRNA in a pre-microRNA, mature or hairpin form. In other embodiments, a combination of oligonucleotides comprising a sequence of one or more disease-associated miRNAs, any pre-miRNA, any fragment, or any combination thereof is envisioned.

MiRNAs can be synthesized to include a modification that imparts a desired characteristic. For example, the modification can improve stability, hybridization thermodynamics with a target nucleic acid, targeting to a particular tissue or cell-type, or cell permeability, e.g., by an endocytosis-dependent or -independent mechanism.

Modifications can also increase sequence specificity, and consequently decrease off-site targeting. Methods of synthesis and chemical modifications are described in greater detail below. If desired, miRNA molecules may be modified to stabilize the miRNAs against degradation, to enhance half-life, or to otherwise improve efficacy. Desirable modifications are described, for example, in U.S. Patent Publication Nos. 20070213292, 20060287260, 20060035254. 20060008822. and 2005028824, each of which is hereby incorporated by reference in its entirety. For increased nuclease resistance and/or binding affinity to the target, the single-stranded oligonucleotide agents featured in the disclosure can include 2′-O-methyl, 2′-fluorine, 2′-O-methoxyethyl, 2′-O-aminopropyl, 2′-amino, and/or phosphorothioate linkages. Inclusion of locked nucleic acids (LNA), ethylene nucleic acids (ENA), e.g., 2′-4′-ethylene-bridged nucleic acids, and certain nucleotide modifications can also increase binding affinity to the target. The inclusion of pyranose sugars in the oligonucleotide backbone can also decrease endonucleolytic cleavage. An oligonucleotide can be further modified by including a 3′ cationic group, or by inverting the nucleoside at the 3′-terminus with a 3-3′ linkage. In another alternative, the 3′-terminus can be blocked with an aminoalkyl group. Other 3′ conjugates can inhibit 3′-5′ exonucleolytic cleavage. While not being bound by theory, a 3′ may inhibit exonucleolytic cleavage by sterically blocking the exonuclease from binding to the 3′ end of the oligonucleotide. Even small alkyl chains, aryl groups, or heterocyclic conjugates or modified sugars (D-ribose, deoxyribose, glucose etc.) can block 3′-5′-exonucleases.

In some embodiments, the miRNA includes a 2′-modified oligonucleotide containing oligodeoxynucleotide gaps with some or all internucleotide linkages modified to phosphorothioates for nuclease resistance. The presence of methylphosphonate modifications increases the affinity of the oligonucleotide for its target RNA and thus reduces the IC₅Q. This modification also increases the nuclease resistance of the modified oligonucleotide. It is understood that the methods and reagents of the present disclosure may be used in conjunction with any technologies that may be developed to enhance the stability or efficacy of an inhibitory nucleic acid molecule.

miRNA molecules include nucleotide oligomers containing modified backbones or non-natural internucleoside linkages. Oligomers having modified backbones include those that retain a phosphorus atom in the backbone and those that do not have a phosphorus atom in the backbone. For the purposes of this disclosure, modified oligonucleotides that do not have a phosphorus atom in their internucleoside backbone are also considered to be nucleotide oligomers. Nucleotide oligomers that have modified oligonucleotide backbones include, for example, phosphorothioates, chiral phosphorothioates, phosphorodithioates, phosphotriesters, aminoalkyl-phosphotriesters, methyl and other alkyl phosphonates including 3-alkylene phosphonates and chiral phosphonates, phosphinates, phosphoramidates, thionophosphoramidates, thionoalkylphosphonates, thionoalkylphosphotriesters, and boranophosphates. Various salts, mixed salts and free acid forms are also included.

A miRNA described herein, which may be in the mature or hairpin form, may be provided as a naked oligonucleotide. In some cases, it may be desirable to utilize a formulation that aids in the delivery of a miRNA or other nucleotide oligomer to cells (see, e.g., U.S. Pat. Nos. 5,656,611, 5,753,613, 5,785,992, 6,120,798, 6,221,959, 6,346,613, and 6,353,055, each of which is hereby incorporated by reference).

In some examples, the miRNA composition is at least partially crystalline, uniformly crystalline, and/or anhydrous (e.g., less than 80, 50, 30, 20, or 10% water). In another example, the miRNA composition is in an aqueous phase, e.g., in a solution that includes water. The aqueous phase or the crystalline compositions can be incorporated into a delivery vehicle, e.g., a liposome (particularly for the aqueous phase), or a particle (e.g., a microparticle as can be appropriate for a crystalline composition). Generally, the miRNA composition is formulated in a manner that is compatible with the intended method of administration. A miRNA composition can be formulated in combination with another agent, e.g., another therapeutic agent or an agent that stabilizes an oligonucleotide agent, e.g., a protein that complexes with the oligonucleotide agent. Still other agents include chelators, e.g., EDTA (e.g., to remove divalent cations such as Mg), salts, and RNAse inhibitors (e.g., a broad specificity RNAse inhibitor). In some embodiments, the miRNA composition includes another miRNA, e.g., a second miRNA composition (e.g., a microRNA that is distinct from the first). Still other preparations can include at least three, five, ten, twenty, fifty, or a hundred or more different oligonucleotide species.

In certain embodiments, the composition comprises an oligonucleotide composition that mimics the activity of a miRNA. In certain embodiments, the composition comprises oligonucleotides having nucleobase identity to the nucleobase sequence of a miRNA, and are thus designed to mimic the activity of the miRNA. In certain embodiments, the oligonucleotide composition that mimics miRNA activity comprises a double-stranded RNA molecule which mimics the mature miRNA hairpins or processed miRNA duplexes.

In some embodiments, the oligonucleotide shares identity with endogenous miRNA or miRNA precursor nucleobase sequences. An oligonucleotide selected for inclusion in a composition of the present disclosure may be one of a number of lengths. Such an oligonucleotide can be from 7 to 100 linked nucleosides in length. For example, an oligonucleotide sharing nucleobase identity with a miRNA may be from 7 to 30 linked nucleosides in length. An oligonucleotide sharing identity with a miRNA precursor may be up to 100 linked nucleosides in length. In certain embodiments, an oligonucleotide comprises 7 to 30 linked nucleosides. In certain embodiments, an oligonucleotide comprises 7, 8, 9, 10, 11, 12, 13, 14, 15, 16, 17, 18, 19, 20, 21, 22, 23, 24, 25, 26, 28, 29, or 30 linked nucleotides. In certain embodiments, an oligonucleotide comprises 19 to 23 linked nucleosides. In certain embodiments, an oligonucleotide is from 40 up to 50, 60, 70, 80, 90, or 100 linked nucleosides in length.

In certain embodiments, an oligonucleotide has a sequence that has a certain identity to a miRNA or a precursor thereof. Nucleobase sequences of mature miRNAs and their corresponding stem-loop sequences described herein are the sequences found in miRBase, an online searchable database of miRNA sequences and annotation. Entries in the miRBase Sequence database represent a predicted hairpin portion of a miRNA transcript (the stem-loop), with information on the location and sequence of the mature miRNA sequence. The miRNA stem-loop sequences in the database are not strictly precursor miRNAs (pre-miRNAs), and may in some instances include the pre-miRNA and some flanking sequence from the presumed primary transcript. The miRNA nucleobase sequences described herein encompass any version of the miRNA, including the sequences described in Release 10.0 of the miRBase sequence database and sequences described in any earlier Release of the miRBase sequence database. A sequence database release may result in the re-naming of certain miRNAs. A sequence database release may result in a variation of a mature miRNA sequence. The compositions of the present disclosure encompass oligomeric compound comprising oligonucleotides having a certain identity to any nucleobase sequence version of a miRNAs described herein.

In certain embodiments, an oligonucleotide has a nucleobase sequence at least 60%, 65%, 70%, 75%, 80%, 85%, 90%, 95%, 97%, 98% or 99% identical to the miRNA over a region of 7, 8, 9, 10, 11, 12, 13, 14, 15, 16, 17, 18, 19, 20, 21, 22, 23, 24, 25, 26, 27, 28, 29, or 30 nucleobases. Accordingly, in certain embodiments the nucleobase sequence of an oligonucleotide may have one or more non-identical nucleobases with respect to the miRNA.

In the sense used in this description, a nucleotide sequence is “substantially homologous” to any of the nucleotide sequences describe herein when its nucleotide sequence has a degree of identity with respect to the nucleotide sequence of at least 60%, advantageously of at least 70%, preferably of at least 85%, and more preferably of at least 95%. Other examples of possible modifications include the insertion of one or more nucleotides in the sequence, the addition of one or more nucleotides in any of the ends of the sequence, or the deletion of one or more nucleotides in any end or inside the sequence. The degree of identity between two polynucleotides is determined using computer algorithms and methods that are widely known for the persons skilled in the art. The identity between two amino acid sequences is preferably determined by using the BLASTN algorithm [BLAST Manual, Altschul, S., et al., NCBI NLM NIH Bethesda, Md. 20894, Altschul, S., et al., J. Mol. Biol. 215: 403-410 (1990)].

In certain embodiments, the composition comprises a nucleic acid molecule encoding a miRNA, precursor, mimic, or fragment thereof. For example, the composition may comprise a viral vector, plasmid, cosmid, or other expression vector suitable for expressing the miRNA, precursor, mimic, or fragment thereof in a desired mammalian cell or tissue.

### Polypeptide

In other related aspects, the agent or cargo is a polypeptide. In various embodiments, the agent or cargo is an isolated polypeptide. In other related aspects, the therapeutic agent includes an isolated polypeptide. For example, In some embodiments, the polypeptide of the invention inhibits or activates a target directly by binding to the target thereby modulating the normal functional activity of the target. In some embodiments, the polypeptide of the invention modulates the target by competing with endogenous proteins. In some embodiments, the polypeptide of the invention modulates the activity of the target by acting as a transdominant negative mutant.

The variants of the polypeptide therapeutic agents may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the polypeptide is an alternative splice variant of the polypeptide of the present invention, (iv) fragments of the polypeptides and/or (v) one in which the polypeptide is fused with another polypeptide, such as a leader or secretory sequence or a sequence which is employed for purification (for example, His-tag) or for detection (for example, Sv5 epitope tag). The fragments include polypeptides generated via proteolytic cleavage (including multi-site proteolysis) of an original sequence. Variants may be post-translationally, or chemically modified. Such variants are deemed to be within the scope of those skilled in the art from the teaching herein.

In one aspect, the invention includes an ionizable LNP molecule comprising or encapsulating one or more agent (e.g., a nucleic acid molecule) for targeted in vivo delivery of the encapsulated agent to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.). In some embodiments, the nucleic acid molecule is a mRNA, siRNA, microRNA, DNA, pDNA, and/or antisense oligonucleotide molecule.

In some embodiments, the mRNA, siRNA, microRNA, DNA, pDNA, and/or antisense oligonucleotide molecule comprises a nucleotide sequence that can alternatively comprise sequence variations with respect to the original nucleotide sequences, for example, substitutions, insertions and/or deletions of one or more nucleotides, with the condition that the resulting polynucleotide encodes a polypeptide according to the invention.

As used herein, an amino acid sequence is “substantially homologous” to any of the amino acid sequences described herein when its amino acid sequence has a degree of identity with respect to the amino acid sequence of at least 60%, advantageously of at least 70%, preferably of at least 85%, and more preferably of at least 95%. The identity between two amino acid sequences is preferably determined by using the BLASTN algorithm (BLAST Manual, Altschul, S., et al., NCBI NLM NIH Bethesda, Md. 20894, Altschul, S., et al., J. Mol. Biol. 215: 403-410 (1990)).

In some embodiments, the composition comprises a plurality of constructs, each construct encoding one or more antigens. In certain embodiments, the composition comprises 1 or more, 2 or more, 5 or more, 10 or more, 15 or more, or 20 or more constructs. In some embodiments, the composition comprises a first construct, comprising a nucleotide sequence encoding an antigen; and a second construct, comprising a nucleotide sequence encoding an adjuvant.

In some embodiments, the construct comprises a plurality of nucleotide sequences encoding a plurality of antigens. In certain embodiments, the construct encodes 1 or more, 2 or more, 5 or more, 10 or more, 15 or more, or 20 or more antigens. In some embodiments, the invention relates to a construct, comprising a nucleotide sequence encoding an adjuvant. For example, In some embodiments, the construct comprises a first nucleotide sequence encoding an antigen and a second nucleotide sequence encoding an adjuvant.

In another particular embodiment, the construct is operatively bound to a translational control element. The construct can incorporate an operatively bound regulatory sequence for the expression of the nucleotide sequence of the invention, thus forming an expression cassette.

### Peptides

In some embodiments, the agent or cargo is a peptide. Thus, in one aspect, a peptide can be incorporated into the LNP. Thus, In some embodiments, the agent or cargo is a peptide. The peptide of the present disclosure may be made using chemical methods. For example, peptides can be synthesized by solid phase techniques (Roberge J Y et al (1995) Science 269: 202-204), cleaved from the resin, and purified by preparative high performance liquid chromatography. Automated synthesis may be achieved, for example, using the ABI 431 A Peptide Synthesizer (Perkin Elmer) in accordance with the instructions provided by the manufacturer.

The peptide may alternatively be made by recombinant means or by cleavage from a longer polypeptide. The composition of a peptide may be confirmed by amino acid analysis or sequencing.

The variants of the peptides according to the present disclosure may be (i) one in which one or more of the amino acid residues are substituted with a conserved or non-conserved amino acid residue (preferably a conserved amino acid residue) and such substituted amino acid residue may or may not be one encoded by the genetic code, (ii) one in which there are one or more modified amino acid residues, e.g., residues that are modified by the attachment of substituent groups, (iii) one in which the peptide is an alternative splice variant of the peptide of the present invention, (iv) fragments of the peptides and/or (v) one in which the peptide is fused with another peptide, such as a leader or secretory sequence or a sequence which is employed for purification (for example, His-tag) or for detection (for example, Sv5 epitope tag). The fragments include peptides generated via proteolytic cleavage (including multi-site proteolysis) of an original sequence. Variants may be post-translationally, or chemically modified. Such variants are deemed to be within the scope of those skilled in the art from the teaching herein.

As known in the art the “similarity” between two peptides is determined by comparing the amino acid sequence and its conserved amino acid substitutes of one peptide to a sequence of a second peptide. Variants are defined to include peptide sequences different from the original sequence, preferably different from the original sequence in less than 40% of residues per segment of interest, more preferably different from the original sequence in less than 25% of residues per segment of interest, more preferably different by less than 10% of residues per segment of interest, most preferably different from the original protein sequence in just a few residues per segment of interest and at the same time sufficiently homologous to the original sequence to preserve the functionality of the original sequence. The present disclosure includes amino acid sequences that are at least 60%, 65%, 70%, 72%, 74%, 76%, 78%, 80%, 90%, or 95% similar or identical to the original amino acid sequence. The degree of identity between two peptides is determined using computer algorithms and methods that are widely known for the persons skilled in the art. The identity between two amino acid sequences is preferably determined by using the BLASTP algorithm [BLAST Manual, Altschul, S., et al., NCBI NLM NIH Bethesda, Md. 20894, Altschul, S., et al., J. Mol. Biol. 215: 403-410 (1990)].

The peptides of the invention can be post-translationally modified. For example, post-translational modifications that fall within the scope of the present disclosure include signal peptide cleavage, glycosylation, acetylation, isoprenylation, proteolysis, myristoylation, protein folding and proteolytic processing, etc. Some modifications or processing events require introduction of additional biological machinery. For example, processing events, such as signal peptide cleavage and core glycosylation, are examined by adding canine microsomal membranes or Xenopus egg extracts (U.S. Pat. No. 6,103,489) to a standard translation reaction.

The peptides of the invention may include unnatural amino acids formed by post-translational modification or by introducing unnatural amino acids during translation.

### Antibodies

In some embodiments, the agent or cargo is an antibody. Thus, in various embodiments, the composition of the invention comprises an antibody, or antibody fragment. In certain embodiments, the antibody targeting domain specifically binds to a target of interest. Such antibodies include polyclonal antibodies, monoclonal antibodies, Fab and single chain Fv (scFv) fragments thereof, bispecific antibodies, heteroconjugates, human and humanized antibodies.

The antibodies may be intact monoclonal or polyclonal antibodies, and immunologically active fragments (e.g., a Fab or (Fab)2 fragment), an antibody heavy chain, an antibody light chain, humanized antibodies, a genetically engineered single chain Fv molecule (Ladner et al, U.S. Pat. No. 4,946,778), or a chimeric antibody, for example, an antibody which contains the binding specificity of a murine antibody, but in which the remaining portions are of human origin. Antibodies including monoclonal and polyclonal antibodies, fragments and chimeras, may be prepared using methods known to those skilled in the art.

Such antibodies may be produced in a variety of ways, including hybridoma cultures, recombinant expression in bacteria or mammalian cell cultures, and recombinant expression in transgenic animals. The choice of manufacturing methodology depends on several factors including the antibody structure desired, the importance of carbohydrate moieties on the antibodies, ease of culturing and purification, and cost. Many different antibody structures may be generated using standard expression technology, including full-length antibodies, antibody fragments, such as Fab and Fv fragments, as well as chimeric antibodies comprising components from different species. Antibody fragments of small size, such as Fab and Fv fragments, having no effector functions and limited pharmokinetic activity may be generated in a bacterial expression system. Single chain Fv fragments show low immunogenicity.

### Chimeric Antigen Receptor (CAR) Agents

In some embodiments, the agent or cargo comprises a nucleic acid sequence encoding a chimeric antigen receptor (CAR). In some embodiments, the agent or cargo comprises an mRNA molecule encoding a CAR. In some embodiments, the agent or cargo comprises a modified nucleoside mRNA molecule encoding a CAR.

In some embodiments, a CAR comprises an extracellular domain capable of binding an antigen, including a tumor or pathogen antigen.

Targets of antigen-specific targeting regions of CARs may be of any kind. In some embodiments, the antigen-specific targeting region of the CAR targets antigens specific for cancer, inflammatory disease, neuronal-disorders, diabetes, cardiovascular disease, infectious diseases or a combination thereof. Examples of antigens that may be targeted by the CARs include but are not limited to antigens expressed on B-cells, antigens expressed on carcinomas, sarcomas, lymphomas, leukemia, germ cell tumors, blastomas, antigens expressed on various immune cells, and antigens expressed on cells associated with various hematologic diseases, autoimmune diseases, and/or inflammatory diseases. The CARs of the disclosure may be capable of redirecting the effector function of the expressing-cells to the target antigen(s).

Antigens that may be targeted by the CARs of the disclosure include but are not limited to any one or more of 4-IBB, 707-AP, 5T4, adenocarcinoma antigen, alpha-fetoprotein, BAFF, B-lymphoma cell, C242 antigen, CA-125, carbonic anhydrase 9 (CA-IX), C-MET, ART-4, BAGE, b-catenin/m, bcr-abl, CAMEL, CAP-1, CCR4, CD 152, CD7, CD 19, CD20, CD200, CD22, CD221, CD23 (IgE receptor), CD28, CD30 (TNFRSF8), CD33, CD4, CD38, CD40, CD44 v6, CD44v7/8, CD51, CD52, CD56, CD74, CD80, CD93, CD123, CD171, CEA, CLPP, CNT0888, CTLA-4, carcinoembryonic antigen, EGP2, EGP40, DR5, ErbB2, ErbB3/4, EGFR, EpCAM, EPV-E6, CD3, CASP-8, CD109, CDK/4, CDC-27, Cyp-B, DAM-8, DAM-10, ELV-M2, ETV6, FAP, fibronectin extra domain-B, folate receptor 1, GAGE, GD2, GD3 ganglioside, glycoprotein 75, GPNMB, G250, Gp100, HAGE, HER2/neu, HGF, HMW-MAA, human scatter factor receptor kinase, hTERT, IGF-1 receptor, IGF-I, IgGI, -I-CAM, IL-13, IL-6, insulin-like growth factor I receptor, integrin α5β1, integrin αvβ, Kappa or light chain, LAGE, Lewis Y, G250/CAIX, Glypican-3, MAGE, MCl-R, mesothelin, MORAb-009, MS4A1, MUC1, MUC16, mucin CanAg, N-glycolylneuraminic acid, NPC-1C, PDGF-R a, PDL192, phosphatidylserine, PSC1, PSMA, NKG2D ligands, RANKL, RON, ROR1, SAGE, SCH 900105, SDC1, SLAMF7, TAG-72, TEL/AML, tenascin C, TGF beta 2, TGF-β, TRAIL-R1, TRAIL-R2, tumor antigen CTAA16.88, VEGF-A, VEGFR-1, VEGFR2, vimentin, B7-H6, IL-13 receptor a2, IL-11 receptor Ra, 8H9, NCAM, Fetal AchR, iCE, MART-1, tyrosinase, WT-1, TEM-1, TEM-2, TEM-3, TEM-4, TEM-5, TEM-6, TEM-7, TEM-8, ROBO-4, and so forth. Other antigens specific for cancer will be apparent to those of skill in the art and may be used in connection with alternate embodiments of the invention.

Particular examples of target antigens include but are not limited to surface proteins found on cancer cells in a specific or amplified fashion (e.g. the IL-14 receptor, CD 19, CD20 and CD40 for B-cell lymphoma, the Lewis Y and CEA antigens for a variety of carcinomas, the Tag72 antigen for breast and colorectal cancer, EGF-R for lung cancer, folate binding protein and the HER-2 protein that is often amplified in human breast and ovarian carcinomas), or viral proteins (e.g. gp120 and gp41 envelope proteins of HIV, envelope proteins from the Hepatitis B and C viruses, the glycoprotein B and other envelope glycoproteins of human cytomegalovirus, the envelope proteins from oncoviruses such as Kaposi's sarcoma-associated Herpes virus). Other targets of the CARs of the disclosure include CD4, where the ligand is the HIV gp120 envelope glycoprotein, and other viral receptors, for example ICAM, which is the receptor for the human rhinovirus, and the related receptor molecule for poliovirus.

In some embodiments, the bispecific chimeric antigen receptors target and bind at least two different antigens. Examples of pairings of at least two antigens bound by the bispecific CARs of the disclosure include but are not limited to any combination with HER2, CD 19 and CD20, CD 19 and CD22, CD20 and -I-CAM, -I-CAM and GD2, EGFR and -I-CAM, EGFR and C-MET, EGFR and HER2, C-MET and HER2 and EGFR and ROR1. Other pairings of antigens specific for cancer will be apparent to those of skill in the art and may be used in connection with alternate embodiments of the disclosure. In yet other embodiments, the bispecific chimeric antigen receptor targets CD 19 and CD20.

Antigens specific for inflammatory diseases that may be targeted by the CARs of the disclosure include but are not limited to any one or more of AOC3 (VAP-1), CAM-3001, CCL11 (eotaxin-1), CD125, CD147 (basigin), CD154 (CD40L), CD2, CD20, CD23 (IgE receptor), CD25 (a chain of IL-2 receptor), CD3, CD4, CD5, IFN-α, IFN-γ, IgE, IgE Fc region, IL-1, IL-12, IL-23, IL-13, IL-17, IL-17A, IL-22, IL-4, IL-5, IL-5, IL-6, IL-6 receptor, integrin a4, integrin α4β7, Lama glama, LFA-1 (CD11a), MEDI-528, myostatin, OX-40, rhuMAb (37, scleroscin, SOST, TGF beta 1, TNF-α or VEGF-A. Other antigens specific for inflammatory diseases will be apparent to those of skill in the art and may be used in connection with alternate embodiments of the invention.

Antigens specific for neuronal disorders that may be targeted by the CARs of the disclosure include but are not limited to any one or more of beta amyloid or MABT5102A. Other antigens specific for neuronal disorders will be apparent to those of skill in the art and may be used in connection with alternate embodiments of the invention.

Antigens specific for diabetes that may be targeted by the CARs of the disclosure include but are not limited to any one or more of L-43 or CD3. Other antigens specific for diabetes or other metabolic disorders will be apparent to those of skill in the art and may be used in connection with alternate embodiments of the invention.

Antigens specific for cardiovascular diseases which may be targeted by the CARs of the disclosure include but are not limited to any one or more of C5, cardiac myosin, CD41 (integrin alpha-lib), fibrin II, beta chain, ITGB2 (CD 18) and sphingosine-1-phosphate. Other antigens specific for cardiovascular diseases will be apparent to those of skill in the art and may be used in connection with alternate embodiments of the disclosure.

Antigens specific for infectious diseases that may be targeted by the CARs of the disclosure include but are not limited to any one or more of anthrax toxin, CCR5, CD4, clumping factor A, cytomegalovirus, cytomegalovirus glycoprotein B, endotoxin, Escherichia coli, hepatitis B surface antigen, hepatitis B virus, HIV-1, Hsp90, Influenza A hemagglutinin, lipoteichoic acid, Pseudomonas aeruginosa, rabies virus glycoprotein, respiratory syncytial virus and TNF-α. Other antigens specific for infectious diseases will be apparent to those of skill in the art and may be used in connection with alternate embodiments of the disclosure.

Additional targets of the CARs of the disclosure include antigens involved in B-cell associated diseases. Yet further targets of the CARs of the disclosure will be apparent to those of skill in the art and may be used in connection with alternate embodiments of the invention.

Other antigens specific for cancer will be apparent to those of skill in the art and may be used in connection with alternate embodiments of the invention.

In some embodiments, the CAR comprises an antigen binding domain. In a particular non-limiting embodiment, the antigen-binding domain is an scFv specific for binding to a surface antigen of a target cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.).

In various embodiments, the CAR can be a “first generation,” “second generation,” “third generation,” “fourth generation” or “fifth generation” CAR (see, for example, Sadelain et al., Cancer Discov. 3(4):388-398 (2013); Jensen et al., Immunol. Rev. 257:127-133 (2014); Sharpe et al., Dis. Model Mech. 8(4):337-350 (2015); Brentjens et al., Clin. Cancer Res. 13:5426-5435 (2007); Gade et al., Cancer Res. 65:9080-9088 (2005); Maher et al., Nat. Biotechnol. 20:70-75 (2002); Kershaw et al., J. Immunol. 173:2143-2150 (2004); Sadelain et al., Curr. Opin. Immunol. (2009); Hollyman et al., J. Immunother. 32:169-180 (2009)).

“First generation” CARs for use in the invention comprise an antigen binding domain, for example, a single-chain variable fragment (scFv), fused to a transmembrane domain, which is fused to a cytoplasmic/intracellular domain of the T cell receptor chain. “First generation” CARs typically have the intracellular domain from the CD3ζ-chain, which is the primary transmitter of signals from endogenous T cell receptors (TCRs). “First generation” CARs can provide de novo antigen recognition and cause activation of both CD4+ and CD8+ T cells through their CD3ζ chain signaling domain in a single fusion molecule, independent of HLA-mediated antigen presentation.

“Second-generation” CARs for use in the invention comprise an antigen binding domain, for example, a single-chain variable fragment (scFv), fused to an intracellular signaling domain capable of activating T cells and a co-stimulatory domain designed to augment T cell potency and persistence (Sadelain et al., Cancer Discov. 3:388-398 (2013)). CAR design can therefore combine antigen recognition with signal transduction, two functions that are physiologically borne by two separate complexes, the TCR heterodimer and the CD3 complex. “Second generation” CARs include an intracellular domain from various co-stimulatory molecules, for example, CD28, 4-1BB, ICOS, OX40, and the like, in the cytoplasmic tail of the CAR to provide additional signals to the cell.

“Second generation” CARs provide both co-stimulation, for example, by CD28 or 4-1BB domains, and activation, for example, by a CD3ζ signaling domain. Preclinical studies have indicated that “Second Generation” CARs can improve the anti-tumor activity of cells. For example, robust efficacy of “Second Generation” CAR modified T cells was demonstrated in clinical trials targeting the CD19 molecule in patients with chronic lymphoblastic leukemia (CLL) and acute lymphoblastic leukemia (ALL) (Davila et al., Oncoimmunol. 1(9):1577-1583 (2012)).

“Third generation” CARs provide multiple co-stimulation, for example, by comprising both CD28 and 4-1BB domains, and activation, for example, by comprising a CD3ζ activation domain.

“Fourth generation” CARs provide co-stimulation, for example, by CD28 or 4-1BB domains, and activation, for example, by a CD3ζ signaling domain in addition to a constitutive or inducible chemokine component.

“Fifth generation” CARs provide co-stimulation, for example, by CD28 or 4-1BB domains, and activation, for example, by a CD3ζ signaling domain, a constitutive or inducible chemokine component, and an intracellular domain of a cytokine receptor, for example, IL-2Rβ.

In various embodiments, the CAR can be included in a multivalent CAR system, for example, a DualCAR or “TandemCAR” system. Multivalent CAR systems include systems or cells comprising multiple CARs and systems or cells comprising bivalent/bispecific CARs targeting more than one antigen.

In the embodiments disclosed herein, the CARs generally comprise an antigen binding domain, a transmembrane domain and an intracellular domain, as described above.

### Adjuvant

In some embodiments, the agent or cargo is an adjuvant. Thus, in various embodiments, the composition comprises an adjuvant. In some embodiments, the composition comprises a nucleic acid molecule encoding an adjuvant. In some embodiments, the adjuvant-encoding nucleic acid molecule is IVT RNA. In some embodiments, the adjuvant-encoding nucleic acid molecule is nucleoside-modified mRNA.

Exemplary adjuvants include, but are not limited to, alpha-interferon, gamma-interferon, platelet derived growth factor (PDGF), TNFα, TNFβ, GM-CSF, epidermal growth factor (EGF), cutaneous T cell-attracting chemokine (CTACK), epithelial thymus-expressed chemokine (TECK), mucosae-associated epithelial chemokine (MEC), IL-12, IL-15, MHC, CD80, CD86 including IL-15 having the signal sequence deleted and optionally including the signal peptide from IgE. Other genes which may be useful adjuvants include those encoding: MCP-I, MIP-la, MIP-Ip, IL-8, RANTES, L-selectin, P-selectin, E-selectin, CD34, GlyCAM-1, MadCAM-1, LFA-I, VLA-I, Mac-1, p150.95, PECAM, ICAM-I, ICAM-2, ICAM-3, CD2, LFA-3, M-CSF, G-CSF, IL-4, mutant forms of IL-18, CD40, CD40L, vascular growth factor, fibroblast growth factor, IL-7, nerve growth factor, vascular endothelial growth factor, Fas, TNF receptor, Fit, Apo-1, p55, WSL-I, DR3, TRAMP, Apo-3, AIR, LARD, NGRF, DR4, DR5, KILLER, TRAIL-R2, TRICK2, DR6, Caspase ICE, Fos, c-jun, Sp-I, Ap-I, Ap-2, p38, p65Rel, MyD88, IRAK, TRAF6, IkB, Inactive NIK, SAP K, SAP-I, JNK, interferon response genes, NFkB, Bax, TRAIL, TRAILrec, TRAILrecDRC5, TRAIL-R3, TRAIL-R4, RANK, RANK LIGAND, Ox40, Ox40 LIGAND, NKG2D, MICA, MICB, NKG2A, NKG2B, NKG2C, NKG2E, NKG2F, TAP 1, TAP2, anti-CTLA4-sc, anti-LAG3-Ig, anti-TIM3-Ig and functional fragments thereof.

### Nucleoside-Modified RNA

In some embodiments, the agent or cargo is a nucleoside-modified RNA. Thus, in one aspect, the composition comprises a nucleoside-modified RNA. Thus, In some embodiments, the agent or cargo is a nucleoside-modified RNA In some embodiments, the composition comprises a nucleoside-modified mRNA. Nucleoside-modified mRNA have particular advantages over non-modified mRNA, including for example, increased stability, low or absent innate immunogenicity, and enhanced translation. Nucleoside-modified mRNA useful in the present disclosure is further described in U.S. Pat. No. 8,278,036, which is incorporated by reference herein in its entirety.

In certain embodiments, nucleoside-modified mRNA does not activate any pathophysiologic pathways, translates very efficiently and almost immediately following delivery, and serve as templates for continuous protein production in vivo lasting for several days (Karikó et al., 2008, Mol Ther 16:1833-1840; Karikó et al., 2012, Mol Ther 20:948-953). The amount of mRNA required to exert a physiological effect is small and that makes it applicable for human therapy.

In certain instances, expressing a protein by delivering the encoding mRNA has many benefits over methods that use protein, plasmid DNA or viral vectors. During mRNA transfection, the coding sequence of the desired protein is the only substance delivered to cells, thus avoiding all the side effects associated with plasmid backbones, viral genes, and viral proteins. More importantly, unlike DNA- and viral-based vectors, the mRNA does not carry the risk of being incorporated into the genome and protein production starts immediately after mRNA delivery. For example, high levels of circulating proteins have been measured within 15 to 30 minutes of in vivo injection of the encoding mRNA. In certain embodiments, using mRNA rather than the protein also has many advantages. Half-lives of proteins in the circulation are often short, thus protein treatment would need frequent dosing, while mRNA provides a template for continuous protein production for several days. Purification of proteins is problematic and they can contain aggregates and other impurities that cause adverse effects (Kromminga and Schellekens, 2005, Ann NY Acad Sci 1050:257-265).

In certain embodiments, the nucleoside-modified RNA comprises the naturally occurring modified-nucleoside pseudouridine. In certain embodiments, inclusion of pseudouridine makes the mRNA more stable, non-immunogenic, and highly translatable (Karikó et al., 2008, Mol Ther 16:1833-1840; Anderson et al., 2010, Nucleic Acids Res 38:5884-5892; Anderson et al., 2011, Nucleic Acids Research 39:9329-9338; Kariko et al., 2011, Nucleic Acids Research 39:e142; Kariko et al., 2012, Mol Ther 20:948-953; Karikó et al., 2005, Immunity 23:165-175).

It has been demonstrated that the presence of modified nucleosides, including pseudouridines in RNA suppress their innate immunogenicity (Karikó et al., 2005, Immunity 23:165-175). Further, protein-encoding, in vitro-transcribed RNA containing pseudouridine can be translated more efficiently than RNA containing no or other modified nucleosides (Karikó et al., 2008, Mol Ther 16:1833-1840). Subsequently, it is shown that the presence of pseudouridine improves the stability of RNA (Anderson et al., 2011, Nucleic Acids Research 39:9329-9338) and abates both activation of PKR and inhibition of translation (Anderson et al., 2010, Nucleic Acids Res 38:5884-5892). A preparative HPLC purification procedure has been established that was critical to obtain pseudouridine-containing RNA that has superior translational potential and no innate immunogenicity (Karikó et al., 2011, Nucleic Acids Research 39:e142). Administering HPLC-purified, pseudourine-containing RNA coding for erythropoietin into mice and macaques resulted in a significant increase of serum EPO levels (Karikó et al., 2012, Mol Ther 20:948-953), thus confirming that pseudouridine-containing mRNA is suitable for in vivo protein therapy.

The present disclosure encompasses RNA, oligoribonucleotide, and polyribonucleotide molecules comprising pseudouridine or a modified nucleoside. In certain embodiments, the composition comprises an isolated nucleic acid encoding an antigen or antigen binding molecule, wherein the nucleic acid comprises a pseudouridine or a modified nucleoside. In certain embodiments, the composition comprises a vector, comprising an isolated nucleic acid encoding an antigen, an antigen binding molecule, an adjuvant, or combination thereof, wherein the nucleic acid comprises a pseudouridine or a modified nucleoside.

In some embodiments, the nucleoside-modified RNA of the invention is IVT RNA. For example, in certain embodiments, the nucleoside-modified RNA is synthesized by T7 phage RNA polymerase. In another embodiment, the nucleoside-modified mRNA is synthesized by SP6 phage RNA polymerase. In another embodiment, the nucleoside-modified RNA is synthesized by T3 phage RNA polymerase.

In some embodiments, the modified nucleoside is m¹acp³Ψ (1-methyl-3-(3-amino-3-carboxypropyl) pseudouridine. In another embodiment, the modified nucleoside is m¹Ψ (1-methylpseudouridine). In another embodiment, the modified nucleoside is Ψm (2′-O-methylpseudouridine. In another embodiment, the modified nucleoside is m⁵D (5-methyldihydrouridine). In another embodiment, the modified nucleoside is m³Ψ (3-methylpseudouridine). In another embodiment, the modified nucleoside is a pseudouridine moiety that is not further modified. In another embodiment, the modified nucleoside is a monophosphate, diphosphate, or triphosphate of any of the above pseudouridines. In another embodiment, the modified nucleoside is any other pseudouridine-like nucleoside known in the art.

In another embodiment, the modified nucleoside of the present disclosure is m⁵C (5-methylcytidine). In another embodiment, the modified nucleoside is m⁵U (5-methyluridine). In another embodiment, the modified nucleoside is m⁶A (N⁶-methyladenosine). In another embodiment, the modified nucleoside is s²U (2-thiouridine). In another embodiment, the modified nucleoside is Ψ (pseudouridine). In another embodiment, the modified nucleoside is Um (2′-O-methyluridine).

In other embodiments, the modified nucleoside is m¹A (1-methyladenosine); m²A (2-methyladenosine); Am (2′-O-methyladenosine); ms²m6A (2-methylthio-N⁶-methyladenosine); i⁶A (N⁶-isopentenyladenosine); ms²i6A (2-methylthio-N⁶isopentenyladenosine); io⁶A (N⁶-(cis-hydroxyisopentenyl)adenosine); ms²io⁶A (2-methylthio-N⁶-(cis-hydroxyisopentenyl) adenosine); g⁶A (N⁶-glycinylcarbamoyladenosine); t⁶A (N⁶-threonylcarbamoyladenosine); ms²t6A (2-methylthio-N⁶-threonyl carbamoyladenosine); m⁶t6A (N⁶-methyl-N⁶-threonylcarbamoyladenosine); hn⁶A(N⁶-hydroxynorvalylcarbamoyladenosine); ms²hn⁶A (2-methylthio-N⁶-hydroxynorvalyl carbamoyladenosine); Ar(p) (2′-O-ribosyladenosine (phosphate)); I (inosine); m¹I (1-methylinosine); m¹Im (1,2′-O-dimethylinosine); m³C (3-methylcytidine); Cm (2′-O-methylcytidine); s²C (2-thiocytidine); ac4C (N⁴-acetylcytidine); fPC (5-formylcytidine); m⁵Cm (5,2′-O-dimethylcytidine); ac⁴Cm (N⁴-acetyl-2′-O-methylcytidine); k²C (lysidine); m¹G (1-methylguanosine); m²G (N²-methylguanosine); m⁷G (7-methylguanosine); Gm (2′-O-methylguanosine); m²₂G (N²,N²-dimethylguanosine); m²Gm (N²,2′-O-dimethylguanosine); m²2Gm (N²,N²,2′-O-trimethylguanosine); Gr(p) (2′-O-ribosylguanosine (phosphate)); yW (wybutosine); _{o2y}W (peroxywybutosine); OHyW (hydroxywybutosine); OHyW* (undermodified hydroxywybutosine); imG (wyosine); mimG (methylwyosine); Q (queuosine); oQ (epoxyqueuosine); galQ (galactosyl-queuosine); manQ (mannosyl-queuosine); preQ₀ (7-cyano-7-deazaguanosine); preQ₁ (7-aminomethyl-7-deazaguanosine); G⁺ (archaeosine); D (dihydrouridine); m⁵Um (5,2′-O-dimethyluridine); s4U (4-thiouridine); m⁵s²U (5-methyl-2-thiouridine); s²Um (2-thio-2′-O-methyluridine); acp³U (3-(3-amino-3-carboxypropyl)uridine); ho⁵U (5-hydroxyuridine); mo⁵U (5-methoxyuridine); cmo⁵U (uridine 5-oxyacetic acid); mcmo⁵U (uridine 5-oxyacetic acid methyl ester); chm⁵U (5-(carboxyhydroxymethyl)uridine)); mchm⁵U (5-(carboxyhydroxymethyl)uridine methyl ester); mcm⁵U (5-methoxycarbonylmethyluridine); mcm⁵Um (5-methoxycarbonylmethyl-2′-O-methyluridine); mcm⁵s2U (5-methoxycarbonylmethyl-2-thiouridine); nm⁵s2U (5-aminomethyl-2-thiouridine); mnm⁵U (5-methylaminomethyluridine); mnm⁵s2U (5-methylaminomethyl-2-thiouridine); mnm⁵se²U (5-methylaminomethyl-2-selenouridine); ncm⁵U (5-carbamoylmethyluridine); ncm⁵Um (5-carbamoylmethyl-2′-O-methyluridine); cmnm⁵U (5-carboxymethylaminomethyluridine); cmnm⁵Um (5-carboxymethylaminomethyl-2′-O-methyluridine); cmnm⁵s²U (5-carboxymethylaminomethyl-2-thiouridine); m⁶₂A (N⁶,N⁶-dimethyladenosine); Im (2′-O-methylinosine); m⁴C (N⁴-methylcytidine); m⁴Cm (N4,2′-O-dimethylcytidine); hm⁵C (5-hy droxymethylcytidine); m³U (3-methyluridine); cm⁵U (5-carboxymethyluridine); m⁶Am (N⁶,2′-O-dimethyladenosine); m⁶₂Am (N⁶,N⁶,O-2′-trimethyladenosine); m^{2,7}G (N²,7-dimethylguanosine); m^{2,2,7}G (N²,N²,7-trimethylguanosine); m³Um (3,2′-O-dimethyluridine); m⁵D (5-methyldihydrouridine); f⁵Cm (5-formyl-2′-O-methylcytidine); m¹Gm (1,2′-O-dimethylguanosine); m¹Am (1,2′-O-dimethyladenosine); τm⁵U (5-taurinomethyluridine); τm⁵s²U (5-taurinomethyl-2-thiouridine)); imG-14 (4-demethylwyosine); imG2 (isowyosine); or ac⁶A (N⁶-acetyladenosine).

In another embodiment, a nucleoside-modified RNA of the present disclosure comprises a combination of 2 or more of the above modifications. In another embodiment, the nucleoside-modified RNA comprises a combination of 3 or more of the above modifications. In another embodiment, the nucleoside-modified RNA comprises a combination of more than 3 of the above modifications.

In another embodiment, between 0.1% and 100% of the residues in the nucleoside-modified of the present disclosure are modified (e.g. either by the presence of pseudouridine or a modified nucleoside base). In another embodiment, 0.1% of the residues are modified. In another embodiment, the fraction of modified residues is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 8%. In another embodiment, the fraction is less than 10%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, 0.1% of the residues of a given nucleoside (i.e., uridine, cytidine, guanosine, or adenosine) are modified. In another embodiment, the fraction of the given nucleotide that is modified is 0.2%. In another embodiment, the fraction is 0.3%. In another embodiment, the fraction is 0.4%. In another embodiment, the fraction is 0.5%. In another embodiment, the fraction is 0.6%. In another embodiment, the fraction is 0.8%. In another embodiment, the fraction is 1%. In another embodiment, the fraction is 1.5%. In another embodiment, the fraction is 2%. In another embodiment, the fraction is 2.5%. In another embodiment, the fraction is 3%. In another embodiment, the fraction is 4%. In another embodiment, the fraction is 5%. In another embodiment, the fraction is 6%. In another embodiment, the fraction is 8%. In another embodiment, the fraction is 10%. In another embodiment, the fraction is 12%. In another embodiment, the fraction is 14%. In another embodiment, the fraction is 16%. In another embodiment, the fraction is 18%. In another embodiment, the fraction is 20%. In another embodiment, the fraction is 25%. In another embodiment, the fraction is 30%. In another embodiment, the fraction is 35%. In another embodiment, the fraction is 40%. In another embodiment, the fraction is 45%. In another embodiment, the fraction is 50%. In another embodiment, the fraction is 60%. In another embodiment, the fraction is 70%. In another embodiment, the fraction is 80%. In another embodiment, the fraction is 90%. In another embodiment, the fraction is 100%.

In another embodiment, the fraction of the given nucleotide that is modified is less than 8%. In another embodiment, the fraction is less than 10%. In another embodiment, the fraction is less than 5%. In another embodiment, the fraction is less than 3%. In another embodiment, the fraction is less than 1%. In another embodiment, the fraction is less than 2%. In another embodiment, the fraction is less than 4%. In another embodiment, the fraction is less than 6%. In another embodiment, the fraction is less than 12%. In another embodiment, the fraction is less than 15%. In another embodiment, the fraction is less than 20%. In another embodiment, the fraction is less than 30%. In another embodiment, the fraction is less than 40%. In another embodiment, the fraction is less than 50%. In another embodiment, the fraction is less than 60%. In another embodiment, the fraction is less than 70%.

In another embodiment, a nucleoside-modified RNA of the present disclosure is translated in the cell more efficiently than an unmodified RNA molecule with the same sequence. In another embodiment, the nucleoside-modified RNA exhibits enhanced ability to be translated by a target cell (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.). In another embodiment, translation is enhanced by a factor of 2-fold relative to its unmodified counterpart. In another embodiment, translation is enhanced by a 3-fold factor. In another embodiment, translation is enhanced by a 5-fold factor. In another embodiment, translation is enhanced by a 7-fold factor. In another embodiment, translation is enhanced by a 10-fold factor. In another embodiment, translation is enhanced by a 15-fold factor. In another embodiment, translation is enhanced by a 20-fold factor. In another embodiment, translation is enhanced by a 50-fold factor. In another embodiment, translation is enhanced by a 100-fold factor. In another embodiment, translation is enhanced by a 200-fold factor. In another embodiment, translation is enhanced by a 500-fold factor. In another embodiment, translation is enhanced by a 1000-fold factor. In another embodiment, translation is enhanced by a 2000-fold factor. In another embodiment, the factor is 10-1000-fold. In another embodiment, the factor is 10-100-fold. In another embodiment, the factor is 10-200-fold. In another embodiment, the factor is 10-300-fold. In another embodiment, the factor is 10-500-fold. In another embodiment, the factor is 20-1000-fold. In another embodiment, the factor is 30-1000-fold. In another embodiment, the factor is 50-1000-fold. In another embodiment, the factor is 100-1000-fold. In another embodiment, the factor is 200-1000-fold. In another embodiment, translation is enhanced by any other significant amount or range of amounts.

In another embodiment, the nucleoside-modified antigen-encoding RNA of the present disclosure induces significantly more adaptive immune response than an unmodified in vitro-synthesized RNA molecule with the same sequence. In another embodiment, the modified RNA molecule exhibits an adaptive immune response that is 2-fold greater than its unmodified counterpart. In another embodiment, the adaptive immune response is increased by a 3-fold factor. In another embodiment the adaptive immune response is increased by a 5-fold factor. In another embodiment, the adaptive immune response is increased by a 7-fold factor. In another embodiment, the adaptive immune response is increased by a 10-fold factor. In another embodiment, the adaptive immune response is increased by a 15-fold factor. In another embodiment the adaptive immune response is increased by a 20-fold factor. In another embodiment, the adaptive immune response is increased by a 50-fold factor. In another embodiment, the adaptive immune response is increased by a 100-fold factor. In another embodiment, the adaptive immune response is increased by a 200-fold factor. In another embodiment, the adaptive immune response is increased by a 500-fold factor. In another embodiment, the adaptive immune response is increased by a 1000-fold factor. In another embodiment, the adaptive immune response is increased by a 2000-fold factor. In another embodiment, the adaptive immune response is increased by another fold difference.

In another embodiment, “induces significantly more adaptive immune response” refers to a detectable increase in an adaptive immune response. In another embodiment, the term refers to a fold increase in the adaptive immune response (e.g., 1 of the fold increases enumerated above). In another embodiment, the term refers to an increase such that the nucleoside-modified RNA can be administered at a lower dose or frequency than an unmodified RNA molecule with the same species while still inducing an effective adaptive immune response. In another embodiment, the increase is such that the nucleoside-modified RNA can be administered using a single dose to induce an effective adaptive immune response.

In another embodiment, the nucleoside-modified RNA of the present disclosure exhibits significantly less innate immunogenicity than an unmodified in vitro-synthesized RNA molecule with the same sequence. In another embodiment, the modified RNA molecule exhibits an innate immune response that is 2-fold less than its unmodified counterpart. In another embodiment, innate immunogenicity is reduced by a 3-fold factor. In another embodiment, innate immunogenicity is reduced by a 5-fold factor. In another embodiment, innate immunogenicity is reduced by a 7-fold factor. In another embodiment, innate immunogenicity is reduced by a 10-fold factor. In another embodiment, innate immunogenicity is reduced by a 15-fold factor. In another embodiment, innate immunogenicity is reduced by a 20-fold factor. In another embodiment, innate immunogenicity is reduced by a 50-fold factor. In another embodiment, innate immunogenicity is reduced by a 100-fold factor. In another embodiment, innate immunogenicity is reduced by a 200-fold factor. In another embodiment, innate immunogenicity is reduced by a 500-fold factor. In another embodiment, innate immunogenicity is reduced by a 1000-fold factor. In another embodiment, innate immunogenicity is reduced by a 2000-fold factor. In another embodiment, innate immunogenicity is reduced by another fold difference.

In another embodiment, “exhibits significantly less innate immunogenicity” refers to a detectable decrease in innate immunogenicity. In another embodiment, the term refers to a fold decrease in innate immunogenicity (e.g., 1 of the fold decreases enumerated above). In another embodiment, the term refers to a decrease such that an effective amount of the nucleoside-modified RNA can be administered without triggering a detectable innate immune response. In another embodiment, the term refers to a decrease such that the nucleoside-modified RNA can be repeatedly administered without eliciting an innate immune response sufficient to detectably reduce production of the recombinant protein. In another embodiment, the decrease is such that the nucleoside-modified RNA can be repeatedly administered without eliciting an innate immune response sufficient to eliminate detectable production of the recombinant protein.

### Combinations

In some embodiments, the composition of the present disclosure comprises a combination of agents described herein. In certain embodiments, a composition comprising a combination of agents described herein has an additive effect, wherein the overall effect of the combination is approximately equal to the sum of the effects of each individual agent. In other embodiments, a composition comprising a combination of agents described herein has a synergistic effect, wherein the overall effect of the combination is greater than the sum of the effects of each individual agent.

A composition comprising a combination of agents comprises individual agents in any suitable ratio. For example, In some embodiments, the composition comprises a 1:1 ratio of two individual agents. However, the combination is not limited to any particular ratio. Rather any ratio that is shown to be effective is encompassed.

### Methods

In another aspect, the present disclosure provides a method of delivering a cargo to a subject in need thereof, the method comprising administering to the subject a therapeutically effective amount of at least one LNP of the present disclosure and/or at least one pharmaceutical composition of the present disclosure.

In certain embodiments, the cargo is at least one selected from the group consisting of a nucleic acid molecule, small molecule, protein, therapeutic agent, antibody, and any combinations thereof.

In certain embodiments, the cargo is a nucleic acid molecule. In certain embodiments, the nucleic acid molecule is a DNA molecule or a RNA molecule. In certain embodiments, the nucleic acid molecule is selected from the group consisting of mRNA, cDNA, pDNA, microRNA, siRNA, modified RNA, antagomir, antisense molecule, targeted nucleic acid, and any combination thereof.

In certain embodiments, the LNP selectively targets at least one cell type of interest.

In certain embodiments, the cell of interest is at least one selected from the group consisting of a tissue cell, muscle cell, or immune cell. In certain embodiments, the cell of interest is at least one selected from the group consisting of an immune cell, endothelial cell, epithelial cell, hematopoietic stem cell (HSC), heart cell, brain cell, bone marrow cell, hepatocyte, liver cell, spleen cell, lung cell, podocyte, and kidney cell.

In another aspect, the present disclosure provides a method of treating, preventing, and/or ameliorating a disease or disorder in a subject in need thereof, the method comprising administering to the subject a therapeutically effective amount of the LNP of the present disclosure and/or at least one pharmaceutical composition of the present disclosure.

In certain embodiments, the disease or disorder is selected from the group consisting of a liver disease or disorder, pulmonary disease or disorder, renal disease or disorder, heart disease or disorder, spleen disease or disorder, and any combinations thereof.

In another aspect, the present disclosure provides a method of inducing an immune response in a subject in need thereof, the method comprising administering to the subject a therapeutically effective amount of at least one LNP of the present disclosure and/or at least one pharmaceutical composition of the present disclosure.

In another aspect, the present disclosure provides a method of treating, preventing, and/or ameliorating cancer in a subject in need thereof, the method comprising administering to the subject a therapeutically effective amount at least one LNP of the present disclosure and/or at least one pharmaceutical composition of the present disclosure.

In certain embodiments, the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer.

In certain embodiments, the subject is further administered at least one additional agent or therapy useful for treating, preventing, and/or ameliorating cancer in the subject.

In certain embodiments, the subject is a mammal. In certain embodiments, the mammal is a human.

The present disclosure provides methods of delivering an agent to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.) of a target subject. Exemplary cells that can be targeted using the LNP compositions of the invention include, but are not limited to, a tissue cell (e.g., liver cell, lung cell, spleen cell, or any combination thereof).

In some embodiments, the agent or cargo is a diagnostic agent to detect at least one marker associated with a disease or disorder. In some embodiments, the agent or cargo is a therapeutic agent for the treatment or prevention of a disease or disorder. Therefore, in some embodiments, the invention provides methods for diagnosing, treating, or preventing a disease or disorder comprising administering an effective amount of the LNP composition comprising one or more diagnostic or therapeutic agents, one or more adjuvants, or a combination thereof.

For example, In some embodiments, the disease or disorder is a liver disease or disorder, pulmonary disease or disorder, renal disease or disorder, cardiovascular condition, such as a heart disease or disorder, spleen disease or disorder, monogenic diseases or disorders, cancer, and any combination thereof.

In some embodiments, the invention relates to methods of treating or preventing monogenic diseases or disorders and diseases or disorders associated therewith in subjects in need thereof, the method comprising administering the LNP composition of the invention.

Exemplary monogenic diseases and disorders that can be treated using the LNP compositions and methods of the invention include, but are not limited to, as sickle cell anemia, Down syndrome, fragileX syndrome, Klinefelter syndrome, Triple-X syndrome, Turner syndrome, Trisomy 18, Trisomy 13, deafness that's present at birth (congenital), familial hypercholesterolemia, hemochromatosis (iron overload), neurofibromatosis type 1 (NF1), Tay-Sachs disease, cystic fibrosis (CF), Huntington disease, Friedreich's ataxia (FA), genetic amyotrophic lateral sclerosis (ALS), hemophilia, inherited retinal disorders or dystrophies, Rett syndrome (RTT), spinal muscular atrophy (SMA), and Duchenne muscular dystrophy (DMD), and any combination thereof.

In some embodiments, the invention relates to methods of treating or preventing liver diseases or disorders and diseases or disorders associated therewith in subjects in need thereof, the method comprising administering the LNP composition of the invention. Exemplary liver diseases or disorders that can be treated using the LNP compositions and methods of the invention include, but are not limited to, hepatitis A, hepatitis B, hepatitis C, autoimmune hepatitis, primary biliary cholangitis, primary sclerosing cholangitis, hemochromatosis, Wilson's disease, alpha-1 antitrypsin deficiency, liver cancer, bile duct cancer, liver adenoma, transthyretin (TTR) based diseases, proprotein convertase subtilisin/kexin type 9 (PCSK9) based diseases, and any combination thereof.

In some embodiments, the invention relates to methods of treating or preventing pulmonary diseases or disorders and diseases or disorders associated therewith in subjects in need thereof, the method comprising administering the LNP composition of the invention. Exemplary pulmonary diseases or disorders that can be treated using the LNP compositions and methods of the invention include, but are not limited to, asthma, chronic obstructive pulmonary disease (COPD), interstitial lung disease (ILD), pulmonary embolism (PE), pulmonary hypertension, pleural effusion, pneumothorax, mesothelioma, obesity hypoventilation syndrome, neuromuscular disorders, bronchitis, chronic bronchitis, acute bronchitis, emphysema, cystic fibrosis, pneumonia, pneumoconiosis, tuberculosis, pulmonary edema, lung cancer, acute respiratory distress syndrome (ARDS), pulmonary lymphangioleiomyomatosis (LAM), and any combination thereof.

In some embodiments, the invention relates to methods of treating or preventing spleen diseases or disorders in subjects in need thereof, the method comprising administering the LNP composition of the invention. Exemplary spleen diseases or disorders that can be treated using the LNP compositions and methods of the invention include, but are not limited to, damaged or ruptured spleen, enlarged spleen, and any combination thereof.

In some embodiments, the invention relates to methods of treating or preventing cardiovascular conditions and diseases or disorders associated therewith in subjects in need thereof, the method comprising administering the LNP composition of the invention. Exemplary cardiovascular conditions that can be treated using the LNP compositions and methods of the invention include, but are not limited to, hypertrophic cardiomyopathy, dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertensive heart disease, arrhythmogenic right ventricular cardiomyopathy (ARVC), tubulointerstitial and glomerular fibrosis, atherosclerosis, varicose veins, cerebral infarcts, or any combination thereof.

In some embodiments, the invention relates to methods of treating or preventing renal diseases or disorders in subjects in need thereof, the method comprising administering the LNP composition of the invention. Exemplary renal diseases or disorders that can be treated using the LNP compositions and methods of the invention include, but are not limited to, renal fibrosis, nephritic syndrome, Alport's syndrome, HIV associated nephropathy, polycystic kidney disease, Fabry's disease, diabetic nephropathy, chronic glomerulonephritis, nephritis associated with systemic lupus); progressive systemic sclerosis (PSS), chronic graft versus host disease, or any combination thereof.

In some embodiments, the invention relates to methods of treating or preventing cancer and diseases or disorders associated therewith in subjects in need thereof, the method comprising administering the LNP composition of the invention.

In some embodiments, the present disclosure provides a method for inducing an immune response in subjects in need thereof, the method comprising administering the LNP composition of the invention. For example, In some embodiments, the method for inducing an immune response in subjects in need thereof is a cancer immunotherapy comprising administering the LNP comprising CAR to the subject to induce an immune response against cancer.

Exemplary cancers that can be treated using the LNP compositions and methods of the invention include, but are not limited to, acute lymphoblastic leukemia, acute myeloid leukemia, adrenocortical carcinoma, appendix cancer, basal cell carcinoma, bile duct cancer, bladder cancer, bone cancer, brain and spinal cord tumors, brain stem glioma, brain tumor, breast cancer, bronchial tumors, burkitt lymphoma, carcinoid tumor, central nervous system atypical teratoid/rhabdoid tumor, central nervous system embryonal tumors, central nervous system lymphoma, cerebellar astrocytoma, cerebral astrocytoma/malignant glioma, cerebral astrocytotna/malignant glioma, cervical cancer, childhood visual pathway tumor, chordoma, chronic lymphocytic leukemia, chronic myelogenous leukemia, chronic myeloproliferative disorders, colon cancer, colorectal cancer, craniopharyngioma, cutaneous cancer, cutaneous t-cell lymphoma, endometrial cancer, ependymoblastoma, ependymoma, esophageal cancer, ewing family of tumors, extracranial cancer, extragonadal germ cell tumor, extrahepatic bile duct cancer, extrahepatic cancer, eye cancer, fungoides, gallbladder cancer, gastric (stomach) cancer, gastrointestinal cancer, gastrointestinal carcinoid tumor, gastrointestinal stromal tumor (gist), germ cell tumor, gestational cancer, gestational trophoblastic tumor, glioblastoma, glioma, hairy cell leukemia, head and neck cancer, hepatocellular (liver) cancer, histiocytosis, hodgkin lymphoma, hypopharyngeal cancer, hypothalamic and visual pathway glioma, hypothalamic tumor, intraocular (eye) cancer, intraocular melanoma, islet cell tumors, kaposi sarcoma, kidney (renal cell) cancer, langerhans cell cancer, langerhans cell histiocytosis, laryngeal cancer, leukemia, lip and oral cavity cancer, liver cancer, lung cancer, lymphoma, macroglobulinemia, malignant fibrous histiocytoma of bone and osteosarcoma, medulloblastoma, medulloepithelioma, melanoma, merkel cell carcinoma, mesothelioma, metastatic squamous neck cancer with occult primary, mouth cancer, multiple endocrine neoplasia syndrome, multiple myeloma, mycosis, myelodysplastic syndromes, myelodysplastic/myeloproliferative diseases, myelogenous leukemia, myeloid leukemia, myeloma, myeloproliferative disorders, nasal cavity and paranasal sinus cancer, nasopharyngeal cancer, neuroblastoma, non-hodgkin lymphoma, non-small cell lung cancer, oral cancer, oral cavity cancer, oropharyngeal cancer, osteosarcoma and malignant fibrous histiocytoma, osteosarcoma and malignant fibrous histiocytoma of bone, ovarian, ovarian cancer, ovarian epithelial cancer, ovarian germ cell tumor, ovarian low malignant potential tumor, pancreatic cancer, papillomatosis, paraganglioma, parathyroid cancer, penile cancer, pharyngeal cancer, pheochromocytoma, pineal parenchymal tumors of intermediate differentiation, pineoblastoma and supratentorial primitive neuroectodermal tumors, pituitary tumor, plasma cell neoplasm, plasma cell neoplasm/multiple myeloma, pleuropulmonary blastoma, primary central nervous system cancer, primary central nervous system lymphoma, prostate cancer, rectal cancer, renal cell (kidney) cancer, renal pelvis and ureter cancer, respiratory tract carcinoma involving the nut gene on chromosome 15, retinoblastoma, rhabdomyosarcoma, salivary gland cancer, sarcoma, sezary syndrome, skin cancer (melanoma), skin cancer (nonmelanoma), skin carcinoma, small cell lung cancer, small intestine cancer, soft tissue cancer, soft tissue sarcoma, squamous cell carcinoma, squamous neck cancer, stomach (gastric) cancer, supratentorial primitive neuroectodermal tumors, supratentorial primitive neuroectodermal tumors and pineoblastoma, T-cell lymphoma, testicular cancer, throat cancer, thymoma and thymic carcinoma, thyroid cancer, transitional cell cancer. transitional cell cancer of the renal pelvis and ureter, trophoblastic tumor, urethral cancer, uterine cancer, uterine sarcoma, vaginal cancer, visual pathway and hypothalamic glioma, vulvar cancer, waldenstrom macroglobulinemia, and wilms tumor.

In various embodiments, the disease or disorder is a disease or disorder associated with at least one cell of interest (e.g., a tissue cell, muscle cell, immune cell, stem cell, HSC, myeloid-lineage cell, lymphoid-lineage cell, blood cell, bone cell, fat cell, endothelial cell, epithelial cell, cancer cell, brain cell, bone marrow cell, nerve cell, connective tissue cell, neuron, neuroglial cell, heart cell, liver cell, hepatocytes, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakaryocyte, embryonic stem cell, adult stem cell, mesenchymal stem cell, hematopoietic stem cell, white adipocyte, and/or brown adipocyte, etc.). For example, In some embodiments, the disease or disorder associated with at least one cell of interest (e.g., a tissue cell, muscle cell, immune cell, stem cell, HSC, myeloid-lineage cell, lymphoid-lineage cell, blood cell, bone cell, fat cell, endothelial cell, epithelial cell, cancer cell, brain cell, bone marrow cell, nerve cell, connective tissue cell, neuron, neuroglial cell, heart cell, liver cell, hepatocytes, spleen cell, lung cell, kidney cell, podocytes, skin cell, keratinocyte, melanocyte, merkel cell, langerhans cell, cartilage cell, chondrocyte, pancreatic cell, skeletal muscle cell, cardiac muscle cell, smooth muscle cell, bone cell, osteoblast, osteoclast, osteocyte, lining cell, bone marrow cell, lymph node cell, white blood cell, granulocyte, neutrophil, eosinophil, basophil, agranulocyte, monocyte, lymphocyte, red blood cell, erythrocyte, platelet, fragments of megakaryocyte, embryonic stem cell, adult stem cell, mesenchymal stem cell, hematopoietic stem cell, white adipocyte, and/or brown adipocyte, etc.) is a liver disease or disorder, pulmonary disease or disorder, renal disease or disorder, heart disease or disorder, spleen disease or disorder, monogenic diseases or disorders, cancer, or any combination thereof.

In some embodiments, the method comprises administering a LNP composition of the invention comprising one or more nucleic acid molecules for treatment or prevention of a disease or disorder (e.g., cancer, liver disease or disorder, pulmonary disease or disorder, renal disease or disorder, heart disease or disorder, spleen disease or disorder, and any combination thereof). In some embodiments, the one or more nucleic acid molecules encode a therapeutic agent for the treatment of the disease or disorder (e.g., cancer, liver disease or disorder, pulmonary disease or disorder, renal disease or disorder, heart disease or disorder, spleen disease or disorder, and any combination thereof).

In some embodiments, the compositions of the invention can be administered in combination with one or more additional therapeutic agent, an adjuvant, or a combination thereof. For example, In some embodiments, the method comprises administering an LNP composition comprising a nucleic acid molecule encoding one or more agent for targeted administration to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.) and a second LNP comprising a nucleic acid molecule encoding one or more adjuvants. In some embodiments, the method comprises administering a single LNP composition comprising a nucleic acid molecule encoding one or more agent for targeted administration to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.) and a nucleic acid molecule encoding one or more adjuvants.

In certain embodiments, the method comprises administering to subject a plurality of LNPs of the invention comprising nucleoside-modified nucleic acid molecules encoding a plurality of agents to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.), adjuvants, or a combination thereof.

In certain embodiments, the method comprises administering the LNP of the invention comprising nucleoside-modified RNA, which provides stable expression of a nucleic acid encoded agent (e.g., a therapeutic agent encoded by a nucleoside modified mRNA molecule) described herein to a cell of interest (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.).

The therapeutic and prophylactic methods of the invention thus encompass the use of pharmaceutical compositions comprising at least one LNP of the invention comprising an agent (e.g., an mRNA, siRNA, microRNA, DNA, pDNA, and/or antisense oligonucleotide molecule) described herein, to practice the methods of the invention. The pharmaceutical compositions useful for practicing the invention may be administered to deliver a dose of from ng/kg/day and 100 mg/kg/day. In some embodiments, the invention envisions administration of a dose which results in a concentration of the compound of the present disclosure from 10 nM and 10 μM in a mammal.

In some embodiments, the invention includes a method comprising administering a combination of LNP compositions described herein. In certain embodiments, the combination has an additive effect, wherein the overall effect of the administering the combination is approximately equal to the sum of the effects of administering each LNP composition. In other embodiments, the combination has a synergistic effect, wherein the overall effect of administering the combination is greater than the sum of the effects of administering each LNP composition.

In some aspects of the invention. the method provides for delivery of compositions for gene editing or genetic manipulation to a target cell (e.g., a tissue cell, muscle cell, immune cell, endothelial cell, epithelial cell, HSC, heart cell, brain cell, bone marrow cell, hepatocytes, liver cell, spleen cell, lung cell, podocytes, and/or kidney cell, etc.) of a subject to treat or prevent a disease or disorder (e.g., a liver disease or disorder, pulmonary disease or disorder, renal disease or disorder, heart disease or disorder, spleen disease or disorder, cancer, and any combination thereof).

### Therapy

In one aspect, the therapeutic compounds or compositions of the invention may be administered prophylactically (i.e., to prevent disease or disorder, such as a liver disease or disorder, pulmonary disease or disorder, renal disease or disorder, heart disease or disorder, spleen disease or disorder, cancer, and any combination thereof) or therapeutically (i.e., to treat disease or disorder, such as a liver disease or disorder, pulmonary disease or disorder, renal disease or disorder, heart disease or disorder, spleen disease or disorder, cancer, and any combination thereof) to subjects suffering from or at risk of (or susceptible to) developing the disease or disorder (e.g., a liver disease or disorder, pulmonary disease or disorder, renal disease or disorder, heart disease or disorder, spleen disease or disorder, cancer, and any combination thereof). Such subjects may be identified using standard clinical methods.

In the context of the present invention, prophylactic administration occurs prior to the manifestation of overt clinical symptoms of disease or disorder (e.g., a liver disease or disorder, pulmonary disease or disorder, renal disease or disorder, heart disease or disorder, spleen disease or disorder, cancer, and any combination thereof), such that the disease or disorder is prevented or alternatively delayed in its progression. In the context of the field of medicine, the term “prevent” encompasses any activity which reduces the burden of mortality or morbidity from a disease. Prevention can occur at primary, secondary and tertiary prevention levels. While primary prevention avoids the development of a disease, secondary and tertiary levels of prevention encompass activities aimed at preventing the progression of a disease and the emergence of symptoms as well as reducing the negative impact of an already established disease by restoring function and reducing disease-related complications.

The composition of the invention can be useful in combination with therapeutic, anti-cancer, and/or radiotherapeutic agents. Thus, the present disclosure provides a combination of the present LNP with therapeutic, anti-cancer, and/or radiotherapeutic agents for simultaneous, separate, or sequential administration. The composition of the invention and the other anticancer agent can act additively or synergistically.

The therapeutic agent, anti-cancer agent, and/or radiation therapy can be administered according to therapeutic protocols well known in the art. It will be apparent to those skilled in the art that the administration of the therapeutic agent, anti-cancer agent, and/or radiation therapy can be varied depending on the disease being treated and the known effects of the anti-cancer agent and/or radiation therapy on that disease. Also, in accordance with the knowledge of the skilled clinician, the therapeutic protocols (e.g., dosage amounts and times of administration) can be varied in view of the observed effects of the administered therapeutic agents (i.e., anti-neoplastic agent or radiation) on the patient, and in view of the observed responses of the disease to the administered therapeutic agents, and observed adverse effects.

### Pharmaceutical Compositions

In another aspect, the present disclosure provides a pharmaceutical composition comprising the LNP of the present disclosure and a pharmaceutically acceptable carrier. The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the description of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the invention is contemplated include, but are not limited to, humans and other primates, mammals including commercially relevant mammals such as non-human primates, cattle, pigs, horses, sheep, cats, and dogs.

Pharmaceutical compositions that are useful in the methods of the invention may be prepared, packaged, or sold in formulations suitable for ophthalmic, oral, rectal, vaginal, parenteral, topical, pulmonary, intranasal, buccal, intravenous, intracerebroventricular, intradermal, intramuscular, or another route of administration. Other contemplated formulations include projected nanoparticles, liposomal preparations, resealed erythrocytes containing the active ingredient, and immunogenic-based formulations.

A pharmaceutical composition of the invention may be prepared, packaged, or sold in bulk, as a single unit dose, or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the invention will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the invention may further comprise one or more additional pharmaceutically active agents.

Controlled- or sustained-release formulations of a pharmaceutical composition of the invention may be made using conventional technology.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, intraocular, intravitreal, subcutaneous, intraperitoneal, intramuscular, intradermal, intrasternal injection, intratumoral, intravenous, intracerebroventricular and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e. powder or granular) form for reconstitution with a suitable vehicle (e.g. sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer systems. Compositions for sustained release or implantation may comprise pharmaceutically acceptable polymeric or hydrophobic materials such as an emulsion, an ion exchange resin, a sparingly soluble polymer, or a sparingly soluble salt.

A pharmaceutical composition of the invention may be prepared, packaged, or sold in a formulation suitable for pulmonary administration via the buccal cavity. Such a formulation may comprise dry particles which comprise the active ingredient and which have a diameter in the range from about 0.5 to about 7 micrometers, and preferably from about 1 to about 6 micrometers. Such compositions are conveniently in the form of dry powders for administration using a device comprising a dry powder reservoir to which a stream of propellant may be directed to disperse the powder or using a self-propelling solvent/powder-dispensing container such as a device comprising the active ingredient dissolved or suspended in a low-boiling propellant in a sealed container. Preferably, such powders comprise particles wherein at least 98% of the particles by weight have a diameter greater than 0.5 micrometers and at least 95% of the particles by number have a diameter less than 7 micrometers. More preferably, at least 95% of the particles by weight have a diameter greater than 1 micrometer and at least 90% of the particles by number have a diameter less than 6 micrometers. Dry powder compositions preferably include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic or solid anionic surfactant or a solid diluent (preferably having a particle size of the same order as particles comprising the active ingredient).

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multi-dose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In some embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butane diol, for example. Other acceptable diluents and solvents include, but are not limited to, Ringer's solution, isotonic sodium chloride solution, and fixed oils such as synthetic mono- or di-glycerides. Other parentally-administrable formulations that are useful include those that comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the invention are known in the art and described, for example in Remington's Pharmaceutical Sciences (1985, Genaro, ed., Mack Publishing Co., Easton, PA), which is incorporated herein by reference.

### Administration/Dosing

The regimen of administration may affect what constitutes an effective amount. The therapeutic formulations may be administered to the patient either prior to or after the onset of a disease or disorder. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present disclosure to a patient, such as a mammal, such as a human, may be carried out using known procedures, at dosages and for periods of time effective to treat a disease or disorder contemplated herein. An effective amount of therapeutic (i.e., composition) necessary to achieve a therapeutic effect may vary according to factors such as the activity of the particular therapeutic employed; the time of administration; the rate of excretion of the composition; the duration of the treatment; other drugs, compounds or materials used in combination with the composition; the state of the disease or disorder, age, sex, weight, condition, general health and prior medical history of the patient being treated, and like factors well-known in the medical arts. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic composition of the disclosure is from about 0.01 mg/kg to 100 mg/kg of body weight/per day of active agent (i.e., nucleic acid). One of ordinary skill in the art would be able to study the relevant factors and make the determination regarding the effective amount of the therapeutic composition without undue experimentation.

The composition may be administered to an animal as frequently as several times daily, or it may be administered less frequently, such as once a day, once a week, once every two weeks, once a month, or even less frequently, such as once every several months or even once a year or less. It is understood that the amount of composition dosed per day may be administered, in non-limiting examples, every day, every other day, every 2 days, every 3 days, every 4 days, or every 5 days. For example, with every other day administration, a 5 mg per day dose may be initiated on Monday with a first subsequent 5 mg per day dose administered on Wednesday, a second subsequent 5 mg per day dose administered on Friday, and so on. The frequency of the dose is readily apparent to the skilled artisan and depends upon a number of factors, such as, but not limited to, type and severity of the disease being treated, and type and age of the animal.

Actual dosage levels of the active ingredients in the pharmaceutical compositions of this disclosure may be varied so as to obtain an amount of the active ingredient that is effective to achieve the desired therapeutic response for a particular patient, composition, and mode of administration, without being toxic to the patient.

A medical doctor, e.g., physician or veterinarian, having ordinary skill in the art may readily determine and prescribe the effective amount of the pharmaceutical composition required. For example, the physician or veterinarian could start doses of the compounds of the disclosure employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic composition to produce the desired therapeutic effect in association with the required pharmaceutical vehicle. The dosage unit forms of the disclosure are dictated by and directly dependent on (a) the unique characteristics of the therapeutic composition and the particular therapeutic effect to be achieved, and (b) the limitations inherent in the art of compounding/formulating such a therapeutic composition for the treatment of a disease or disorder in a patient.

In certain embodiments, the compositions of the disclosure are administered to the patient in dosages that range from one to five times per day or more. In other embodiments, the compositions of the disclosure are administered to the patient in range of dosages that include, but are not limited to, once every day, every two days, every three days to once a week, and once every two weeks. It will be readily apparent to one skilled in the art that the frequency of administration of the various combination compositions of the disclosure will vary from subject to subject depending on many factors including, but not limited to, age, disease or disorder to be treated, gender, overall health, and other factors. Thus, the disclosure should not be construed to be limited to any particular dosage regime and the precise dosage and composition to be administered to any patient will be determined by the attending physician taking all other factors about the patient into account.

The amount of active agent of the composition(s) of the disclosure for administration may be in the range of from about 1 μg to about 7,500 mg, about 20 μg to about 7,000 mg, about 40 μg to about 6,500 mg, about 80 μg to about 6,000 mg, about 100 μg to about 5,500 mg, about 200 μg to about 5,000 mg, about 400 μg to about 4,000 mg, about 800 μg to about 3,000 mg, about 1 mg to about 2,500 mg, about 2 mg to about 2,000 mg, about 5 mg to about 1,000 mg, about 10 mg to about 750 mg, about 20 mg to about 600 mg, about 30 mg to about 500 mg, about 40 mg to about 400 mg, about 50 mg to about 300 mg, about 60 mg to about 250 mg, about 70 mg to about 200 mg, about 80 mg to about 150 mg, and any and all whole or partial increments there-in-between.

In some embodiments, the dose of active agent (i.e., nucleic acid) present in the composition of the disclosure is from about 0.5 μg and about 5,000 mg. In some embodiments, a dose of active agent present in the composition of the disclosure used in compositions described herein is less than about 5,000 mg, or less than about 4,000 mg, or less than about 3,000 mg, or less than about 2,000 mg, or less than about 1,000 mg, or less than about 800 mg, or less than about 600 mg, or less than about 500 mg. or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

In certain embodiments, the present disclosure is directed to a packaged pharmaceutical composition comprising a container holding a therapeutically effective amount of the composition of the disclosure, alone or in combination with a second pharmaceutical agent; and instructions for using the compound to treat, prevent, or reduce one or more symptoms of a disease or disorder in a patient.

The term “container” includes any receptacle for holding the pharmaceutical composition or for managing stability or water uptake. For example, in certain embodiments, the container is the packaging that contains the pharmaceutical composition, such as liquid (solution and suspension), semisolid, lyophilized solid, solution and powder or lyophilized formulation present in dual chambers. In other embodiments, the container is not the packaging that contains the pharmaceutical composition, i.e., the container is a receptacle, such as a box or vial that contains the packaged pharmaceutical composition or unpackaged pharmaceutical composition and the instructions for use of the pharmaceutical composition. Moreover, packaging techniques are well known in the art. It should be understood that the instructions for use of the pharmaceutical composition may be contained on the packaging containing the pharmaceutical composition, and as such the instructions form an increased functional relationship to the packaged product. However, it should be understood that the instructions may contain information pertaining to the compound's ability to perform its intended function, e.g., treating, preventing, or reducing a disease or disorder in a patient.

### Administration

Routes of administration of any of the compositions of the disclosure include inhalational, oral, nasal, rectal, parenteral, sublingual, transdermal, transmucosal (e.g., sublingual, lingual, (trans)buccal, (trans)urethral, vaginal (e.g., trans- and perivaginally), (intra)nasal, and (trans)rectal), intravesical, intrapulmonary, intraduodenal, intragastrical, intrathecal, epidural, intrapleural, intraperitoneal, subcutaneous, intramuscular, intradermal, intra-arterial, intravenous, intrabronchial, inhalation, and topical administration.

Suitable compositions and dosage forms include, for example, tablets, capsules, caplets, pills, gel caps, troches, emulsions, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present disclosure are not limited to the particular formulations and compositions that are described herein.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Injectable formulations may also be prepared, packaged, or sold in devices such as patient-controlled analgesia (PCA) devices. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen-free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form in a recombinant human albumin, a fluidized gelatin, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### EXPERIMENTAL EXAMPLES

Various embodiments of the present application can be better understood by reference to the following Examples which are offered by way of illustration. The scope of the present application is not limited to the Examples given herein.

### Materials and Methods

### Chemicals and Reagents for Synthesis

3-Aminopropyl pentamethyldisiloxane (97%, Gelest), 3-aminopropyl methylbis(termethylsiloxy)silane (97%, Gelest), 3-aminopropyltris(termethylsiloxy)silane (95%, Gelest), 1,3-bis(3-aminopropyl) tetramethyldisiloxane (97%, Gelest), 1,3-bis(2-aminoethylaminomethyl) tetramethyldisiloxane (technical grade, Gelest), aminopropyl terminated polvdimethylsiloxane (10-15 cSt, Gelest), aminopropyl terminated polydimethylsiloxane (50-60 cSt, Gelest), vinyl pentamethyldisiloxane, (Gelest), 1,3-divinyl tetramethyldisiloxane (97%, Gelest), 1,3,5-trivinyl-1,3,5-trimethylcyclotrisiloxane (Gelest), 1,3,5,7-tetravinyl-1,3,5,7-tetramethylcyclotetrasiloxane (Gelest), 2-mercaptoethylamine (95%, Sigma-Aldrich), 2,2′-azobis(2-methylpropionitrile) (AIBN, 98%, Sigma-Aldrich), di-tert-butyl decarbonate (95%, TCI), sodium 2-mercaptoethanesulfonate (98%, Sigma-Aldrich), 1,2-epoxyhexane (C6, 97%, Sigma-Aldrich), 1,2-epoxyoctane (C8, 96%, TCI), 1,2-epoxydecane (C10, 97%, TCI), 1,2-epoxy-9-decene (C10V. 96%, Sigma-Aldrich), 2-ethylhexyl glycidyl ether (C11b, 98%, Sigma-Aldrich), epoxy-branched dodecane (C12b, Wuxi AppTec), 1,2-epoxy-dodecane (C12, 90%, Sigma-Aldrich), 1,2-epoxytetradecane (C14, technical grade, 85%, Sigma-Aldrich), epoxybranched tetradecane (C14b, Wuxi AppTec), 1,2-epoxyhexadecane (C16, technical grade, 85%, Sigma-Aldrich), 1,2-epoxyoctadecane (C18, technical grade, 85%, TCI), nonyl acrylate (09, 95%, Sigma-Aldrich), lauryl acrylate (012, 95%, Sigma-Aldrich), tetradecyl acrylate (014, 95%, TCI), hexadecyl acrylate (016, 90%, TCI), stearyl acrylate (018, 97%, TCI), acryloyl chlorid (97%, Sigma-Aldrich), n-octylamine (98%, TCI), 1-aminodecane (98%, TCI), dodecylamine (97%, TCI), tetradecylamine (96%, TCI), hexadecylamine (95%, TCI), trifluoroacetic acid (TFA, 98%, Thermo Scientific), 6-(p-toluidinyl)naphthalene-2-sulfonic acid (TNS, Sigma-Aldrich), triethylamine (TEA, anhydrous, 99.5%, Sigma-Aldrich), D-Lin-MC3-DMA (98%, MedChemExpress), 1,2-dioleoyl-sn-glycero-3-phosphoethanolamine (DOPE, AvantiPolarLipids), 1,2-distearoyl-sn-glycero-3-phosphocholine (DSPC, AvantiPolarLipids), cholesterol (Sigma-Aldrich) and 1,2-dimyristoyl-sn-glycero-3-phosphoethanolamine-N-[methoxy(polyethyleneglycol)-2000](C14-PEG2000, AvantiPolarLipids) and 31,1′-dioctadecyl-3,3,3′,3′-tetramethylindodicarbocyanine, 4-chlorobenzenesulfonate salt (DiD, ThermoFisher) were used as received. Organic solvents were purchased from Fisher Scientific. Chloroform-d (CDCl₃), DMSO-d₆ and methanol-d₄ (MeOH-d₄) were purchased from Acros Organics.

### Nucleic Acids and Other Reagents for Biological Assays

Luciferase mRNA were provided by Prof Drew Weissman. CleanCap® Cre mRNA and CleanCap® Cas9 mRNA were purchased from TriLink Biotechnologies. GFP single guide RNA were obtained from Axolabs GmbH. Mouse transthyretin (mTTR) single guide RNA were purchased from Axolabs GmbH as previously described. Luciferase 1000 Assay System (Ref E4550) and CellTiter-Glo Luminescent Cell Viability (Ref. G7572) were purchased from Promega Corporation. Alanine Transaminase (ALT) Colorimetric Activity 10 Assay Kit (Item. 700260) and Aspartate Aminotransferase (AST) Colorimetric Activity Assay Kit (Item. 701640) for liver toxicity markers were purchased from Cayman Chemical. Urea Assay Kit (BUN, Item: ab83362) and Creatinine Assay Kit (Item: ab65340) for kidney toxicity markers were purchased from abcam. Prealbumin ELISA Kit (Cat #OKIA00111) for TTR reduction was purchased from aviva systems biology. ISH kit for TTR mRNA (LS 2.5 Probe-Mm-Ttr, Cat #424178) was purchased from ACD Bio. Antibodies for flow cytometry including antimouse CD31 antibody (AF488, Cat #102514; PE, Cat #102508), CD45 antibody (BV421, Cat #103134), F4/80 antibody (BV421, Cat #123137; AF647, Cat #123122), CD3 antibody (AF700, Cat #100216), CD19 antibody (AF488, Cat #115521), CD11c antibody (APC, Cat #117309), CD326 antibody (EpCAM, AF647, Cat #118212) and Live/Dead staining Draq7 (Cat #424001) were purchased from Biolegend.

### Cell Culture

Dulbecco's Modified Eagle Medium (DMEM) was purchased from Gibco containing high glucose, L-glutamine, phenol red, and without sodium pyruvate and HEPES. Trypsin-EDTA (0.25%), penicillin streptomycin (P/S) were purchased from Gibco. Fetal bovine serum (FBS) was purchased from Sigma-Aldrich. HeLa and HepG2 cells were cultured in DMEM supplemented with 10% FBS and 1% P/S. GFP-HepG2 cells were cultured in DMEM supplemented with 10% FBS and 1% P/S.

### Animal Studies

C57BL/6, C57BL/6-Tg(CAG-EGFP)1Osb/J, and B6.Cg-Gt(ROSA)26Sortm14(CAG-tdTomato)Hze/J (Ai14) mice were purchased from Jackson Laboratory.

### Instruments

¹H NMR spectrum were performed on a NEO 400 MHz spectrometer. LC-MS was performed on an Agilent LCMS system equipped with UV-Vis and evaporative light scattering detectors (ELSD). Flash chromatography was performed on a Teledyne IscoCombiFlash Rf-200i chromatography system equipped with UV-Vis and evaporative light scattering detectors (ELSD). LNPs were formulated by a Pump33DS syringe pump (Harvard Apparatus, Holliston, MA) and/or a NanoAssemblr Ignite (Precision Nanosystems, Vancouver, Canada). Particle size and zeta potentials were measured by Dynamic Light Scattering (DLS) with Malvern Zetasizer Nano ZS. Particle morphology was measured by Cryo-TEM. Zeiss LSM 710 Confocal was used to evaluate the GFP knocking out on cell levels. Leica SP8 microscope was used for immunofluorescence of liver and lung tissues. Flow cytometry was performed using an LSR II, LSRFortessa, and Symphony A3 Lite machine (BD Biosciences). In vitro luminescent intensity, cell viability, ALT qualification, AST qualification, TNS assay, BUN, Creatinine tests, and serum mTTR protein qualification were quantified using an Infinite M Plex plate reader (Tecan, Morrisville, NC).

Formulation of Siloxane-Based Ionizable Lipids into Nanoparticles (SiLNPs)

All LNPs used for small batch in vitro and in vivo studies were prepared as follows. An ethanol phase containing all lipids and an aqueous phase containing mRNA (Fluc mRNA, Cre mRNA, or Cas9 mRNA/sgRNA) were mixed using a microfluidic device to formulate LNPs. The ethanol phase contained siloxane lipidoids, 1,2-dioleoyl-sn-glycero-3-phosphoethanolamine (DOPE), cholesterol and 1,2-dimyristoyl-sn-glycero-3-phosphoethanolamine-N-[methoxy(polyethyleneglycol)-2000](C14-PEG2000) with a fixed molar ratio of 35%, 16%, 46.5% and 2.5%, respectively. Aqueous phase was composed of mRNA dissolved in 10 mM citrate buffer. The ethanol and aqueous phases were mixed at a flow rate of 1.8 mL/min and 0.6 m/min (3:1) using Pump33DS syringe pumps. LNPs were dialyzed in 1×PBS using a microdialysis cassette (20,000 MWCO, Thermo Fisher Scientific, Waltham, MA) for 2 h and then filtered through a 0.22 m filter. Zetasizer Nano was used to measure the Z-average diameters, polydispersity index (PDI) and Zeta potential. mRNA concentration and encapsulation efficiency in each LNP formulation were measured using a modified Quant-iT RiboGreen (ThermoFisher) assay on a plate reader. Note that DLin-MC3-DMA (MC3) LNP was formulated according to a similar protocol, but the ethanol phase contained MC3 lipid, 1,2-distearoyl-sn-glycero-3-phosphocholine (DSPC), cholesterol, and C14-PEG2000 with a fixed molar ratio of 50%, 10%, 38.5, and 1.5%, respectively. LNPs for large batch in vivo gene editing studies were formulated on a NanoAssemblr Ignite device. The ethanol phase and an aqueous phase were prepared as above, which was then mixed at a total flow rate of 12 mL/min (aqueous/ethanol flow rate ratio of 3:1) using a NanoAssemblr Ignite system. Then the mixture was dialyzed in 1×PBS using a microdialysis cassette (20,000 MWCO, Thermo Fisher Scientific, Waltham, MA) for 2 h. Resultant LNPs were concentrated with an Amicon Ultra 50 K MWCO (Merk Millipore, Burlington, MA) and filtrated through a 0.22 m filter.

In Vitro Fluc mRNA LNP Library Screening

In a white transparent 96-well plate, HepG2 cells were seeded at a density of 5×10³ cells per well in 100 μL growth medium (DMEM, 10% FBS, 1% P/S), and were incubated at 37° C. in 5% CO₂. The medium was exchanged for fresh growth medium, and then LNPs were treated at a dose of 10 ng Fluc mRNA per well. Luciferase expression was measured 24 h after LNP transfection using a Luciferase Assay System (Promega) according to the manufacturer's protocol. The luminescent signal was normalized to medium treated cells. Cell viability was measured using a CellTiter-Glo Luminescent Cell Viability Assay (Promega), in which the luminescence was normalized to growth medium treated cells according to the manufacturer's protocol. For mRNA dose response studies, LNPs were treated with 10 ng, 20 ng, 40 ng, 80 ng, and 160 ng Fluc mRNA per well.

### In Vitro GFP Knockout Study

In a transparent 6-well plate, GFP-HepG2 cells were seeded at a density of 2×10⁴ cells per well in 2 mL growth medium (DMEM, 10% FBS, 1% P/S), and were incubated at 37° C. in 5% CO₂. The medium was exchanged for fresh growth medium, and then LNPs were treated at a dose of 400, 800, 1200, 1600, 2000, 4000, and 6000 ng Cas9 mRNA/GFP sgRNA (4:1, 3:1, 2:1, 1:1) per well. Medium treated group and Lipofectamine CRISPR MAX with the same Cas9 mRNA/GFP sgRNA dose were used as negative and positive controls, respectively. GFP knockout was measured 7 days after LNP incubation using LSR II flow cytometry. Representative GFP signal knockout was imaged using a Zeiss LSM 710 confocal microscope. The editing rate was calculated by the main GFP fluorescent intensity normalized to growth medium treated groups.

In Vivo Fluc mRNA LNP Delivery

In certain embodiments, animal procedures were performed on female C57BL/6 mice aged 6-8 weeks. Mice were administered a single intravenous Fluc mRNA via tail vein injection. Luciferase expression was evaluated using an IVIS Spectrum imaging system (Caliper Life Sciences) 6 h post-injection. Mice were then injected with D-luciferin (PerkinElmer) at a dose of 150 mg kg⁻¹ by intraperitoneal (i.p.) injection. After 10 min incubation under anesthesia, bioluminescence intensity was quantified by measuring photon flux in the region of interest where signal emanated using Living IMAGE Software provided by Caliper. Ex vivo imaging was performed on heart, liver, spleen, lung, and kidney after resection.

In Vivo Cre mRNA LNP Delivery

B6.Cg-Gt(ROSA)26Sortm14(CAG-tdTomato)Hze/J (Ai14) mice were administered a single intravenous dose of Cre mRNA LNPs at a dosage of 0.3 mg kg⁻¹ via tail vein injection. To test the tdTomato+ cells in different cell types of organs, cell isolation and staining were conducted after 3 days post-injection, followed by flow cytometry measurements. Liver cell isolation and staining: mice were first anesthetized using isoflurane, then were perfused with DMEM medium containing collagen IV (0.5 mg mL⁻¹) and 1×PBS containing 0.1% BSA and 0.2% EDTA, respectively. After that, the liver was collected and grinded into small pieces to release liver cells. The obtained cell suspension was then centrifuged (5 min, 500 g) and lysed by ACK lysis buffer (ThermoFisher) (1 mL) for 10 min. Afterwards, single-cell suspensions were obtained by centrifugation (5 min, 500 g) and resuspended in 1×PBS (200 μL). The antibodies used were: anti-mouse Alexa Fluor 488 CD31 antibody (1:200, Biolegend, Cat #102514), Brilliant Violet 421 CD45 antibody (1:200, Biolegend, Cat #103134), AF647 F4/80 antibody (1:200, Biolegend, Cat #123122). The obtained single-cell suspensions were stained at 4° C. for 30 min by each of the above antibodies (3 μL), and afterwards were centrifuged, washed, centrifuged and resuspended in Draq7 dyed 1×PBS (1 mL, 0.1%) for flow cytometry analysis. The liver cells were analyzed using a Symphony A3 Lite machine. Lung cell isolation and staining: mice were firstly anesthetized by isoflurane, then were perfused with DMEM medium containing collagen IV (0.5 mg mL-1) and 1×PBS containing 0.1% BSA and 0.2% EDTA, respectively. Afterwards the lung was collected and grinded into small pieces to release lung cells. The obtained cell suspension was then centrifuged (5 min, 500 g) and lysed by ACK lysis buffer (ThermoFisher) (1 mL) for 10 min. Afterwards, single-cell suspensions were obtained by centrifugation (5 min, 500 g) and resuspended in 1×PBS (200 μL). The antibodies used were: anti-mouse Alexa Fluor 488 CD31 antibody (1:200, Biolegend, Cat #102514), Brilliant Violet 421 CD45 antibody (1:200, Biolegend, Cat #103134), AF647 CD326 antibody (1:200, EpCAM, Biolegend, Cat #118212). The obtained single-cell suspensions were stained at 4° C. for 30 min by each of the above antibodies (3 μL), and afterwards were centrifuged, washed, centrifuged and resuspended in Draq7 dyed 1×PBS (1 mL, 0.1%) for flow cytometry analysis. The lung cells were analyzed using a LSRForessa machine. Spleen cells isolation and staining: mice were euthanized by cervical dislocation and the spleen was collected and grinded into small pieces to release spleen cells. The obtained cell suspension was then centrifuged (5 min, 500 g) and lysed by ACK lysis buffer (ThermoFisher) (1 mL) for 10 min. Afterwards, single-cell suspensions were obtained by centrifugation (5 min, 500 g) and resuspended in 1×PBS (200 L). The antibodies used were: anti-mouse BV421 F4/80 antibody (1:200, Biolegend, Cat #123137), AF700 CD3 antibody (1:200, Biolegend, Cat #100216), AF488 CD19 antibody (1:200, Biolegend, Cat #115521), APC CD11c antibody (1:200, Biolegend, Cat #117309). The obtained single cell suspensions were stained at 4° C. for 30 min by each of the above antibodies (3 μL), and afterwards were centrifuged, washed, centrifuged and resuspended in 1×PBS (1 mL) for flow cytometry analysis. The spleen cells were analyzed using a LSR II flow machine.

In Vivo CRISPR-Cas9 mTTR Editing in C57BL 6 Mice

To preform liver mTTR gene knockout in vivo, wild-type C57BL/6 mice were i.v. administered with Si₄-C14b LNP co-formulating by Cas9 mRNA and mTTR sgRNA at a total dose of 1.0, 2.0, and 3.0 mg kg⁻¹ (4:1, mRNA:sgRNA, wt:wt) (n=3-5 per group). MC3 LNPs encapsulating the same cargo were i.v. injected as positive control and PBS was i.v. injected as negative control. Blood was collected 1 day before injection and 7 days after injection, and serum was separated for serum TTR protein detection using an ELISA assay kit. The indel of TTR was analyzed by NGS analysis. Liver tissue from the PBS group and liver-targeted Si₄-C14b treated groups was analyzed using in situ hybridization (ISH). To test the toxicity of Si₄-C14b LNPs delivering Cas9 mRNA/mTTR sgRNA, a high dose of RNA (3.0 mg kg⁻¹) was selected for i.v. injection. The blood was collected at 6 h and 24 h, and the serum was separated for liver function (ALT and AST) and renal function (BUN and Creatinine) test. PBS treated group was used as a negative control.

### In Vivo CRISPR-Cas9 EGFP Editing in the C57BL 6-Tg(CAG-EGFP)1Osb/J Transgene Mice Model

Si₅—N14 LNP loading Cas9 mRNA/EGFP sgRNA (4:1, wt:wt) was i.v. injected into a C57BL/6-Tg(CAG-EGFP)1Osb/J (GFP) mouse model with at a total RNA dose of 2.0 mg kg⁻¹ (4 injections, dose: 0.5 mg kg⁻¹ per injection). 7 days post-injection, pieces of the lung were collected and grinded to release lung cells. The single cell isolation and staining was similar to the Cre mRNA delivery protocol described elsewhere herein. GFP knockout in lung cell types was quantified using a LSRForessa machine. Fluorescence-activated cell sorting (FACS) was performed on a BD FACSAria Fusion Sorter (BD Biosciences). Lung tissue section was prepared for immunostaining using a Leica DiM8 fluorescent microscopy. Editing efficiency of sorted lung ECs was further evaluated using qPCR.

### Immunofluorescence

For tissue sections, mouse liver or lung were obtained and transported to the laboratory on ice according to methods known to those skilled in the art. Freshly dissected tissues were fixed, embedded and cut into 7 μm thick cryosections, and then postfixed with 3.2% PFA. Afterward, tissue sections were blocked in PBS+1% BSA, 5% donkey serum, 0.1% Triton X-100, and 0.02% sodium azide for 1 h at room temperature. Then, slides were incubated with primary antibodies (CD31 1:200, BioLegend, Cat #102502; ERG 1:2000, Abcam, Cat #ab92513; F4/80, Cell Signaling Technology, Cat #30325S; GFP antibody, ROCKLAND, Cat #600101215; VECad, R&D system, Cat #AF1002) overnight at 4° C. After, slides were washed and incubated with fluorophore-conjugated secondary antibodies (Alexa Fluor™ 647-conjugated donkey antigoat, 1:1000, Thermo Fisher Scientific, Cat #2045332; Alexa Fluor™ 488-conjugated donkey anti-goat, 1:1000, Thermo Fisher Scientific, Cat #1869589; Alexa Fluor™ 488-conjugated donkey anti-rabbit, 1:1000, Thermo Fisher Scientific, Cat #1810471; CF 568-conjugated donkey anti-rabbit, 1:1000, Sigma-Aldrich, Cat #16C0829; Alexa Fluor™ 647-conjugated donkey anti-rabbit, 1:1000, Thermo Fisher Scientific, Cat #2083195) for 2 h. At last, slides were washed and incubated with 1 μM 4′,6-diamidino-2-phenylindole (DAPI) for 5 min, and mounted using ProLong Gold (Life Sciences, #P36930). Standard multiplex immunofluorescent images were taken with a Leica Dmi8 microscope and analyzed with LAS X software (Leica).

### In Vivo Toxicity Evaluation

To evaluate the in vivo toxicity of SiLNPs, representative liver-, lung-, and spleen-targeted SiLNPs were formulated and i.v. injected with a high dose of Fluc mRNA (liver: 3.0 mg kg⁻¹, lung: 0.5 mg kg⁻¹, and spleen: 1.0 mg kg⁻¹). PBS was i.v. injected as the negative control. After 12 h, whole blood was drawn and the serum was isolated. Next, the liver function (ALT and AST) was measured using individual assay kits according to manufacturer's protocols, and H&E staining was performed on the tissues (heart, liver, spleen, lung, and kidney).

### TTR On-Target DNA Sequencing

DNA was extracted using the Qiagen Puregene Tissue Kit (Cat. No. 158063) and quantified using a Nanodrop 2000. PCR amplification of the TTR target site was carried out using Q5 High-Fidelity DNA Polymerase (New England Biolabs M0491) and the following primer sequences: mTTR-exon2-F, 5′-CGGTTTACTCTGACCCATTTC-3′ (SEQ ID NO:1) and mTTR-exon2-R, 5′-GGGCTTTCTACAAGCTTACC-3′ (SEQ ID NO:2). Deep sequencing of the TTR amplicons and determination of the on-target indel frequency was performed as described elsewhere herein.

### Example 1: Chemical Synthesis of Exemplary Lipidoids and Precursors Thereof

### Synthesis of 2-((2-(1,1,3,3,3-pentamethyldisiloxanyl)ethyl)thio)ethanamine

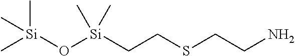

Vinyl pentamethyldisiloxane (1.74 g, 10 mmol, 1.0 equiv), 2-mercaptoethylamine (1.157 g, 15 mmol, 1.5 equiv), and 2,2′-azobis(2-methylpropionitrile) (AIBN, 24.6 mg, 0.15 mmol, 0.015 equiv) were dissolved in methanol (MeOH, 25 mL). And then the mixture was heated to reflux for 24 h. The solvent was removed under vacuum to afford a viscous crude product, which was further purified by flash chromatography (silica gel, DCM/MeOH=10/1) to obtain the title compound as a light-yellow liquid. 1H NMR (400 MHz, MeOD-d4), δ 2.83-2.78 (m, 2H), 2.69-2.57 (m, 4H), 0.95-0.87 (m, 2H), 0.07 (s, 15H). LC-MS (m/z): Calcd for [M+H]+: 251.1. Found: 251.2.

### Synthesis of 2,2′-(((1,1,3,3-tetramethyldisiloxane-1,3-diyl) bis(ethane-2,1-diyl))bis(sulfanediyl)) diethanamine

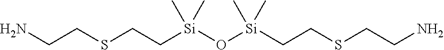 [SMILES: C[Si](C)(CCSCCN)O[Si](C)(C)CCSCCN]

1,3-divinyl tetramethyldisiloxane (1.864 g, 10 mmol, 1.0 equiv), 2-mercaptoethylamine (2.314 g, 30 mmol, 3.0 equiv), and 2,2′-azobis(2-methylpropionitrile) (AIBN, 32.8 mg, 0.2 mmol, 0.02 equiv) were dissolved in methanol (MeOH, 25 mL). And then the mixture was heated to reflux for 24 h. The solvent was removed under vacuum to afford a viscous crude product. The final pure component was further purified by flash chromatography (silica gel, DCM/MeOH=4/1) as a light-yellow liquid. ¹H NMR (400 MHz, DMSO-d₆), δ 2.82-2.76 (m, 4H), 2.74-2.64 (m, 8H), 0.87-0.79 (m, 4H), 0.08 (s, 12H). LC-MS (m/z): Calcd for [M+2H]+: 342.1. Found: 342.2.

### Synthesis of tert-butyl (2-mercaptoethyl)carbamate

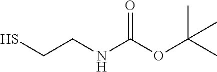

2-Mercaptoethylamine (1.534 g, 20 mmol, 1.0 equiv) and di-tert-butyl decarbonate (4.8 g, 22 mmol, 1.1 equiv) were dissolved in methanol (MeOH, 25 mL) in a round-bottom flask and the mixture was cooled to 0° C. on an ice bath. The reaction was then allowed to warm to room temperature overnight. After the reaction, the solvent was removed under vacuum to afford a white solid crude product. The final pure component was further purified by flash chromatography (silica gel, DCM/Hexane=3/1) as colorless liquid. ¹H NMR (400 MHz, CDCl₃), δ 3.35-3.30 (m, 2H), 2.70-2.64 (m, 1H), 1.46 (s, 9H). LC-MS (m/z): Calcd for [M+H]+: 177.1. Found: 177.1.

### Synthesis of 2,2′,2″-(((2,4,6-trimethyl-1,3,5,2,4,6-trioxatrisilinane-2,4,6-triyl)tris(ethane-2,1-diyl))tris(sulfanediyl))triethanoamine (Compound 2)

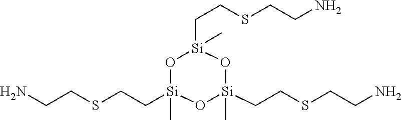 [SMILES: C[Si]1(CCSCCN)O[Si](C)(CCSCCN)O[Si](C)(CCSCCN)O1]

1,3,5-trivinyl-1,3,5-trimethylcyclotrisiloxane (287 mg, 1.11 mmol, 1.0 equiv), tert-butyl (2-mercaptoethyl)carbamate (885.4 mg, 5 mmol, 5.0 equiv) and 2,2′-azobis(2-methylpropionitrile) (AIBN, 8.2 mg, 0.05 mmol, 0.045 equiv) were dissolved in ethanol (EtOH, 25 mL). And then the mixture was heated to reflux for 24 h. The solvent was removed under vacuum to afford a solid crude product (tri-tert-butyl ((((2,4,6-trimethyl-1,3,5,2,4,6-trioxatrisilinane-2,4,6-triyl)tris(ethane-2,1-diyl))tris(sulfanediyl))tris(ethane-2,1-diyl))tricarbamate) (i.e., compound 1), which was further purified by flash chromatography (silica gel, DCM/MeOH=20/1) as a light-yellow solid. Compound 1: ¹H NMR (400 MHz, CDCl₃), δ 3.34-3.26 (m, 6H), 2.84-2.76 (m, 6H), 2.71-2.56 (m, 6H), 1.44 (s, 27H), 1.02-0.91 (m, 6H), 0.21 (s, 9H). LC-MS (m/z): Calcd for [M+H]+: 790.3. Found: 790.4.

Next, compound 1 (500 mg, 0.633 mmol, 1.0 equiv) and trifluoroacetic acid (TFA, 705 μL, 9.5 mmol, 15.0 equiv) were dissolved in dichloromethane (DCM, 10 mL). The mixture was cool to 0° C. and reacted for 4 h. Then the supernatant was removed under vacuum. After that, sodium hydroxide (NaOH, pH=10, 2 mL) was added to dissolve the insoluble salt, 2,2′,2″-(((2,4,6-trimethyl-1,3,5,2,4,6-trioxatrisilinane-2,4,6-triyl)tris(ethane-2,1-diyl))tris(sulfanediyl))triethanamine (i.e., compound 2) was further obtained by centrifuge (14000 rpm, ×10 min). Compound 2: 1H NMR (400 MHz, MeOD-d₄), δ 3.13-3.07 (m, 6H), 2.85-2.79 (m, 6H), 2.73-2.65 (m, 6H), 1.03-0.94 (m, 6H), 0.22 (s, 9H). LC-MS (m/z): Calcd for [M+3H]+: 492.2. Found: 492.2.

### Synthesis of 2,2′,2″,2′″-(((2,4,6,8-tetramethyl-1,3,5,7,2,4,6,8-tetraoxatetrasilocane-2,4,6,8-tetrayl)tetrakis(ethane-2,1-diyl))tetrakis(sulfanediyl))tetraethanamine (Compound 4)

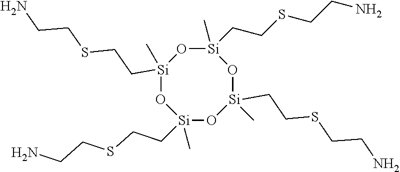 [SMILES: C[Si]1(CCSCCN)O[Si](C)(CCSCCN)O[Si](C)(CCSCCN)O[Si](C)(CCSCCN)O1]

Compound 4 was prepared in a manner analogous to that which was used to prepare compound 2, with the synthesis of compound 3 (i.e., tetra-tert-butyl ((((2,4,6,8-tetramethyl-1,3,5,7,2,4,6,8-tetraoxatetrasilocane-2,4,6,8-tetrayl)tetrakis(ethane-2,1-diyl))tetrakis(sulfanediyl))tetrakis(ethane-2,1-diyl))tetracarbamate) as an intermediate.

Compound 3: ¹H NMR (400 MHz, DMSO-d₆), δ 3.19-3.16 (m, 8H), 3.11-3.03 (m, 8H), 2.61-2.55 (m, 8H), 1.38 (s, 36H), 0.90-0.82 (m, 8H), 0.14 (s, 12H). LC-MS (m/z): Calcd for [M+H]+: 1053.4. Found: 1053.5.

Compound 4: ¹H NMR (400 MHz, MeOD-d₄), δ 2.99-2.91 (m, 8H), 2.77-2.71 (m, 8H), 2.70-2.63 (m, 8H), 1.02-0.94 (m, 8H), 0.21 (s, 12H). LC-MS (m/z): Calcd for [M+4H]+: 656.2. Found: 656.4.

### Synthesis of 2-((2-(4,6-bis(2-((2-aminoethyl)thio)ethyl)-2,4,6-trimethyl-1,3,5,2,4,6-trioxatrisilinan-2-yl)ethyl)thio)ethane-I-sulfonic acid (Compound 6)

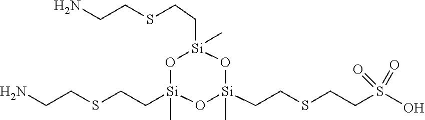 [SMILES: C[Si]1(CCSCCN)O[Si](C)(CCSCCN)O[Si](C)(CCSCCS(=O)(=O)O)O1]

1,3,5-trivinyl-1,3,5-trimethylcyclotrisiloxane (430.5 mg, 1.665 mmol, 1.0 equiv), tert-butyl (2-mercaptoethyl)carbamate (707.6 mg, 3.996 mmol, 2.4 equiv), sodium 2-mercaptoethanesulfonate (328.1 mg, 1.998 mmol, 1.2 equiv) and 2,2′-azobis(2-methylpropionitrile) (AIBN, 12.3 mg, 0.075 mmol, 0.045 equiv) were dissolved in a mixed solution of ethanol/H₂O (20 mL, 3/1). The mixture was heated to reflux for 24 h. The solvent was removed under vacuum to afford a solid crude 2-((2-(4,6-bis(2-((2-((tert-butoxycarbonyl)amino)ethyl)thio)ethyl)-2,4,6-trimethyl-1,3,5,2,4,6-trioxatrisilinan-2-yl)ethyl)thio)ethane-1-sulfonic acid (i.e., compound 5), which was further purified by flash chromatographic (silica gel, DCM/MeOH=10/1) as a light-yellow solid. Compound 5: ¹H NMR (400 MHz, CDCl₃), δ 3.37-3.28 (m, 6H), 3.05-2.82 (m, 6H), 2.74-2.57 (m, 6H), 1.48 (s, 18H), 1.05-0.90 (m, 6H), 0.19 (s, 9H). LC-MS (m/z): Calcd for [M+H]+: 755.2. Found: 755.1.

Then, compound 5 (492 mg, 0.633 mmol, 1.0 equiv) and trifluoroacetic acid (TFA, 705 μL, 9.5 mmol, 15.0 equiv) were dissolved in dichloromethane (DCM, 10 mL). The mixture was cooled to 0° C. and reacted for 4 h. Then the supernatant was removed under vacuum. After that, sodium hydroxide (NaOH, pH=10, 2 mL) was added to dissolve the insoluble salt, the product (i.e., compound 6) was obtained by centrifuge (14000 rpm, ×10 min). ¹H NMR (400 MHz, MeOD-d₄), δ 3.23-3.12 (m, 6H), 2.93-2.82 (m, 6H), 2.75-2.65 (m, 6H), 1.04-0.94 (m, 6H), 0.21 (s, 9H). LC-MS (m/z): Calcd for [M+2H]+: 556.1. Found: 556.2.

### Synthesis of α,β-Unsaturated Alkyl Amides (i.e., Amide-Bond Based Alkyl Tails)

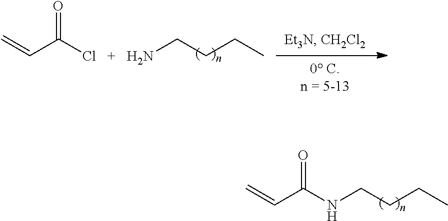

The following procedure was utilized to prepare each of compounds N8, N10, N12, N14, and N16. For the sake of brevity, only the synthesis of N8 is provided an example (i.e., n=5): n-octylamine (3.8775 g, 30 mmol, 1.0 equiv) and triethylamine (3.642 g, 36 mmol, 1.2 equiv) were dissolved anhydrous DCM (30 mL) and cool to 0° C. Acryloyl chloride (2.93 mL, 36 mmol, 1.2 equiv) was dropwise into the above mixture. The reaction was conducted overnight at room temperature. Then the solvent was removed under vacuum to afford a solid crude product. N8 was further purified by flash chromatographic (silica gel, DCM/MeOH=40/1) as a light-yellow solid.

N-octylacrylamide (N8): ¹H NMR (400 MHz, CDCl₃), δ 6.33-6.25 (d, 1H), 6.16-6.07 (m, 1H), 5.68-5.61 (d, 1H), 3.39-3.31 (m, 2H), 1.61-1.49 (m, 2H), 1.41-1.21 (m, 8H), 0.95-0.87 (m, 3H). LC-MS (m/z): Calcd for [M+H]+: 184.2. Found: 184.2.

N-decylacrylamide (N10): ¹H NMR (400 MHz, CDCl₃), δ 6.32-6.24 (d, 1H), 6.18-6.08 (m, 1H), 5.66-5.60 (d, 1H), 3.37-3.28 (m, 2H), 1.59-1.49 (m, 2H), 1.40-1.22 (m, 10H), 0.93-0.85 (m, 3H). LC-MS (m/z): Calcd for [M+H]+: 212.2. Found: 212.2.

N-dodecylacrylamide (N12): ¹H NMR (400 MHz, CDCl₃), δ 6.34-6.24 (d, 1H), 6.17-6.07 (m, 1H), 5.68-5.61 (d, 1H), 3.39-3.29 (m, 2H), 1.61-1.49 (m, 2H), 1.40-1.21 (m, 12H), 0.94-0.84 (m, 3H). LC-MS (m/z): Calcd for [M+H]+: 240.2. Found: 240.2.

N-tetradecylacrylamide (N14): ¹H NMR (400 MHz, CDCl₃), δ 6.32-6.25 (d, 1H), 6.16-6.05 (m, 1H), 5.67-5.60 (d, 1H), 3.38-3.30 (m, 2H), 1.61-1.49 (m, 2H), 1.40-1.24 (m, 14H), 0.98-0.85 (m, 3H). LC-MS (m/z): Calcd for [M+H]+: 268.2. Found: 268.2.

N-hexadecylacrylamide (N16): ¹H NMR (400 MHz, CDCl₃), δ 6.34-6.25 (d, 1H), 6.16-6.06 (m, 1H), 5.69-5.62 (d, 1H), 3.41-3.32 (m, 2H), 1.62-1.52 (m, 2H), 1.38-1.26 (m, 16H), 0.95-0.86 (m, 3H). LC-MS (m/z): Calcd for [M+H]+: 296.2. Found: 296.2.

### Synthesis of Siloxane-Based Ionizable Lipid Libraries

The siloxane-based ionizable lipid library (252 ionizable lipids) was prepared by nucleophilic addition and/or Michael addition (i.e., [1,4]-conjugate addition) reactions between the 12 different siloxane-based amine cores and 21 different alkyl tail precursors (e.g., epoxides, α,β-unsaturated amides and/or esters). An exemplary synthesis of Si₁—C12 is provided herein, however, other lipidoids were prepared in an analogous manner following procedures known to those skilled in the art of organic synthesis.

2-((2-(1,1,3,3,3-pentamethyldisiloxaneyl)ethyl)thio)ethan-1-amine (Si₁) (2.05 g, 10 mmol, 1 equiv) and 2-dodecyloxirane (C12) (4.42 g, 24 mmol, 2.4 equiv) were added in a glass vial equipped with a stir bar dissolved in ethanol. The reaction was stirred at 80° C. for three days. The crude product was afforded by removing the solvents and was used to screen the library for Fluc mRNA delivery in vitro without further purification. The top performing liver-, lung-, and spleen-targeted siloxane lipids were purified by CombiFlash Rf-200i chromatography.

### Exemplary Chemical Structure and ¹H-NMR data for 14,28-bis(2-hydroxy-12-methyltridecyl)-2,20,20,22,22,40-hexamethyl-21-oxa-17,25-dithia-14,28-diaza-20,22-disilatetracontane-12,30-diol (Si5-N14b)

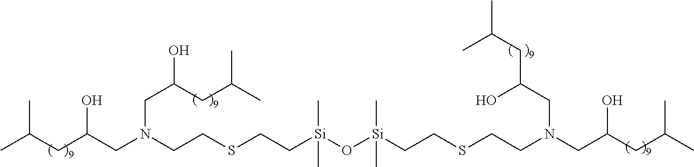

Si_{b}-C14b: ¹H NMR (400 MHz, MeOD-d₄), δ 3.73-3.61 (m, 4H), 2.95-2.37 (m, 20H), 1.57-1.47 (m, 12H), 1.43-1.15 (m, 68), 0.96-0.87 (m, 24H), 0.16 (s, 12H). LC-MS (m/z): Calcd for [M+H]+: 1190.1. Found: 1190.0.

### Exemplary Chemical Structure and ¹H-NMR data for 3,3′,3″,3′″-(((1,3,3-tetramethyldisiloxane-1,3-diyl)bis(propane-3,1-diyl))bis(azanetriyl))tetrakis(N-tetradecylpropanamide) (Si5-N14)

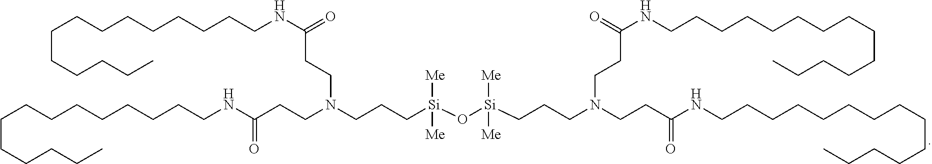 [SMILES: CCCCCCCCCCCCCCNC(=O)CCN(CCC[Si](C)(C)O[Si](C)(C)CCCN(CCC(=O)NCCCCCCCCCCCCCC)CCC(=O)NCCCCCCCCCCCCCC)CCC(=O)NCCCCCCCCCCCCCC]

Si₅—N14: ¹H NMR (400 MHz, MeOD-d₄), δ 3.22-3.15 (m, 8H), 2.90-2.82 (m, 4H), 2.79-2.73 (m, 4H), 2.66-2.58 (m, 4H0, 2.50-2.33 (m, 8H), 1.64-1.24 (m, 100H), 0.97-0.87 (m, 12H), 0.62-0.50 (m, 4H), 0.11 (s, 12H). LC-MS (m/z): Calcd for [M+H]+: 1318.2. Found: 1318.1.

Exemplary Chemical Structure and ¹H-NMR data for 2-((2-(4,6-bis(2-((2-(bis(2-hydroxydecyl)amino)ethyl)thio)ethyl)-2,4,6-trimethyl-1,3,5,2,4,6-trioxatrisilinan-2-yl)ethyl)thio)ethane-1-sulfonic acid (Si₁₂—C10)

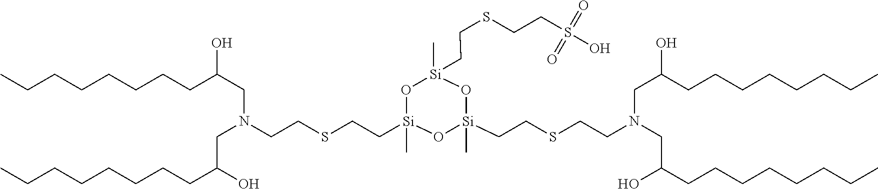 [SMILES: CCCCCCCCC(O)CN(CCSCC[Si]1(C)O[Si](C)(CCSCCN(CC(O)CCCCCCCC)CC(O)CCCCCCCC)O[Si](C)(CCSCCS(=O)(=O)O)O1)CC(O)CCCCCCCC]

Si₁₂—C10: ¹H NMR (400 MHz, MeOD-d₄), δ 3.71-3.40 (m, 16H), 2.77-2.45 (m, 4H), 1.64-1.22 (m, 66H), 0.95-0.86 (m, 18H), 0.21 (s, 9H). LC-MS (m/z): Calcd for [M+H]+: 1181.1. Found: 1181.0.

### Example 2: Design and Development of Siloxane Lipidoids

Siloxane-based nanomaterials exhibit specific functions for drug delivery and nanomedicine due to their low toxicity, high stability, viability, and relative hydrophobicity. Here, these siloxane structures were leveraged to synthesize novel ionizable lipids with moieties that can be functionalized to enable specific chemistries to be performed. By modulating siloxane amine cores and tail structures, a series of siloxane lipidoids were designed and synthesized (FIGS. 1D-1E).

To explore the use of siloxane structures for LNP-based RNA delivery, a library of siloxane lipidoids was prepared, wherein each lipidoid comprises ionizable siloxane amine heads and hydrophobic tails, synthesized by nucleophilic addition and/or [1,4]-conjugate addition reactions. The combinatorial reaction between various siloxane amines and each of alkylepoxides, alkylesters, and alkylamides (Cy/Oy/Ny) enabled the synthesis of 252 siloxane ionizable lipids (see FIGS. 1D-1E and Example 1), denoted as Six-Cy/Six-Oy/Six-Ny, where ‘x’ indicates the order of siloxane amine heads in this study and ‘y’ represents the length of different alkyl chains. To broaden the siloxane ionizable lipid architectures, a series of linear, cyclic, and functionalized siloxane amine heads with various silicon atoms were selected and synthesized. The chemical design of siloxane lipidoids is unique, wherein siloxane amine cores were varied with main chain silicon atom numbers, main topologies, amine numbers per head unit, attached alkyl chain structures with variable tail length, different tail types (i.e., epoxide-, α,β-unsaturated ester-, and α,β-unsaturated amide-derived aliphatic groups), and numbers of tails per lipidoid. It has been contemplated that this combinatorial siloxane-based ionizable lipid library could extend the chemical diversity of ionizable lipid formulations for diverse nucleic acid delivery applications.

### Example 3: In Vitro SiLNP mRNA Delivery

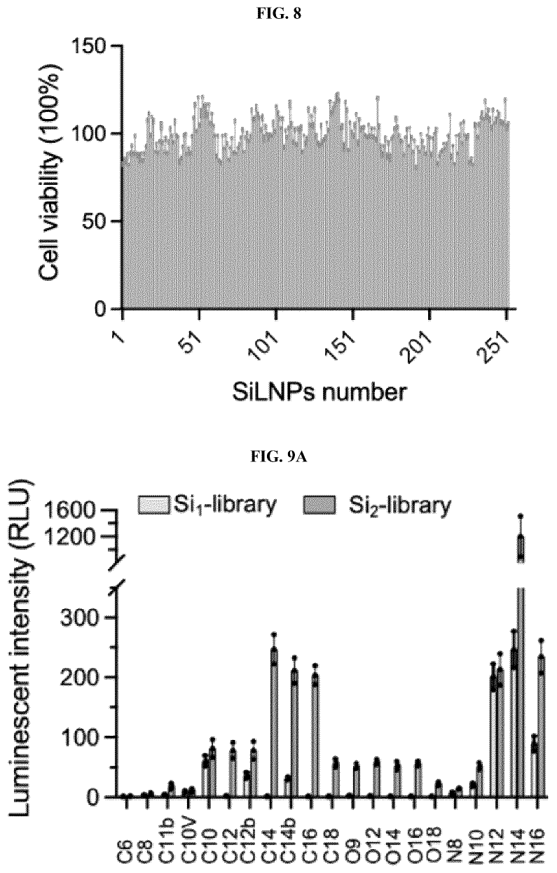

To study the SAR of mRNA delivery, SiLNPs delivering firefly luciferase (Fluc) mRNA were used to transfect human liver carcinoma cells (HepG2). SiLNPs were formulated using siloxane lipidoids, the phospholipid DOPE, cholesterol and lipid-anchored poly(ethylene glycol) (C14PEG2000) (35:16:46.5:2.5 molar ratio) and were mixed with Fluc mRNA via perfusion through a microfluidic mixing device designed with herringbone features (Table 1). The resulting SiLNPs comprised a range of LNP sizes (50 nm-200 nm) (Table 1), had desirable monodispersity as indicated by polydispersity index (PDI) (approximately 70% of SiLNPs have a PDI lower than 0.2) (Table 1), and mRNA encapsulation efficiencies ranging from 60% to 93% (Table 1). Additionally, all SiLNPs exhibited low cytotoxicity (cell viability >80%) (FIG. 8).

TABLE 1
Particle size, PDI, encapsulation
efficiency (EE) of exemplary SiLNPs

| | | Polydispersity | Encapsulation |
|---|---|---|---|
| | | Index | Efficiency |
| Name | Size (nm) | (PI) | (%) |
| Si1-C6 | 75.4 ± 4.3 | 0.10 ± 0.02 | 67.6 ± 2.4 |
| Si1-C8 | 86.0 ± 4.1 | 0.11 ± 0.05 | 79.7 ± 2.7 |
| Si1-C11b | 77.5 ± 3.5 | 0.16 ± 0.07 | 87.3 ± 2.0 |
| Si1-C10V | 64.4 ± 2.7 | 0.25 ± 0.11 | 73.7 ± 2.1 |
| Si1-C10 | 90.7 ± 4.2 | 0.17 ± 0.07 | 92.9 ± 1.9 |
| Si1-C12 | 51.9 ± 2.6 | 0.20 ± 0.11 | 76.4 ± 4.4 |
| Si1-C12b | 61.7 ± 3.1 | 0.07 ± 0.01 | 67.1 ± 2.9 |
| Si1-C14 | 60.0 ± 3.1 | 0.14 ± 0.04 | 69.1 ± 1.1 |
| Si1-C14b | 70.4 ± 3.0 | 0.20 ± 0.07 | 66.3 ± 1.3 |
| Si1-C16 | 64.1 ± 3.1 | 0.14 ± 0.07 | 69.3 ± 1.8 |
| Si1-C18 | 80.1 ± 2.2 | 0.17 ± 0.12 | 76.0 ± 2.6 |
| Si1-O9 | 71.2 ± 3.1 | 0.21 ± 0.02 | 65.2 ± 2.1 |
| Si1-O12 | 79.7 ± 2.4 | 0.21 ± 0.08 | 73.4 ± 2.1 |
| Si1-O14 | 78.8 ± 2.9 | 0.25 ± 0.14 | 62.4 ± 1.5 |
| Si1-O16 | 86.6 ± 2.2 | 0.22 ± 0.07 | 61.1 ± 1.7 |
| Si1-O18 | 90.0 ± 2.6 | 0.06 ± 0.01 | 67.9 ± 1.9 |
| Si1-N8 | 85.8 ± 2.9 | 0.19 ± 0.06 | 66.8 ± 1.8 |
| Si1-N10 | 91.1 ± 2.8 | 0.16 ± 0.07 | 63.7 ± 3.0 |
| Si1-N12 | 90.2 ± 2.1 | 0.31 ± 0.06 | 62.5 ± 1.9 |
| Si1-N14 | 101.5 ± 5.9 | 0.18 ± 0.04 | 66.8 ± 1.1 |
| Si1-N16 | 84.0 ± 3.1 | 0.17 ± 0.08 | 64.2 ± 2.9 |
| Si2-C6 | 80.7 ± 2.6 | 0.08 ± 0.05 | 69.9 ± 2.0 |
| Si2-C8 | 80.3 ± 2.7 | 0.20 ± 0.04 | 84.5 ± 2.1 |
| Si2-C11b | 87.7 ± 2.1 | 0.12 ± 0.05 | 74.6 ± 1.2 |
| Si2-C10V | 84.8 ± 3.4 | 0.11 ± 0.02 | 86.3 ± 2.7 |
| Si2-C10 | 76.0 ± 3.0 | 0.19 ± 0.02 | 88.7 ± 1.1 |
| Si2-C12 | 90.2 ± 3.3 | 0.11 ± 0.05 | 76.2 ± 2.6 |
| Si2-C12b | 102.9 ± 4.1 | 0.16 ± 0.05 | 76.6 ± 2.8 |
| Si2-C14 | 97.0 ± 3.4 | 0.19 ± 0.06 | 71.3 ± 1.2 |
| Si2-C14b | 94.4 ± 2.7 | 0.27 ± 0.03 | 86.9 ± 1.9 |
| Si2-C16 | 92.9 ± 2.7 | 0.24 ± 0.03 | 83.3 ± 1.3 |
| Si2-C18 | 90.0 ± 2.5 | 0.10 ± 0.03 | 75.3 ± 1.6 |
| Si2-O9 | 89.6 ± 4.0 | 0.15 ± 0.03 | 70.8 ± 1.6 |
| Si2-O12 | 86.5 ± 2.5 | 0.08 ± 0.03 | 74.9 ± 2.8 |
| Si2-O14 | 88.4 ± 3.2 | 0.14 ± 0.03 | 83.5 ± 1.8 |
| Si2-O16 | 92.2 ± 3.2 | 0.20 ± 0.03 | 85.4 ± 1.9 |
| Si2-O18 | 88.9 ± 2.8 | 0.27 ± 0.06 | 81.0 ± 1.5 |
| Si2-N8 | 92.1 ± 3.3 | 0.11 ± 0.03 | 69.8 ± 1.7 |
| Si2-N10 | 94.0 ± 4.1 | 0.08 ± 0.02 | 68.7 ± 1.1 |
| Si2-N12 | 81.1 ± 2.4 | 0.23 ± 0.03 | 76.0 ± 2.8 |
| Si2-N14 | 80.1 ± 2.6 | 0.31 ± 0.03 | 72.2 ± 1.9 |
| Si2-N16 | 74.1 ± 2.8 | 0.32 ± 0.03 | 61.6 ± 2.0 |
| Si3-C6 | 66.8 ± 2.1 | 0.08 ± 0.02 | 84.1 ± 2.2 |
| Si3-C8 | 87.9 ± 3.3 | 0.06 ± 0.01 | 83.6 ± 2.6 |
| Si3-C11b | 81.1 ± 3.5 | 0.13 ± 0.04 | 81.2 ± 2.2 |
| Si3-C10V | 71.3 ± 2.7 | 0.18 ± 0.04 | 82.2 ± 2.2 |
| Si3-C10 | 76.0 ± 2.5 | 0.12 ± 0.06 | 84.7 ± 2.9 |
| Si3-C12 | 79.8 ± 2.8 | 0.07 ± 0.02 | 86.3 ± 1.5 |
| Si3-C12b | 85.8 ± 3.2 | 0.12 ± 0.06 | 67.2 ± 1.7 |
| Si3-C14 | 72.5 ± 3.3 | 0.08 ± 0.03 | 62.8 ± 1.7 |
| Si3-C14b | 89.4 ± 4.4 | 0.03 ± 0.01 | 65.1 ± 2.8 |
| Si3-C16 | 74.3 ± 1.6 | 0.04 ± 0.01 | 73.8 ± 1.6 |
| Si3-C18 | 70.8 ± 2.7 | 0.16 ± 005 | 66.0 ± 1.7 |
| Si3-O9 | 70.5 ± 3.2 | 0.06 ± 0.01 | 73.4 ± 1.6 |
| Si3-O12 | 71.7 ± 3.1 | 0.10 ± 0.04 | 88.2 ± 1.3 |
| Si3-O14 | 67.8 ± 2.0 | 0.09 ± 0.04 | 85.6 ± 1.7 |
| Si3-O16 | 65.7 ± 2.5 | 0.10 ± 0.08 | 85.4 ± 1.4 |
| Si3-O18 | 67.4 ± 2.4 | 0.05 ± 0.03 | 92.0 ± 2.3 |
| Si3-N8 | 82.1 ± 3.4 | 0.11 ± 0.05 | 82.4 ± 2.6 |
| Si3-N10 | 77.3 ± 1.8 | 0.18 ± 0.04 | 84.1 ± 1.3 |
| Si3-N12 | 79.0 ± 2.8 | 0.23 ± 0.04 | 78.3 ± 1.6 |
| Si3-N14 | 78.5 ± 2.0 | 0.22 ± 0.13 | 68.7 ± 1.3 |
| Si3-N16 | 70.5 ± 2.9 | 0.34 ± 0.04 | 60.0 ± 1.9 |
| Si4-C6 | 77.2 ± 4.1 | 0.05 ± 0.01 | 85.1 ± 1.2 |
| Si4-C8 | 93.3 ± 4.5 | 0.21 ± 0.09 | 80.0 ± 1.9 |
| Si4-C11b | 77.5 ± 3.3 | 0.08 ± 0.05 | 80.4 ± 1.1 |
| Si4-C10V | 75.7 ± 3.2 | 0.17 ± 0.01 | 77.5 ± 2.3 |
| Si4-C10 | 87.8 ± 6.5 | 0.08 ± 0.02 | 81.8 ± 1.5 |
| Si4-C12 | 86.1 ± 3.4 | 0.15 ± 0.04 | 84.2 ± 1.4 |
| Si4-C12b | 73.9 ± 2.5 | 0.06 ± 0.03 | 73.4 ± 1.2 |
| Si4-C14 | 76.6 ± 3.5 | 0.15 ± 0.04 | 85.3 ± 2.5 |
| Si4-C14b | 97.4 ± 6.2 | 0.24 ± 0.05 | 80.3 ± 2.0 |
| Si4-C16 | 80.5 ± 3.2 | 0.10 ± 0.02 | 82.4 ± 1.7 |
| Si4-C18 | 76.1 ± 3.1 | 0.23 ± 0.06 | 769 ± 1.1 |
| Si4-O9 | 79.1 ± 2.4 | 0.15 ± 0.03 | 73.8 ± 1.4 |
| Si4-O12 | 78.8 ± 4.8 | 0.04 ± 0.01 | 78.1 ± 1.8 |
| Si4-O14 | 97.5 ± 8.2 | 0.02 ± 0.01 | 68.6 ± 1.4 |
| Si4-O16 | 64.6 ± 2.5 | 0.04 ± 0.02 | 82.3 ± 1.6 |
| Si4-O18 | 88.9 ± 5.3 | 0.16 ± 003 | 72.8 ± 2.2 |
| Si4-N8 | 96.2 ± 5.6 | 0.11 ± 0.04 | 77.8 ± 2.1 |
| Si4-N10 | 95.5 ± 6.0 | 0.17 ± 0.02 | 77.0 ± 1.5 |
| Si4-N12 | 78.0 ± 3.0 | 0.28 ± 0.05 | 71.2 ± 1.2 |
| Si4-N14 | 82.8 ± 4.3 | 0.31 ± 0.05 | 68.6 ± 1.0 |
| Si4-N16 | 91.9 ± 4.2 | 0.36 ± 0.08 | 63.3 ± 2.8 |
| Si5-C6 | 90.5 ± 2.7 | 0.12 ± 0.04 | 82.6 ± 2.4 |
| Si5-C8 | 81.9 ± 3.5 | 0.05 ± 0.02 | 82.8 ± 1.3 |
| Si5-C11b | 96.2 ± 5.4 | 0.04 ± 0.02 | 88.0 ± 1.3 |
| Si5-C10V | 77.1 ± 2.0 | 0.17 ± 0.02 | 77.5 ± 2.0 |
| Si5-C10 | 88.3 ± 2.6 | 0.06 ± 0.02 | 72.4 ± 2.1 |
| Si5-C12 | 95.1 ± 5.0 | 0.05 ± 0.02 | 67.5 ± 2.4 |
| Si5-C12b | 89.4 ± 2.3 | 0.05 ± 0.03 | 64.2 ± 2.5 |
| Si5-C14 | 87.0 ± 1.6 | 0.01 ± 0.01 | 64.9 ± 1.8 |
| Si5-C14b | 111.8 ± 3.1 | 0.20 ± 0.04 | 67.5 ± 1.7 |
| Si5-C16 | 90.6 ± 2.7 | 0.07 ± 0.02 | 62.9 ± 2.4 |
| Si5-C18 | 95.7 ± 3.1 | 0.15 ± 0.02 | 71.4 ± 2.4 |
| Si5-O9 | 86.9 ± 1.6 | 0.28 ± 0.02 | 66.2 ± 1.8 |
| Si5-O12 | 93.7 ± 4.0 | 0.20 ± 0.06 | 70.1 ± 1.1 |
| Si5-O14 | 83.7 ± 3.4 | 0.09 ± 0.07 | 89.4 ± 2.1 |
| Si5-O16 | 100.5 ± 6.8 | 0.31 ± 0.06 | 67.1 ± 1.1 |
| Si5-O18 | 107.2 ± 4.5 | 0.10 ± 0.06 | 73.5 ± 1.6 |
| Si5-N8 | 113.6 ± 5.2 | 0.13 ± 0.07 | 68.1 ± 1.1 |
| Si5-N10 | 115.5 ± 4.9 | 0.27 ± 0.04 | 60.6 ± 2.0 |
| Si5-N12 | 120.6 ± 4.9 | 0.10 ± 0.04 | 67.0 ± 1.8 |
| Si5-N14 | 90.5 ± 6.7 | 0.21 ± 0.03 | 85.7 ± 4.8 |
| Si5-N16 | 104.9 ± 5.0 | 0.10 ± 0.04 | 65.5 ± 3.8 |
| Si6-C6 | 96.0 ± 3.3 | 0.04 ± 0.02 | 73.9 ± 1.6 |
| Si6-C8 | 95.9 ± 1.1 | 0.06 ± 0.01 | 77.4 ± 1.9 |
| Si6-C11b | 105.2 ± 3.9 | 0.26 ± 0.03 | 85.3 ± 2.6 |
| Si6-C10V | 84.3 ± 4.7 | 0.10 ± 0.04 | 84.7 ± 2.4 |
| Si6-C10 | 87.6 ± 2.0 | 0.07 ± 0.05 | 85.7 ± 3.3 |
| Si6-C12 | 92.0 ± 1.9 | 0.05 ± 0.03 | 76.6 ± 2.1 |
| Si6-C12b | 76.7 ± 4.1 | 0.08 ± 0.08 | 77.2 ± 2.2 |
| Si6-C14 | 86.5 ± 2.8 | 0.05 ± 0.03 | 75.6 ± 2.3 |
| Si6-C14b | 94.6 ± 3.4 | 0.07 ± 0.03 | 88.4 ± 4.8 |
| Si6-C16 | 96.4 ± 1.1 | 0.04 ± 0.03 | 76.3 ± 2.3 |
| Si6-C18 | 85.5 ± 4.8 | 0.06 ± 0.03 | 78.3 ± 1.6 |
| Si6-O9 | 94.1 ± 3.2 | 0.04 ± 0.03 | 72.3 ± 1.7 |
| Si6-O12 | 93.2 ± 5.3 | 0.11 ± 0.03 | 79.2 ± 1.4 |
| Si6-O14 | 93.9 ± 4.9 | 0.04 ± 0.03 | 74.0 ± 1.3 |
| Si6-O16 | 90.3 ± 4.3 | 0.04 ± 0.01 | 84.6 ± 2.4 |
| Si6-O18 | 86.0 ± 6.4 | 0.05 ± 0.04 | 82.4 ± 1.9 |
| Si6-N8 | 93.4 ± 4.3 | 0.13 ± 0.03 | 81.9 ± 1.8 |
| Si6-N10 | 78.8 ± 4.5 | 0.08 ± 0.05 | 73.5 ± 1.2 |
| Si6-N12 | 93.9 ± 5.2 | 0.17 ± 0.04 | 73.6 ± 1.3 |
| Si6-N14 | 86.1 ± 3.1 | 0.08 ± 0.01 | 73.9 ± 3.3 |
| Si6-N16 | 93.9 ± 3.6 | 0.15 ± 0.04 | 77.4 ± 1.6 |
| Si7-C6 | 129.0 ± 6.5 | 0.07 ± 0.04 | 66.8 ± 4.1 |
| Si7-C8 | 126.8 ± 4.9 | 0.14 ± 0.03 | 63.5 ± 2.0 |
| Si7-C11b | 158.6 ± 4.1 | 0.04 ± 0.02 | 64.5 ± 1.5 |
| Si7-C10V | 148.4 ± 3.7 | 0.07 ± 0.03 | 64.4 ± 3.0 |
| Si7-C10 | 133.4 ± 3.8 | 0.04 ± 0.02 | 60.4 ± 2.5 |
| Si7-C12 | 130.0 ± 6.2 | 0.08 ± 0.05 | 67.7 ± 2.6 |
| Si7-C12b | 155.1 ± 6.5 | 0.06 ± 0.03 | 63.7 ± 1.7 |
| Si7-C14 | 164.2 ± 5.9 | 0.07 ± 0.04 | 62.1 ± 3.1 |
| Si7-C14b | 90.8 ± 4.1 | 0.08 ± 0.03 | 63.4 ± 2.0 |
| Si7-C16 | 157.3 ± 8.7 | 0.10 ± 0.04 | 70.3 ± 3.5 |
| Si7-C18 | 114.2 ± 5.4 | 0.08 ± 0.06 | 62.7 ± 2.1 |
| Si7-O9 | 128.1 ± 3.2 | 0.15 ± 0.05 | 60.8 ± 2.0 |
| Si7-O12 | 166.5 ± 4.3 | 0.10 ± 0.04 | 64.3 ± 1.4 |
| Si7-O14 | 93.1 ± 3.9 | 0.06 ± 0.03 | 65.7 ± 3.0 |
| Si7-O16 | 106.4 ± 4.1 | 0.23 ± 0.14 | 62.8 ± 1.4 |
| Si7-O18 | 93.6 ± 1.8 | 0.35 ± 0.05 | 61.8 ± 1.5 |
| Si7-N8 | 104.0 ± 4.1 | 0.25 ± 0.05 | 65.9 ± 2.4 |
| Si7-N10 | 91.1 ± 4.7 | 0.23 ± 0.05 | 72.5 ± 1.4 |
| Si7-N12 | 88.6 ± 3.9 | 0.21 ± 0.04 | 88.4 ± 3.4 |
| Si7-N14 | 118.5 ± 5.8 | 0.41 ± 0.03 | 75.5 ± 2.9 |
| Si7-N16 | 114.3 ± 5.4 | 0.32 ± 0.05 | 62.6 ± 1.4 |
| Si8-C6 | 197.6 ± 7.4 | 0.24 ± 0.10 | 63.0 ± 2.5 |
| Si8-C8 | 189.8 ± 3.1 | 0.15 ± 0.03 | 61.0 ± 2.2 |
| Si8-C11b | 179.5 ± 2.5 | 0.21 ± 0.08 | 65.7 ± 2.1 |
| Si8-C10V | 158.5 ± 8.1 | 0.21 ± 0.12 | 92.2 ± 2.6 |
| Si8-C10 | 188.3 ± 5.8 | 0.10 ± 0.04 | 61.8 ± 5.1 |
| Si8-C12 | 192.9 ± 8.2 | 0.19 ± 0.07 | 70.4 ± 3.3 |
| Si8-C12b | 175.0 ± 3.6 | 0.28 ± 0.03 | 79.9 ± 1.9 |
| Si8-C14 | 144.5 ± 6.1 | 0.20 ± 0.06 | 62.7 ± 1.9 |
| Si8-C14b | 153.9 ± 2.6 | 0.30 ± 0.07 | 74.4 ± 3.7 |
| Si8-C16 | 174.8 ± 4.7 | 0.19 ± 0.06 | 77.4 ± 2.5 |
| Si8-C18 | 166.3 ± 7.6 | 0.22 ± 0.07 | 64.2 ± 6.5 |
| Si8-O9 | 172.8 ± 8.1 | 0.26 ± 0.07 | 76.0 ± 2.0 |
| Si8-O12 | 167.5 ± 4.0 | 0.22 ± 0.05 | 79.5 ± 2.1 |
| Si8-O14 | 173.0 ± 3.8 | 0.38 ± 0.09 | 66.5 ± 1.9 |
| Si8-O16 | 152.9 ± 5.3 | 0.21 ± 0.07 | 67.7 ± 1.1 |
| Si8-O18 | 159.6 ± 5.3 | 0.22 ± 0.06 | 64.8 ± 1.8 |
| Si8-N8 | 119.9 ± 8.6 | 0.21 ± 0.08 | 63.9 ± 1.0 |
| Si8-N10 | 128.9 ± 4.2 | 0.14 ± 0.05 | 68.1 ± 0.9 |
| Si8-N12 | 110.3 ± 4.0 | 0.35 ± 0.02 | 73.9 ± 1.0 |
| Si8-N14 | 114.7 ± 5.3 | 0.21 ± 0.04 | 73.1 ± 3.3 |
| Si8-N16 | 106.0 ± 3.7 | 0.32 ± 0.07 | 65.8 ± 1.2 |
| Si9-C6 | 154.9 ± 4.6 | 0.14 ± 0.02 | 64.3 ± 1.3 |
| Si9-C8 | 164.9 ± 4.5 | 0.15 ± 0.02 | 60.3 ± 1.8 |
| Si9-C11b | 130.4 ± 5.7 | 0.15 ± 0.03 | 61.9 ± 1.2 |
| Si9-C10V | 125.7 ± 4.7 | 0.12 ± 0.01 | 64.2 ± 1.0 |
| Si9-C10 | 161.6 ± 7.8 | 0.14 ± 0.02 | 61.8 ± 1.1 |
| Si9-C12 | 156.9 ± 8.0 | 0.14 ± 0.07 | 64.7 ± 2.0 |
| Si9-C12b | 148.7 ± 6.3 | 0.10 ± 0.04 | 66.8 ± 1.3 |
| Si9-C14 | 164.8 ± 7.8 | 0.17 ± 0.06 | 64.4 ± 4.4 |
| Si9-C14b | 167.4 ± 10.2 | 0.17 ± 0.07 | 68.0 ± 1.5 |
| Si9-C16 | 155.2 ± 4.8 | 0.19 ± 0.05 | 65.8 ± 1.3 |
| Si9-C18 | 150.2 ± 8.0 | 0.24 ± 0.09 | 65.0 ± 1.4 |
| Si9-O9 | 129.4 ± 6.7 | 0.19 ± 0.05 | 64.2 ± 2.1 |
| Si9-O12 | 167.8 ± 4.1 | 0.21 ± 0.12 | 65.9 ± 3.6 |
| Si9-O14 | 161.0 ± 6.5 | 0.17 ± 0.09 | 66.0 ± 1.8 |
| Si9-O16 | 155.2 ± 2.7 | 0.18 ± 0.09 | 63.7 ± 1.6 |
| Si9-O18 | 127.7 ± 7.5 | 0.16 ± 0.04 | 66.4 ± 2.7 |
| Si9-N8 | 118.6 ± 5.1 | 0.38 ± 0.09 | 65.3 ± 3.9 |
| Si9-N10 | 105.9 ± 3.5 | 0.28 ± 0.02 | 75.6 ± 2.1 |
| Si9-N12 | 102.2 ± 6.5 | 0.15 ± 0.04 | 77.4 ± 1.4 |
| Si9-N14 | 115.7 ± 6.0 | 0.31 ± 0.05 | 71.1 ± 2.1 |
| Si9-N16 | 92.7 ± 5.7 | 0.12 ± 0.06 | 63.2 ± 2.3 |
| Si10-C6 | 67.6 ± 2.5 | 0.05 ± 0.03 | 77.7 ± 2.5 |
| Si10-C8 | 76.0 ± 2.9 | 0.05 ± 0.03 | 70.3 ± 1.0 |
| Si10-C11b | 73.2 ± 1.3 | 0.10 ± 0.05 | 85.3 ± 1.1 |
| Si10-C10V | 84.9 ± 2.6 | 0.14 ± 0.05 | 72.3 ± 1.7 |
| Si10-C10 | 109.8 ± 4.1 | 0.06 ± 0.03 | 61.7 ± 2.2 |
| Si10-C12 | 75.1 ± 3.3 | 0.09 ± 0.05 | 63.1 ± 2.0 |
| Si10-C12b | 82.4 ± 2.3 | 0.06 ± 0.02 | 63.0 ± 1.7 |
| Si10-C14 | 65.6 ± 2.6 | 0.02 ± 0.02 | 71.1 ± 1.1 |
| Si10-C14b | 93.2 ± 3.5 | 0.08 ± 0.06 | 63.5 ± 2.4 |
| Si10-C16 | 67.1 ± 3.0 | 0.08 ± 0.03 | 66.3 ± 3.0 |
| Si10-C18 | 72.6 ± 2.1 | 0.05 ± 0.02 | 64.5 ± 1.7 |
| Si10-O9 | 79.5 ± 2.0 | 0.13 ± 0.04 | 69.9 ± 1.0 |
| Si10-O12 | 133.8 ± 5.3 | 0.18 ± 0.07 | 63.8 ± 1.1 |
| Si10-O14 | 114.0 ± 3.6 | 0.06 ± 0.04 | 61.1 ± 2.2 |
| Si10-O16 | 104.7 ± 6.2 | 0.13 ± 0.05 | 65.1 ± 1.7 |
| Si10-O18 | 105.4 ± 5.3 | 0.05 ± 0.01 | 61.1 ± 6.6 |
| Si10-N8 | 81.1 ± 2.2 | 0.20 ± 0.05 | 69.4 ± 3.7 |
| Si10-N10 | 115.4 ± 8.0 | 0.14 ± 0.01 | 63.3 ± 2.5 |
| Si10-N12 | 108.5 ± 7.2 | 0.09 ± 0.02 | 63.1 ± 1.1 |
| Si10-N14 | 86.1 ± 3.7 | 0.05 ± 0.03 | 62.1 ± 1.4 |
| Si10-N16 | 91.1 ± 4.9 | 0.05 ± 0.05 | 68.2 ± 1.4 |
| Si11-C6 | 97.7 ± 4.7 | 0.19 ± 0.04 | 64.9 ± 1.0 |
| Si11-C8 | 123.4 ± 5.9 | 0.16 ± 0.02 | 66.3 ± 1.7 |
| Si11-C11b | 150.4 ± 7.8 | 0.20 ± 0.08 | 65.0 ± 1.1 |
| Si11-C10V | 198.8 ± 7.2 | 0.20 ± 0.04 | 61.2 ± 1.6 |
| Si11-C10 | 108.6 ± 7.0 | 0.14 ± 0.01 | 66.7 ± 1.1 |
| Si11-C12 | 117.9 ± 7.9 | 0.13 ± 0.02 | 68.0 ± 0.9 |
| Si11-C12b | 142.7 ± 6.1 | 0.19 ± 0.01 | 62.0 ± 1.5 |
| Si11-C14 | 110.8 ± 4.4 | 0.13 ± 0.04 | 65.6 ± 3.4 |
| Si11-C14b | 125.7 ± 5.4 | 0.22 ± 0.04 | 66.8 ± 2.9 |
| Si11-C16 | 133.8 ± 6.8 | 0.15 ± 0.02 | 67.5 ± 2.1 |
| Si11-C18 | 126.8 ± 6.0 | 0.18 ± 0.05 | 66.0 ± 1.2 |
| Si11-O9 | 153.5 ± .71 | 0.21 ± 0.04 | 65.6 ± 1.4 |
| Si11-O12 | 127.5 ± 4.7 | 0.20 ± 0.08 | 67.9 ± 5.3 |
| Si11-O14 | 104.7 ± 4.5 | 0.15 ± 0.02 | 72.7 ± 1.6 |
| Si11-O16 | 119.4 ± 6.2 | 0.16 ± 0.03 | 69.6 ± 2.4 |
| Si11-O18 | 92.7 ± 4.7 | 0.19 ± 0.03 | 67.0 ± 1.4 |
| Si11-N8 | 83.6 ± 1.7 | 0.09 ± 0.04 | 83.4 ± 2.1 |
| Si11-N10 | 103.8 ± 7.1 | 0.10 ± 0.05 | 86.2 ± 1.7 |
| Si11-N12 | 66.9 ± 1.5 | 0.28 ± 0.03 | 74.9 ± 2.8 |
| Si11-N14 | 48.9 ± 4.5 | 0.42 ± 0.05 | 67.4 ± 2.4 |
| Si11-N16 | 54.1 ± 2.1 | 0.30 ± 0.04 | 67.2 ± 6.3 |
| Si12-C6 | 82.5 ± 5.2 | 0.19 ± 0.07 | 63.8 ± 1.5 |
| Si12-C8 | 92.0 ± 7.2 | 0.23 ± 0.04 | 70.6 ± 2.9 |
| Si12-C11b | 82.9 ± 2.4 | 0.25 ± 0.03 | 67.2 ± 1.4 |
| Si12-C10V | 75.1 ± 3.8 | 0.05 ± 0.03 | 63.9 ± 2.5 |
| Si12-C10 | 92.3 ± 4.5 | 0.36 ± 0.04 | 77.3 ± 3.9 |
| Si12-C12 | 57.3 ± 2.2 | 0.06 ± 0.04 | 64.4 ± 1.2 |
| Si12-C12b | 100.3 ± 6.9 | 0.40 ± 0.06 | 59.7 ± 2.3 |
| Si12-C14 | 75.9 ± 2.8 | 0.06 ± 0.03 | 82.7 ± 1.3 |
| Si12-C14b | 62.3 ± 1.9 | 0.16 ± 0.02 | 76.2 ± 1.9 |
| Si12-C16 | 103.6 ± 4.8 | 0.05 ± 0.03 | 79.7 ± 2.3 |
| Si12-C18 | 86.1 ± 3.5 | 0.18 ± 0.03 | 73.9 ± 1.3 |
| Si12-O9 | 98.3 ± 8.4 | 0.22 ± 0.04 | 66.5 ± 0.9 |
| Si12-O12 | 80.6 ± 2.0 | 0.37 ± 0.07 | 65.6 ± 2.9 |
| Si12-O14 | 86.6 ± 2.7 | 0.29 ± 0.04 | 67.9 ± 1.6 |
| Si12-O16 | 99.1 ± 8.4 | 0.27 ± 0.06 | 65.1 ± 2.1 |
| Si12-O18 | 132.2 ± 3.2 | 0.11 ± 0.05 | 64.4 ± 2.2 |
| Si12-N8 | 150.7 ± 6.4 | 0.29 ± 0.08 | 68.2 ± 1.0 |
| Si12-N10 | 95.0 ± 3.9 | 0.20 ± 0.05 | 65.9 ± 1.5 |
| Si12-N12 | 82.4 ± 2.2 | 0.04 ± 0.01 | 62.9 ± 1.9 |
| Si12-N14 | 103.6 ± 7.1. | 0.26 ± 0.07 | 61.2 ± 1.1 |
| Si12-N16 | 122.1 ± 6.5 | 0.36 ± 0.03 | 65.2 ± 1.8 |

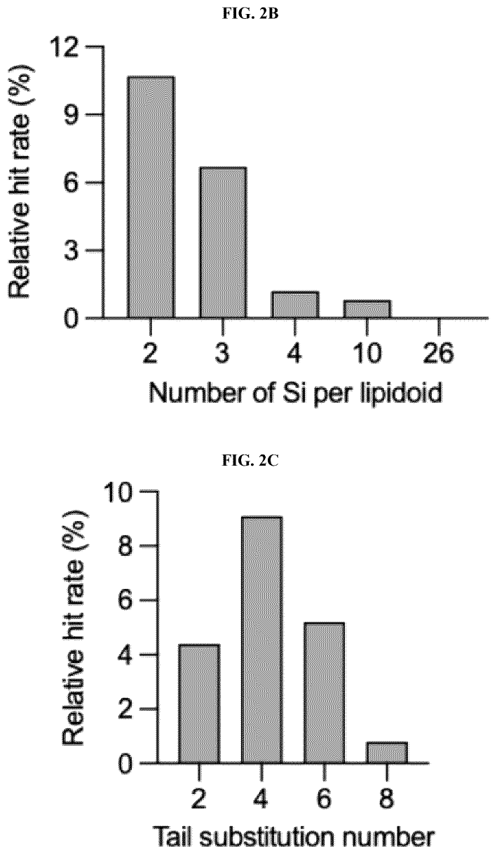

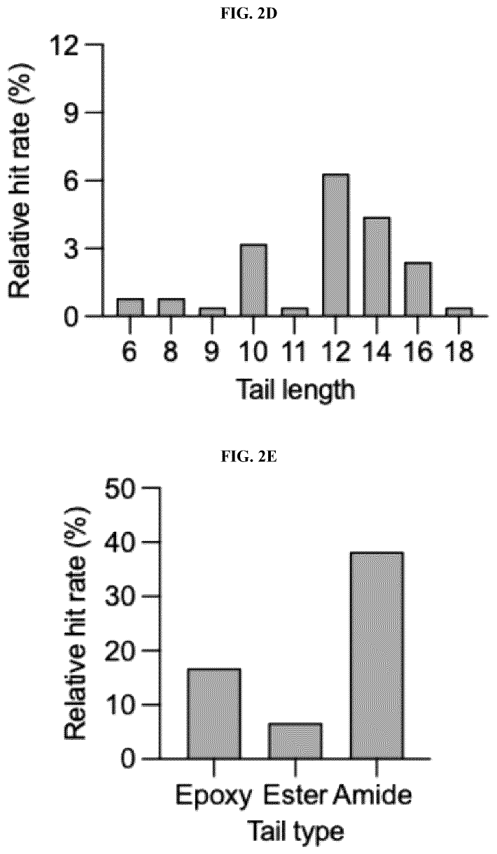

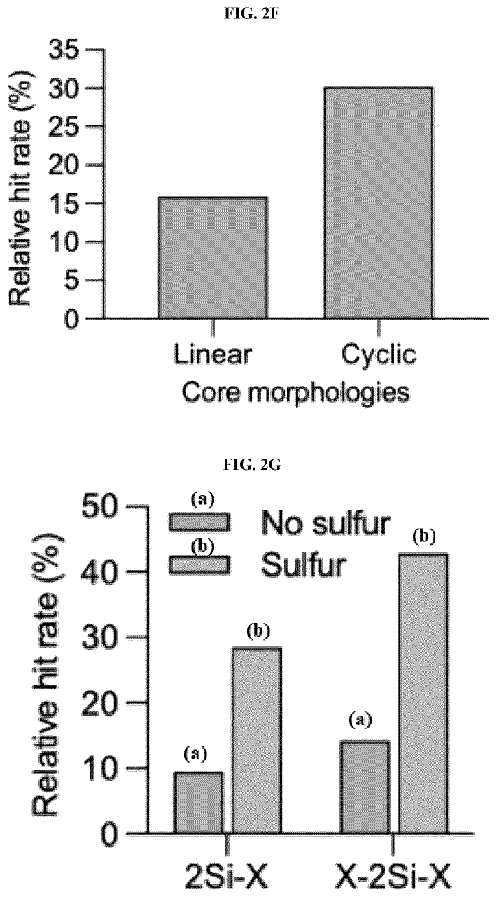

From the in vitro screening in HepG2 cells, a heat map was generated of siloxane-related factors that influenced mRNA delivery activity by calculating the relative hit rate (relative light units, RLU >200) of different siloxane lipidoid parameters to evaluate which factors are most important for mRNA delivery in vitro (FIG. 2A). Siloxane lipidoids with two silicon groups per lipidoid exhibited the highest mRNA delivery efficacy, with a hit rate of ˜10% over the whole library (FIG. 2B). It was hypothesized that siloxane lipidoids with a greater number of silicon groups (i.e., >2) had a relatively higher hydrophobicity, making it difficult to encapsulate hydrophilic RNA compounds for efficient delivery. Additionally, it was observed that siloxane lipidoids synthesized from alkyl-amines with four substitution sites exhibited higher mRNA delivery than amines with other amounts of substitution sites (FIG. 2C). Importantly, amine tail length was very influential for mRNA delivery, where chain lengths ranging from 10-16 reached hit rates of up to ˜17% (FIG. 2D). The use of different electrophile tail types (e.g., epoxide, ester, amide) also impacted mRNA delivery, wherein SiLNPs that incorporated tails with amide groups increased the hit rate up to 38% in the amide bond-associated library (FIG. 2E). These observations are in accordance with previously reported LNP systems, wherein efficacy generally correlated with polyamide cores, tail substitution sites, and tail diversity.

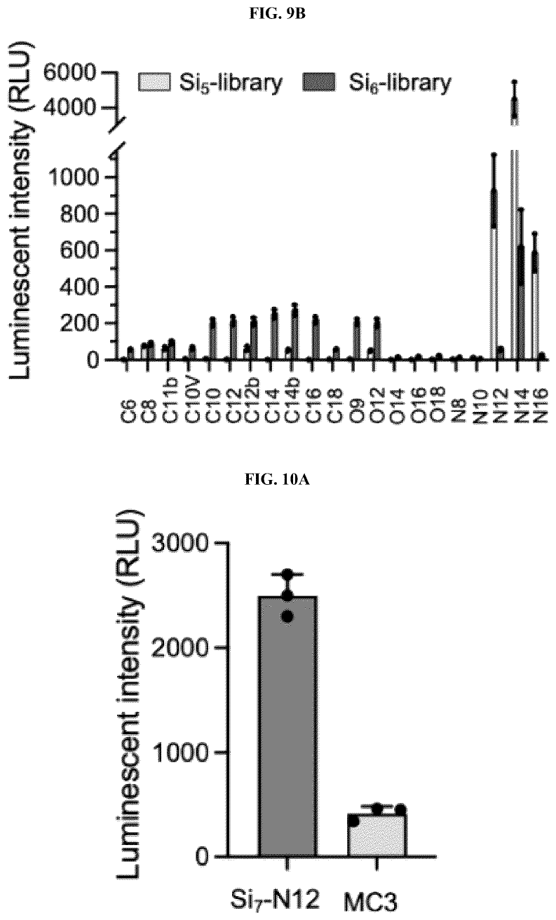

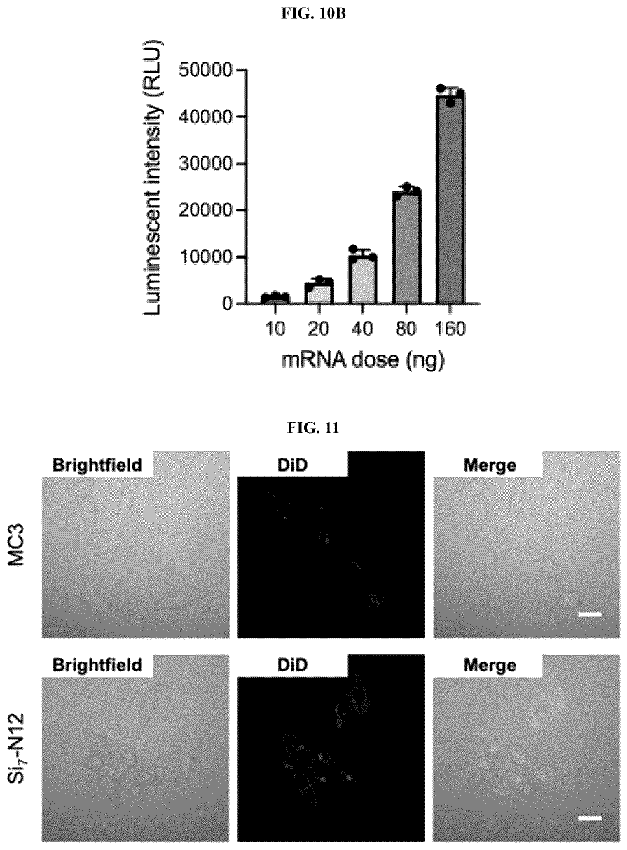

Morphologies have been shown to affect interactions between lipids and RNA, where siloxane lipidoids with cyclic structures demonstrated a ˜30% hit rate compared to linear structures which demonstrated a ˜15% hit rate (FIG. 2F). Moreover, by introducing sulfur atoms to functionalize the siloxane amine cores, greater mRNA delivery was achieved compared to lipids without sulfur substitution (FIG. 2G and FIGS. 9A-9B), which may be due to the antioxidant ability of the sulfur moiety. Within this library, siloxane lipidoid candidate Si7-N12 LNP demonstrated the highest transfection capability, mediating up to 6-fold greater protein expression compared MC3 LNP (FIG. 10A). This lead SiLNP (i.e., Si7-N12) also demonstrated dose-dependent mRNA transfection of HepG2 cells, showing increased luminescent intensity with increased Fluc mRNA dosage (FIG. 10B).

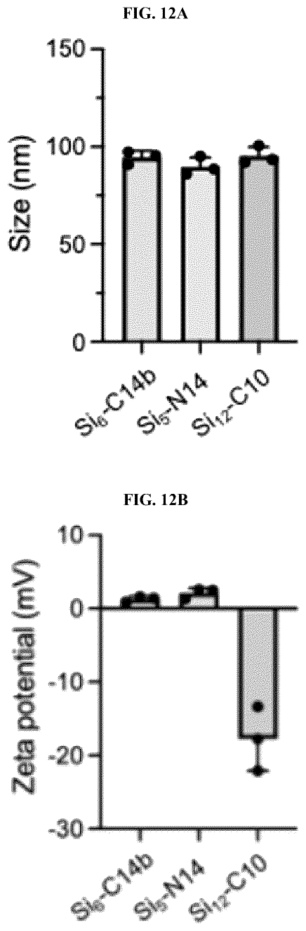

Furthermore, Si₇—N12 LNPs and MC3 LNPs were stained with 1,1′-dioctadecyl-3,3,3′,3′-tetramethylindodicarbocyanine, 4-chlorobenzenesulfonate salt (DiD) fluorescent dye, which showed that Si₇—N12 LNPs exhibited higher uptake into cells than MC3 LNPs (FIG. 11), suggesting that the hydrophobic siloxane domain aided in efficient cellular uptake of LNPs. These studies highlight the potential for the incorporation of siloxane lipidoids into LNP formulations to enhance mRNA efficacy in vitro, motivating further exploration of SiLNP delivery potency in vivo.

### Example 4: Siloxane Moiety Incorporation Improves Intracellular Uptake and Endosomal Escape

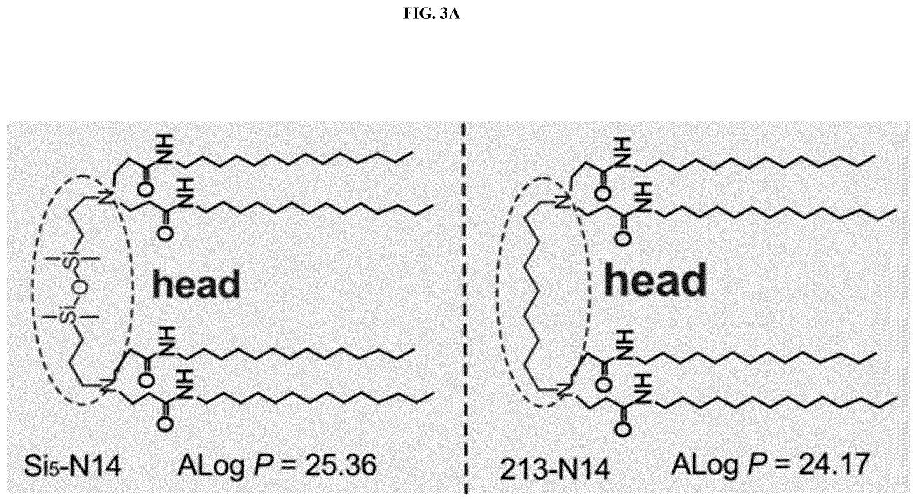

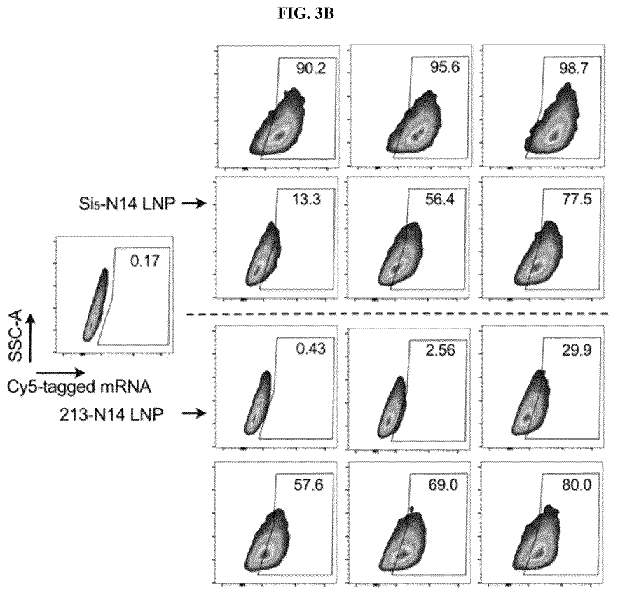

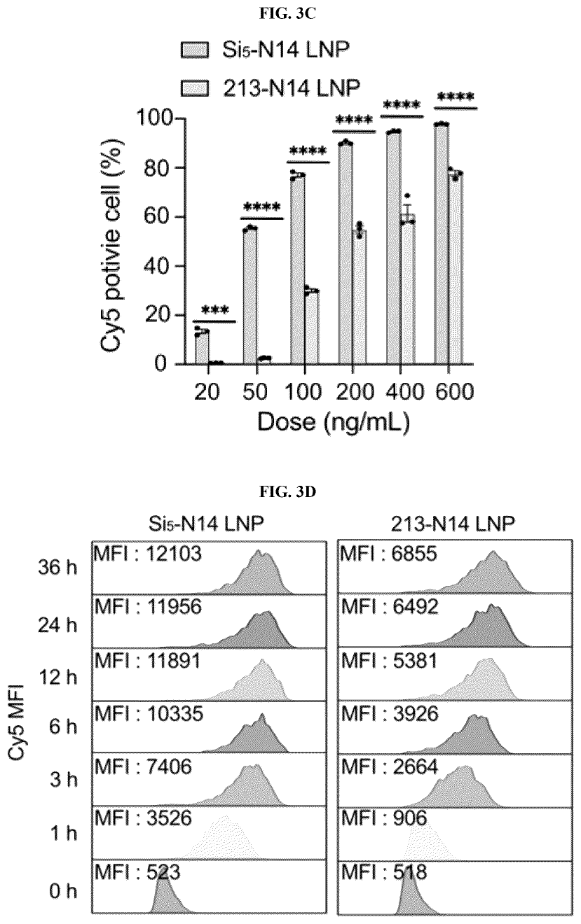

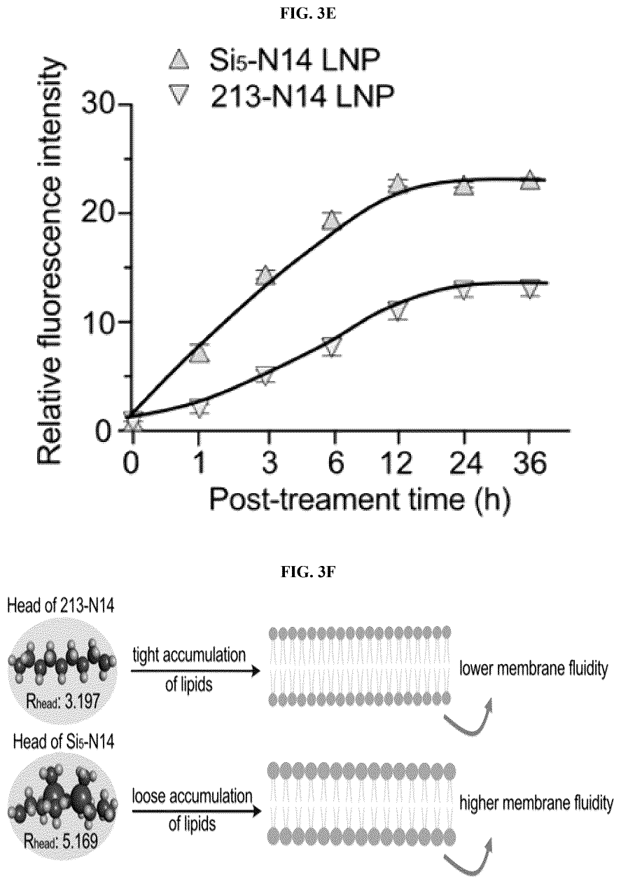

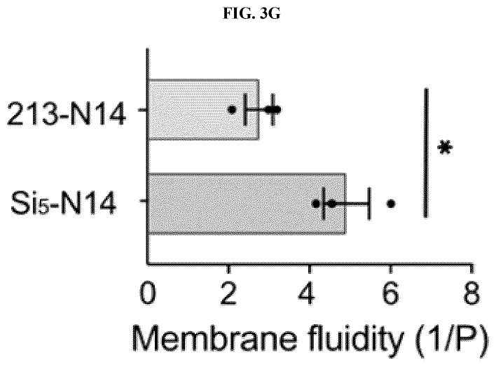

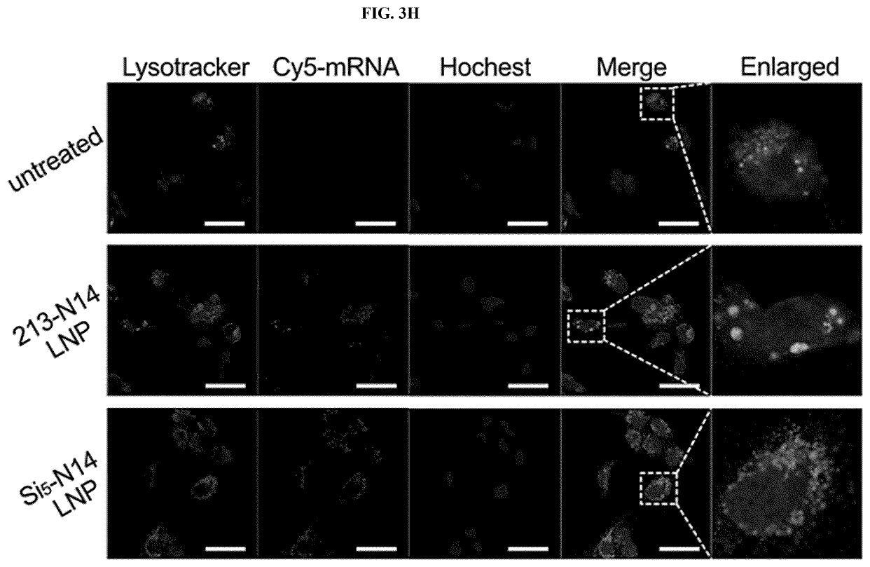

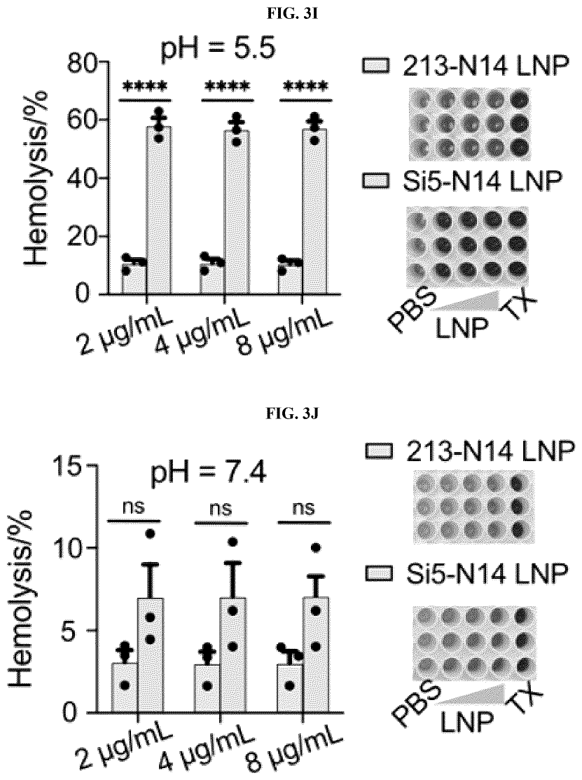

In order to elucidate the precise role of the siloxane moiety for intracellular mRNA delivery, it was considered imperative to use lipidoids with identical tail structures and head length, but lacking siloxane domains, as controls. However, prepared compounds wherein the siloxane moiety is substituted with alkyl groups in the same position on Si7-, Si8-, and Si10-based amine cores is synthetically challenging. Therefore, the 213-N14 lipidoid was synthesized as a reference for Si5-N14 lipidoid (FIG. 3A). These two lipidoids have analogous tail structures and amine head lengths, but differ solely in the presence or absence of the siloxane moiety.

TABLE 2
Characterization of exemplary LNPs
(Si₅-N14 and 213-N14) comprising Cy5 mRNA

| | | Encapsulation | Zeta |
|---|---|---|---|
| | | Efficiency | Potential |
| LNP | Size (nm) | (%) | (mV) |
| Si₅-N14 | 91.2 ± 5.3 | 89.4 | 3.3 ± 1.4 |
| 213-N14 | 105.1 ± 4.8 | 96.9 | 4.9 ± 1.2 |

To evaluate the efficacy of these two lipidoids for mRNA delivery, an endothelial cell line, immortalized human lung microvascular endothelial cells (iMVECs), was employed, given that blood vessels constitute the primary site of interaction subsequent to systemic administration.

First, intracellular uptake was investigated in vitro by Si5-N14 and 213-N14 LNPs formulating with Cy5-tagged mRNA (Table 2). Si5-N14 LNPs demonstrated a significantly faster cellular uptake rate than 213-N14 LNPs, as indicated by the higher Cy5-positive cell populations at different mRNA dose after 3 h treatment (FIGS. 3B-3C). In particular, at a low mRNA dose of 20 ng/mL, Si5-N14 LNPs exhibited almost 30 times higher Cy5 mRNA uptake than 213-N14 LNPs (FIG. 3B).

Furthermore, Si5-N14 LNPs induced a higher level of particles endocytosis compared to 213-N14 LNP, as demonstrated by a significantly greater Cy5 mean fluorescence intensity MFI (FIG. 3D). The endocytosis capacity was further plotted to demonstrate faster and greater endocytosis of Si5-N14 LNPs than 213-N14 LNPs (FIG. 3E), which could be attributed to the relatively higher hydrophobic performance (higher A Log P value) of Si5-N14 that mediates greater cellular internalization (FIG. 3A). These findings highlight the critical role of the siloxane domain in improving the cellular internalization of mRNA cargos.

Based on the larger atomic radius of silicon compared to carbon, it was hypothesized that the incorporation of a siloxane-based amine head into lipidoids may result in a looser accumulation of lipids across the membrane, which in turn could increase the membrane fluidity and promote mRNA transfection efficacy (FIG. 3F). To test this hypothesis, molecular dynamics simulations were conducted, which showed that the head radius of Si5-N14 (R=5.169) was larger than that of 213-N14 (R=3.197), indicating the potential for increasing membrane fluidity after the incorporation of siloxane moiety (FIG. 3F).

To evaluate membrane fluidity, a fluorescence probe was used to measure the reciprocal polarization (1/P), which reflected the fluidity of the membrane. The experimental results showed that Si5-N14 (1/P=4.87) had higher fluidity than 213-N14 (1/P=2.72) (FIG. 3G). Moreover, membrane fluidity can affect the fusion of endosomal membranes, which is essential for the endosomal escape of genetic cargos. Confocal laser scanning microscope (CLSM) imaging revealed that Si5-N14 LNPs were more efficient in escaping from endosomes than 213-N14 LNPs, as indicated by the greater cytosolic distribution of Cy5 mRNA (red) and less co-localization between Cy5 mRNA and endosomes (green) in Si5-N14 LNPs treated cells (FIG. 3H).

Next, whether or not the improved endosomal escape of Si5-N14 lipidoid also enhances membrane-disruptive activities was investigated. Hemolysis assay demonstrated that Si5-N14 LNPs exhibited significantly stronger hemolysis than 213-N14 LNPs at both neutral and acidic pH (FIGS. 31-3J). These results together strongly validate the importance of siloxane domain aiding in cellular internalization and endosomal escape for mRNA delivery.

### Example 5: SiLNPs Enable Structure-Guided Tissue-Specific mRNA Delivery In Vivo

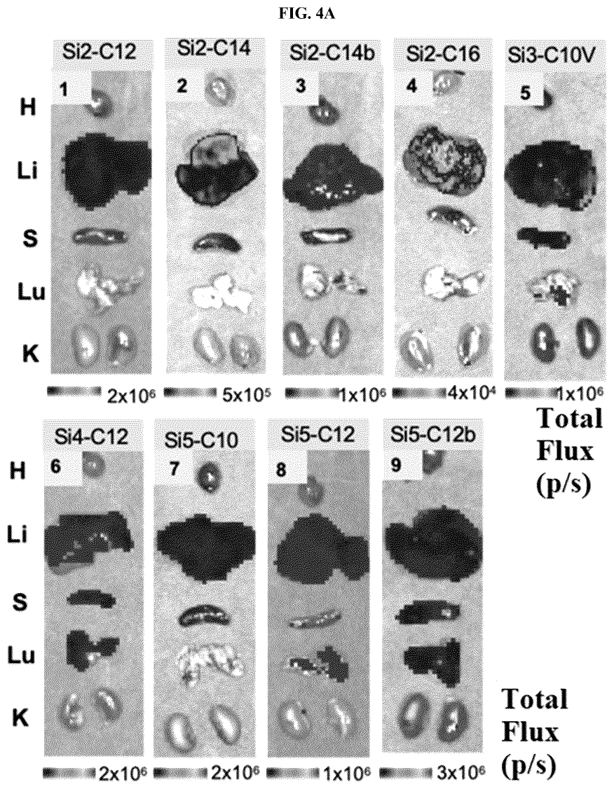

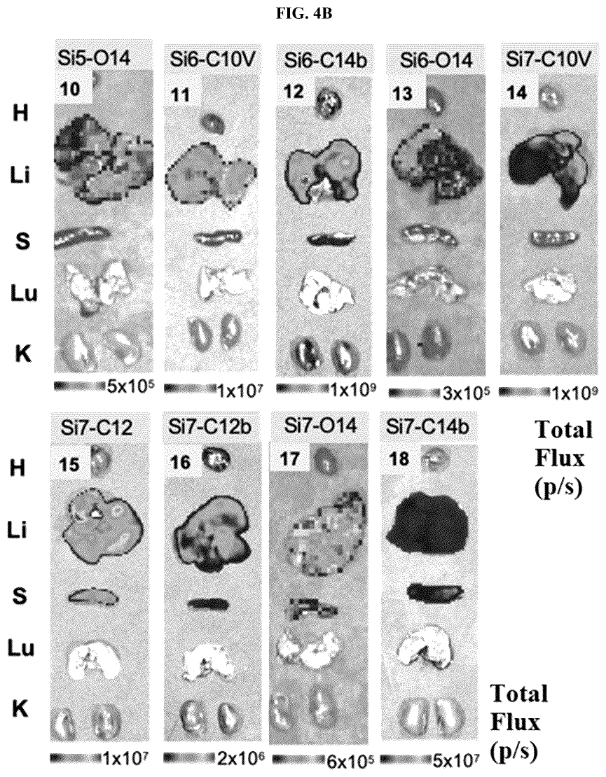

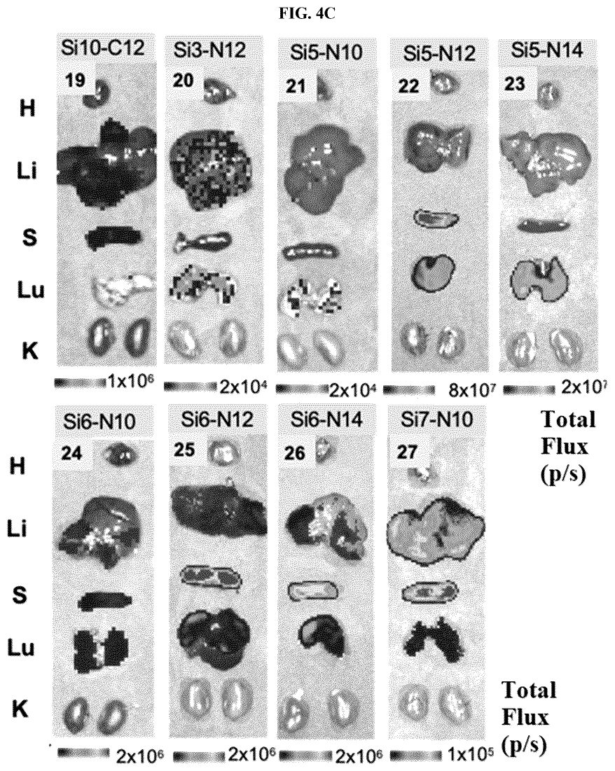

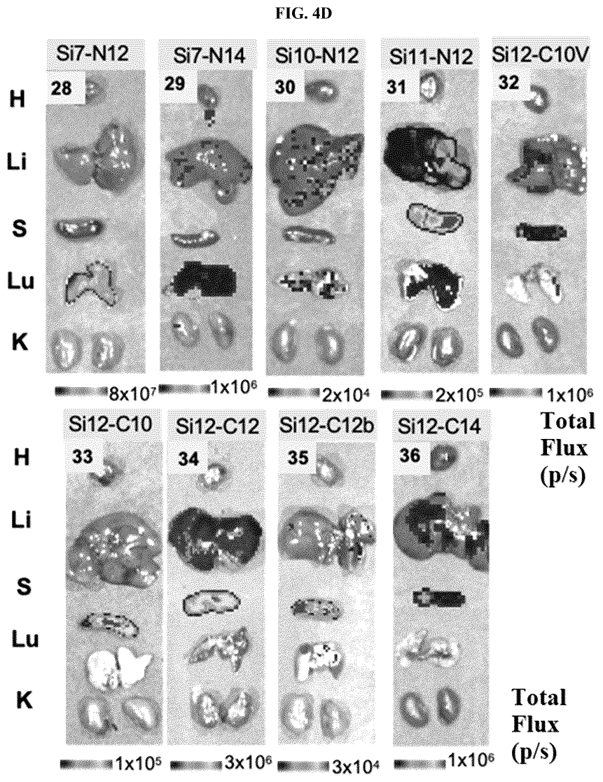

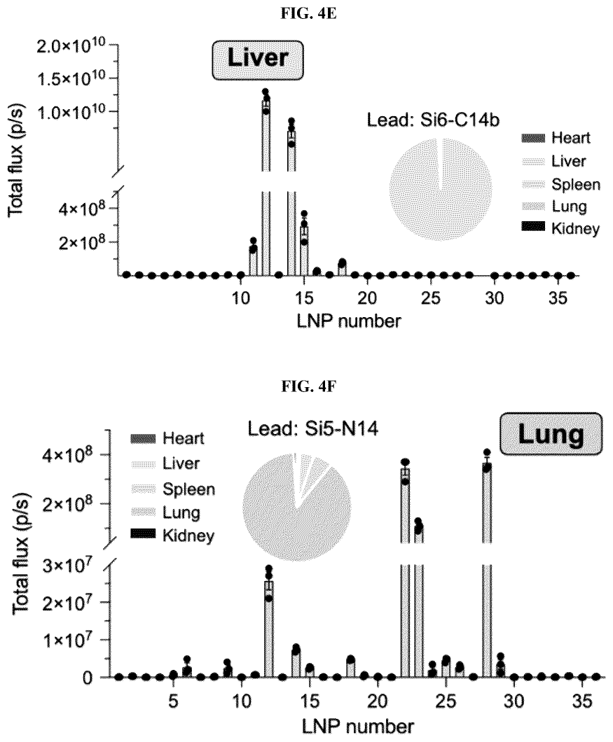

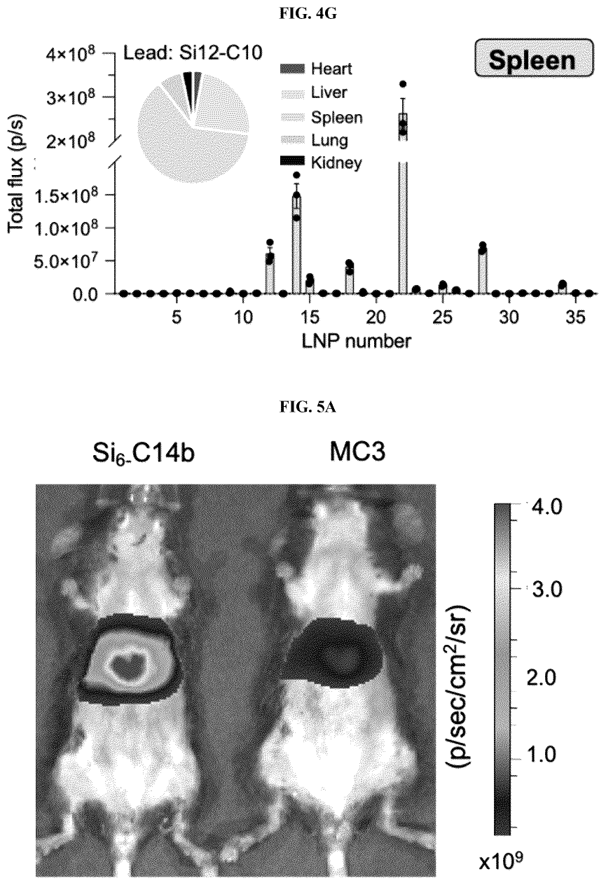

To further evaluate the potential of SiLNPs for mRNA delivery in vivo, 36 top-performing SiLNPs were selected from the in vitro screen and in vivo delivery of Fluc mRNA to wild-type C57BL/6 mice was quantified at an mRNA dose of 0.25 mg kg⁻¹. Mice were injected intraperitoneally (i.p.) with a luciferin substrate 6 h post SiLNP intravenous (i.v.) injection, and organs (i.e., heart, liver, spleen, lung, and kidney) were isolated to quantify Fluc activity using an in vivo imaging system (IVIS). The ex vivo mRNA delivery activity of 23 representative SiLNPs were demonstrated. Interestingly, an organ-selective mRNA delivery profile was shown by altering the siloxane-based amine head and alkyl chain structures (FIGS. 4A-4D and Table 3), demonstrating the possibility of tuning SiLNP tissue targeting from the liver to the lung and spleen. This represents the first demonstrations of ionizable lipid-like materials achieving tissue-specific mRNA delivery by only altering the ionizable lipid-like material structure.

TABLE 3
Organ (liver, lung, and spleen) luminescence
quantification (FIGS. 4A-4D)

| | Live [total | Lung [total | Spleen [total |
|---|---|---|---|
| LNP | flux (p/s)] | flux (p/s)] | flux (p/s)] |
| 1 | 6.76E6 ± 1.6E6 | 6.6E4 ± 4.6E4 | 6.6E4 ± 2.3E4 |
| 2 | 4.1E6 ± 8.1E5 | 3.1E5 ± 2.0E4 | 5.2E4 ± 1.7E3 |
| 3 | 4.4E5 ± 2.9E5 | 4.9E4 ± 2.5E4 | 2.4E4 ± 1.9E4 |
| 4 | 3.1E5 ± 3.2E4 | 1.2E4 ± 1.7E3 | 2.1E4 ± 2.4E3 |
| 5 | 6.4E6 ± 3.5E6 | 6.6E5 ± 3.4E5 | 1.0E6 ± 2.7E5 |
| 6 | 4.4E6 ± 2.3E6 | 2.7E6 ± 1.8E6 | 6.7E5 ± 3.4E5 |
| 7 | 2.4E6 ± 1.4E6 | 3.5E4 ± 2.3E4 | 2.8E4 ± 1.3E4 |
| 8 | 9.3E5 ± 3.5E5 | 2.1E5 ± 9.7E4 | 5.6E4 ± 4.6E4 |
| 9 | 6.0E6 ± 2.3E6 | 2.5E6 ± 1.5E6 | 2.4E6 ± 1.5E6 |
| 10 | 4.1E6 ± 4.4E6 | 1.5E4 ± 1.9E4 | 1.6E5 ± 1.4E5 |
| 11 | 1.7E8 ± 3.1E7 | 7.1E5 ± 4.5E4 | 6.9E5 ± 6.1E4 |
| 12 | 1.1E10 ± 1.5E9 | 2.5E7 ± 4.1E6 | 6.1E7 ± 1.4E7 |
| 13 | 4.1E6 ± 2.2E6 | 1.9E4 ± 1.7E3 | 3.1E5 ± 4.6E4 |
| 14 | 7.0E9 ± 1.7E9 | 7.4E6 ± 6.5E5 | 1.4E8 ± 3.2E7 |
| 15 | 2.9E8 ± 8.6E7 | 2.5E6 ± 3.5E5 | 2.1E7 ± 5.5E6 |
| 16 | 2.8E7 ± 4.5E6 | 8.3E4 ± 5.2E3 | 7.2E5 ± 4.5E4 |
| 17 | 5.8E6 ± 2.3E6 | 3.1E4 ± 1.9E3 | 5.8E5 ± 7.3E4 |
| 18 | 7.7E7 ± 1.0E7 | 4.7E6 ± 3.1E5 | 4.1E7 ± 7.3E6 |
| 19 | 4.9E6 ± 1.3E6 | 5.0E5 ± 2.7E5 | 1.9E6 ± 1.4E6 |
| 20 | 1.9E5 ± 7.5E4 | 1.6E5 ± 1.1E5 | 4.4E4 ± 1.9E4 |
| 21 | 6.6E4 ± 1.1E4 | 4.6E4 ± 2.6E4 | 93.E3 ± 4.0E3 |
| 22 | 7.1E6 ± 9.1E5 | 3.4E8 ± 4.6E7 | 2.6E8 ± 5.8E7 |
| 23 | 5.3E6 ± 8.1E5 | 1.1E8 ± 2.0E7 | 7.2E6 ± 6.6E5 |
| 24 | 2.9E6 ± 1.8E6 | 1.9E6 ± 1.3E6 | 7.2E5 ± 2.5E5 |
| 25 | 4.7E6 ± 8.3E5 | 4.6E6 ± 6.4E5 | 1.3E7 ± 2.0E6 |
| 26 | 3.4E6 ± 6.5E5 | 2.7E6 ± 5.5E5 | 5.5E6 ± 4.0E5 |
| 27 | 1.7E6 ± 8.3E5 | 1.3E5 ± 1.5E4 | 5.0E5 ± 4.1E4 |
| 28 | 4.5E6 ± 4.5E5 | 3.6E8 ± 3.7E7 | 6.8E7 ± 5.1E6 |
| 29 | 3.6E4 ± 1.2E4 | 3.4E6 ± 2.2E6 | 2.6E4 ± 1.0E4 |
| 30 | 1.5E5 ± 2.3E4 | 4.3E4 ± 2.5E4 | 2.3E4 ± 1.0E4 |
| 31 | 1.3E6 ± 2.0E5 | 1.7E5 ± 1.5E4 | 8.6E5 ± 1.5E5 |
| 32 | 1.9E6 ± 1.3E6 | 2.1E5 ± 1.6E5 | 6.8E5 ± 2.7E5 |
| 33 | 2.1E5 ± 3.6E4 | 6.2E4 ± 5.0E3 | 3.4E5 ± 1.5E4 |
| 34 | 6.6E6 ± 4.5E5 | 3.9E5 ± 3.6E4 | 1.4E7 ± 2.1E6 |
| 35 | 1.5E5 ± 2.5E4 | 5.6E4 ± 4.0E3 | 1.0E6 ± 3.1E5 |
| 36 | 1.4E6 ± 5.9E5 | 1.6E5 ± 6.1E4 | 4.8E5 ± 4.3E5 |

Siloxane lipidoids with epoxide/ester-based tails formulated into SiLNPs mainly delivered mRNA to liver, which is in accordance with previous findings featuring non-siloxane structures for hepatic mRNA delivery, whereas the top-performing liver SiLNPs (e.g., Si6-C14b) exhibited luciferase expression primarily in the liver (˜98%) compared to other organs (FIG. 4E).

By replacing epoxide-based tail structures with amide-linked tail structures, SiLNPs altered tissue tropism to lung tissues, where a siloxane lipidoid (i.e., Si5-N14) comprising two silicon atoms, two tertiary amines, four amide-linked 14 carbon tails resulted in the most efficient lung-specific mRNA delivery (FIGS. 4A-4D), with luciferase expression predominantly in lung tissues (˜90%) (FIG. 3F). It was noted that the hydrophobicity, polarity and saturation of alkyl tails affects organ-tropic mRNA delivery efficacy in vivo, and thus changing their properties could alter the protein corona composition on the surface of LNPs which could affect their biodistribution. Although previous work showed amide-linked tails could enhance lung-tropism for RNA delivery, mRNA expression in other organs was still present, which may result from the relatively low cellular uptake rate of these LNPs. When a siloxane element was incorporated into the LNP formulation, the increased hydrophobicity led to faster cellular uptake (FIG. 11), which may have contributed to lung specificity in blood circulation. Thus, after evaluating amine bonds in the tail structures of siloxane lipidoids, Si5-N14 demonstrated the highest protein expression predominantly in the lung with high selectivity and transfection efficacy.

It has been shown that introducing additional negatively charged phospholipids as a fifth component could assist in splenic mRNA delivery, however, the integration of these negatively charged phospholipids with poor solubility into LNP formulations is challenging.

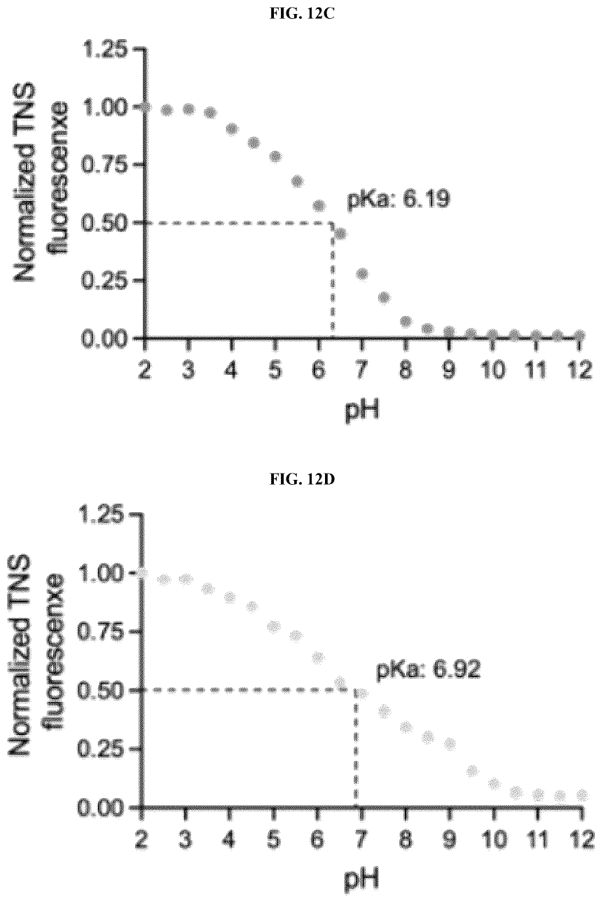

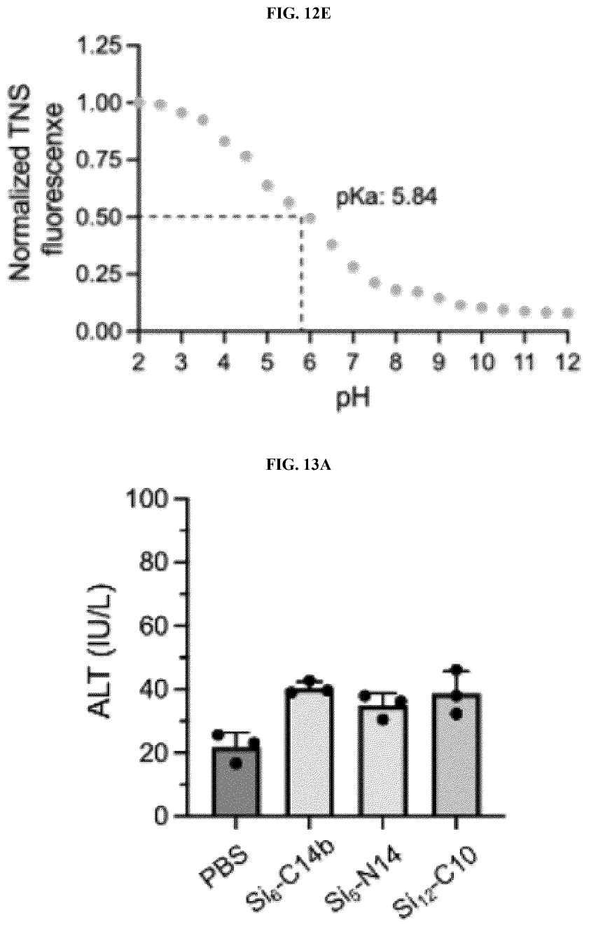

Thus, engineering ionizable lipids with negatively charged groups represents another approach to endow the resulting LNP formulations with a negative charge, potentially enabling splenic RNA delivery. To this end, a cyclic siloxane structure with multiple reaction sites was designed to attach both a negatively charged alkylsulfonic acid group and amine head-alkyl tails (FIG. 1D), showing exclusive spleen-tropism in vivo (FIGS. 4A-4D). Through an in vivo screen, siloxane lipidoid (Si12-C10) was identified with good solubility and a negative charge that led to highly efficient spleen-specific mRNA delivery (FIGS. 4G and 10 FIGS. 12A-112E).

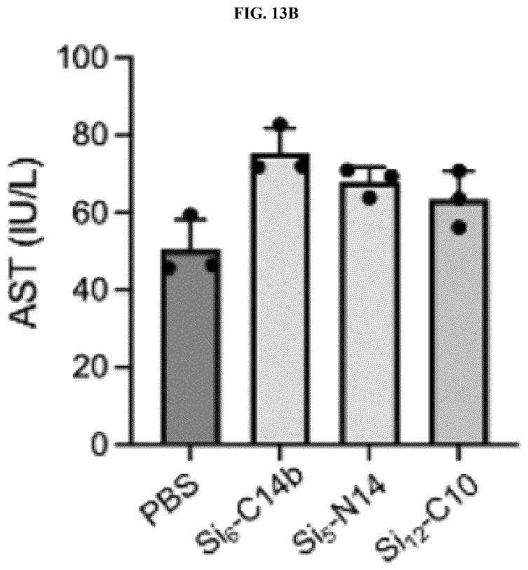

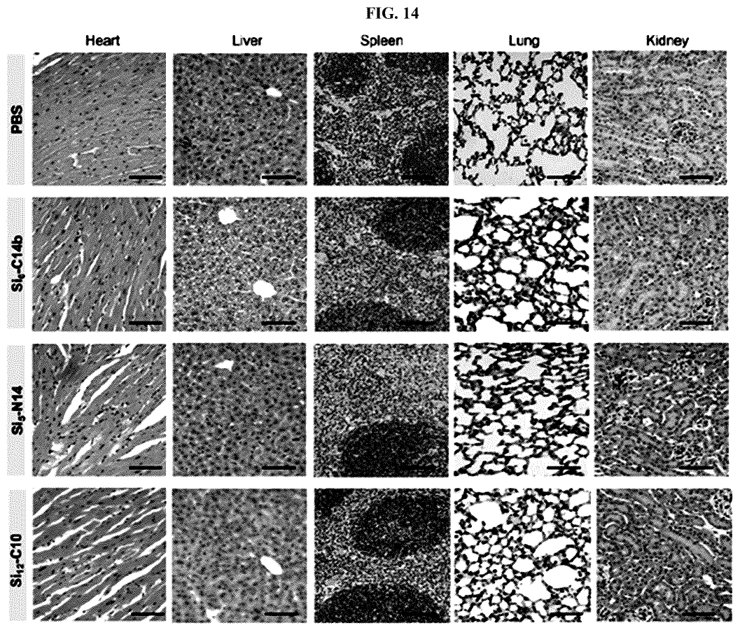

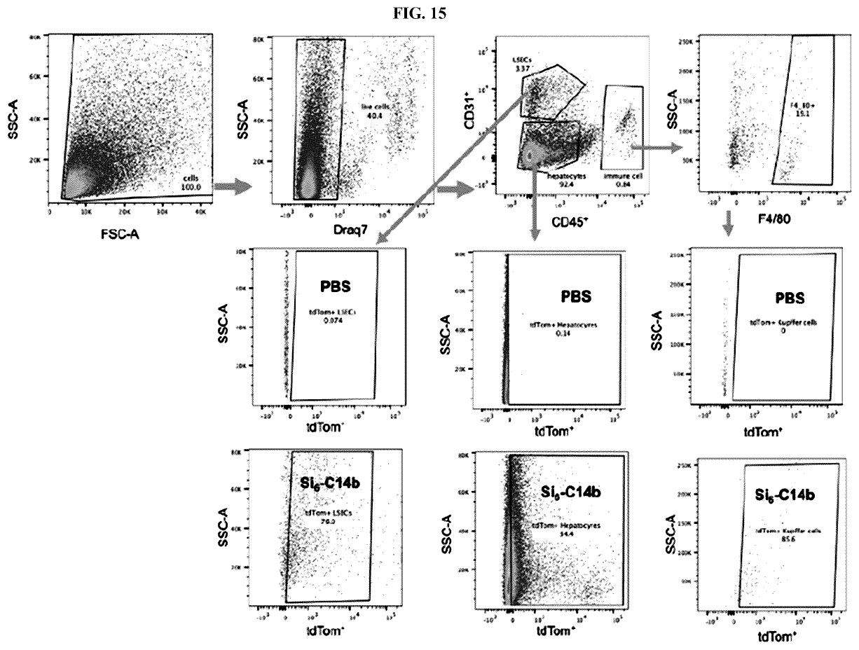

According to this structure-guided tissue-specific mRNA delivery by SiLNPs in vivo, these results suggest that siloxane lipidoids with epoxide-/ester-based tails promoted mRNA delivery to the liver, lipidoids with amide-linked tails promoted mRNA delivery to the lung, and negatively charged lipidoids aided in mRNA delivery to the spleen. After quantifying their ex vivo luminescent intensity (FIGS. 4A-4D), Si6-C14b, Si5-N14, and Si12-C10 were identified as the lead liver-targeting, lung-targeting, and spleen-targeting SiLNPs, respectively, as they achieved potent and selective mRNA delivery to their respective organs. These selected siloxane lipidoids were purified and the resulting SiLNPs had desirable hydrodynamic diameter (90 nm to 100 nm) for endocytosis, suitable surface zeta potential (roughly neutral charge for Si6-C14b and Si5-N14; negative charge for Si12-C10 LNP), and optimal pKa (5.84 to 6.92) for endosomal escape (FIGS. 12A-12E). In addition, the toxicity of these liver, lung, and spleen targeted SiLNPs were evaluated by systemic administration with higher dosages, where all SiLNPs showed minimal toxicity as evaluated by liver enzyme levels alanine transaminase (ALT) and alanine aspartase (AST) compared to PBS injected control group (FIGS. 13A-13B). To further investigate their biosafety, tissue section histology of the main organs (e.g., heart, liver, spleen, lung, and kidney) was performed, which showed negligible in vivo toxicity (FIG. 14).

### Example 6: Liver-Specific SiLNPs Enable In Vivo CRISPR-Cas9 Editing to Reduce Serum mTTR Levels

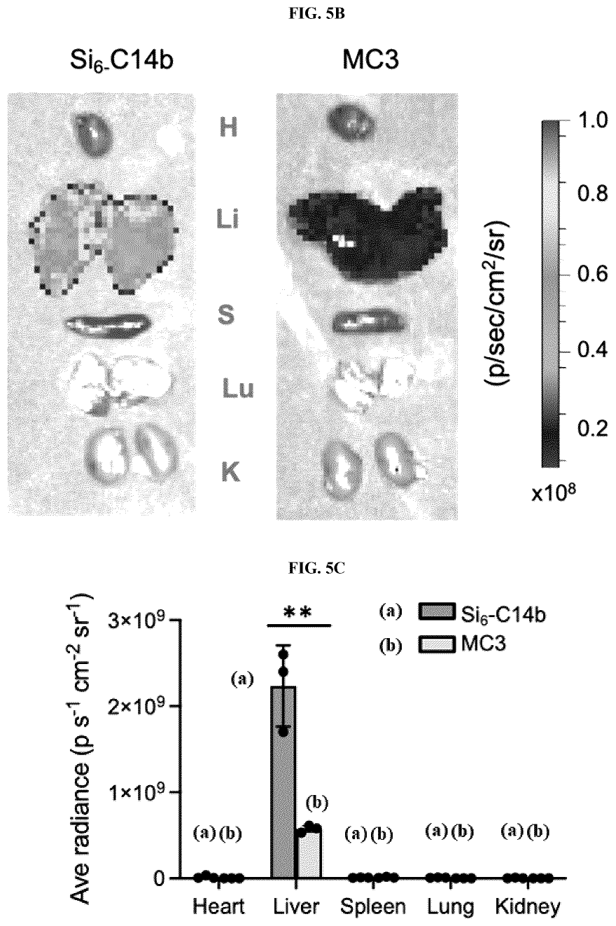

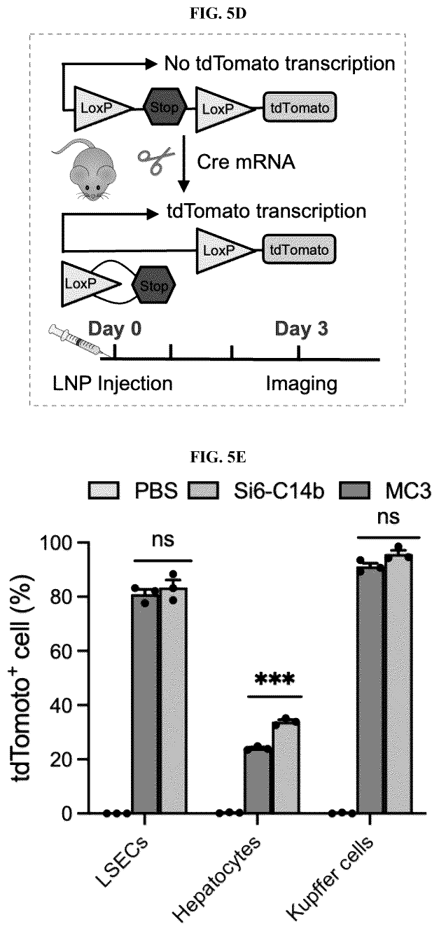

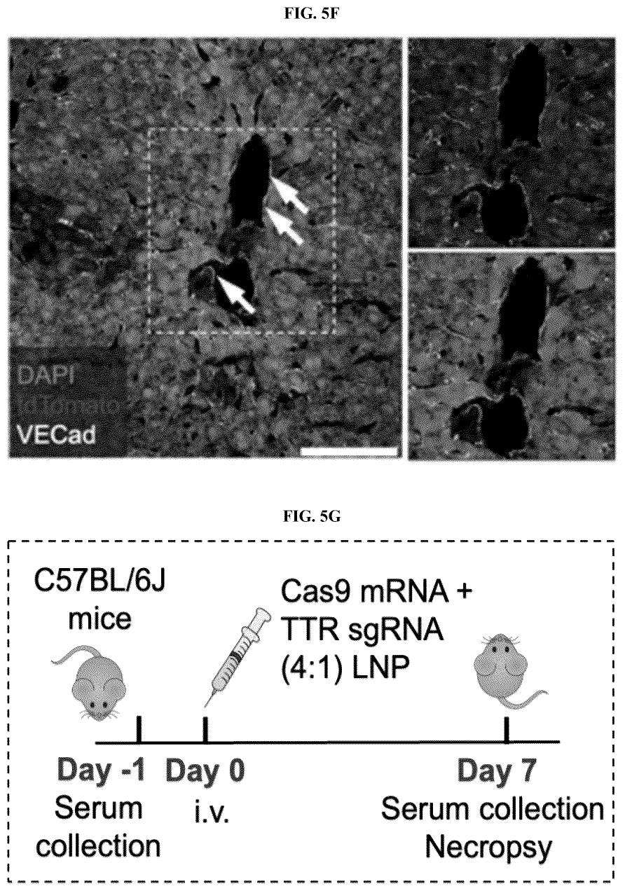

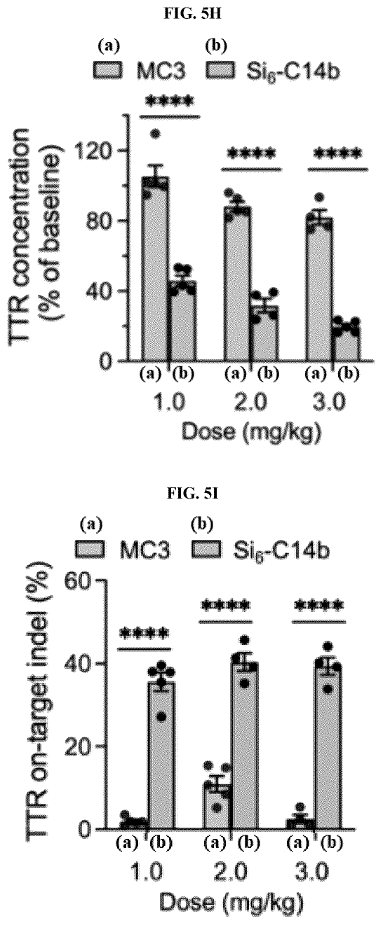

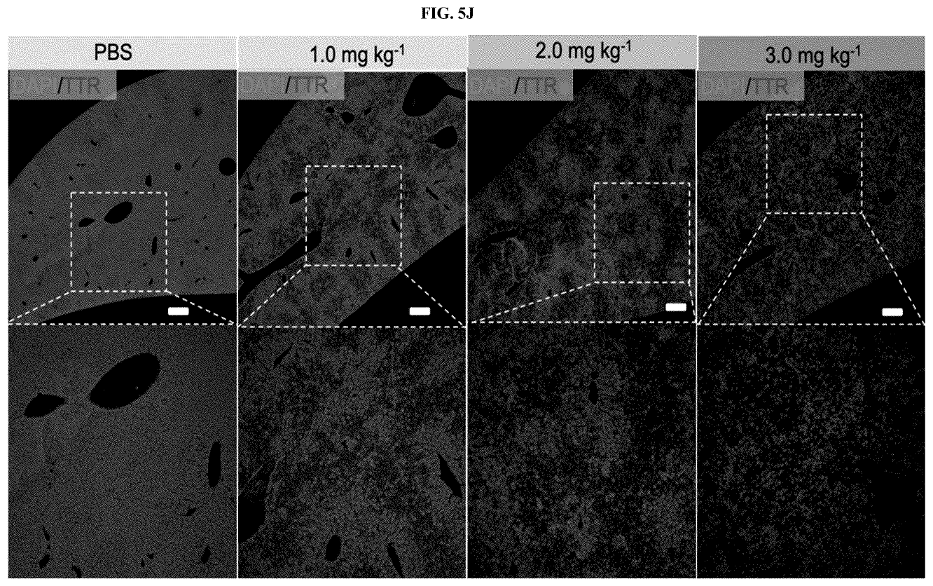

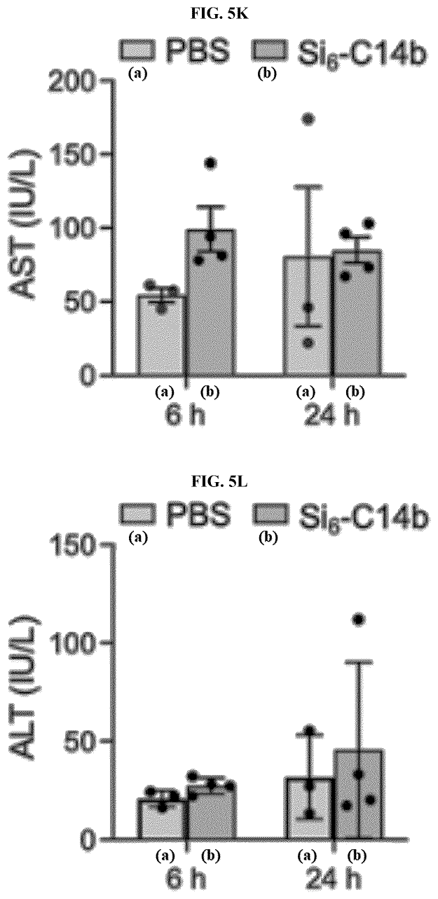

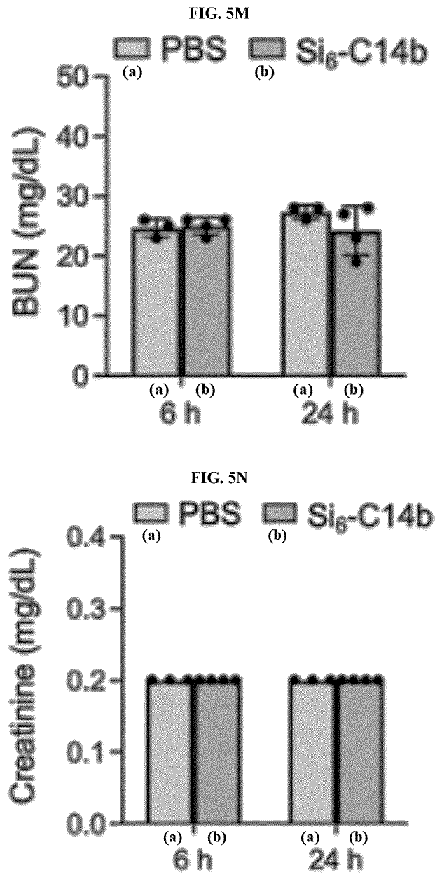

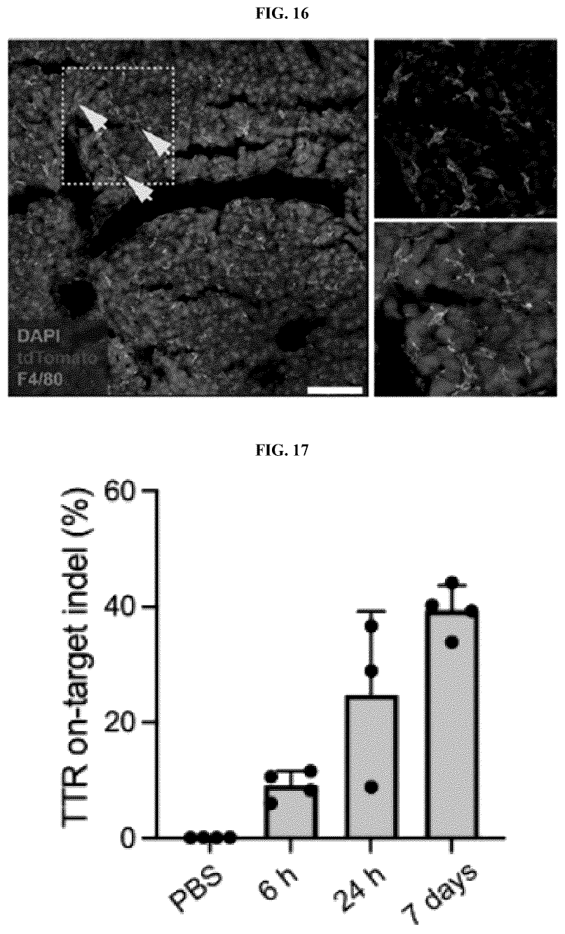

After demonstrating that SiLNPs mediate tissue-specific mRNA delivery, hepatic mRNA delivery by liver-targeted Si₆-C14b LNPs was further investigated. Initially, the potency of luciferase-encoding mRNA LNPs for liver-specific Si₆-C14b was explored, and compared to MC3 LNPs (positive control) that were formulated with the phospholipid 1,2-distearoyl-sn-glycero-3-phosphocholine (DSPC), cholesterol, and C14PEG2000. Compared to the MC3 LNP, Si₆-C14b LNPs showed 8-fold higher mRNA delivery efficacy at an mRNA dose of 0.15 mg kg⁻¹ in vivo (FIGS. 5A-5C). To further verify the transfection of certain cell types in the liver and the capability to induce liver-targeted gene editing using Si₆-C14b LNPs, the activatable Cre-LoxP mice (Ai14 mice) model that expresses Lox-stop-Lox tdTomato throughout the whole body was used. In this model, the translated Cre protein deletes the stop cassette, and then activates tdTomato fluorescence only in transfected cells following intracellular delivery of Cre-recombinase mRNA (Cre mRNA) (FIG. 5D). Following a single administration of 0.3 mg kg⁻¹ Cre mRNA, highly efficient liver gene editing was observed (FIG. 5E). Liver-specific Si₆-C14b LNPs mediated mRNA delivery to ˜35% of hepatocytes, ˜70% of liver sinusoidal endothelial cells (LSECs), and ˜82% of Kupffer cells (FIGS. 5E-5F and FIGS. 15-16). These results demonstrate the potential of Si₆-C14b LNPs for diverse mRNA liver therapeutic applications.

Although these initial in vivo experiments were conducted with a low dose of mRNA, high dose administrations could be required for certain therapeutic applications. Efficient disease treatments in murine therapeutic models may be possible through administration of mRNA-SiLNPs at higher dose, for instance, CRISPR-Cas9 based gene editing. However, successful co-delivery of Cas9 mRNA and single guide RNA (sgRNA) in vivo still remains a challenge due to the large size Cas9 mRNA and the complexity of co-delivery.

To evaluate Si6-C14b LNPs as a platform for CRISPR-Cas9 editing applications we formulated Si6-C14b LNPs were formulated with Cas9 mRNA and sgRNA targeting mouse transthyretin (TTR) gene in the liver, and administered the LNPs at doses of 1.0, 2.0, and 3.0 mg kg⁻¹ of total RNA (mRNA/sgRNA, 4/1, wt/wt) (SEQ ID NOs:3-4). Gene editing efficacy was quantified by examining serum TTR protein concentration and on-target DNA sequencing 7 days post-injection (FIG. 5G). To demonstrate the advantages of SiLNPs for gene editing, MC3 LNPs co-delivering Cas9 mRNA/TTR sgRNA were included as a positive control. With increased RNA dosing, decreased serum TTR concentration was achieved, with high (˜70%) knockout of serum TTR levels at the dose of 2.0 mg kg⁻¹ with Si₆-C14b LNPs whereas MC3 LNPs showed minimal (˜12%) knockout of serum TTR levels (FIG. 5H). For both Si₆-C14b and MC3 LNPs, increasing the RNA dose to 3.0 mg kg⁻¹ further decreased serum TTR levels compared to the 2.0 mg kg⁻¹ dose treatment group. On-target indel frequencies for the TTR gene were further quantified, where ˜40% editing of on-target DNA was observed in the liver following Si₆-C14b LNPs treatment, while MC3 LNPs edited only ˜10% of on-target DNA (FIG. 5I). In situ hybridization (ISH) analysis of liver sections further confirmed dose-dependent knockout of TTR transcript (FIG. 5J).

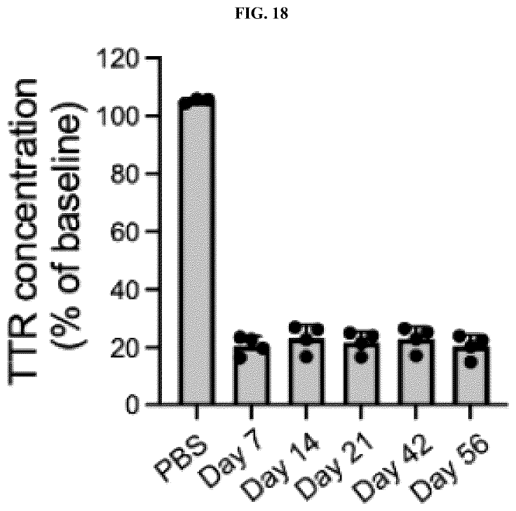

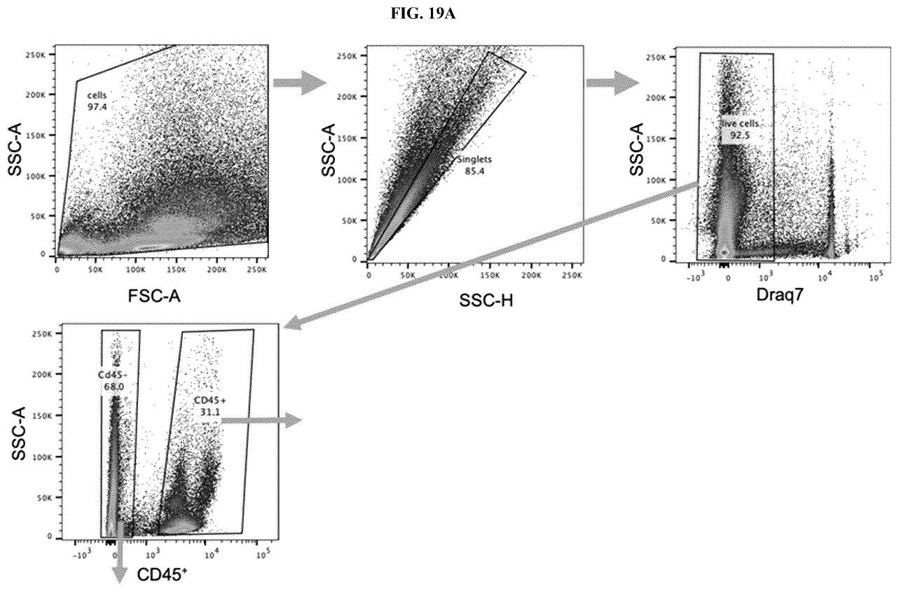

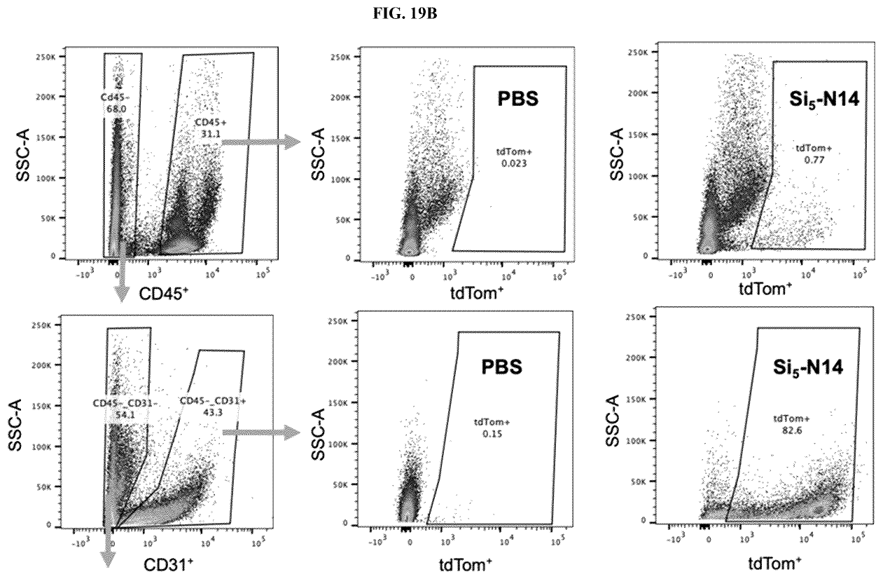

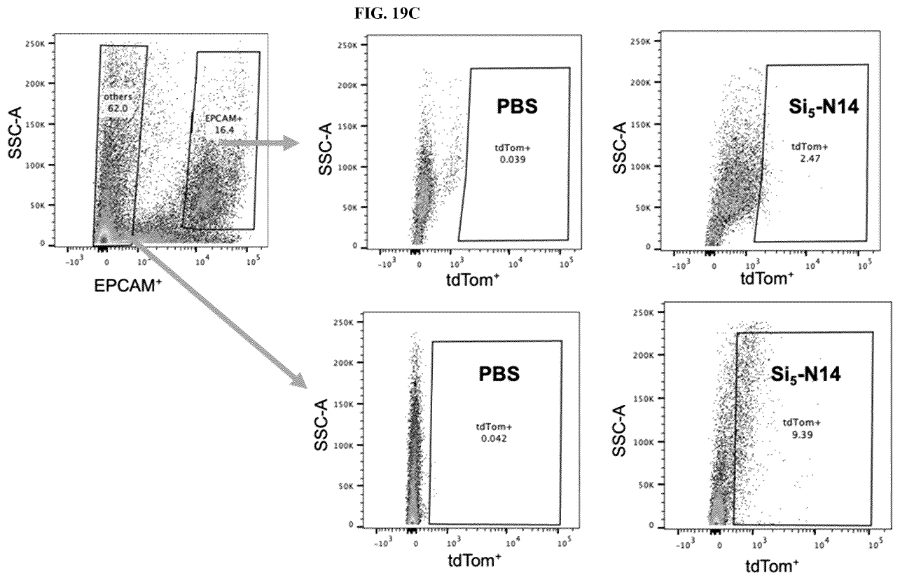

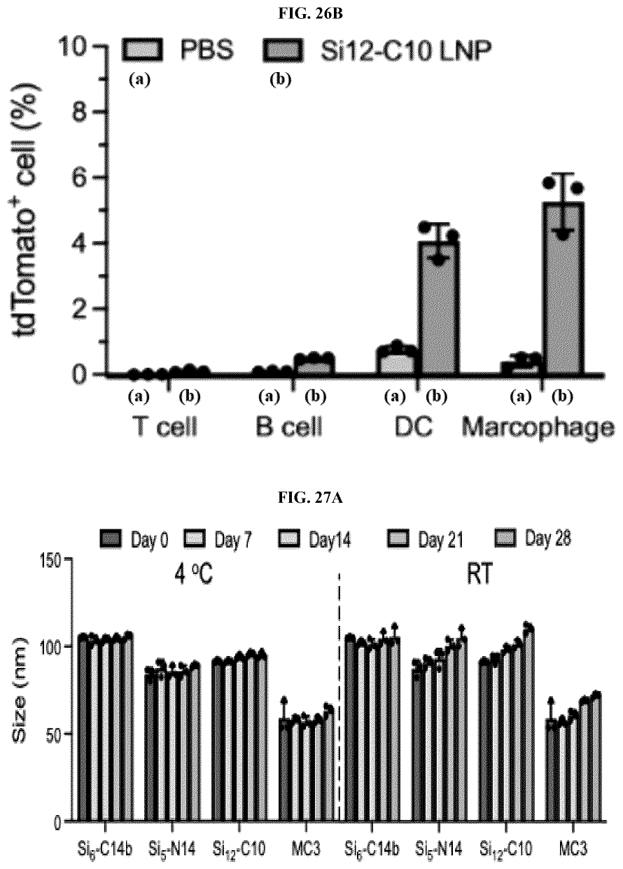

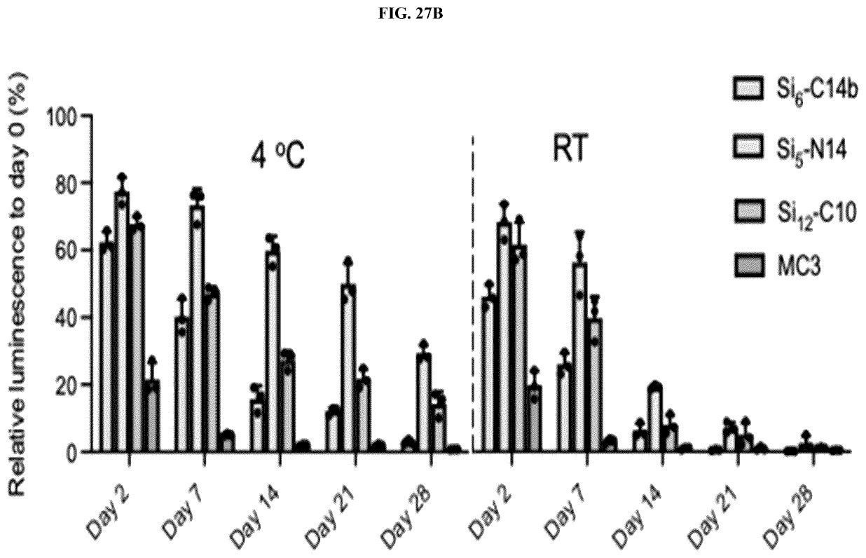

Next, the time-dependence of TTR editing was observed by measuring on-target DNA sequencing at 6 h, 24 h, and 7 days post-injection at an RNA dose of 3.0 mg kg⁻¹. Editing was detected 6 h post-injection, and its efficacy increased with increased post-injection time (FIG. 17). Importantly, it was found that editing was detected for at least 56 days after a single administration dose (FIG. 18). These results indicate both the potential to control gene knockout by varying the total RNA dose, and the capability of a single LNP dose to generate therapeutically-relevant knockout of target genes over a prolonged period of time (e.g., 8 weeks). Conversely, MC3 LNPs displayed limited gene editing even at high RNA doses (FIGS. 5H-51), which indicates the necessity of the development of novel LNP libraries for challenging therapeutic applications. Moreover, hematological analysis of liver function enzymes and kidney toxicity demonstrated that liver-targeted Si6-C14b LNPs showed negligible in vivo toxicity at 3.0 mg kg⁻¹ dose of Cas9 mRNA/TTR sgRNA (FIGS. 5K-5N). These results suggest the potential of SiLNP formulations for liver-specific protein replacement and gene correction therapies.

### Example 7: Lung-Specific SiLNPs for CRISPR-Cas9 Editing in the Lung

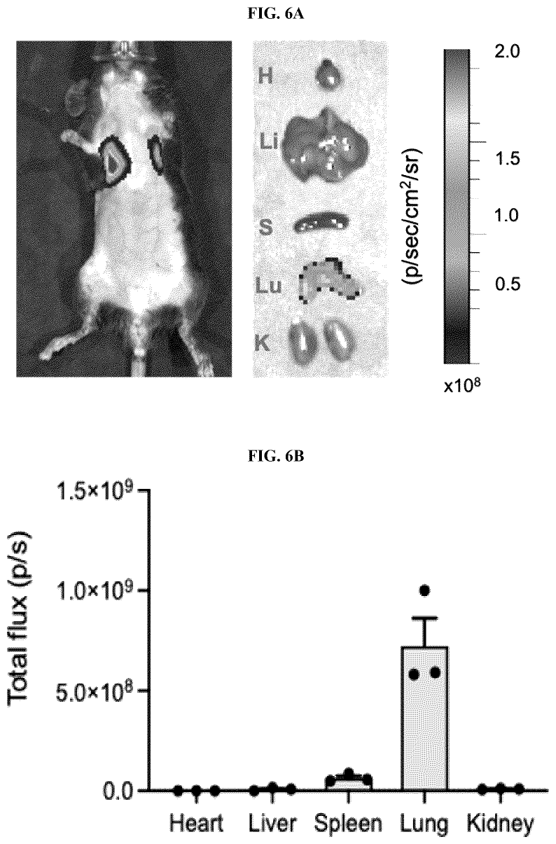

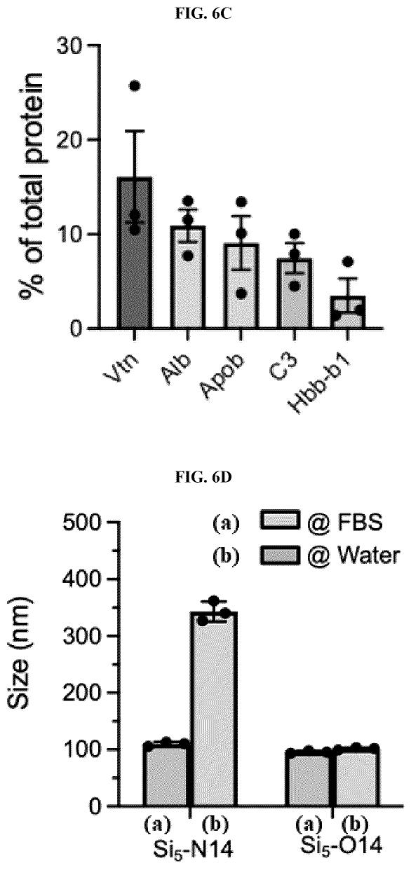

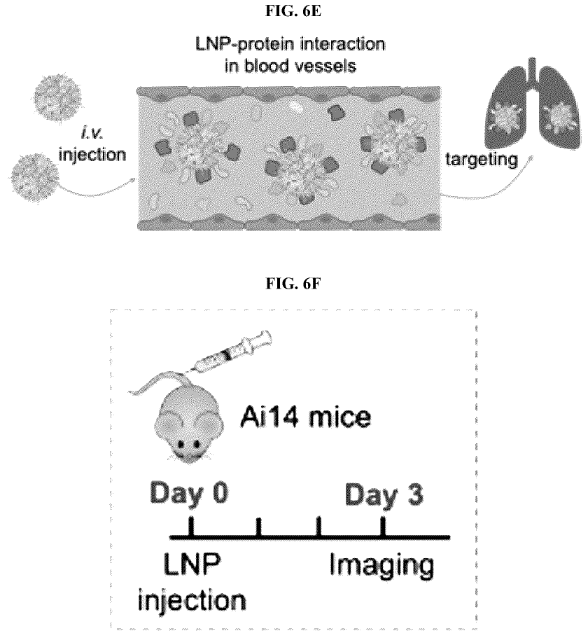

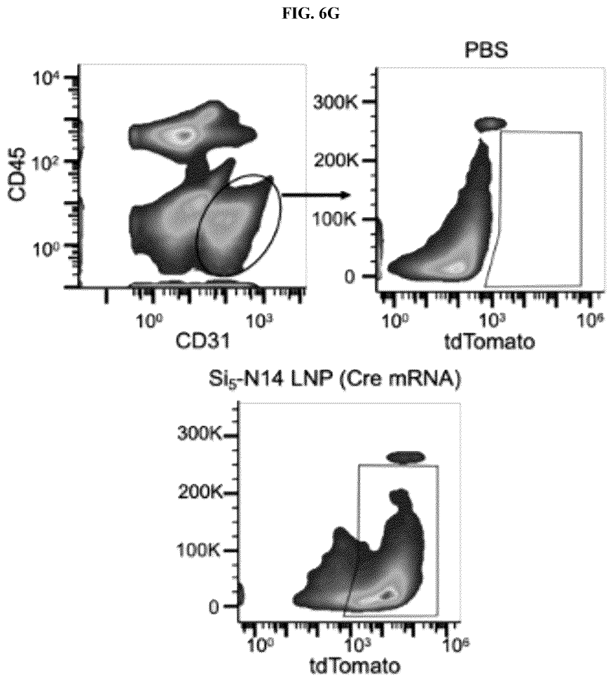

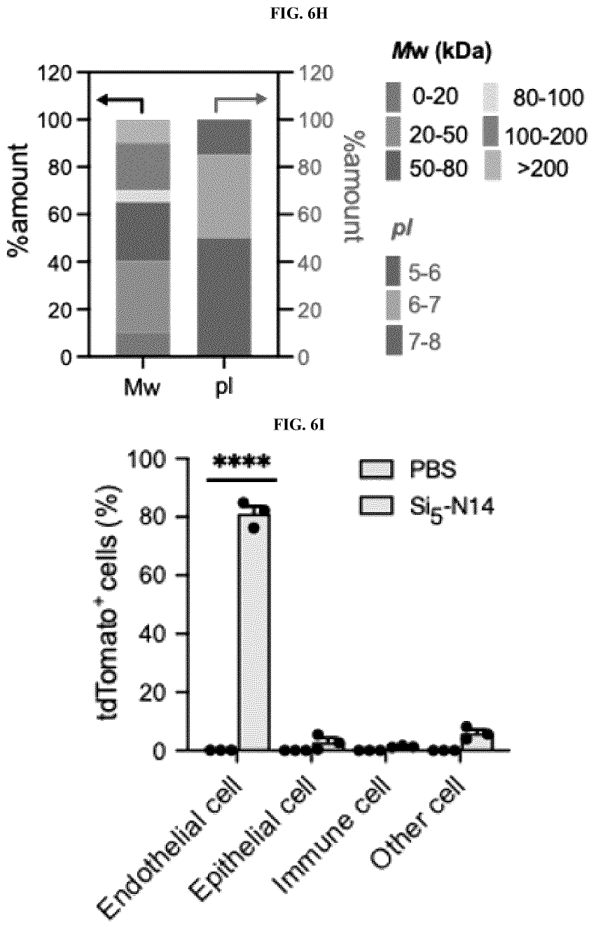

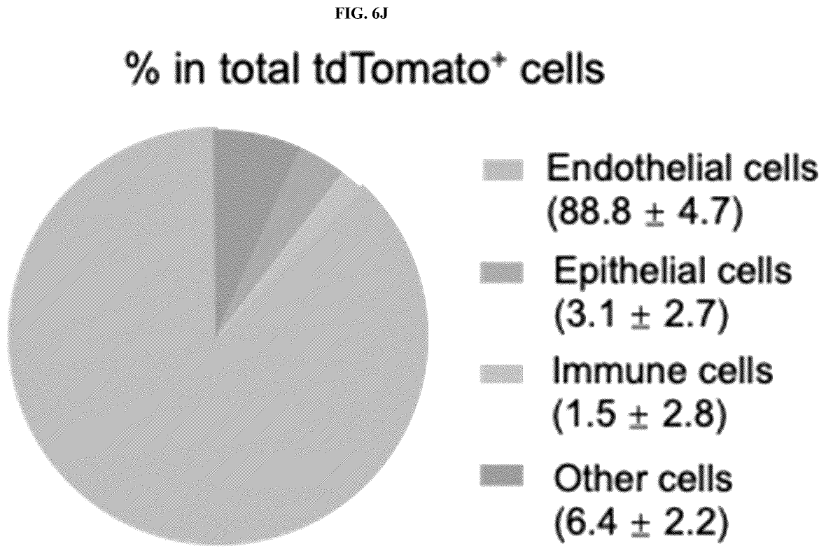

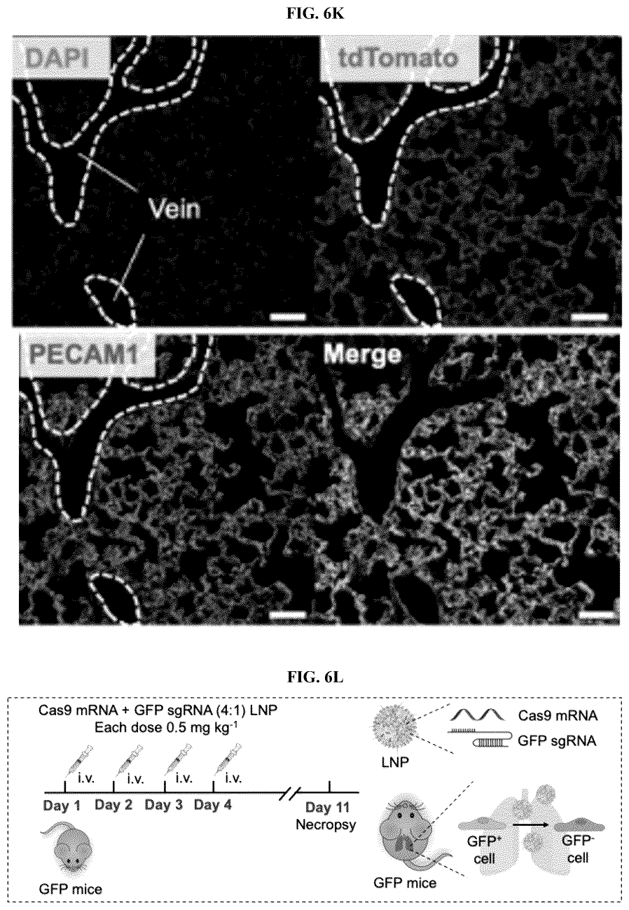

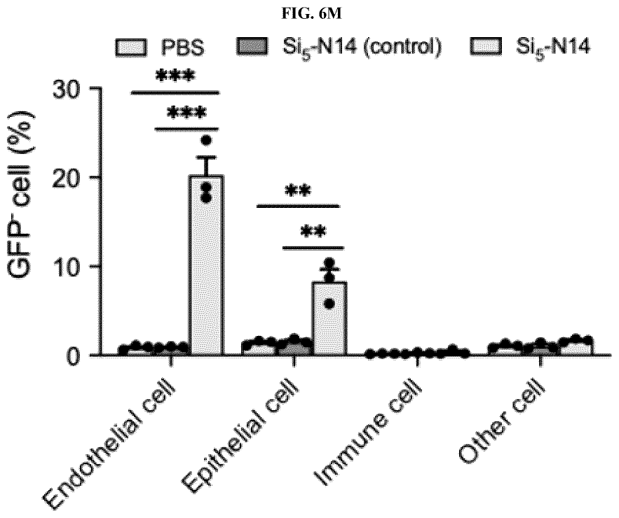

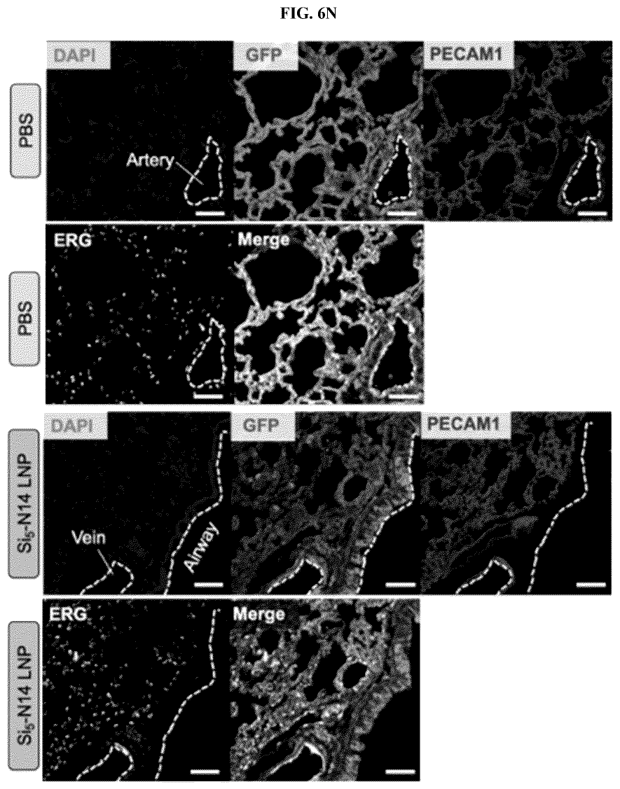

After developing liver-targeted SiLNPs, SiLNPs were evaluated for lung-targeted mRNA delivery. When the structure of siloxane lipidoids were further altered by incorporating two silicon atoms, two tertiary amines, and four amide-linked C14 alkyl chains, the resulting Si₅—N14 LNPs exhibited lung-specific mRNA delivery (FIGS. 6A-6B). Although engineering LNP formulations with targeted antibodies or incorporation of additional positively charged SORT molecules enable lung-selective mRNA delivery in vivo, the design of four-component LNPs to avoid toxicity of cationic components for pulmonary targeting with high efficacy is still challenging. The potential mechanism of Si5-N14 LNPs for lung specific mRNA delivery were evaluated.

To explore the potential mechanism of lung targeting by Si5-N14 LNPs, proteins bound to Si5-N14 LNPs were identified and quantified (FIG. 6E). Mouse plasma was incubated with Si5-N14 LNPs at 37° C. for 1 h, then proteins were isolated for proteomics analysis. The top 20 most abundant corona proteins were noted (Supplementary Table 7), where the top five proteins in the corona of Si5-N14 LNP are Vitronectin (Vtn), Serum albumin (Alb), Apolipoprotein B-100 (Apob), Complement C3 (C3), and Hemoglobin subunit beta-1 (Hbb-b1).

Among them, Vtn was identified as the most highly enriched protein at an average abundance of 16.1%, demonstrating 320-fold enrichment compared to native mouse plasma (FIG. 6C). Vtn can bind its cognate receptor, αvβ integrin, which is highly expressed by the pulmonary endothelium, providing a plausible explanation as to why Si5-N14 LNPs mediate lung specificity. Additionally, the minimal binding of apolipoprotein E (ApoE) (˜0.3% of the protein corona composition of Si5-N14 LNPs) could promote extrahepatic mRNA delivery compared to previously reported liver-tropic mRNA delivery systems.

The top 20 corona proteins were further classified according to their molecular weight (Mw), where 70% of proteins in the protein corona of Si5-N14 LNP were smaller than 100 kDa (FIG. 6H). The proteins were further characterized based on their isoelectric point (pI), where 85% of proteins in the corona of Si5-N14 LNPs have a negative charge (pI<7) in a physiological environment (pH=7.4). However, Si5-N14 LNPs exhibit a nearly neutral but extremely low positive surface charge, as determined by zeta potential measurements (i.e., Si5-N14 LNP—85.7±4.8 (EE %), 90.5±6.7 nm (size), 0.21±0.03 (PDI), and 2.72±0.71 mV (zeta potential)), suggesting surface charge may not be the only factor that affects LNP interaction with proteins in biological fluids. Together, these findings illustrate that various proteins with different Mw and pI in the corona of Si5-N14 LNPs collectively promote mRNA delivery to the lungs.

Interestingly, it was observed that the size of Si5-N14 LNPs showed an obvious change after incubation in a serum protein media, such as fetal bovine serum (FBS), which increased 3-fold from ˜100 nm to ˜300 nm (FIG. 6D). As control samples, the hydrodynamic size of Si₅—O14 LNPs did not show any noticeable change following incubation in FBS (FIG. 6D). It was assumed these differences were due to: (1) relatively hydrophobic siloxane structures compared to traditional alkyl-based amine cores, (2) the tendency of amide bonds on the Si₅—N14 LNP surface were to form hydrogen bonds between Si₅—N14 LNPs and proteins in the serum, and (3) hydrophobic interactions among Si₅—N14 LNPs in serum.

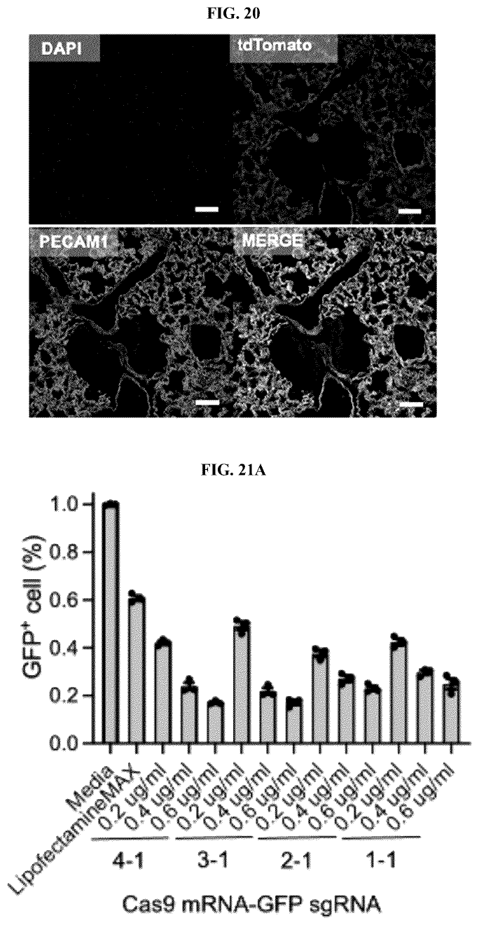

It was predicted that the enlarged Si₅—N14 LNPs would accumulate rapidly in the pulmonary capillaries for specific LNP targeting in the lung due to their rapid size change in serum conditions (FIG. 6E). Differing from previous theories that the protein corona on the LNP surface could remodel the properties of LNPs and dominate lung-selective mRNA delivery, the findings described herein provide another potential mechanism for lung-targeting mRNA delivery. To further analyze the transfected cells in the lung, Cre mRNA were delivered by Si₅—N14 LNPs in a Ai14 mouse model in vivo (FIG. 6F), which mediated highly specific endothelial cells (ECs) transfection (˜88% of ECs) (FIG. 6G and FIGS. 61-6J and FIGS. 19A-19C). Immunostaining of the lung demonstrated that Cre mRNA mainly activated the capillary endothelial cells of targeted microvascular in the lung, while transfection of the large vessels and airway was very weak (FIG. 6K and FIG. 20).

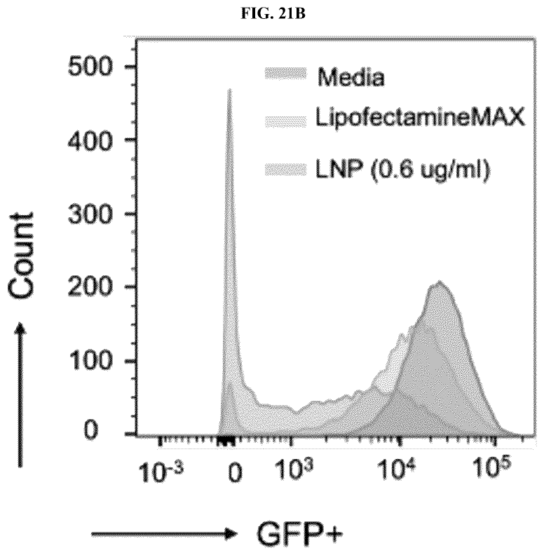

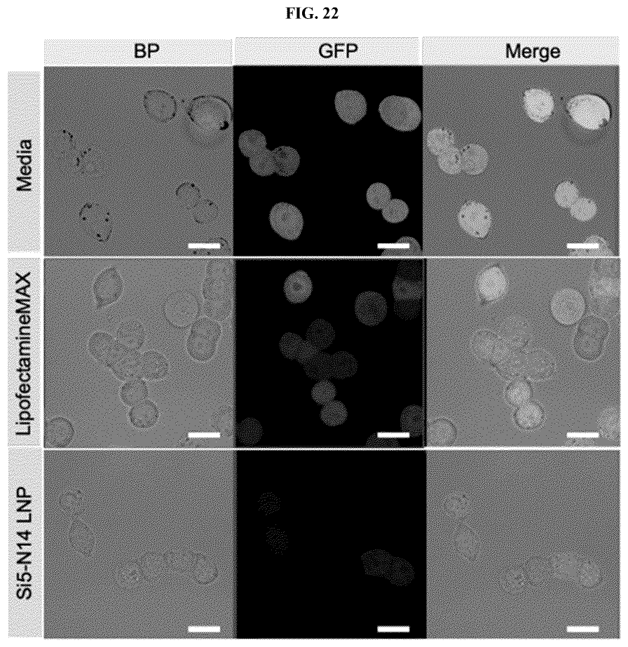

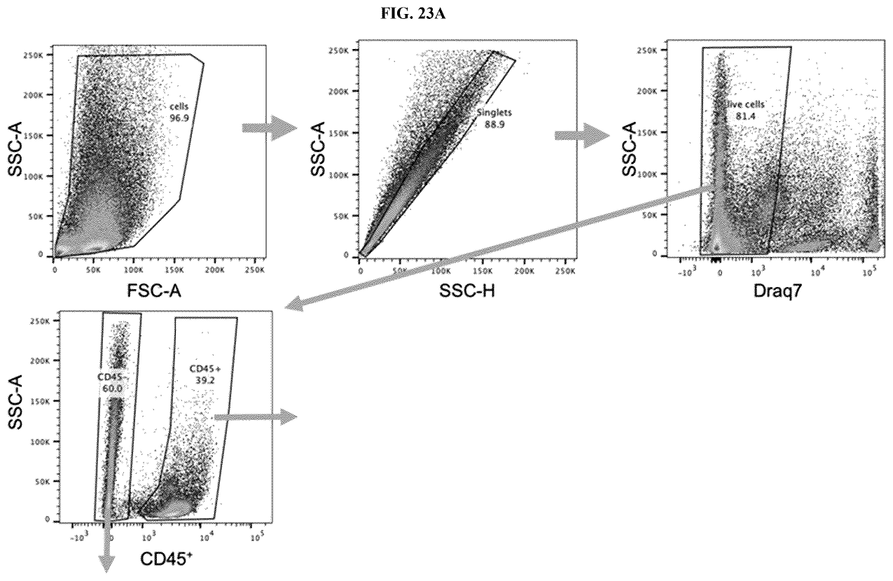

Next, co-delivery of Cas9 mRNA and sgRNA by Si₅—N14 LNPs to enable CRISPR-Cas9 genome editing in the lung was evaluated. To test this, Si₅—N14 LNPs encapsulating Cas9 mRNA (SEQ ID NO:3) and GFP sgRNA (SEQ ID NO:4) with different weight ratios (e.g., 4:1, 3:1, 2:1, and 1:1) were formulated to investigate the knockout efficiency of GFP in GFP-HepG2 cells (FIGS. 21A-21B). Lipofectamine CRISPR MAX carriers loaded with the same cargo were used as positive controls. Gene editing efficacy on the cell level was highly dependent on the Cas9 mRNA/GFP sgRNA ratio, indicating a total RNA concentration of 0.6 μg mL⁻¹ and ratio of 4:1 contributed to most effective GFP knockout in GFP-HepG2 cells (FIGS. 21A-21B). Compared with the positive control, Si₅—N14 LNPs edited over 60% of GFP-HepG2 cells. Subsequent confocal imaging was conducted to further validate editing, in which Si₅—N14 LNPs exhibited a much weaker GFP fluorescent signal compared with media-cultured groups, implying potent GFP knockout (FIG. 22).

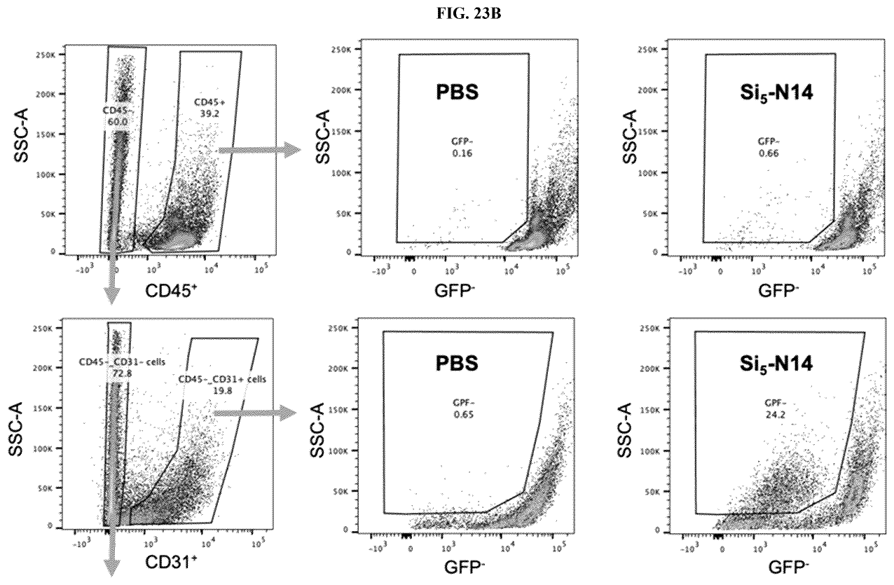

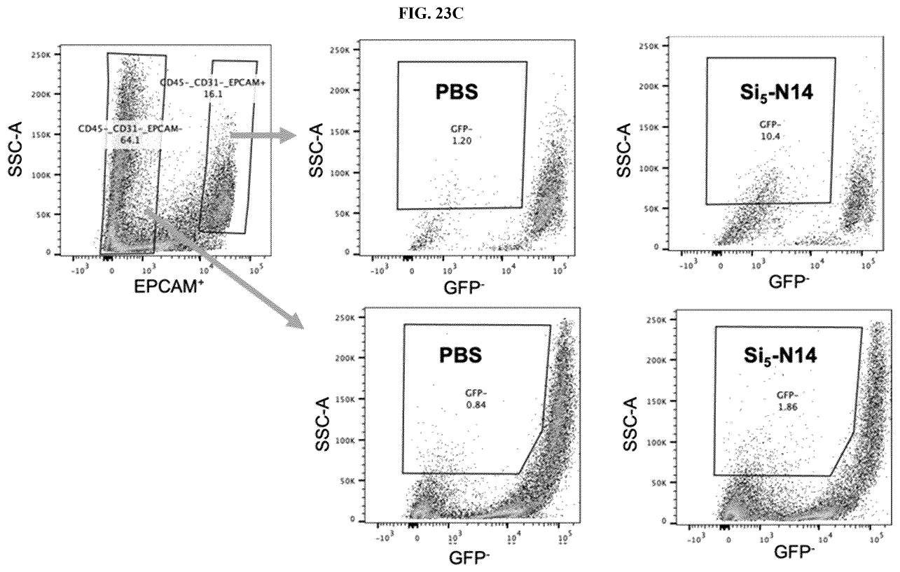

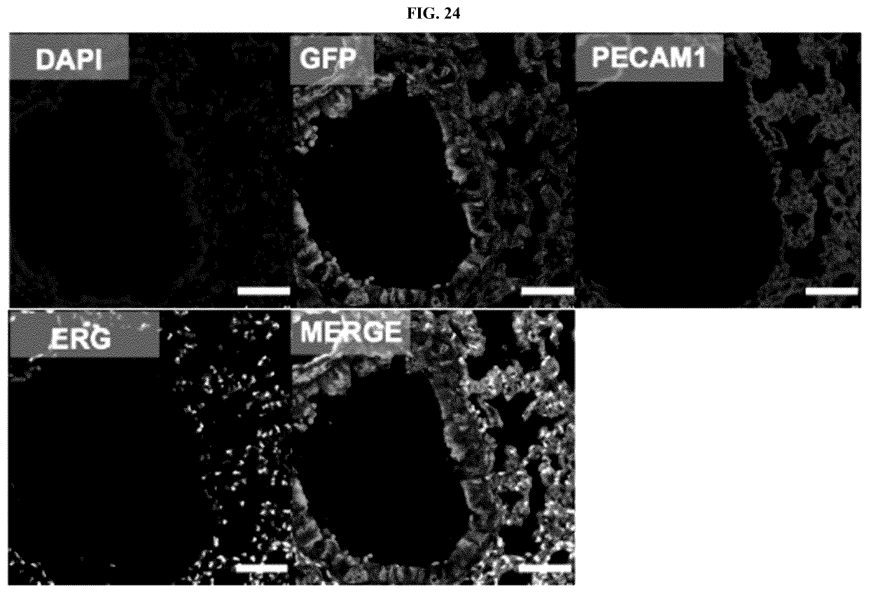

This optimized RNA ratio between Cas9 mRNA and GFP sgRNA (4:1) was used in Si₅—N14 LNPs to assess in vivo CRISPR-Cas9 gene editing in a GFP mouse model. Si₅—N14 LNPs co-delivering Cas9 mRNA and GFP sgRNA were repeatedly dosed (i.e., 4 times; 0.5 mg kg⁻¹ per dose) by i.v. injection, and then lung tissues were dissected for further evaluation of GFP knockout 7 days post-injection (FIG. 6L). Flow cytometry of cell populations was conducted to explore the GFP negative cell populations. Nearly −20% of the endothelial cells and ˜8% of the epithelial cells in the lung were editing (FIG. 6M and FIGS. 23A-23C). Immunostaining of the lung clearly showed decreased GFP signal in endothelial cells of microvasculature, rather than large vasculature and airway (FIG. 6N and FIG. 14). In addition to lung endothelial cells, CRISPR-Cas9-based genome editing also occurred in lung epithelial cells, which may contribute to continuous long-term editing in lung tissues.

In consideration of the high editing efficiency of endothelial cells, the endothelial cells from the lung were sorted to evaluate the editing efficacy by quantitative real-time PCR (qPCR), which demonstrated that GFP expression was significantly decreased in sorted ECs after SiLNPs mediated CRISPR-Cas9 editing (FIG. 6O). These results demonstrated that Si5-N14 LNPs enabled lung-targeted RNA delivery and lung-specific genome editing in endothelial cells, which could potentially be utilized for pulmonary vascular therapy and lung endothelial regeneration.

### Example 8: SiLNPs Enable Therapeutic Endothelial Repair for Lung Regeneration

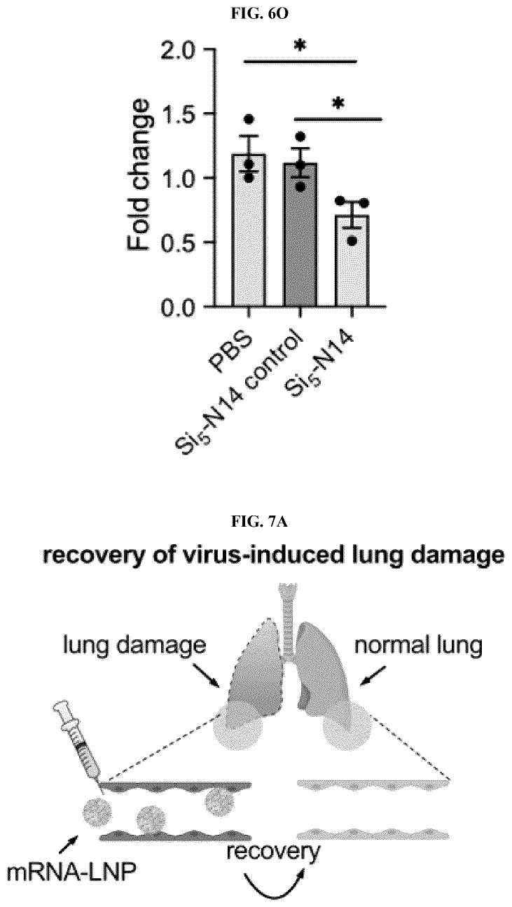

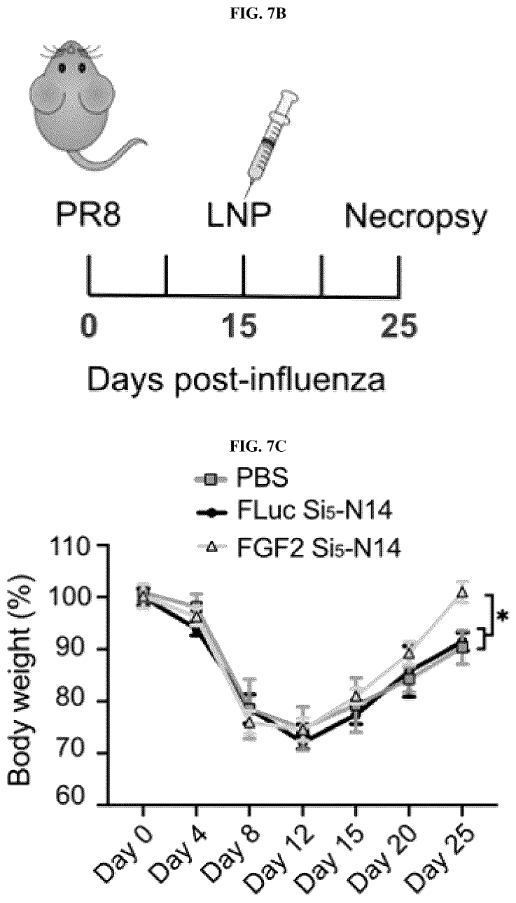

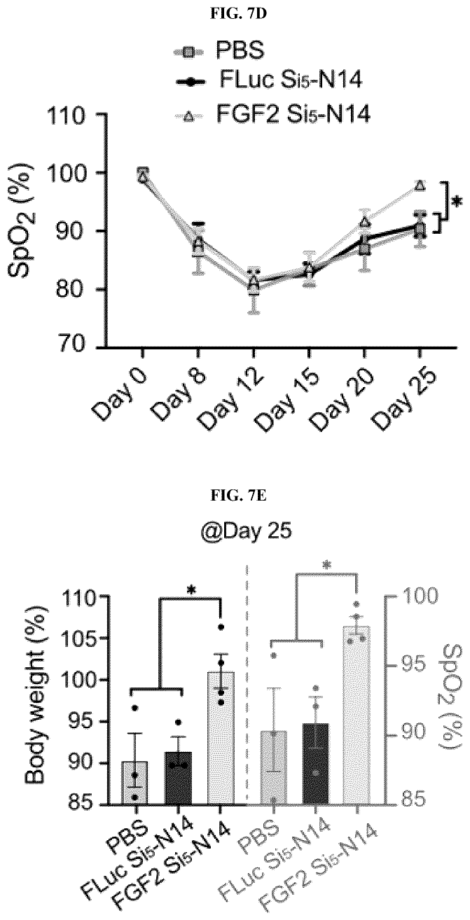

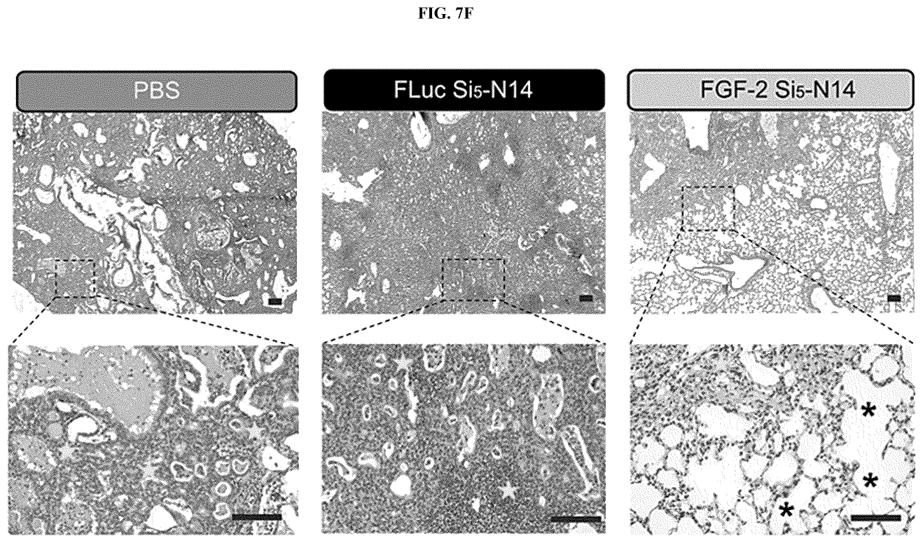

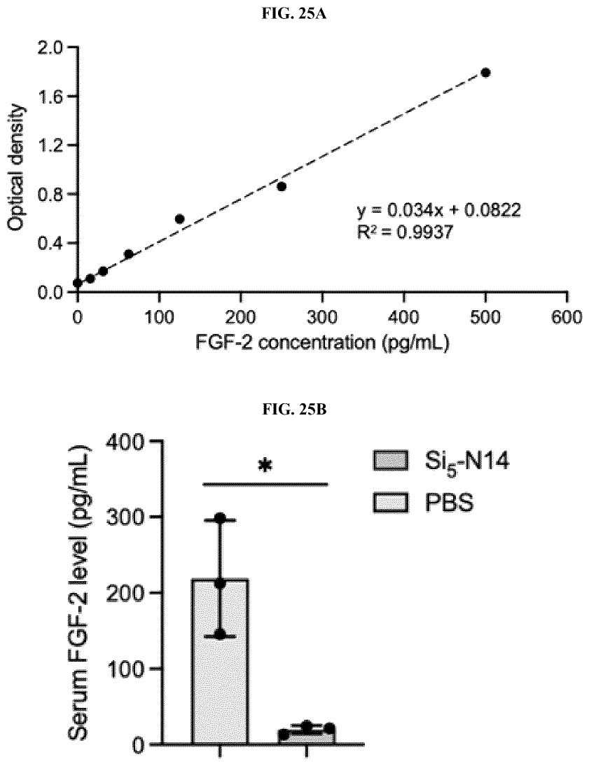

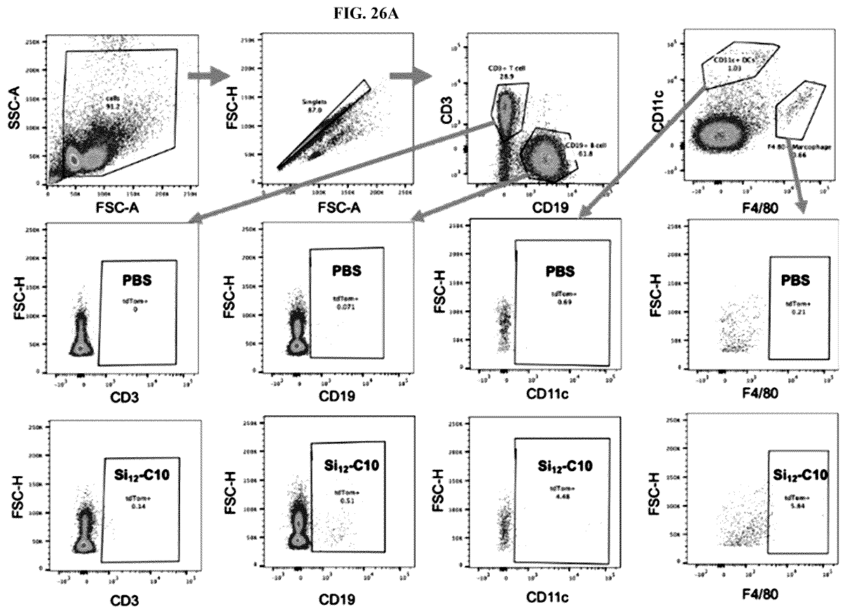

The results described herein demonstrated that Si5-N14 LNPs bind to Vtn, potentially driving efficient pulmonary endothelium targeting (FIG. 6C). Subsequently, the therapeutic potential of Si5-N14 LNPs for treating vascular-related diseases was evaluated, as nearly 30% of cells in lung tissue are endothelial cells. Studies were conducted utilizing an influenza-induced lung vasculature damage model. It was evaluated whether endothelial overexpression of fibroblast growth factor-2 (FGF-2) accelerates the recovery of lung function (FIGS. 7A-7B). Successful FGF-2 expression was confirmed by collecting the serum from mice treated with Si5-N14 LNPs encapsulating FGF-2 mRNA (FIGS. 25A-25B).

Previous studies have demonstrated that mRNA-LNPs induced exacerbation of inflammation in a pre-existing inflammation mode. To avoid any potential inflammation exacerbation, dexamethasone was injected (i.p.) to mice before administrating of therapeutic mRNA-LNP for lung function recovery. In the lung damage model, influenza-infected C57BL/6J mice were treated with FGF-2 mRNA Si5-N14 LNPs or control groups (PBS or FLuc mRNA Si5-N14 LNPs) 15 days post-infection and lungs were harvested on day 25 (FIG. 7B).

Treatment with FGF-2 mRNA LNPs improved lung function, evidenced by improved recovery of body weight and increased blood oxygen levels compared to LNP control groups (FIG. 7C-7E). Histopathological evaluation of lungs showed less inflammation and improved remodeling with FGF-2 mRNA Si5-N14 LNP treatment compared to respective controls, as evidenced by less destruction of alveolar architecture and leukocyte infiltration (FIG. 7F). Collectively, these results indicated that the delivery of endothelial FGF-2 through Si5-N14 LNPs significantly enhances angiogenic repair, highlighting the great potential of SiLNPs as a vehicle for therapeutic targeted lung regeneration.

Sequence Listing

SEQ ID NO: 1 (mTTR-exon2-F)
CGGTTTACTCTGACCCATTTC
SEQ ID NO: 2 (mTTR-exon2-R)
GGGCTTTCTACAAGCTTACC
SEQ ID NO: 3 (GFP sgRNAª)
gsgsgsCGAsGsGfsAfsGfsCfUGfUfUCAfCfCGgUUUUAGagcua
gaaauagcaaGUUaAaAuAaggcuaGUccGUUAucAAcsususgsasa
sasasasgugGscascscsgsasgsuscgsgsusgscsususususu
SEQ ID NO: 4 (mTTR sgRNA)
UUACAGCCACGUCUACAGCA
ªNucleotides without modifications are in uppercase (A, U, C, G). Nucleotides with 2'OMe modifications are in lowercase (a, u, c, g) . Nucleotides with 2'F modifications are described as uppercase plus f (Af, Uf, Cf, Gf) . Phosphorothioate bonds are described as ″s″.

### Enumerated Embodiments

The following exemplary embodiments are provided, the numbering of which is not to be construed as designating levels of importance:

Embodiment 1 provides a compound of Formula (I), or a salt, solvate, stereoisomer, or isotopologue thereof:

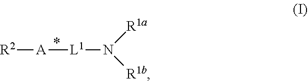

wherein:
A is

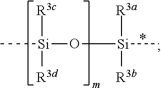

R² is selected from the group consisting of

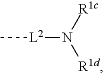

optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{1a}, R^{1b}, R^{1c}, and R^{1d}, if present, are each independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
L¹ and L² are each independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{3a}, R^{3b}, R^{3c}, and R^{3d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl,
   wherein two occurrences of R^{3c} or two occurrences of R_{3d} can combine with the atoms to which they are bound to form an optionally substituted 6-10 membered cyclosiloxane, or
   wherein R^{3a} and R^{3c}, R^{3a} and R^{3d}, R^{3b} and R^{3c}, or R^{3b} and R^{3d} can combine with the atoms to which they are bound to form an optionally substituted 6-10 membered cyclosiloxane;
each occurrence of R^{A} is independently selected from the group consisting of optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl; and
m is an integer ranging from 0 to 50.

Embodiment 2 provides the compound of Embodiment 1, wherein A is selected from the group consisting of:

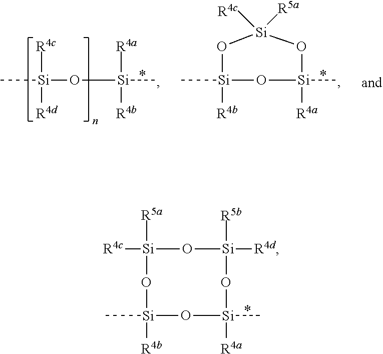

wherein:
R^{4a}, R^{4b}, R^{4c}, and R^{4d}, if present, are each independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{5a} and R^{5b}, if present, are each independently selected from the group consisting of

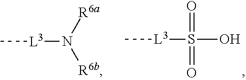

optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of L³ is independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₅ cycloalkylenyl, and optionally substituted C₂-C₈ heterocyloalkylenyl;
each occurrence of R^{6a} and R^{6b} is independently selected from the group consisting of H, optionally substituted C₁-C₂₈ alkyl, optionally substituted C₂-C₂₈ heteroalkyl, optionally substituted C₃-C₅ cycloalkyl, optionally substituted C₂-C₈ heterocycloalkyl, optionally substituted C₂-C₂₈ alkenyl, and optionally substituted C₂-C₂₈ alkynyl; and
n is an integer ranging from 0 to 30.

Embodiment 3 provides the compound of Embodiment 2, wherein each occurrence of R^{4a}, R^{4b}, R^{4c}, and R^{4d}, if present, is independently selected from the group consisting of Me and OSiMe₃.

Embodiment 4 provides the compound of Embodiment 1 or 2, wherein A is selected from the group consisting of

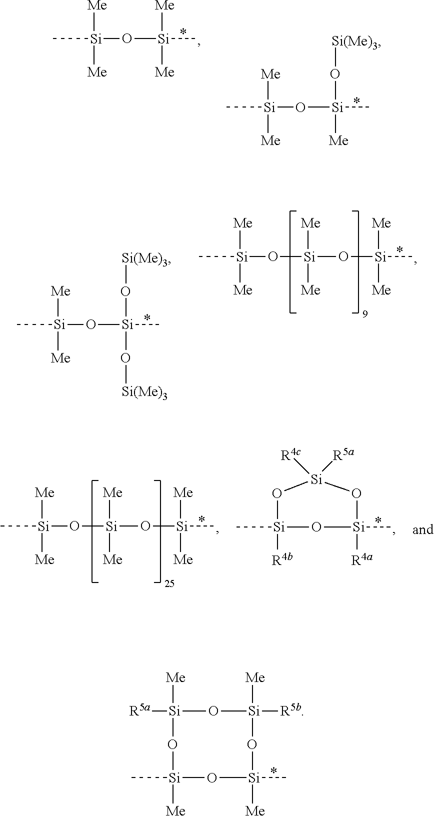

Embodiment 5 provides the compound of any one of Embodiments 1-4, wherein each occurrence of L¹, L², and L³, if present, is independently selected from the group consisting of —(CH₂)₁₋₅S(CH₂)₁₋₅—, —(CH₂)₁₋₅—, and —(CH₂)₁₋₅N(R^{6a})(CH₂)₁₋₅—.

Embodiment 6 provides the compound of any one of Embodiments 1-5, wherein each occurrence of L¹, L², and L³, if present, is independently selected from the group consisting of —(CH₂)₃—, —(CH₂)₂S(CH₂)₂—, and —(CH₂)NR^{6a}(CH₂)₂—.

Embodiment 7 provides the compound of any one of Embodiments 1-6, which is selected from the group consisting of.

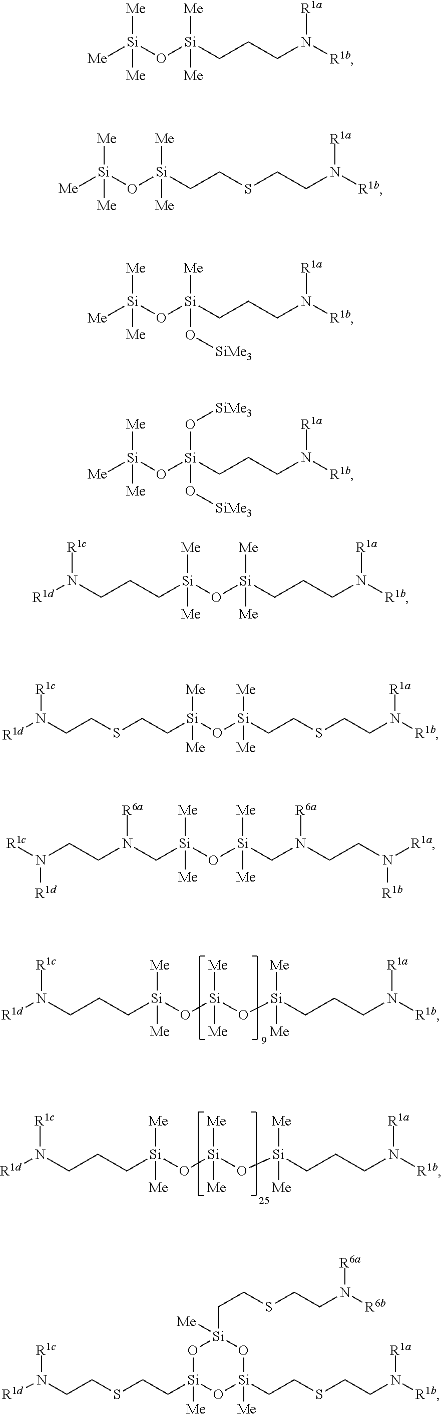

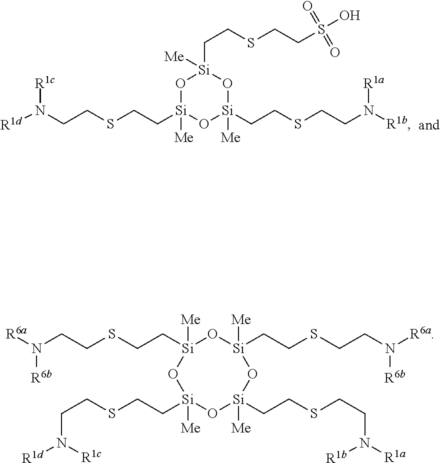

Embodiment 8 provides the compound of any one of Embodiments 1-7, wherein R^{1a}, R^{1b}, R^{1c}, and R^{1d} are each independently selected from the group consisting of —CH₂CHOH-(optionally substituted C₁-C₂₀ alkyl), —CH₂CHOH-(optionally substituted C₁-C₂₀ heteroalkyl), —CH₂CHOH-(optionally substituted C₂-C₂₀ alkenyl), —CH₂CH₂C(═O)O(optionally substituted C₁-C₂₀ alkyl), and —CH₂CH₂C(═O)NH(optionally substituted C₁-C₂₀ alkyl).

Embodiment 9 provides the compound of any one of Embodiments 2-8, wherein each occurrence of R^{6a} and R_{6b}, if present, is independently selected from the group consisting of —CH₂CHOH-(optionally substituted C₁-C₂₀ alkyl), —CH₂CHOH-(optionally substituted C₁-C₂₀ heteroalkyl), —CH₂CHOH-(optionally substituted C₂-C₂₀ alkenyl), —CH₂CH₂C(═O)O(optionally substituted C₁-C₂₀ alkyl), and —CH₂CH₂C(═O)NH(optionally substituted C₁-C₂₀ alkyl).

Embodiment 10 provides the compound of any one of Embodiments 1-9, wherein each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aralkyl, optionally substituted aryl, optionally substituted heteroaryl, and optionally substituted cyclosiloxane, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂OR′, S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 11 provides the compound of any one of Embodiments 1-10, wherein R^{1a}, R^{1b}, R^{1c}, and R^{1d} are each independently selected from the group consisting of

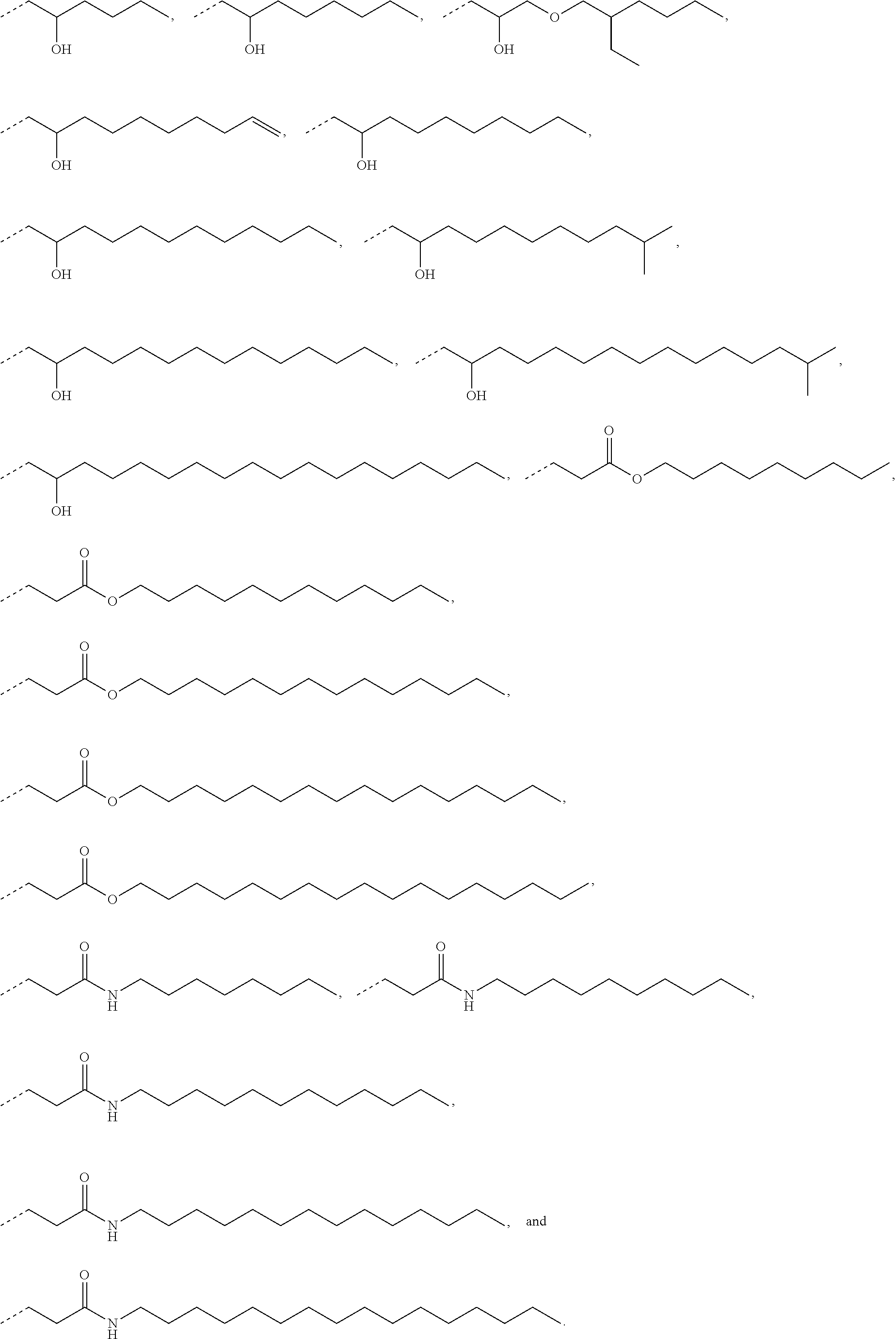

Embodiment 12 provides the compound of any one of Embodiments 2-11, wherein each occurrence of R^{6a} and R^{6b}, if present, is independently selected from the group consisting of

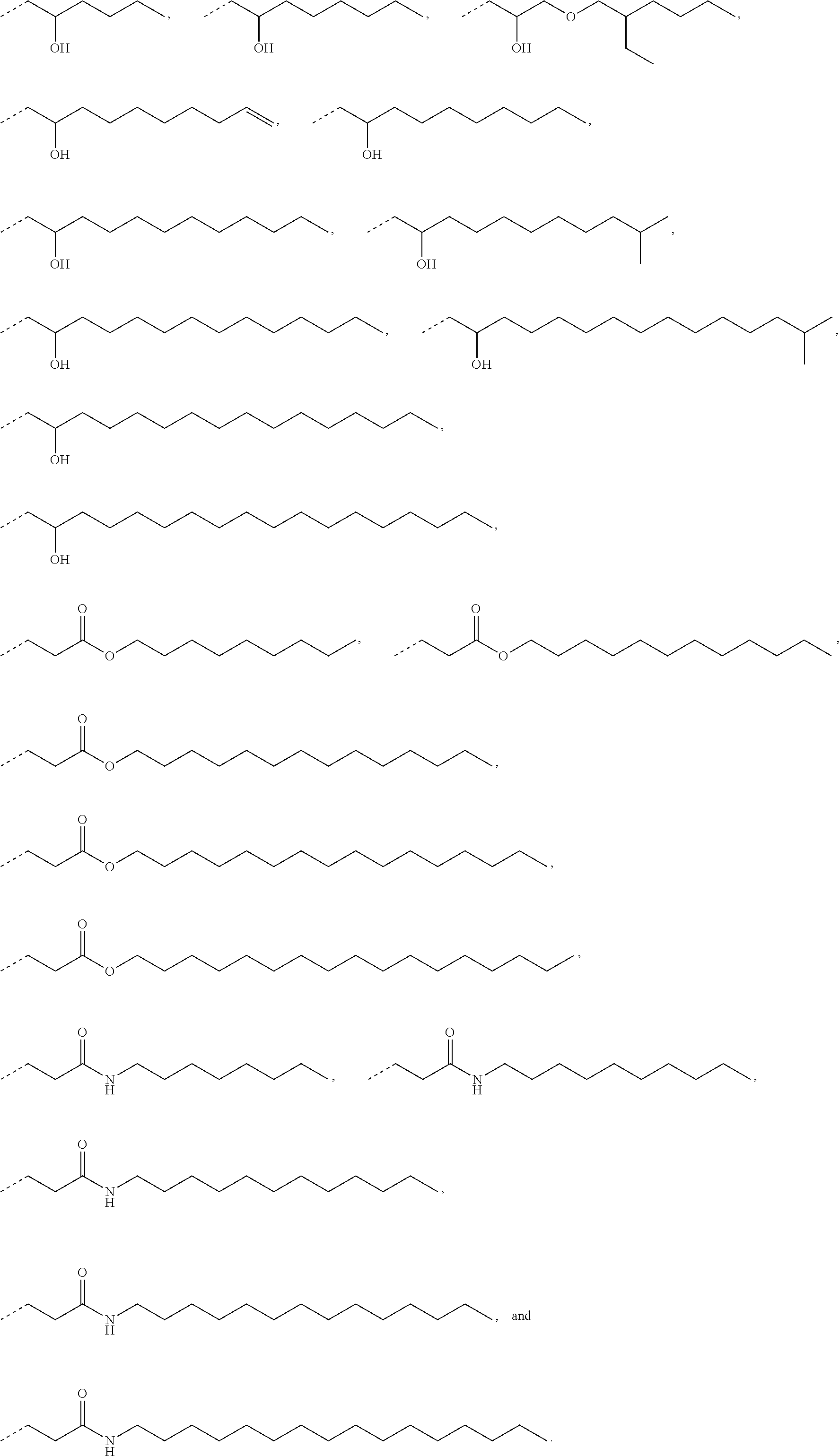

Embodiment 13 provides the compound of any one of Embodiments 1-12, which is selected from the group consisting of:

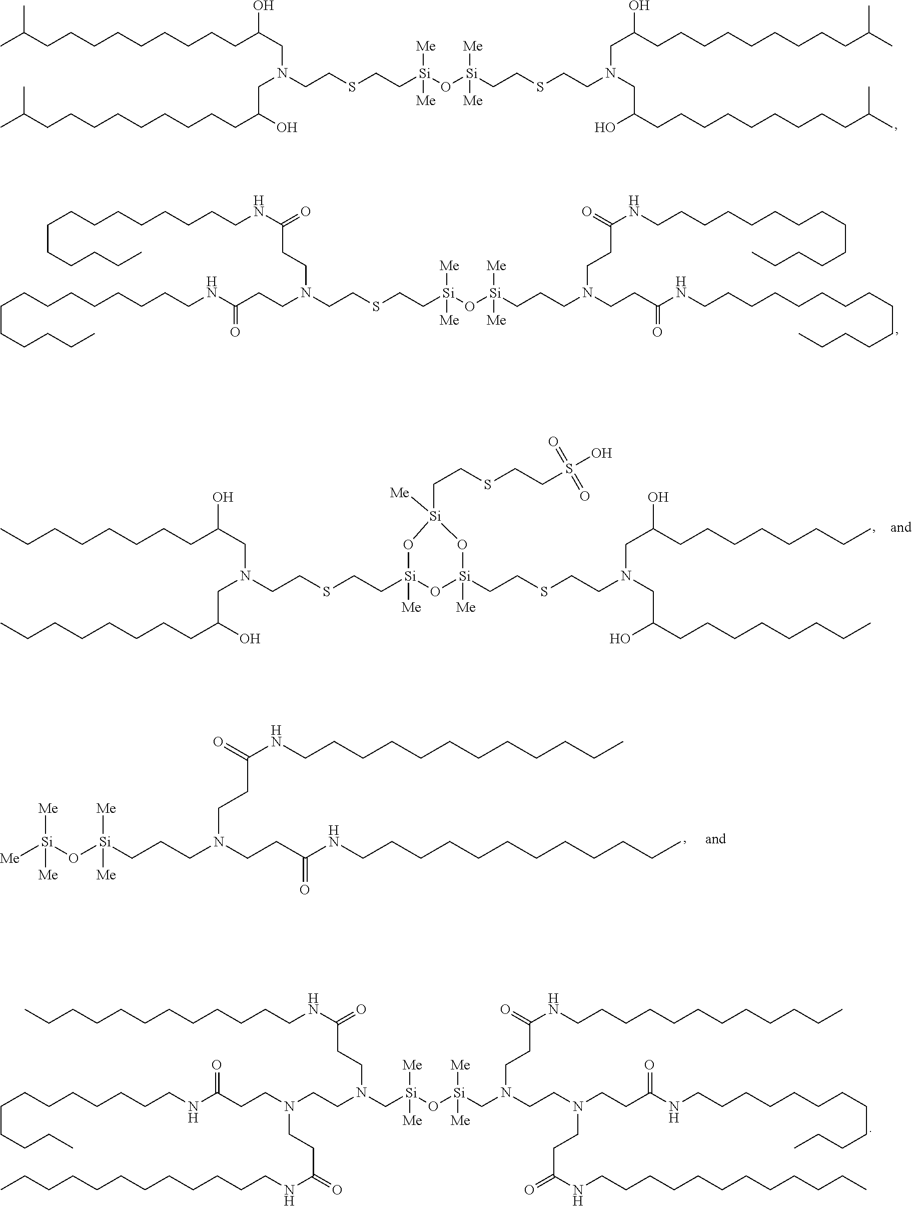

Embodiment 14 provides a lipid nanoparticle (LNP) comprising:
(a) at least one compound of Formula (I), or a salt, solvate, stereoisomer, or isotopologue thereof:

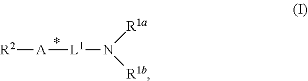

wherein:
A is

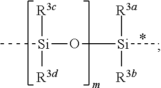

R² is selected from the group consisting of

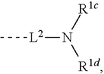

optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of R^{1a}, R^{1b}, R^{1c}, and R^{1d}, if present, is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
L¹ and L² are each independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{3a}, R^{3b}, R^{3c}, and R^{3d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl,
   wherein two occurrences of R^{3c} or two occurrences of R^{3d} can combine with the atoms to which they are bound to form an optionally substituted 6-10 membered cyclosiloxane, or
   wherein R^{3a} and R^{3c}, R^{3a} and R^{3d}, R^{3b} and R^{3c}, or R^{3b} and R^{3d} can combine with the atoms to which they are bound to form an optionally substituted 6-10 membered cyclosiloxane;
each occurrence of R^{A} is independently selected from the group consisting of optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl; and
m is an integer ranging from 0 to 50;
(b) at least one neutral phospholipid;
(c) at least one cholesterol lipid; and
(d) at least one selected from the group consisting of polyethylene glycol (PEG) and a PEG-conjugated lipid.

Embodiment 15 provides the LNP of Embodiment 14, wherein the LNP further comprises at least one cargo.

Embodiment 16 provides the LNP of Embodiment 15, wherein the cargo is at least partially encapsulated by the LNP.

Embodiment 17 provides the LNP of Embodiment 15 or 16, wherein the cargo is at least one selected from the group consisting of a nucleic acid molecule, small molecule, protein, therapeutic agent, antibody, and any combinations thereof.

Embodiment 18 provides the LNP of any one of Embodiments 15-17, wherein the cargo is a nucleic acid molecule.

Embodiment 19 provides the LNP of Embodiment 17 or 18, wherein the nucleic acid molecule is a DNA molecule or a RNA molecule.

Embodiment 20 provides the LNP of any one of Embodiments 17-19, wherein the nucleic acid molecule is selected from the group consisting of mRNA, cDNA, pDNA, microRNA, siRNA, modified RNA, antagomir, antisense molecule, targeted nucleic acid, and any combination thereof.

Embodiment 21 provides the LNP of any one of Embodiments 14-20, wherein A is selected from the group consisting of:

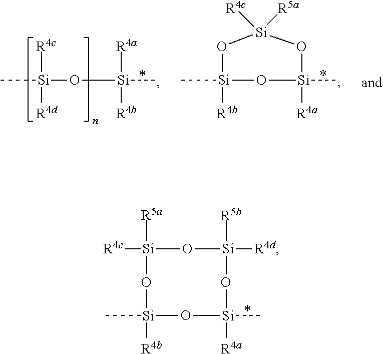

wherein:
R^{4a}, R^{4b}, R^{4c}, and R^{4d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{5a} and R^{5b}, if present, are each independently selected from the group consisting of

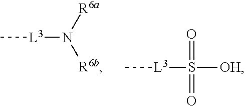

optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of L³ is independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{6a} and R^{6b} is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂s alkenyl, and optionally substituted C₂-C₂s alkynyl; and
n is an integer ranging from 0 to 30.

Embodiment 22 provides the LNP of any one of Embodiments 14-21, wherein each occurrence of R^{4a}, R^{4b}, R^{4c}, and R^{4d}, if present, is independently selected from the group consisting of Me and OSiMe₃.

Embodiment 23 provides the LNP of any one of Embodiments 14-22, wherein A¹ is selected from the group consisting of

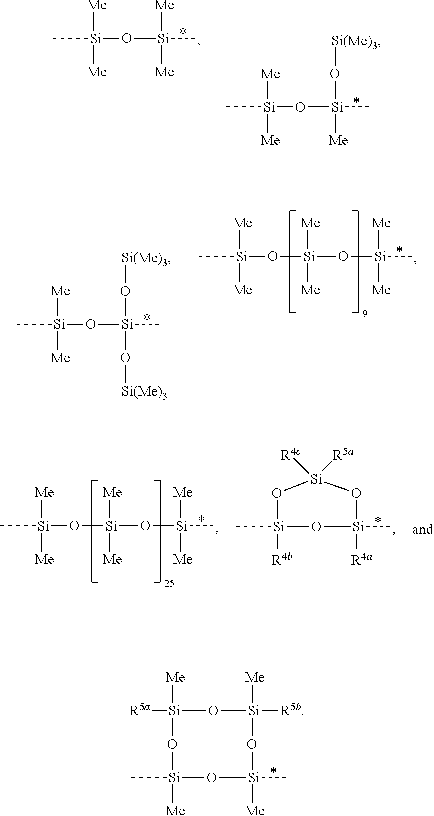

Embodiment 24 provides the LNP of any one of Embodiments 14-23, wherein each occurrence of L¹, L², and L³, if present, is independently selected from the group consisting of —(CH₂)₁₋₅S(CH₂)₁₋₅—, —(CH₂)₁₋₅—, and —(CH₂)₁₋₅N(R^{6a})(CH₂)₁₋₅—.

Embodiment 25 provides the LNP of any one of Embodiments 14-24, wherein each occurrence of L¹, L², and L³, if present, is independently selected from the group consisting of —(CH₂)₃—, —(CH₂)₂S(CH₂)₂—, and —(CH₂)NR^{6a}(CH₂)₂—.

Embodiment 26 provides the LNP of any one of Embodiments 14-25, which is selected from the group consisting of

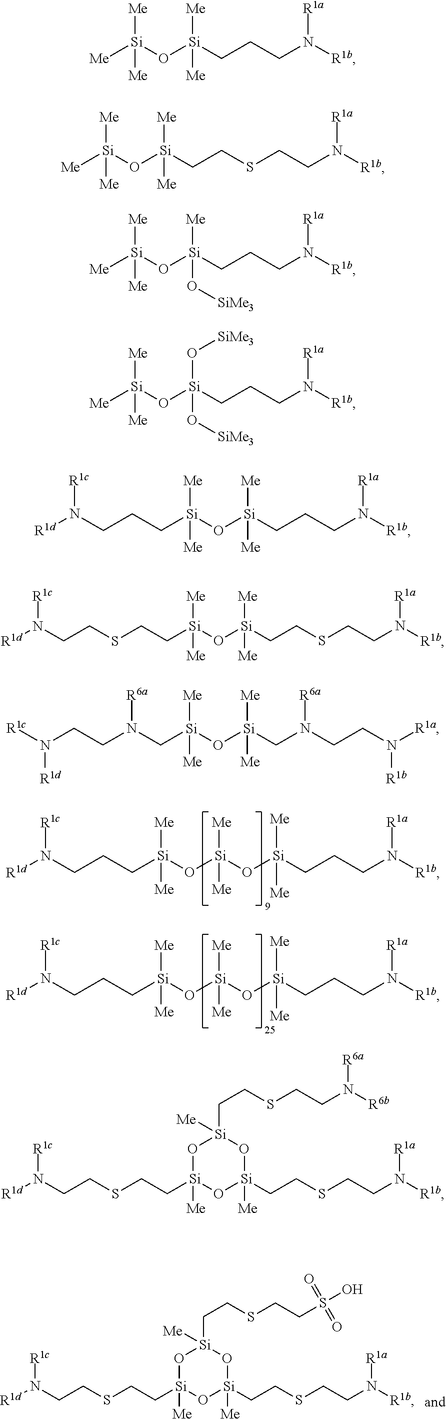

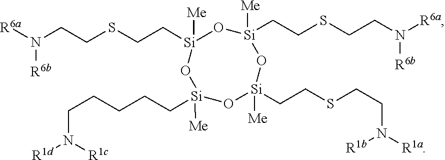

Embodiment 27 provides the LNP of any one of Embodiments 14-26, wherein R^{1a}, R^{1b}, R^{1c}, and R^{1d} are each independently selected from the group consisting of —CH₂CHOH-(optionally substituted C₁-C₂₀ alkyl). —CH₂CHOH-(optionally substituted C₁-C₂₀ heteroalkyl), —CH₂CHOH-(optionally substituted C₂-C₂₀ alkenyl), —CH₂CH₂C(═O)O(optionally substituted C₁-C₂₀ alkyl), and —CH₂CH₂C(═O)NH(optionally substituted C₁-C₂₀ alkyl).

Embodiment 28 provides the LNP of any one of Embodiments 14-27, wherein each occurrence of R^{6a} and R^{6b}, if present, is independently selected from the group consisting of —CH₂CHOH-(optionally substituted C₁-C₂₀ alkyl), —CH₂CHOH-(optionally substituted C₁-C₂₀ heteroalkyl), —CH₂CHOH-(optionally substituted C₂-C₂₀ alkenyl), —CH₂CH₂C(═O)O(optionally substituted C₁-C₂₀ alkyl), and —CH₂CH₂C(═O)NH(optionally substituted C₁-C₂₀ alkyl).

Embodiment 29 provides the LNP of any one of Embodiments 14-28, wherein each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aralkyl, optionally substituted aryl, optionally substituted heteroaryl, and optionally substituted cyclosiloxane, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂OR′, S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

Embodiment 30 provides the LNP of any one of Embodiments 14-29, wherein R^{1a}, R^{1b}, R^{1c}, and R^{1d} are each independently selected from the group consisting of

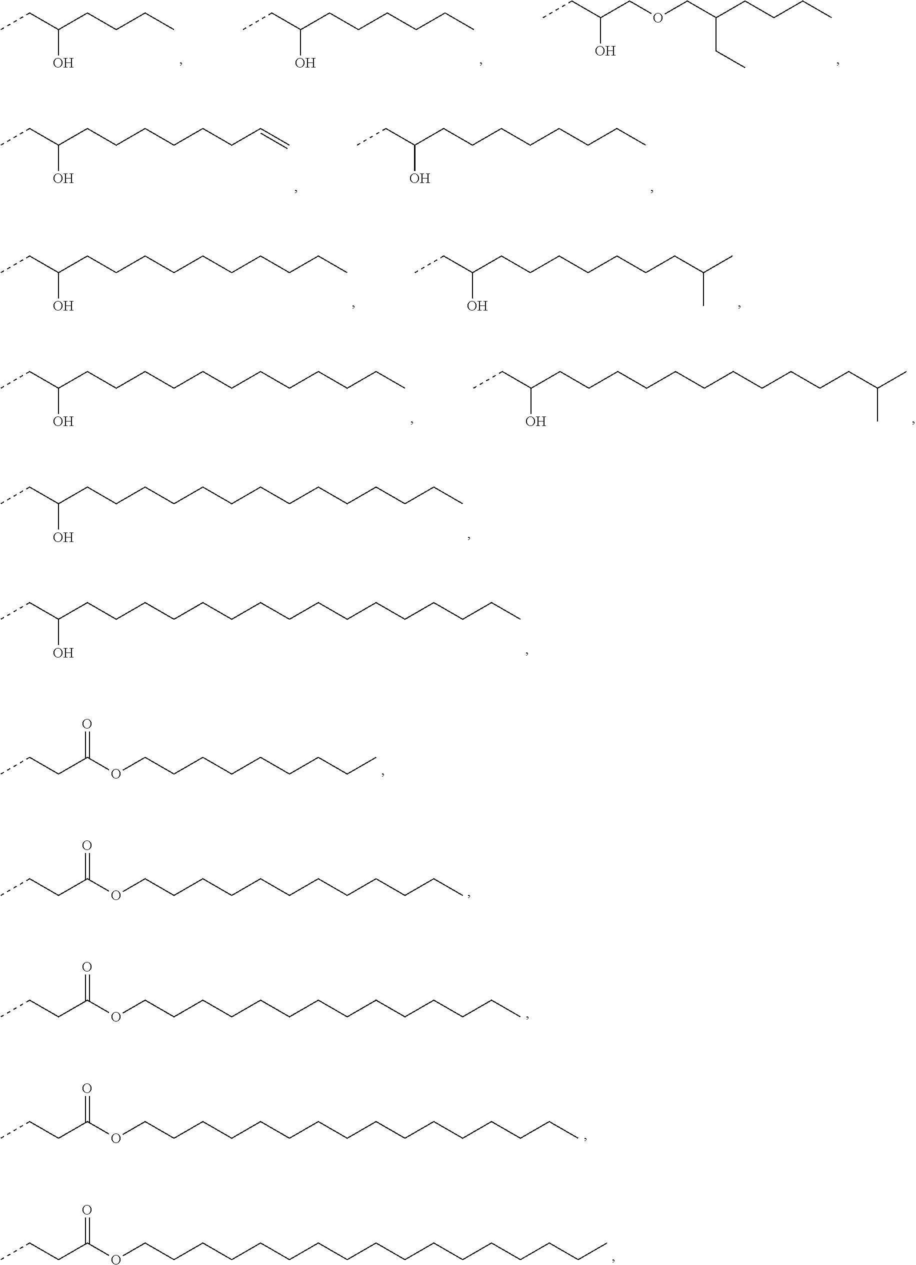

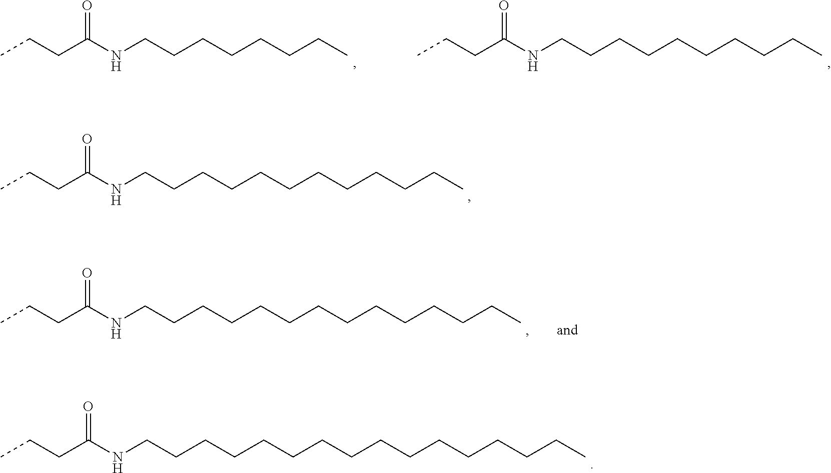

Embodiment 31 provides the LNP of any one of Embodiments 14-30, wherein each occurrence of R^{6a} and R^{6b}, if present, is independently selected from the group consisting of

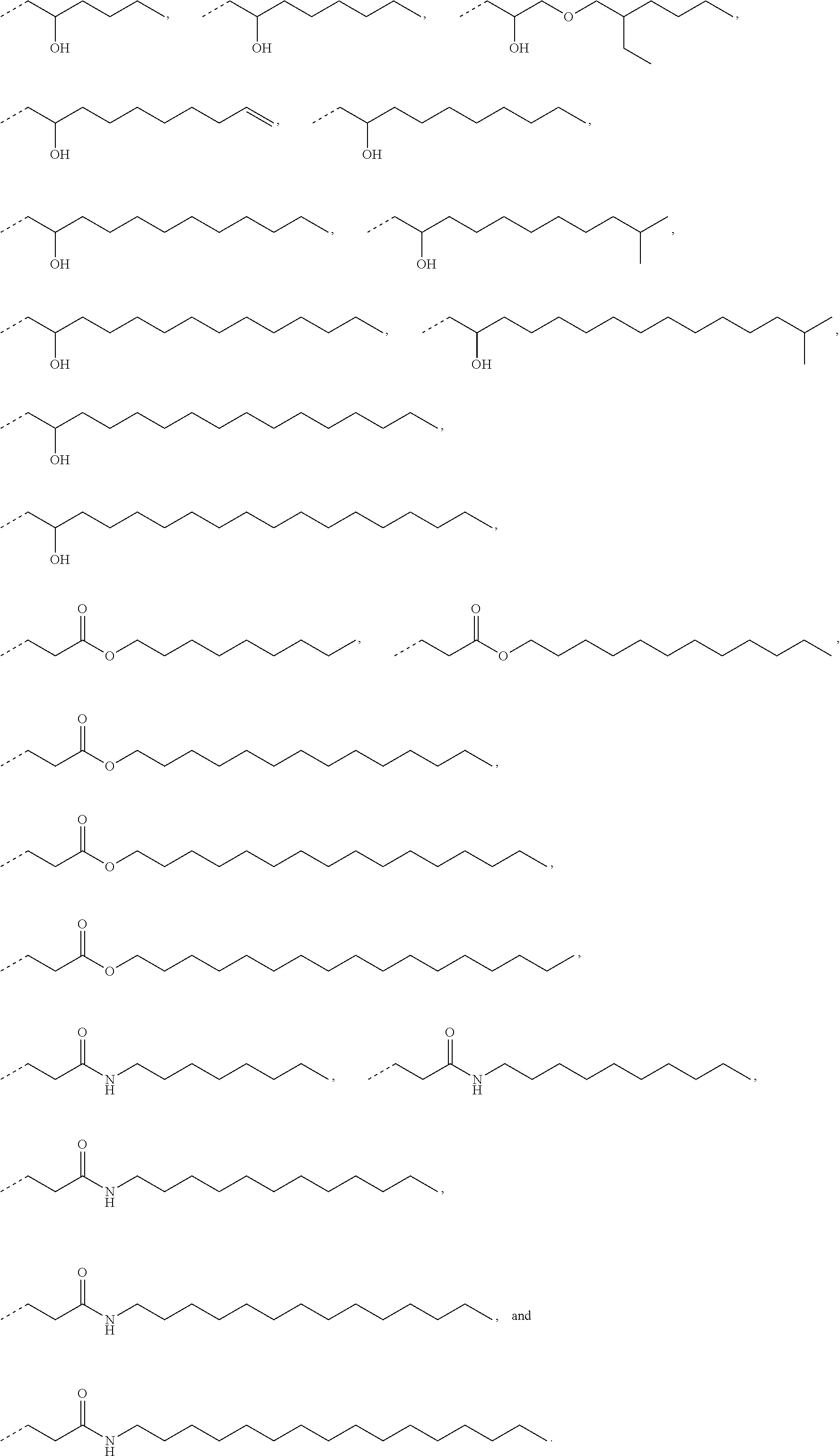

Embodiment 32 provides the LNP of any one of Embodiments 14-31, which is selected from the group consisting of.

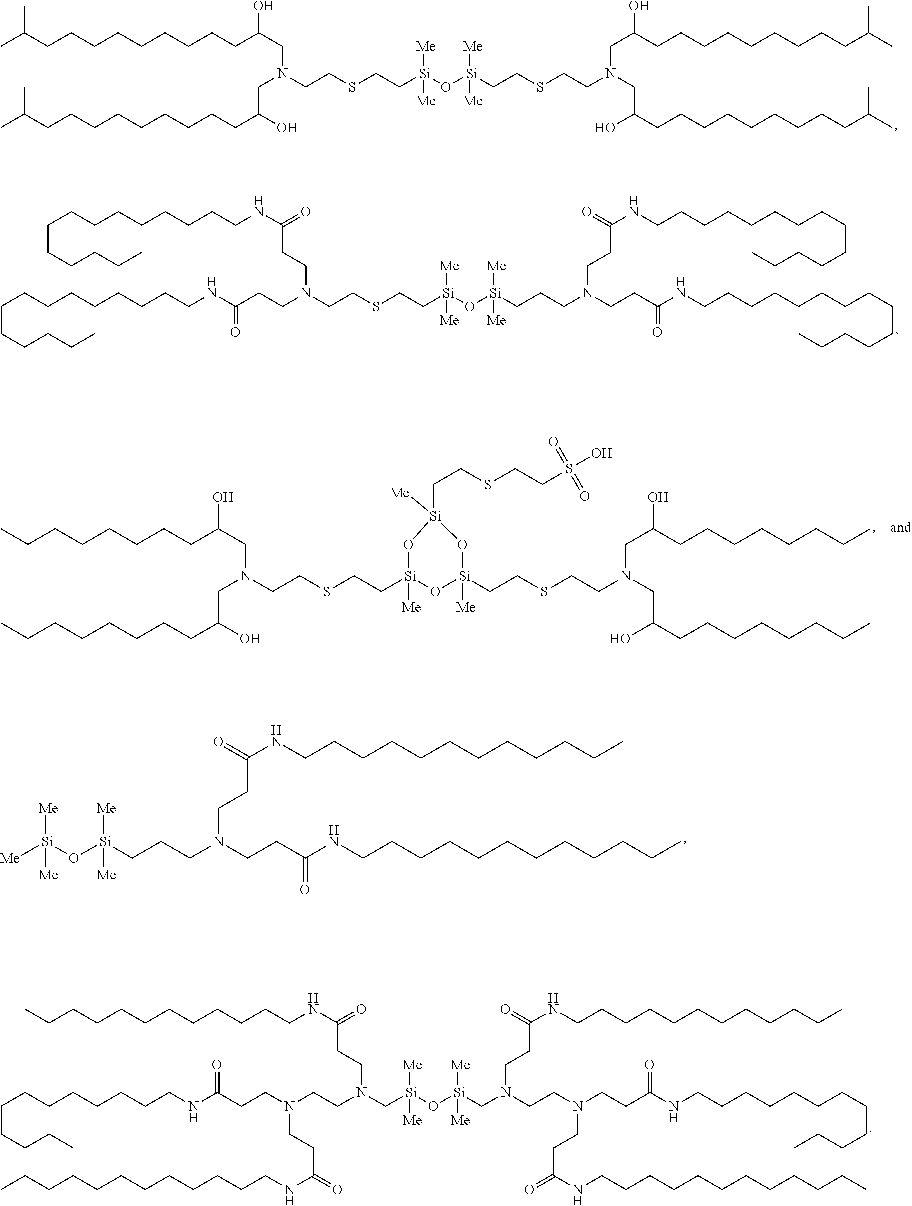

Embodiment 33 provides the LNP of any one of Embodiments 14-32, wherein the compound of Formula (I) comprises about 1 mol % to 99 mol % of the LNP.

Embodiment 34 provides the LNP of any one of Embodiments 14-33, wherein the compound of Formula (I) comprises about 35 mol % of the LNP.

Embodiment 35 provides the LNP of any one of Embodiments 14-34, wherein the neutral phospholipid is at least one selected from the group consisting of dioleoylphosphatidylethanolamine (DOPE), distearoylphosphatidylcholine (DSPC), dioleoylphosphatidylcholine (DOPC), distearoyl-phosphatidylethanolamine (DSPE), stearoyloleoylphosphatidylcholine (SOPC), 1-stearioyl-2-oleoyl-phosphatidyethanol amine (SOPE), and N-(2,3-dioleoyloxy)propyl)-N,N,N-trimethylammonium chloride (DOTAP).

Embodiment 36 provides the LNP of any one of Embodiments 14-35, wherein the neutral phospholipid is dioleoylphosphatidylethanolamine (DOPE).

Embodiment 37 provides the LNP of any one of Embodiments 14-36, wherein the at least one neutral phospholipid comprises about 10 mol % to about 45 mol % of the LNP.

Embodiment 38 provides the composition of any one of Embodiments 14-37, wherein the at least one neutral phospholipid comprises about 16 mol % of the LNP.

Embodiment 39 provides the LNP of any one of Embodiments 14-38, wherein the cholesterol lipid is cholesterol.

Embodiment 40 provides the LNP of any one of Embodiments 14-39, wherein the cholesterol lipid comprises about 5 mol % to about 50 mol % of the LNP.

Embodiment 41 provides the LNP of any one of Embodiments 14-40, wherein the cholesterol lipid comprises about 46.5 mol % of the LNP.

Embodiment 42 provides the LNP of any one of Embodiments 14-41, wherein the polyethylene glycol (PEG) or PEG-conjugated lipid comprises C14PEG2000.

Embodiment 43 provides the LNP of any one of Embodiments 14-42, wherein the polyethylene glycol (PEG) or PEG-conjugated lipid comprises about 0.5 mol % to about 12.5 mol % of the LNP.

Embodiment 44 provides the LNP of any one of Embodiments 14-43, wherein the polyethylene glycol (PEG) or PEG-conjugated lipid comprises about 2.5 mol % of the LNP.

Embodiment 45 provides the LNP of any one of Embodiments 14-44, wherein the LNP has a ratio of (a):(b):(c):(d) of about 35:16:46.5:2.5.

Embodiment 46 provides the LNP of any one of Embodiments 14-45, wherein the LNP selectively targets at least one cell type of interest.

Embodiment 47 provides the LNP of Embodiment 46, wherein the cell of interest is at least one selected from the group consisting of a tissue cell, muscle cell, or immune cell.

Embodiment 48 provides the LNP of Embodiment 46 or 47, wherein the cell of interest is at least one selected from the group consisting of an immune cell, endothelial cell, epithelial cell, hematopoietic stem cell (HSC), heart cell, brain cell, bone marrow cell, hepatocyte, liver cell, spleen cell, lung cell, podocyte, and kidney cell.

Embodiment 49 provides a pharmaceutical composition comprising the LNP of any one of Embodiments 14-48 and at least one pharmaceutically acceptable carrier.

Embodiment 50 provides a method of delivering a cargo to a subject in need thereof, the method comprising administering to the subject a therapeutically effective amount of at least one LNP of any one of Embodiments 14-48 and/or the pharmaceutical composition of Embodiment 49.

Embodiment 51 provides the method of Embodiment 50, wherein the cargo is at least one selected from the group consisting of a nucleic acid molecule, small molecule, protein, therapeutic agent, antibody, and any combinations thereof.

Embodiment 52 provides the method of Embodiment 50 or 51, wherein the cargo is a nucleic acid molecule.

Embodiment 53 provides the method of Embodiment 50 or 52, wherein the nucleic acid molecule is a DNA molecule or a RNA molecule.

Embodiment 54 provides the method of any one of Embodiments 51-53, wherein the nucleic acid molecule is selected from the group consisting of mRNA, cDNA, pDNA, microRNA, siRNA, modified RNA, antagomir, antisense molecule, targeted nucleic acid, and any combination thereof.

Embodiment 55 provides the method of any one of Embodiments 50-54, wherein the LNP selectively targets at least one cell type of interest.

Embodiment 56 provides the method of Embodiment 55, wherein the cell of interest is at least one selected from the group consisting of a tissue cell, muscle cell, or immune cell.

Embodiment 57 provides the method of Embodiment 55 or 56, wherein the cell of interest is at least one selected from the group consisting of an immune cell, endothelial cell, epithelial cell, hematopoietic stem cell (HSC), heart cell, brain cell, bone marrow cell, hepatocyte, liver cell, spleen cell, lung cell, podocyte, and kidney cell.

Embodiment 58 provides a method of treating, preventing, and/or ameliorating a disease or disorder in a subject in need thereof, the method comprising administering to the subject a therapeutically effective amount of the LNP of any one of Embodiments 14-48 and/or the pharmaceutical composition of Embodiment 49.

Embodiment 59 provides the method of Embodiment 58, wherein the disease or disorder is selected from the group consisting of a liver disease or disorder, pulmonary disease or disorder, renal disease or disorder, heart disease or disorder, spleen disease or disorder, and any combinations thereof.

Embodiment 60 provides a method of inducing an immune response in a subject in need thereof, the method comprising administering to the subject a therapeutically effective amount of at least one LNP of any one of Embodiments 14-48 and/or the pharmaceutical composition of Embodiment 49.

Embodiment 61 provides a method of treating, preventing, and/or ameliorating cancer in a subject in need thereof, the method comprising administering to the subject a therapeutically effective amount at least one LNP of any one of Embodiments 14-48 and/or the pharmaceutical composition of Embodiment 49.

Embodiment 62 provides the method of Embodiment 61, wherein the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer.

Embodiment 63 provides the method of Embodiment 61 or 62, wherein the subject is further administered at least one additional agent or therapy useful for treating, preventing, and/or ameliorating cancer in the subject.

Embodiment 64 provides the method of any one of Embodiments 50-63, wherein the subject is a mammal.

Embodiment 65 provides the method of Embodiment 64, wherein the mammal is a human.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A compound of Formula (I), or a salt, solvate, stereoisomer, or isotopologue thereof: 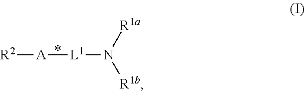
wherein:
A is 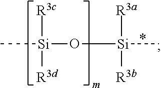
R² is selected from the group consisting of 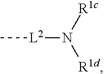
optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{1a}, R^{1b}, R^{1c}, and R^{1d}, if present, are each independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
L¹ and L² are each independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{3a}, R^{3b}, R^{3c}, and R^{3d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl,
wherein two occurrences of R³⁰ or two occurrences of R^{3d} can combine with the atoms to which they are bound to form an optionally substituted 6-10 membered cyclosiloxane, or
wherein R^{3a} and R^{3b}, R^{3a} and R^{3d}, R^{3b} and R^{3c}, or R^{3b} and R^{3d} can combine with the atoms to which they are bound to form an optionally substituted 6-10 membered cyclosiloxane;
each occurrence of R^{A} is independently selected from the group consisting of optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl; and
m is an integer ranging from 0 to 50.

2. The compound of claim 1, wherein A is selected from the group consisting of: 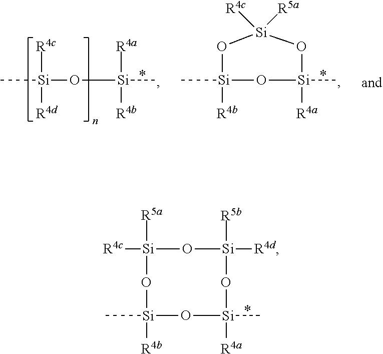
wherein:
R^{4a}, R^{4b}, R^{4c}, and R^{4d}, if present, are each independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
R^{5a} and R^{5b}, if present, are each independently selected from the group consisting of 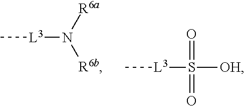
optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of L³ is independently selected from the group consisting of optionally substituted C₁-C₁₂ alkylenyl, optionally substituted C₂-C₁₂ alkenylenyl, optionally substituted C₂-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{6a} and R^{6b} is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl; and
n is an integer ranging from 0 to 30.

3. (canceled)

4. The compound of claim 1, wherein A is selected from the group consisting of

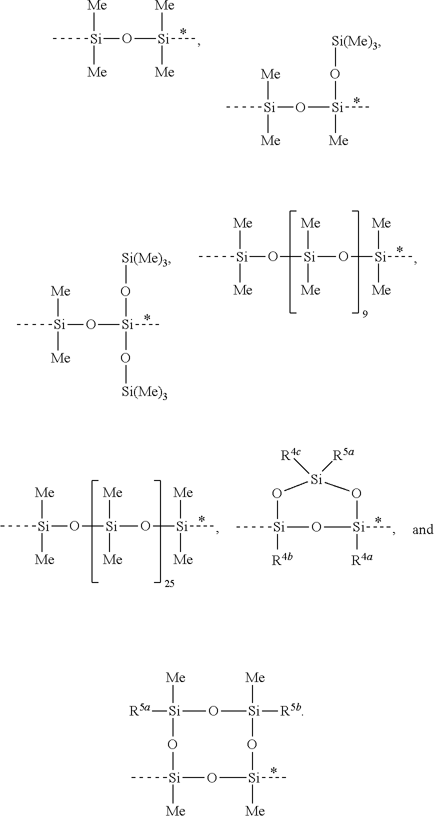

5. The compound of claim 1, wherein each occurrence of L¹, L², and L³, if present, is independently selected from the group consisting of —(CH₂)₁₋₅S(CH₂)₁₋₅—, —(CH₂)₁₋₅—, and —(CH₂)₁₋₅N(R^{6a})(CH₂)₁₋₅—,
optionally wherein each occurrence of L¹, L², and L³, if present, is independently selected from the group consisting of —(CH₂)₃—, —(CH₂)₂S(CH₂)₂—, and —(CH₂)NR^{6a}(CH₂)₂—.

6. (canceled)

7. The compound of claim 1, which is selected from the group consisting of:

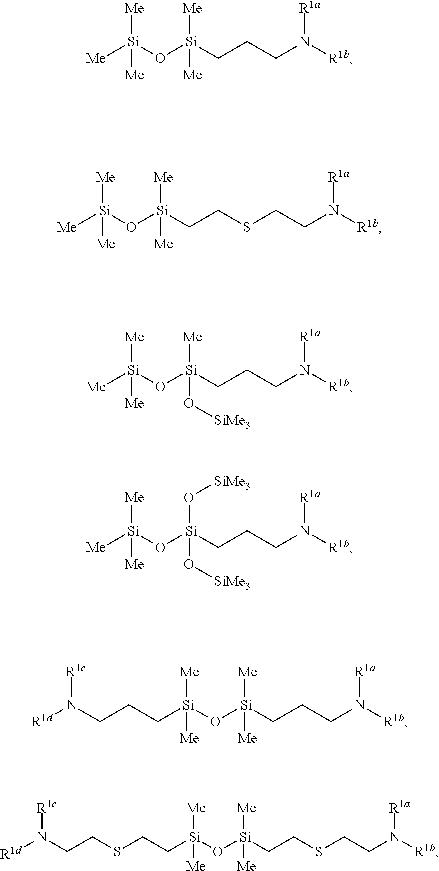

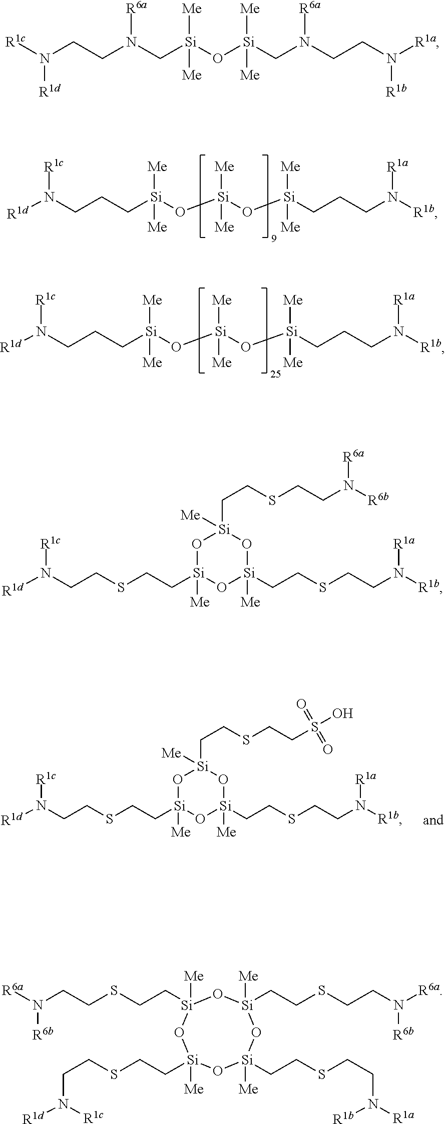

8. The compound of claim 1, wherein R^{1a}, R^{1b}, R^{1c}, and R^{1d} are each independently selected from the group consisting of —CH₂CHOH-(optionally substituted C₁-C₂₀ alkyl), —CH₂CHOH-(optionally substituted C₁-C₂₀ heteroalkyl), —CH₂CHOH-(optionally substituted C₂-C₂₀ alkenyl), —CH₂CH₂C(═O)O(optionally substituted C₁-C₂₀ alkyl), and —CH₂CH₂C(═O)NH(optionally substituted C₁-C₂₀ alkyl),
optionally wherein R^{1a}, R^{1b}, R^{1c}, and R^{1d} are each independently selected from the group consisting of

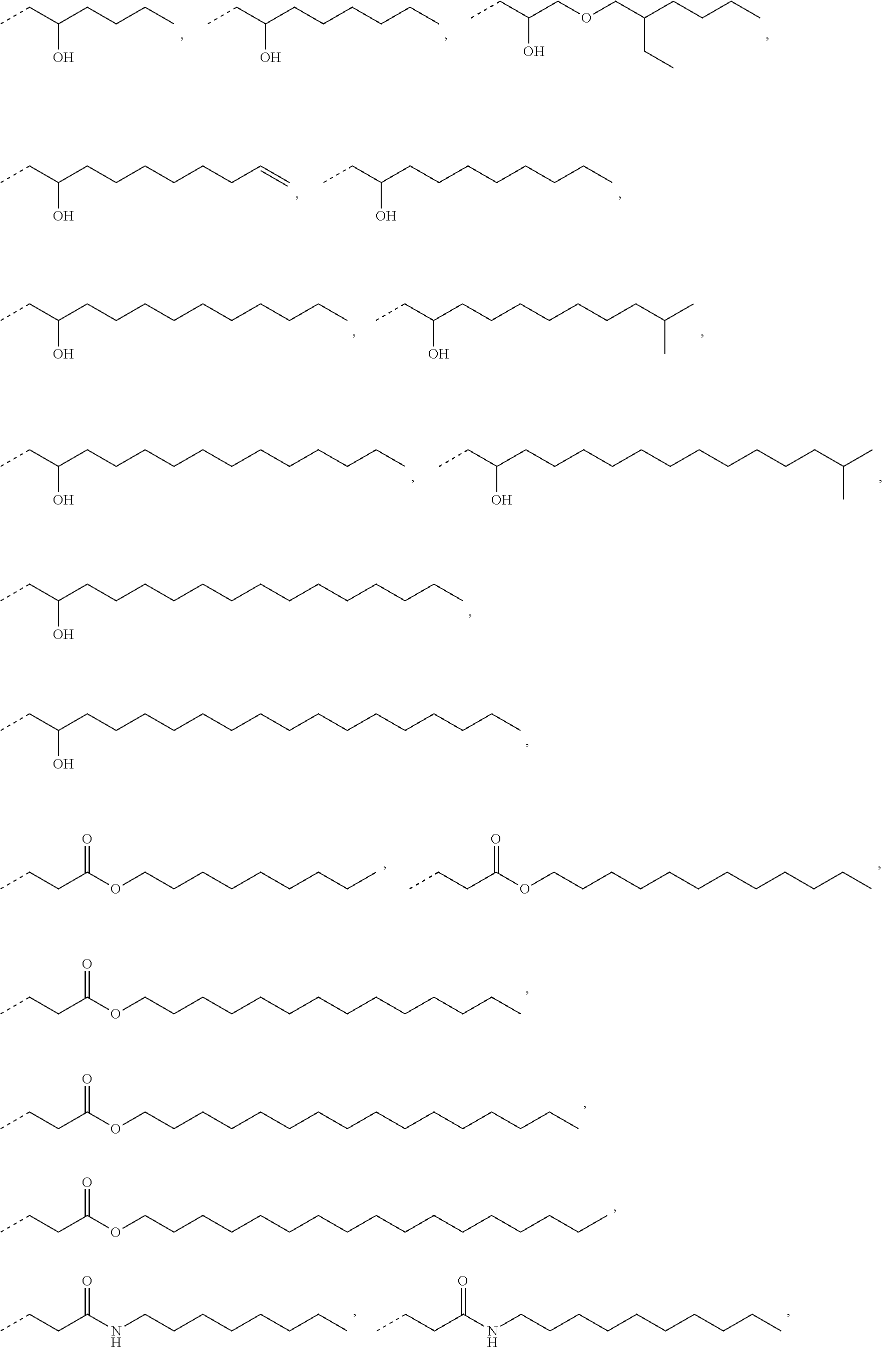

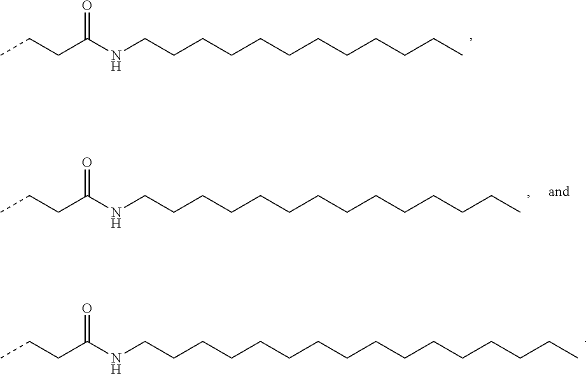

9. The compound of claim 2, wherein each occurrence of R^{6a} and R^{6b}, if present, is independently selected from the group consisting of —CH₂CHOH-(optionally substituted C₁-C₂₀ alkyl), —CH₂CHOH-(optionally substituted C₁-C₂₀ heteroalkyl), —CH₂CHOH-(optionally substituted C₂-C₂₀ alkenyl), —CH₂CH₂C(═O)O(optionally substituted C₁-C₂₀ alkyl), and —CH₂CH₂C(═O)NH(optionally substituted C₁-C₂₀ alkyl),
optionally wherein each occurrence of R^{6a} and R^{6b}, if present, is independently selected from the group consisting of

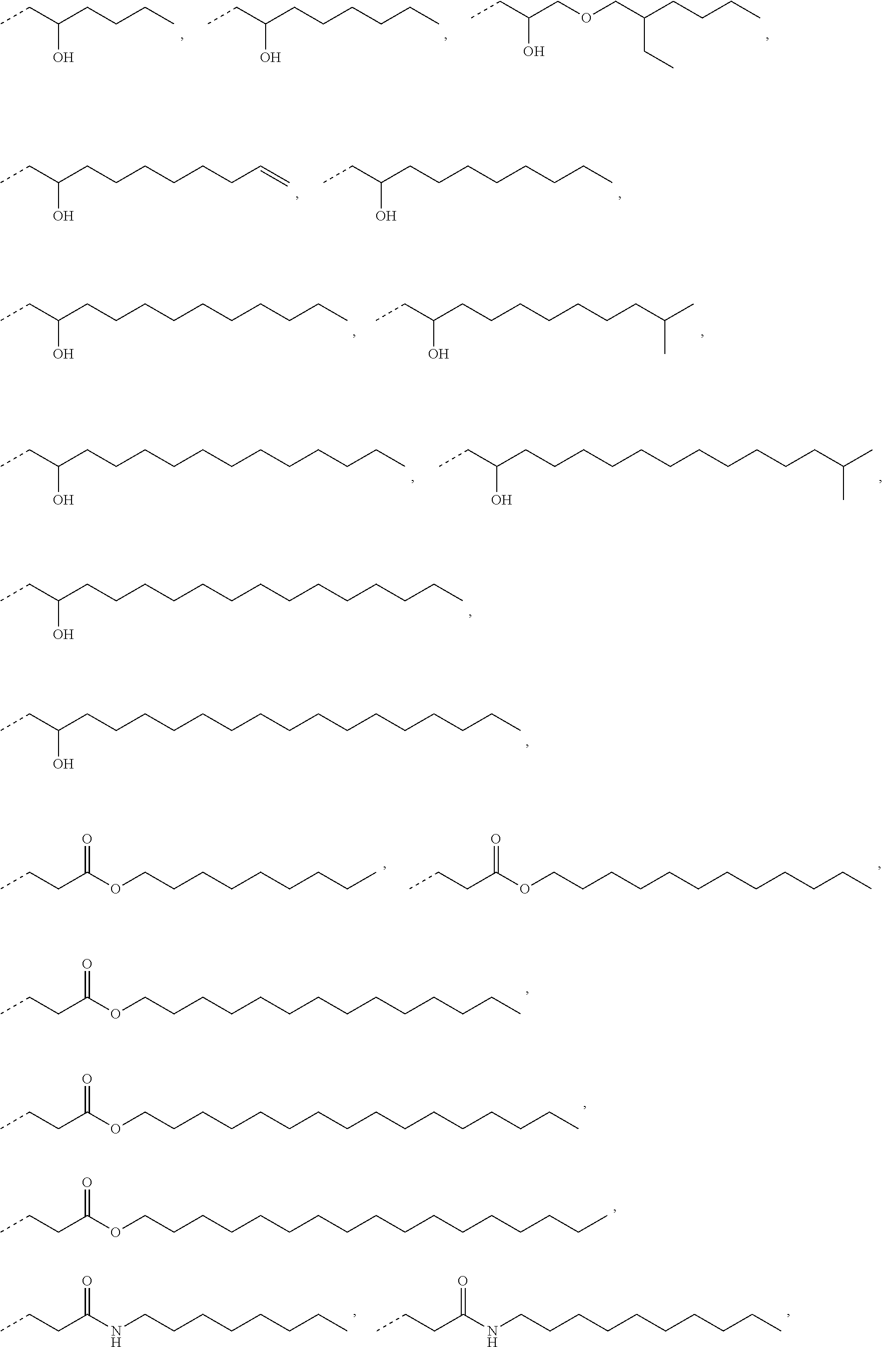

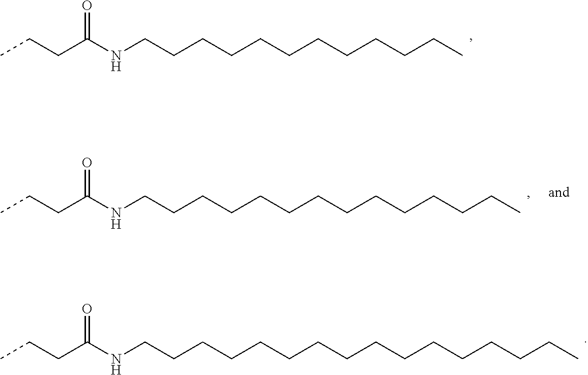

10. The compound of claim 1, wherein each occurrence of optionally substituted alkyl, optionally substituted alkylenyl, optionally substituted heteroalkyl, optionally substituted heteroalkylenyl, optionally substituted cycloalkyl, optionally substituted cycloalkylenyl, optionally substituted heterocycloalkyl, optionally substituted heterocycloalkylenyl, optionally substituted alkenyl, optionally substituted alkenylenyl, optionally substituted alkynyl, optionally substituted aralkyl, optionally substituted aryl, optionally substituted heteroaryl, and optionally substituted cyclosiloxane, if present, is independently optionally substituted with at least one substituent selected from the group consisting of C₁-C₆ alkyl, C₃-C₈ cycloalkyl, C₁-C₆ haloalkyl, C₁-C₃ haloalkoxy, phenoxy, halogen, CN, NO₂, OH, N(R′)(R″), C(═O)R′, C(═O)OR′, OC(═O)OR′, C(═O)N(R′)(R″), S(═O)₂OR′, S(═O)₂N(R′)(R″), N(R′)C(═O)R″, N(R′)S(═O)₂R″, C₂-C₈ heteroaryl, and phenyl optionally substituted with at least one halogen, wherein each occurrence of R′ and R″ is independently selected from the group consisting of H, C₁-C₆ alkyl, C₃-C₈ cycloalkyl, C₁-C₆ haloalkyl, benzyl, and phenyl.

11-12. (canceled)

13. The compound of claim 1, which is selected from the group consisting of:

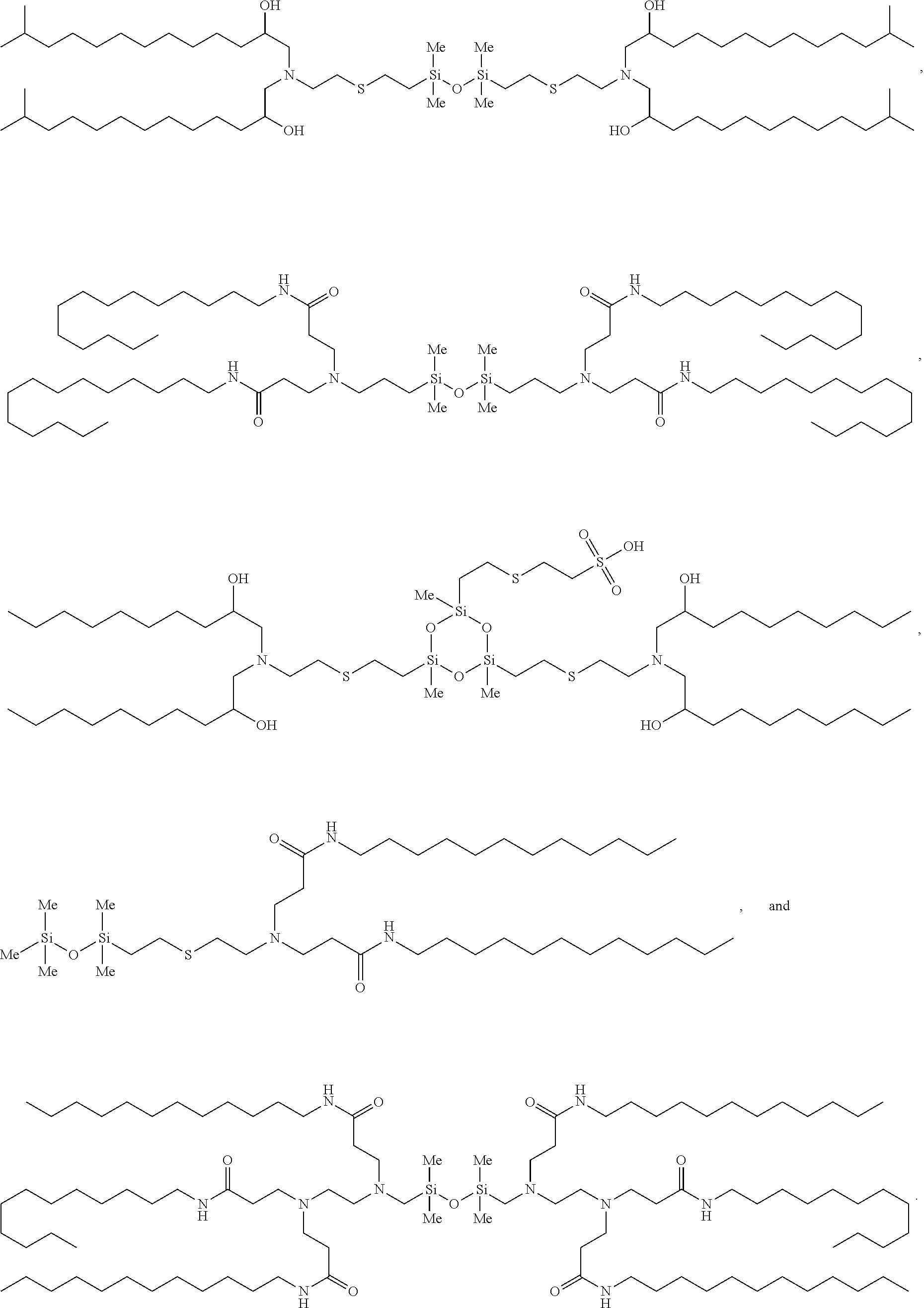

14. A lipid nanoparticle (LNP) comprising:
(a) at least one compound of Formula (I), or a salt, solvate, stereoisomer, or isotopologue thereof: 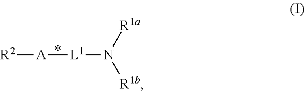
wherein:
A is 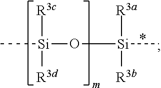
R² is selected from the group consisting of 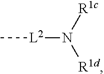
optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl;
each occurrence of R^{1a}, R^{1b}, R^{1c}, and R^{1d}, if present, is independently selected from the group consisting of H, optionally substituted C₁-C₂₈ alkyl, optionally substituted C₂-C₂₈ heteroalkyl, optionally substituted C₃-C₈ cycloalkyl, optionally substituted C₂-C₈ heterocycloalkyl, optionally substituted C₂-C₂₈ alkenyl, and optionally substituted C₂-C₂₈ alkynyl;
L¹ and L² are each independently selected from the group consisting of optionally substituted C₁-C₁₂ alkylenyl, optionally substituted C₂-C₁₂ alkenylenyl, optionally substituted C₁-C₁₂ alkynylenyl, optionally substituted C₁-C₁₂ heteroalkylenyl, optionally substituted C₃-C₈ cycloalkylenyl, and optionally substituted C₂-C₈ heterocyloalkylenyl;
each occurrence of R^{3a}, R^{3b}, R^{3c}, and R^{3d}, if present, is independently selected from the group consisting of optionally substituted C₁-C₆ alkyl, OSiR^{A}₃, optionally substituted C₃-C₈ cycloalkyl, optionally substituted C₇-C₁₀ aralkyl, optionally substituted C₆-C₁₀ aryl, and optionally substituted C₂-C₁₀ heteroaryl,
wherein two occurrences of R³⁰ or two occurrences of R^{3d} can combine with the atoms to which they are bound to form an optionally substituted 6-10 membered cyclosiloxane, or
wherein R^{3a} and R^{3c}, R^{3a} and R^{3d}, R^{3b} and R^{3c}, or R^{3b} and R^{3d} can combine with the atoms to which they are bound to form an optionally substituted 6-10 membered cyclosiloxane;
each occurrence of R^{A} is independently selected from the group consisting of optionally substituted C₁-C₆ alkyl, optionally substituted C₃-C₈ cycloalkyl, optionally substituted C₇-C₁₀ aralkyl, optionally substituted C₆-C₁₀ aryl, and optionally substituted C₂-C₁₀ heteroaryl; and
m is an integer ranging from 0 to 50;
(b) at least one neutral phospholipid;
(c) at least one cholesterol lipid; and
(d) at least one selected from the group consisting of polyethylene glycol (PEG) and a PEG-conjugated lipid.

15. The LNP of claim 14, wherein at least one of the following applies:
(a) the LNP further comprises at least one cargo; and
(b) the LNP selectively targets at least one cell type of interest.

16. The LNP of claim 15, wherein at least one of the following applies:
(a) the cargo is at least partially encapsulated by the LNP;
(b) the cell of interest is at least one selected from the group consisting of a tissue cell, a muscle cell, or immune cell, optionally wherein the cell of interest is at least one selected from the group consisting of an immune cell, endothelial cell, epithelial cell, hematopoietic stem cell (HSC), heart cell, brain cell, bone marrow cell, hepatocyte, liver cell, spleen cell, lung cell, podocyte, and kidney cell:
(c) the cargo is at least one selected from the group consisting of a nucleic acid molecule, small molecule, protein, therapeutic agent, antibody, and any combinations thereof, optionally wherein the nucleic acid molecule is selected from the group consisting of mRNA, cDNA, pDNA, microRNA, siRNA, modified RNA, antagomir, antisense molecule, targeted nucleic acid, and any combination thereof.

17-31. (canceled)

32. The LNP of claim 14, wherein at least one of the following applies:
(a) the compound of formula (I) is selected from the group consisting of: 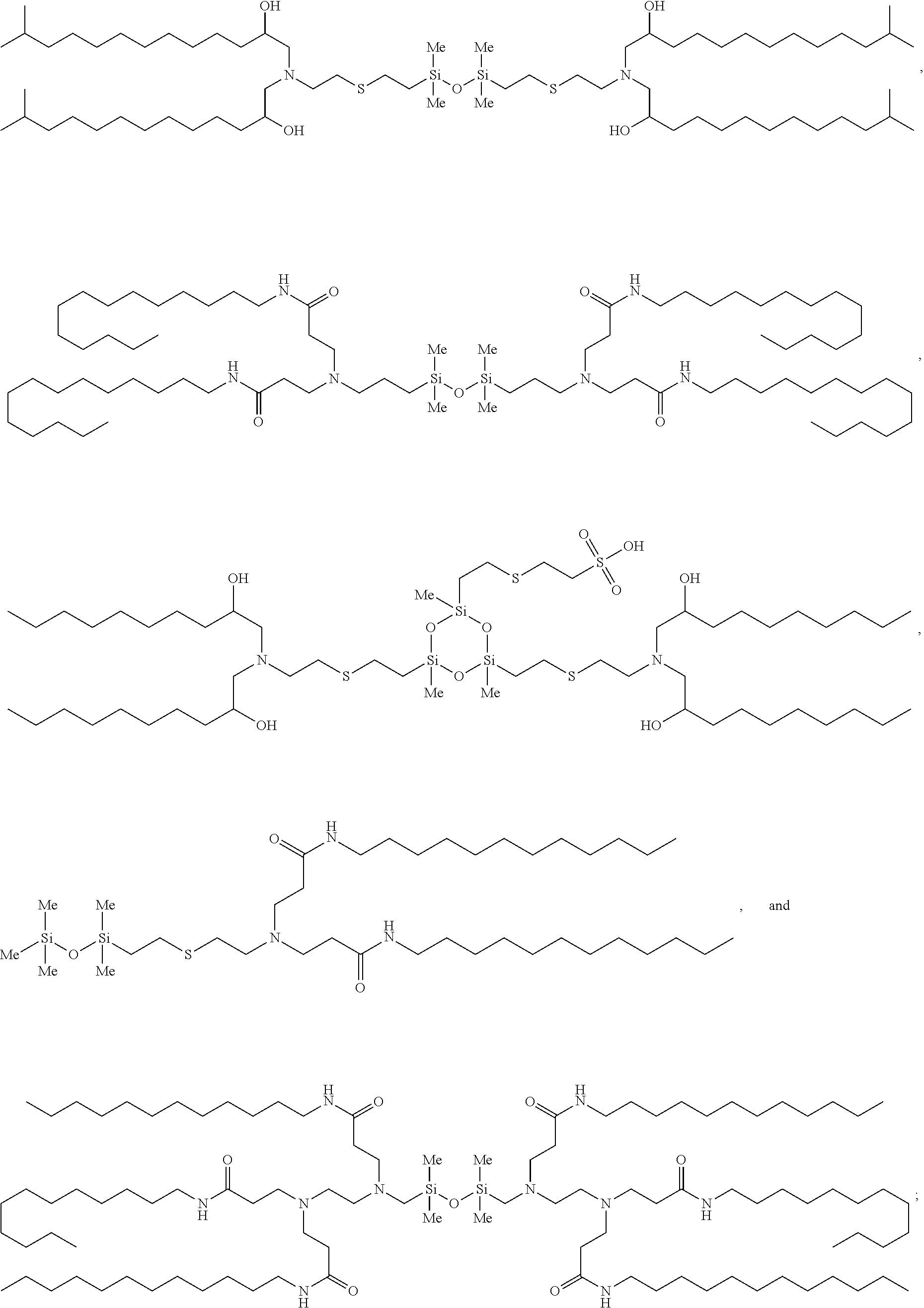
(b) the neutral phospholipid is at least one selected from the group consisting of dioleoylphosphatidylethanolamine (DOPE), distearoylphosphatidylcholine (DSPC), dioleoylphosphatidylcholine (DOPC), distearoyl-phosphatidylethanolamine (DSPE), stearoyloleoylphosphatidylcholine (SOPC), 1-stearioyl-2-oleoyl-phosphatidyethanolamine amine (SOPE), and N-(2,3-dioleoyloxy)propyl)-N,N,N-trimethylammonium chloride (DOTAP), optionally wherein the neutral phospholipid is dioleoylphosphatidylethanolamine (DOPE);
(c) the cholesterol lipid is cholesterol; and
(d) the polyethylene glycol (PEG) or PEG-conjugated lipid comprises C14PEG2000.

33. The LNP of claim 14, wherein at least one of the following applies:
(a) the compound of Formula (I) comprises about 1 mol % to 99 mol % of the LNP, optionally wherein the compound of Formula (I) comprises about 35 mol % of the LNP;
(b) the at least one neutral phospholipid comprises about 10 mol % to about 45 mol % of the LNP, optionally wherein the at least one neutral phospholipid comprises about 16 mol % of the LNP;
(c) the cholesterol lipid comprises about 5 mol % to about 50 mol % of the LNP, optionally wherein the cholesterol lipid comprises about 46.5 mol % of the LNP;
(d) the polyethylene glycol (PEG) or PEG-conjugated lipid comprises about 0.5 mol % to about 12.5 mol % of the LNP, optionally wherein the polyethylene glycol (PEG) or PEG-conjugated lipid comprises about 2.5 mol % of the LNP; and
(e) the LNP has a ratio of (a):(b):(c):(d) of about 35:16:46.5:2.5.

34-48. (canceled)

49. A pharmaceutical composition comprising the LNP of claim 14 and at least one pharmaceutically acceptable carrier.

50. A method of delivering a cargo to a subject in need thereof, the method comprising administering to the subject a therapeutically effective amount of at least one LNP of claim 14 and/or a pharmaceutical composition thereof.

51. The method of claim 50, wherein at least one of the following applies:
(a) the cargo is at least one selected from the group consisting of a nucleic acid molecule, small molecule, protein, therapeutic agent, antibody, and any combinations thereof, optionally wherein the cargo is a nucleic acid molecule, optionally wherein the nucleic acid molecule is a DNA molecule or RNA molecule, and optionally wherein the nucleic acid molecule is selected from the group consisting of mRNA, cDNA, pDNA, microRNA, siRNA, modified RNA, antagomir, antisense molecule, targeted nucleic acid, and any combination thereof; and
(b) the LNP selectively targets at least one cell type of interest, optionally wherein the cell of interest is at least one selected from the group consisting of a tissue cell, muscle cell, or immune cell, and optionally wherein the cell of interest is at least one selected from the group consisting of an immune cell, endothelial cell, epithelial cell, hematopoietic stem cell (HSC), heart cell, brain cell, bone marrow cell, hepatocyte, liver cell, spleen cell, lung cell, podocyte, and kidney cell.

52-57. (canceled)

58. A method of treating, preventing, and/or ameliorating a disease or disorder in a subject in need thereof, the method comprising administering to the subject a therapeutically effective amount of the LNP of claim 14 and/or a pharmaceutical composition thereof, optionally wherein the disease or disorder is selected from the group consisting of a liver disease or disorder, pulmonary disease or disorder, renal disease or disorder, heart disease or disorder, spleen disease or disorder, and any combinations thereof.

59. (canceled)

60. A method of inducing an immune response in a subject in need thereof, the method comprising administering to the subject a therapeutically effective amount of at least one LNP of claim 14 and/or a pharmaceutical composition thereof.

61. A method of treating, preventing, and/or ameliorating cancer in a subject in need thereof, the method comprising administering to the subject a therapeutically effective amount at least one LNP of claim 14 and/or a pharmaceutical composition thereof, optionally wherein at least one of the following applies:
(a) the cancer is at least one selected from the group consisting of pancreatic cancer, colorectal cancer, bladder cancer, breast cancer, prostate cancer, renal cancer, hepatocellular cancer, lung cancer, ovarian cancer, cervical cancer, gastric cancer, esophageal cancer, head and neck cancer, melanoma, neuroendocrine cancer, CNS cancer, brain cancer, bone cancer, soft tissue sarcoma, non-small cell lung cancer, small-cell lung cancer, or colon cancer;
(b) the subject is further administered at least one additional agent or therapy useful for treating, preventing, and/or ameliorating cancer in the subject; and
(c) the subject is a mammal, optionally wherein the mammal is a human.

62-65. (canceled)


SEQUENCE LISTING (4 sequences)

SEQ ID NO: 1 - DNA, 21 bp, synthetic construct
  source 1..21: mol_type = other DNA
cggtttactctgacccatttc

SEQ ID NO: 2 - DNA, 20 bp, synthetic construct
  source 1..20: mol_type = other DNA
gggctttctacaagcttacc

SEQ ID NO: 3 - RNA, 104 nt, synthetic construct
  source 1..104: mol_type = other RNA
  modified_base 1..3: mod_base = OTHER; note = 2'-Methoxy
  modified_base 21: mod_base = OTHER; note = 2'-Methoxy
  modified_base 28..42: mod_base = OTHER; note = 2'-Methoxy
  modified_base 46: mod_base = OTHER; note = 2'-Methoxy
  modified_base 48: mod_base = OTHER; note = 2'-Methoxy
  modified_base 50: mod_base = OTHER; note = 2'-Methoxy
  modified_base 52..57: mod_base = OTHER; note = 2'-Methoxy
  modified_base 60..61: mod_base = OTHER; note = 2'-Methoxy
  modified_base 66..67: mod_base = OTHER; note = 2'-Methoxy
  modified_base 70..84: mod_base = OTHER; note = 2'-Methoxy
  modified_base 85..104: mod_base = OTHER; note = 2'-Methoxy
  modified_base 8..11: mod_base = OTHER; note = 2'-fluoro
  modified_base 13..14: mod_base = OTHER; note = 2'-fluoro
  modified_base 17..18: mod_base = OTHER; note = 2'-fluoro
gggcgaggagctgttcaccggttttagagctagaaatagcaagttaaaataaggctagtccgttatcaactstsgsaaaaagtggcaccgagtcggtgcttttt

SEQ ID NO: 4 - RNA, 20 nt, synthetic construct
  source 1..20: mol_type = other RNA
ttacagccacgtctacagca